SEQUENCE LISTING (4 sequences)

SEQ ID NO: 1 - protein, 525 aa, Homo sapiens
  VARIANT (1)..(23): missing in isoform alpha II
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL

SEQ ID NO: 2 - protein, 393 aa, homo sapiens [sequence omitted]

SEQ ID NO: 3 - DNA, 45675 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggtacggagcggaccacccctcctgggcccctgcccgggtcccgaccctctttgccggcgccgggcggggccggcggcgagtgaatgaattaggggtccccggaggggcgggtggggggcgcgggcgcggggtcggggcgggctgggtgagaggggtctgcaggggggaggcgcgcggacgcggcggcgcggggagtgaggaatgggcggtgcggggctgaggagggtgaggctggaggcggtcgccgctggtgctgcttcctggacggggaaccccttccttcctcctccccgagagccgcggctggaggcttctggggagaaactcgggccgggccggctgcccctcggagcggtggggtgcggtggaggttactcccgcggcgccccggcctcccctccccctctccccgctcccgcacctcttgcctccctttccagcactcggctgcctcggtccagccttccctgctgcatttggcatctctaggacgaaggtataaacttctccctcgagcgcaggctggacggatagtggtccttttccgtgtgtaggggatgtgtgagtaagaggggaggtcacgttttggaagagcataggaaagtgcttagagaccactgtttgaggttattgtgtttggaaaaaaatgcatctgcctccgagttcctgaatgctcccctcccccatgtatgggctgtgacattgctgtggccacaaaggaggaggtggaggtagagatggtggaagaacaggtggccaacaccctacacgtagagcctgtgacctacagtgaaaaggaaaaagttaatcccagatggtctgttttgcttggtcaagttaaacccgaagaaaacccgcagagcagaagcaaggctttttccttgctagttgagtgtagacagcaatagcaaaaatagtacttgaagtttaatttacctgttcttgtcctttcccctatttcttatgtattaccctcatcccctcgtctcttttatactaccctcattttgcagatgtgttctacatctcaagagttattacagtactccaaaacagcacttacatgattttttaaacttacagaggaattgtagcaatccaccagctaaccgcctgaaatagacttaaacatgtgcatctcctttttttttttttttttgagacacagtctcgctctgttgcccaggctggagtgcaatggcgcggtatcggctcactgaaacctccgcctcctgggttcaagcaattctcctgcctcagcctcccgagtagctgggactagtaggtgcacgccaccatgcccagctaatttttgtatttttagtagagacagagtttcatcatgttggtcaggatggtctccatctgctctgttgcccaggctggagtgcagtggcgccgtctcggctcactgcaacctctgcctcctgcattcaagcaattctcctgcctcagcctcccgaataactgggattacaggtgtctgctgccatgcccggctaattttttgtatttttagtagagacgggggtttcaccatgttggtcaggctggtctagaactcctgacctcgtgatctgcccgcctcggcctcccacagtggcatgtgcatcttatagctgaagtctaagccttcttaaatcttgagatccatcaaaacagacaggttttctaattgttatacaatgtatatgttatgtttataatagaaatcattttacaaataagttataaatgggaaaggtctatttgtaattatcagctcagaattaaccataaaactggtgtcactgaagtgactgaggtccaaaatgctgactctgcatgttatagactacagatatcaaatatggttgctaacaatagtttactttgagactgtagccatccacagtatatttgcttttaagagatggtagatggtaattcagttttatgaaaaataaaaatgaattttcttccattacaaaattgttggattcgagtccagtccactccttactagcttttctaactctcggtgagggatcccctcccagcccatgatcttcatttggtaagactcctttggaacccagttctctctagtggatttaaatgtgatttggttttaaaaatctcattcaaggaattttttttttttctggaaacaaccaccgcataaacaagtaaaccggaagatacatgtggctctgaattcatatatatacacaaactctaatccaatgtctgtccacagtatttcctaggctagtaaactttttggccttaacgacccctctaccctctttgtttttttgagagagagagtctcactctgtcacccaggccggaatgcagtggcgcgatctcggcccgctactacctccgactctcaggctcaagcgattctcccgcctcagcttcccgagtagccgggattacaggctcccgccaccgggctaattgtatttttagatacgggatttcaccatgttggccaggctggtctcgacctcctgacctcaggtgatccgcccgcctaagcctcccaaagtgctgggattacaggccaccacacccggcctacactcttaaaaattatcgaaggggccgggcacattggctcttatctgtaatcccagcactttgggagactgaggcgggaggatcgcttgaggccaggagttggagaccagcgtactcaacatagtgagaccttgttataaagaaaaaaaaaatccaggattaaaaaaaatctttgatttgtttgggatttattaatatttaccgtattggaaattaaaacaattttttaaaatgtattcatttaaaaataataagcccattacttggtaacatgaataaaatattttatgaaaaataactattttccaaaacaaaaccaaaacttagaaaagtggtattgtttcacacttcagtaaatctctttaatgatgtggcttaatagaagatatggattcttatatctgcatctgcattcaatctattatgatcacacatctggaaaacttgtgaaagaatgggagttaaaagggtaaaggacatcttaatgttattatgaaaacagttttgacctcttgcacaccagaaaagtcttagtaacctgaggggttcctagaccacattttgagaactgttttaggctatgcaaactggttggggggaggttggggtaggcagagagctagaagatacattttagtgtaattctcctcatctattcctaattgctttggcctacatttgaaataaagcgtggaggcaaacgggataagatacatgtttgtagtggttgttaacttcaccctagacaagcagccaataagtctaggtagagcagagtaaggcggggaactatgccgtgaccgtgtgtgatacaatttttctagcctgtggtgctttttgcggcagggcttaggagtaaggttagtatgttatcatttgggaaaccaaattattattttgggtcttcagtcaattatgatgctgtgtatatttagtgtttatctacaatatatgcacattcattaatttggagctactcatcctataataaatagttgtgcatttactcccatttttttctgcatttctctccttatttataattatgtgttacatgagggaaaggaggtgaaattaaacattcatattatttcaaaaaatttgaaacaactaactaaaaaatatgttttattttctgtatggtgtttgttatacaatctgtcaatattcatgcacctcttgggagacagtgtatgaaaagcaaagagtaacagtcacatggattactgattactgagatatattcacttgcatctttttttttttttgagacggagtggctctgtcgcccaggctggagtgcagtggcgtgatctcggctcactgcaagctccgcctcctgggttcacgccattcttctgcctcagcctcccaagtagctgggactacaggcgcccgccaccacgcccggctaatttttttatatttttagtagagacggggtttcaccgggttagccaggatggtcttgatctcctgacctcgtgatccaccctcctcggcctcccaaagtgctaggattataggcgtgagccaccgtgcccggctcacttgcatctcttaacagctgttttcttactaaaaacagtgtttatctctaatctttttgtttgtttgtttgttttgagatggagtcttactccgtcacccaatctggagtgcagtggcgtgatctgggctcactgcaacctctgcctcccgggttcaagtgattctccttcctcagcctccccagtagctaggactacaggagagcgccaccacgcctgattaatttttgtatttttagtagagagagggtttcaccatattggccaggctggtcttgaactcctggcctcaggtgatccacccgccttggcctctgaaagtgctgggattacaggcatgagccgccgcacccggctttctaatctttatctttttttgtgcagcggtgatacaggattatgtattgtactgaacagttaattcggagttctcttggtttttagctttattttccccagagatttttttttttttttttttttttgagacggagtcttgctctatcgccaggctggagtgcagtggcgccatctcggctcattgcaacctcggactcctattttccccagagatatttcacacattaaaatgtcgtcaaatattgttcttctttgcctcagtgtttaaatttttatttccccatgacacaatccagctttatttgacactcattctctcaactctcatctgattcttactgttaatatttatccaagagaactactgccatgatgctttaaaagtttttctgtagctgttgcatattgacttctaacacttagaggtgggggtccactaggaaaactgtaacaataagagtggagatagctgtcagcaacttttgtgagggtgtgctacagggtgtagagcactgtgaagtctctacatgagtgaagtcatgatatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagtacttattacaagataattatgctgaaagttaagttatctgaaatgtaccttgggtttcaagttatatgtaaccattaatatgggaactttactttccttgggagtatgtcagggtccatgatgttcactctctgtgcattttgattggaagtgtatttcagagtttcgtgagagggtagaaatttgtatcctatctggacctaaaagacaatctttttattgtaacttttatttttatgggtttcttggtattgtgacatcatatgtaaaggttagatttaattgtactagtgaaatataattgtttgatggttgatttttttaaacttcatcagcagtattttcctatcttcttctcaacattagagaacctacaactaccggataaattttacaaaatgaattatttgcctaaggtgtggtttatataaaggtactattaccaactttacctttgctttgttgtcatttttaaatttactcaaggaaatactaggatttaaaaaaaaattccttgagtaaatttaaattgttatcatgtttttgaggattattttcagatttttttagtttaatgaaaatttaccaaagtaaagaccagcagcagaatgataagtaaagacctgtaagacaccttgaaggtcatggagtagaacttccatcccaagcagatgaggatttatttaatctcaaagacctccaggaggggacattccccaactgtccttgttaactcattttcagaacatatttattagcatattttacatgtaatttggatcttcatgttaaatttaacatcagtggagatggaaaataagcatatcgccttgtctttgaaatagccctatattgttagattgtttcttaggcttctttaccctgggttaagcagtcctaatactttagcatttattctacatctagtgtactaatttaaaaaaatcagttctgaaaaatttctaagaactttcttcaagttccaagctgtgaaatctagaacaggtcaaagtgccttattaacgtactgtactgtgtagtgtcttgaagagacactttgcgctgaggcaagttctgagggcattgggtggccttgggaagatatttatgcagtttagaacctggagaattgattagataactaatcataaggaaacgtcacatatttttggtactataaaaaagtggagaaataatgcctatttgcaaagatttgatttaaacatagaaacaactttatttggcttccaattttaagaatttacagcagtaaaggggaacagtctaattgaagtagactgcctatgcaatagtctctgtatatttacttttgacaagttaattcaatgtgtactatagttttgtttctttgaagaggtttgaatagtgcacccattttaatctgtattgcaaattcagggttacttggcagactctactatttaaatcagatgtaaaaggaagttttaatataattcactttatgcctgaaagttttcctgggattttggaaggtgattttactggaaatgctgtctgtcttccctgaaaatctgagaaattccattacactttgtttccaatcagaggtcatgagtgctatatgagtatatacagcatgacgtcatgaatgtgataaagtgggttaggaaaccttttgctaatgattgttaaaatgcaatataaatgttgaagaaataaagctaacagttaagcctttatttgggcggaaggctgaaaaagtttataaacttaaacctataactctgcttatgatttctgccaaaccagaagacttgactctgggaagcattggttacctgtgaactttgaaactgacggtccctgacgtagtttagtcacctgggaaaaggtatctgagattatctcttatctcccaagttacagtgagtctctgagggaactgacacattacattaagttcttggtgtagttaaactgtaagaaaggcaggagaacttagtagttaaatagttggttaaatggaaatgctgactccatgttattgtaaaaagttaaaaatttaggaggatatggggatttcactgccattgcaggttttgattggtatttaccaatccgtgtgggtcagagagaaaattagaaaggatatgactgcacattttggaattattagcagtttttctacatttaaaatggaaataaattttttaaaaatttaaatcaagtaatactgtattttttggtgatttagatttttcaaaatttacactaagagatagtaaggagggtggctattgtttctttcaataatgtctctgagaggttgtaactcatctaaggatacgtagctaataagtggtaggatttcaatttaaattctctgagaccaagttaagtagaatttgcactgtactcttgtataactttttaaaactgaaaattagctatctttcaaattaagaaaatatttactaatggagactaattcagatttgtaagtataccaaaatttgaacttagcctgctatctaatggcaacttagtggcagaggtatgatgtaaaatcattcaggtatgacacatagatggagtatgtttgtattcgaggctgtgcacataatcacctttacttgtattgtgaagtatatattgttatcttttatgaagcccactaaagagataatgaaatacctcgttattagggcaagattattgaaaactcaaaatagcccccaaacacaatacttggctagaaatatatacctttatagttcagagatcatttattatcaaaaccctgaagttttttttctaaggtaaaatttggtggaagaggaaaagtctcgttttaaaaaaatgtaggtagttacagagatcagaatgattagttgatcacttaccaaatatatattaagtatctactgtatataatatgctagtaagaataaatatagcaggaagtattttttcccaggctctaattgtttgacatcagcatgcttttattgtggcacttataattcagttcaagtattatgcccctctttgatggaacagtttcctattcagtaaggaagaccagattaatcattggattggtttgtttcatctttagtgttctgagctgtagagtatttatttaccaaggtttattttaatttttattttatttttatttttccatgttcattgtagaattcattttacctacgaatgaagtatgtagattatagagagaaaatttgtaaaattaaactgatactgaagactggtataagaaaagccttatgtaatttgtaagctgctattcttctgagtttatacatatatctttagtaatcaatgagggatggttgggtgactgccctccaggggacatttggcaacatctggagatgtttttggttgccacaacttggggagagagtactgctactggcatctattgagtagatgctattactttaaatggcaaagctgcagttacctttgcaccaacctaatattaaacttcctgcagtgcacgggaaagcccccacaacagggttatctgaccccaaacctcaatggtgttaagatccaaaccttgatatgttaacctgtagctttaaacatcctttaaattgtcaaattcatgtccctgacataaggtttatgttagattttcaagtataacaaagatttaaactttaacttttgtacgttaatgatatgttagcttactccagtcttctattaaaacattctgtttttaaaatcagagacacacagcaattttataaatcatttctcttcaaggctgtgaagctctccccacttttgtgagtgccctctactggtcaaattatttgctttataacaagtaacagtgaaatcctaagtttgtgtagtttcgctgtttaaattatgggtggcatcaatttataaatatattcgttttatttaaaagtcttatatgattgatttcgtatcatttttgctctctgctaatattaatataaagattactgtctgtattagttaggcctaactaagtaggtgagtatagtgaactaagaaaggaaacgaggcagtatataagaaaatagggtggttcagttgttaacacttactgagcttactttgttgaagggactaaaaggcagcagtgtggctctctgagcttctttgcatgcactcaggagctgcttaatggagtccaaggcttggtggtgtgttacaggggatgataggagggtcctattcagaagtggcaaattgtgaaagtgcacattttgtagagttttataggactgtagaatagttgtgagcacctgatttttagaataaacagaaaactcaggtactgtatttaggtcaaattaagaataagtatttattaagacctgaatataaaactttactggtcatggtttttttctaccttgggtttttataaatccaaagatttaaaaacatacaaatggaagttggtaatggaattaagtgaaaggaaaaaatgattttatggtttggaatctcctaagattctggttttaacaatacaactaattccttaatcctagaaatgttcttcactgcccactttgtaccatgcagtcttcctgtgggctagagatacactgaggcgcaaaacagaccagattcctgccttcatggagcttattagttttaggtatctctagatttcttgtaatacctattacaatgcctgcacatcagttcattcatgtgggttcaacgtagtactcagtacatggcaaattcaagttttacttttcggaacttcatggatttttttcctcagaatatcttttatccataattggttgaatctgtagatgcagtacccatggatatggatggcccactttattttgaagagcagtgtttctaggcaatcatgctaattatatatgacttaatttagaggctttatacttaagagcattacatttctggcgtctcttaaccattattatttcataatgtgtaggttatggaacagttaaattattgggatcttaatatagaaattagtagaaataagccagatatggtggctcatgcctgtaatcttagcactttgggaggctgaggctattcgctgtactattttttactacttttctataggtttgaaattttttcaaaataaaacattgaaaaaagtaaggtaggtagtgtgtccctccttaatcctttcaaatattttattttcactatttctattaatttttttttttgtttttgagatggagtctcgctctgttgcccaggctggagtgcagtggcgcgatcttggctcactgcagcctccacctcctgggttccagccattctcctgcctcagcctcctgggtagctggtattacaggcatgcaccaccacacccaattactttttgtatttttagtagagacggggtttcaccatgttggccaggctagtctcgaactcctgacctcgtgatctgcccgcctcagcagtgtcactgcttctagaccgttttcaaggcacagagcttagaaatgcatgttactaagaaatcaagagttaactatttttcaccttctttctcccgcagtgagaaccctggttctaccctgtttctccttgtgtaaattttaatgctaaactatacacttgtgaaataaaaatgataatgtcattcttaaattatggatcttgcagtgttatctaagtaacatagattgagtgatttaactttaggtttccttatttgtggaatttggataaatatttttcacccttgagaaaagtgagactcctttctcatcatcagagtatccttaaaccattaaggcaaacatttgggaaaaaactgagctatctggctgcataaaaattaagttttctttaacaaagatagaagacaaatgaaaacctagaaaaaccatttggttcaagtaacaggaagctatcttatatatgaattagagaaaagcaaacacacaaatagaaaaaaagggatggggggtactaaagatataaatagcttgtctaccaaaaaagaaataaaataaataacatgaacatataaaaagacacttacttcatgaatgtgatgcaagttcaaacaataaataacatttctgtactttcatattggctaaggttaaaatgataactgctaggaagggtatggagaagtgtgcgccttgcactgtagtgggagtatagaccctcagactttatggaggtcagtctggaaatatgtttcaaaatgtaaactacatgtcctttgaccaggtaattcaacttcttgaaatttatccaaggatttaattggataaatgtttaagatgtatatataagaatgtttactgcagtgttgtttatgattttaaaaaaatggaaatcatcttcatgtctaccaatagagaatgggtgaataaattatggtatgtccatatatacaaattacatagttgttggaaatattaggtagatttagatatactgatgttcaaaaatgtccattatgtaagtgaagctgggtcacagcaccttgtgttgagtatgatttcatctagaaacaaaattactccctcatcctttgttgtgttttagttttttaaaataagcttataccattgggctgggggaaaagtaaatactcgttttggagagagaaaagggcactaaagtttcagataccgttagattatttcatgcttatttttcaagcctcaataaattacataattcacatgtagtcttggattaaggaaattgctattaaggctaaataaataatatgagaggtatataatataaaatatgaacattatattggcattaagattggatccacggtcattccagcctctcattcttacctggacttcaagtgatcacttgtgggcaaatgccatctgacttgaacaggttacacatgtatgctcattatatcgttattttcaaaatttgtcatataaattttccttgagttcattcagatttttgaactagttttttctcttgggagtagtacacacttaattctctctagtactaagctaatgttcaccattcttataattttaagtatccagcatttagtaaagaagtctttgttttctttatccttacttttagtgaatgtcttagtttttaattgaaaattctgccatgaaaataagctctttaacatcttcactccctaatcaaaacagaaatccttcatagccttcagttgtagctatccttccctgtgatttgtccagctccattatatttattttgaaatatggtgaccagttttgcaaaattatttcaactgtaggtgcccagtgattttgtaaggagaagatactgtttctgaacagttctcagtagccagtggcctgcccctactttttggcctgcgtgtagtatataaaataatgcagttaactttttatagcacttttcattttataaagagattttcatggtctttaatattaatctatgtataaagtcctgtatgcagttttacctactttcacagctgaaggaacaatagcttagagaagatgtgagataaagtagtttgcccaagcccatagcacaaataagtgaagttcttcggctgtccatggatcgaagactcccaagtctatctctagcctggacttctgtcctgagcaccagacatgtatgtatatcaagatgcctgcaggtcatatccaccaggacaacccatgagtacagggaattcaacatgcccaatatcactcatcttttccttcgccctcccctttgtactcatcccctgtcggtaagctctgttattttaaaaaattgaaatgtattcacatagcatacaatttacacttttcaagtgtacatggtttttagtatattcacaagggttgtgcagtcattactactaattccagaatgttattatcaccccaaaagtcccacatccattagcagccactccccaatcccttctcccaccagcctctaaaaactgctaatttttccatctctgtggatttgtccactctgattatttcatataaagagaatcgtacagacgtggccttttgtgtctggcatcctccacacaggatgatattttcagagttcgtctatgtttttgcttgttgatcattccttcattcctttttctggctgaataatactctgttatatggatataccttattttgtttatctgttcatttgatgggcatttgagtgatttcctctttttggcaattttgaataatgccactataaacatttatgtacacgtttttgtgtgaccatatgttttcacttctctcgggtgtatatctaaggtacagttgctgggttatatggtagctctgtctttgactttttgaggaactgccaagtggttttggtagtgattgtactgtttacattcctaccaacaattttacctaagtatttctcaaatctatttaatcttttcggtccatactgctgttgctgccttagttcagattttgtcatttcttgtaataattcgtagctcatctcccagtctctgctcccctctctccctccctcccccttcttctctctcttatttccacccatttttaacatttatagaagtcaaaagtctagttcagaaagcagaaaccatactagatatttcagcacagagaactaattaggtgttggaagactgaaaggcaaaaaaacactgaagtaacacagtaacatcaagaatgggcactactcctaagattcagggaatgctgggaagatttggggtttatcagaactggaagctcagaggaggggccccttgtcgctgaggcttaatccctgcagaggtgcctttggctgctactggtgaatctgagtgggtatgatgagtcagtgtctgggaagggccaaaacattttgtccctttctataatttgtcatgataatgctagtaatgaatctgatctcccttcctattttaaaaaccttttagtgattttgtataggatgaagtttaaaactccttacttaatatacacatgaccctccgtaagctggcccctgcttgattgtccagtttcacttcttggtgcttattctaaggcctctaagccttagagatcctctaagcctttgagatccccaaaccctggactgcggactggtacccacctgtgtggcctgtgaggaactgggctgcacagccggaaggaggtgagcattacttgccttagctcctgtcagatcggcagcattagattctaataggagcgtgaaccgtgttgtgaactgcccatgcaaggatctaggttgcatactccttaggagaatctaactaatgcttgatggtctgaggtgaaacagtttcatcctgaaatcacccccaactcggtccttggaaaaattgtcttccacgaaactggtccctgatgccggaaaagttggggaccgctgttctaagctaaagttatatggagctccttggttctgtgtcctcaacatgctgttctatgttttttacattctgtttgctccttcctgcttggaatgtccttcccctccccgtctttcttaatgcatacaaagttgatctctcctgtgtgccaccattgtacttcgtcttgcatatggtgttacattcattttattttaattatttatttacgttcatgtctcttccactcaccttagttgcttgaggtcagaaactatataatgtgtgacacggaatgtgacacctagattttcaataagtgtttctatgatacaagggagactgatgtgggtagatgggaatgaactcatcaacctctgtttacataccctaaattccctgtttcttccctattataattctgacagtctacaaccgtctttgatggcttataaacggaaagtgcggaacacatcattctacagtgaatttaaataacctttcggaagagtaacgtaaagtacttgagcattaattgagtaaaagtttctcatcttttcctacaggtgttattaagcagtatgtaaaaagtccttacaatacttaatacattaagaaaacatacaatttcaagaggaaatccccgagtaatacattattgacattttcagcagttctagttatattgagaagagcatctcatggaattggcagaatgaagatggagattaaatgagatgatgtttgtaatatgcttatgacagtatctggcatataagtaagggctcagtaaatgttgactgctgtaattactattaatagtaatatgattacctttagtaaaagttattagtttctttaggttttttgtttactacaatatagtaaacaaaatctatacttggaatgtatatattgttttgttttgatacatggaatatgtctctgtgtcagagtcactgcctgagttggaaaacccatactcgagtatgttaaaaggtgaacacactgaataatttagttattaattataatggaaaaatgacaaacttgatgttctggttaatgaggttatcttatcttgaatgagttagcttttaaattcctcaaaataaaggcatttaataaaccaggaaacacttcattaaaaaaattatgcaagtcagtgtaaaagaagattaaaattccacatgggcaaaggacacacgttggcgataaatatgcagataagaaaaaaaacctatataacattattactcctcaaagaaattggtatgaaaacaataaaaatgtgtagcttatcaaaccaacaaaaatttaaaaatatgaaatccattttaagtaatgataaaatgggtgcactcttagtgctttatagaatagtagtataatgaacctcatgtgtgtaccaaccagctctttcatatcttaacatttagcaacatttgatttagctctttcttttttccaagatagaaaagttaatattgttgaagactcctgcattcttttccctagtcttattttcttccctcccataaatgtgttaaaatctctgtgtgtattgttttggttgtatttttacataaaactttacatattatataaaatttaattgaaggtaaaatttattaaattattcttaatatatattgtaatttaaaaattaacagcttcattgtcttgataaaatttatggtatcttaaacatgtgcttgtttttctaagagaacattgaaacatagattttaaaacaaattgttgaaagattaaaaaatctgcctttgcacactgttacattgaaagtggggcatttgtcgtgaacattcatttcaaatatgtagtatcttcagaatatttgagaaggatttgtattatataattgaaaaatctgttaaattgtatttatgttaactgcttaattctaataaaatttccattcattttttagtatctgcatatatttacatcaaatggattcattcacttatttaagaggcagtactaattacctatagcgttcaagactgttaggtagagggtgtgtagtggtgagtacaacaggcgtgagccctaccaacacggagtttaaagcctagtagaggatatagacttaaacaatttcacaagtaaatacataattacaaattataatacatgctatgaaggaaacataggaggtaccagagaaggaagagtgctttgcatttttatttttaagaccgaagagtgctattggaggactttgagcaagtgaatgacatgatctaacctaccttcgttcattcattcattcattcattttcttccttcctggctcaagcagtcctcccacctgagctccccaaatagctgggactacaggtacacactaccacacctaatttttttttgtattttttgtatttttgatgggattttaccatgttggccaggctggtcttgaactcttgacctcaggtgatccacctgtctcggcctcccaaggtgttgggattataggtgcctagcccatggtgcctagccctaacctacatttataaactatcacttgctgctgtgtggagactatattgtgagattaacagcagggatacctgctaggaagcaattgctgcagattgcctgagacaaaatagttatcatggactagggggatggtggtggtggtggtggtaggtggttggatgtaggatatattttgaagataggtaaatggtgcaagattatgggtcagttttaaatgcttaagtaaattttctttgtaagacattttaggatgccatgttaagaatctctttataactgtcatttaaaaaaaaaccacatattttcttagcataatttcccatagtaacattactatgtcaaaggctatgaacatttgaatgactttagataaatactgtaattgctttccaaaaatattgtgcttattatgtcaccagaaatgtttgaattctgtctacaattcagtcttgccagtatagtacatttcatttagaaaaattttttactatgtagatggaaaaaataatattttagctgggagtggggggactatggggaataactttccttcatttaatattttattgtgagttagtttaagttactttattttatcgtagtttcctaaggctacaaattagtaaccttggtaacttatgtacctaatttaaaagtttacttttttgaaaggctggaaatactaattaaaaacgtaacaccttcatccttgtctttgctccattattaactagtttcattacagaatctctgtgttttaaaatcagatgggttttcataaccagtactttctcagagtggtaaatttaaaaaaatatataaagagaataaataatatttgttgagaatacttcaaataatgtgaagagttattaacttacagcaggagttggcaaacttttctataaagggccatatgggtctttgtcacaaagtcttgggtttttgtttttgtttttttaaacagctatttaactattcctagctaatgggcaatacaaaaacagtgggcaagatttggcctgtgggcagtagcttgctgaaaccttatttagactctaaattttttgaaagagtctacattgatgcatatttttttttcttcctccaaatacagttgacccttgaacaacatgcgtttgagtgaccatgggtccacttgtgatacacgtttttttcccaaccaaatgcagatatggagggctgacttttcatatacctggatgttcctgggccaactgtaggactagaggctgggggggtcttggaaccaatgccgtgtgtataccagggatgactgtttcttatggcctgacctgaagttggaacagaatctttattaatatataatttttgttgcgtttgttttctctttatatttatccattctttttagatcgtatttcatttaacactttttcttctttagtttttaccaagttgcactgaaaatagctcagtgactaattgcacttctaagagtgaggaccctagttaaaattaactctaaaaatactgaatttttaacctaaaccttttatttctaatcaacagtattatttatgagtaggttatagattactttgaaacggaatgtgtctcagaactttgctatcgatatttttaaggtctggtagggaaaagataataggaatgagatttatcagtgaataggggactgctttcccagtttctcggtcgcactggtgtattcaccatggaagcatcttatgaaatatgtacataaactactaatatcccacattacaggttgactattctttatctgaaatgcttaggacctagaagtatttttggattttggtttttcagagtagggatactcagcctacattggtaagtaaagaatgtgaggtgacaggctgggcgcgatggttgacgcctgtaatcccagcactttgggaggccgaggcggatcacctgaggtcaggagttgaagaccagcctggccaatctgtactaaaaatacaaaaattagctggacacagtggcacgtgccagtagtcccagctactcaggaggctgaggtaggagaatcgcttgaacctgggaggcggaggttgcagtgactcgagatcgtgtcactgccctccagcctaggcaacagagcaagactccatctcaaaaaaaaaaaaaaaaaaaaaaaaaaagaatgtgaggtggcagcaataggtaggaagagtctttggtcagctttacatgctctgtagccatgcctgggtaatgggttgactctaagactctgtgctttgctcccacctcctgctttttcattactctttagaatggtttttaatttgtgatctataggagttctttcaagtatttaataagagaataggctaaattaagtaaatgtcaactgaatgctcaaatctctactaaagagcctcttatttagaaaataaatatccatcttttttttctgactggtgagataattaatttttattacagatggtttggaaaataccatatgctttaaaagataagcacaaaattatagtctaatatgtaggttttcatactttaaaaaattgaaaaccaaagaaaaacatttaacatagcatctagtacaaagaaaagagataagcaagagataaatgtcttttttgggacagagttttgctgttgttgcccaggctggagtgcaatggcacaatctcagctcaccgtaacctccacctcccgggttcaagtgattctcctgcctcagcctcccgagtagctgggattacagtcatgcaccaccaggcccaggtaattttgtatgtttagtagagatggggtttctccgtgttggtcaggctgatctcaaactcccgacctcaggtgatctgcccaccttggcctcccaaagtgctgggattacagacatgagccatcgcacccggccaagataaatgtcttttaaattatctccattaaagacataacctttataacattttgatgtatatattaccagtttttaaacacatagtagatttgtataaatacataaacacatattattgtgatcatgctgcacttagacatctttatattctccttatactgtaaacattttgaaatactttactaacaacatttgtaatgaccattctttctctctttctccctctgatagaatggtctacagagtaattcataaactaaacatactttagaggctgggcgcagtggctcatgcctgtaatcccagcactttgagaggctgaggcgtgcagatcacgaggtcaggagttagagaccagcctgactaacatggtgaaaccccatctctactaaaaaaacagtacaaaaattagccgggcgtggtggcgtgcacctagaatcccagctactcaagaggctgaggcaggagaatcactcgagcccaggaggcagaggttgtagtgagccgagattgcaccacagcactccagcctgggcgacagagcgagactccatctcaaaaaaaaaaaaaaaagatacattaatactatagcctacatgtggaacattaagaaaataattgcttttatgtttatgctttatacctgttgttagccctgcttcttatttcatgatttcatggcttcacattgtaacatccctttaccatattttttgaggactgttttggcagaatgtgtgaaatcttgagcagaagtattacccaaaagtcagaagaaaatcagatttttatttcaagattctgttaaagttacccactcccttcttttacttaatcttatagttgcagttctctctctttttagaaaagaaaaaagaggcccctcaggatttgcagatgaaacaatattgctctttagagatatccatctggctgttagattatttttccacagttttcagaagtggatgaggccattagaatcttgagtattgcccatttccttatgtgtgcctttgactatagataaaatagatgcatgacaattatttataagttgattgatttttcttgtcatttaaatcatcttgaataatagagttggtagagctatcccatttttgaaattattttgttttgtcaataactttttgttaccagcatgtacacttgcattgttgactctccatataatacctttaaaaaatttttttttgtggtaaaatatgcataacataaagtttaccatggtagttttctttcatttgttttgtttttgtttttttgagacggagccttgctctgttgccaggctggagtgcagtggagcgatcttggctcactgcaacctccgcctcccgggttcaagcaattcccctgcctcagcctcctgagtagctgggactacaggcgcccgccaccacgcccggctaatattttgtattttaatagagatggggtttcaccatgttggccaggatgttcttgatctcctgacctcatgatccgcccacctcggcctcccaaagtgttgggattgcaagtgtgagccaccgcgcctagaccatggtagttaattttaagtgttcaattcagtgaccttaagtgtgttcataatgttgtgcaaccatcaccatgttgtctaaccattagcactatctgttttgagaacttttttttatcatcccaaattagaattctgtacctgtcaaatagtccccagtaatcctccctcccccagcccctggtaatctgtagtctacttttcgtctttttgaatttgcctattttaggttcctcatataagtggaattatgtggtatttgtccttttgtgttggcttacttcatttagcataatgttttcaaggttcatctgtgttgtagcatgtatatacaggttgaagcatccgttatccaaaatggttgtgaccagaagtggtttggatttcagattttttttttggattttggaatattcatagatacttaactggttcagcatccctcgtccaaaaatccaaaatcagatggagctcagtggctcatgcttgtaatcccaacacgttgggtggccaaggcaggaggatcgcttgagcccaggagttcaaccagcctgagcaacacaagaccctatctctccaaaaaaaaaaaaaaaaaaaaaaagatgaaagaaaaaaaaatccaaaatcaaatgctccagtgagcatttccttttagcatcatgtcaggctctaaaagttacaggttttggagcattttggatttcagatttttggattaacctgcattaatgctcaacctatatgaaattttattcctttttatggctgaataatgttccactgtatgtatatactacattttgtttatccattcatctgttaacagacacttaagttatttccacattttgggtattataaatagtgctgctgcgaacattggtgtacatgtatctgtttgagtccctgtttttagttattttggttatatacctaggaatggaattgctgatcatatggtaattctgtgtttaactttttgaggaactaccactgttttccacaatggcatcaccattttacattcccaccagcaatgcacaaagatttcagtgtctgtatccttgctaacacttattttccattttttgagtttttttgttttgtttttttaataatagccaatcctaatgggtatgtggtagcatctcatggttttgattttattttcctgactattgatgatgttgagcatcttttcaggtgcttagtggccatttgtccgtcatctttggagcaggaacaatgtcttttcaagtcctttgcccatttttaaattgaattttttgttgttgagttgtatataacaccttttttgaagtaaaaggtgcactgtaataatccagactgtgtttctcccttctcaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattataggtgggtttaaattgaatataataagctgacattaaggagtaattatagtttttattttttgagtctttgctaatgccatgcatataatatttaataaaaatttttaaataatgtttatgaggtaggtaatatccctgttttataaatgaagttcttgggggattagagcagtggagtaacttgctccagactgcatcggtagtggtggtgctgggattgaaacctaggcctgtttgactccacagccttctgtactcttgactattctacaaaagcaagactttaaactttttagatacatcattaaaaaagaaaaccataaaaaagaatatgaaaagatgatttgagatggtgtcactttaacagtcttaaaagcaatcgtgtgtatagcatagaattgcttggattggataaacagtggcattatatattttaaaaaataaaagttttgaaagattgaagaatttgggcattacagttctcttaaatctgacaaagctgcataaaactattaaaataatcattattatactattttatattctatttctttgagggtttagttttccaaaaactacatattaagcaaatgaatcactcagtggctatgtcatataataacgagttagcctagttataagaagtttaacattttatttaagaacattgttacagcatgtttactgtatagtctagtaatagaggaaaagacatttgggtgggtggtagtggtagtatttttatagaggagttaccaaatttcagctctattatccaagtttacccagctaatggtgttcggaaccgggaatttgagccaattctgactctgttgtctgctctgctccttcttttgtgctgtgtctttgaaagtcacctaaaattgtgagggaatgtaatttcaccccaaatttagagtttatgcacttgttatattgaaaatgattaacatgtagaagggcttttaatggaataagtggtgtagtaacttcagtgttgcctacctagaaatcaaaatctttctagttgtccactttgttttttgaaaaagtaatatgaaaattatgttaatgctttaattcaggtttttgtaaaatattttttatctttacacatttaacatacgtttctaaaattatagtctgttatatagcactttgggtctagaatttttcagtagtttctgttttactattatgatctacctgcatattaacctattaggttatagttttactatacttctaggtatttgatcttttgagagagatacaaggtttctgtttaaaaaggtaaagaaacaaaataactagtagaagaaggaaggaaaatttggtgtagtggaaactaggaattacattgttttctttcagccaaattttatgacaaaagttgtggacaggttttgaaagatatttgtgttactaatgactgtgctataacttttttttctttcccagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacaggtaagtaacactgaaataaatacagatctgttttctgcaaaatcataactgttatgtcatttaatatatcagtttttctctcaattatgctatactaggaaataaaacaatatttagtaaatgtttttgtctcttgagagggcattgcttcttaatccagtgtccatggtactgcttttggctttggtttctttctacattgaaaatttctcttcaattctgagcacatgttaacatttagaattcaagaggtggggatttttttttcccatggttacatatatatatatatatatatatatatatatatatatatatatatatataaagaacagggcaacaaatttttgcgttttctatttcggtagtacttttaaaccattatgtcatgtttctaggttaaacgttgttgtatttgaagaattttactttggcagaatttttttgaggatgtgtttatttctggagaaaggtctcattaaagaaagacaatacccagaaagccaacagaaattctgttactcatttaatgcatttttctgacaaaaattattgccagagagaacctgaattttgtttcaaaaatcatctttgttttaaaaatgactttttcttcaggtaaaataaaataatttcagttgctattatttaacctgtttgtatgaagagtttaacatataggaaatgaatacataaagataggaaggaattaattgttatatgtagtcatatgtctcttaatgacagggatactttctaagaaatacattgttaggtgattttgtcattgtgcaaacatcatagaatatacttacacaaaccttggtagtataacctactatacacctgggatatgtagtatagtctcttgccccagggatacaaacctgtacagtatgtaactgtactaatgactataaggcaattgttaacacaatggtaagttttgtgtgtctaaacctacacttgggctaccctaagtttatatatttttttaaatttctgttcaataataaattaaccttactttactgtaactttttaaactttttaatttttcctaacattttgacttttgtaatacagcttaaaacacacattatacagctatacaaatttttctttccttatatctttattctgtaagcttttttccatatttaaaattttttgtttgtttttacttattaaacttttttgttaaaaactaagacatgcatgcacattaacctaggcctacacagggtcaggaccatcaatatcattgtcttccacttccacatcttgtcccactggaagatcttcaggggcagtaacacacgtggagctgtcatctcctataataacattgccttcttttggaatacctcctgaaggacctatccaaggctgtttatagttaacttttttttttttttttttttttttttagtaaataggaggagtacactataaaataacaatataggtgctataccattatacaactgacagtgcagtaggtttgtttacaccagcatcaccacaaacacgtgagcaatgtgtcgtactacagtgttaggatggctataacatcactaagcaataggaacttttaaactccattataatcttatgggaccactatcacatatgcaatctcctgtggaccaaaatgtcattatgtggtacatgactgtactaagaaattgatccatctatattccatcaatttgtttagggctttttctggttacatttacctgtgagcccagaaaaccagttttgtagaaattaacttctgtaatgctaggagttaaaaaaaattgctgaacaacttttacattgttaaacatttaaaaacaagcgttctagaagtttatcaaatttcataaaggtgcaaaaatgtaaatgtaaatcattatccagctaatatatatgttgtatttccctagtaggagagcatatgtacctcttcctagttatacaaatttgatatatagtaaagaaacagtaaattctacttcaagtcattttgggaggattaaaaactgaatttctctagtttgaccattgtacagatttatctggcaattttactaaaacctgatttataggttaaacttggtgtatatcatatatcactttactttagaggaattaagatttcacataaatccatttccaggttccaaagaccaggaagaggcttggtttttgtttttctttttactgtctttacagtctccttgacttttcttaggagagaaggtactgagaaaacatgattctaatatttattattttttcttccaacattttcttatgaaacattttcaaatacaaaattgagttttatttaaaacatttgcaaatatactacctagattctaccattgttgttttatatttgctttacttacaacttttaaaagatgctttttataccactgaacattttagcttacatttcacaaagaaaagaaaaaatttaagagactttgcataatgttttaaggggttgcagtaaagaagtgcttcttatattttcttatgcatacaaatcagctgggcttattaaaatccagattctaattcagaaggtttaggtggggaccgagtctgcatttctaacaaactcctaggtggtatttttcttggtacttggaccatactttgagtagaaaagcagtagaggacataaaaagagtcttgttagtcccactttgttgctgtccacttctcatttgataatatcctaaaatagctgtgtctcctttttggtggttgtatgattactacctcagaagtactaattgattcttgctatttgaccttaatactttaatataacacagcattcatatttgatcagaaaactatctggcttccttttataagagatttttaggttttatacagttttgtggccttgggtttttttgtttgatttgtttttttgaaggtatataatatgtaagtagataaacaaatttgatttgtagacatttttatgtggatcatctaattaaaaatggagggatacagtatgaaagaatacttgtacttcttaacagagcactcaacctttcttttacatcctgtttcactgatgttattatgtaatttatgttgctaaactataaattagatatttaatttctgttctttgatttccttttattattaaatggacttgttgatttgcctagaaattaatttgcctttcaaaagtcttattaatcttcctccgttgaaattaatttgatatttgcatgcttctggaagactttaaagagctattccgagtaactgtagagattataaaatgaaatatgggaattttaataaattttacatctccagttactggtgaaaatgtcaagtcctcctttctgcagagtattttgttactcatctgttattcagcttatttatttatttatttatttatttatttttctttctttcttgttttttttttttgagacggagtcttgctttgtcgcccaggctggagtacagtggtgggatcttggctcactgcaggctccgcctcccgggttcacaccattcttctgcctcagcctcccaagtagctgggactacaggcacccgccaccatgccttgctaaatttttgtatttttagtagagacgggtttcactgtgttagccaggatggtctcgatctcttgacctcgtgatccacctgcctcggcctcccaaagtgctgggattacaggcatgagccaccgcgcctggcccttatttgttttttaaacaaaattagtgtgcatatccttgttgtattttatcggcaagttgttttatgccctaacttttggggtcttgatcatgagcctaaaacacgtaaacacccaaaaagaattatattccggttaaaggaacaaaacattcatttagaagttctcatccatgtaaatcagaggctggcaaatattttctgtaaagggccaagatagtaaatgttttaggctttgagggccacaagtggtatctgttgcatttttttttaattatgaccctttaaaatgcaaaaatcgttgttagcttgtgcatagtataaaaataggctggccgcatgctgtggctcatgcctgtaatcccagaaatgaggtgggaagccgaggtgggcacaccacctgaggtcaggagttcgaggccagcctggccaacgtggttgaaaccccgtctctactaaaaatacaaaacttagccaggcgtggtggcgggtgcctgttatcctggctactcaaggggctgaggcagtagaattgcttgaacctgagaggcagaggctgtagtgagcccagatcaagccagtgcacaccagcctggacgaccgagcgagactctgtctcaaaaaaaaaaaaaaaaggctgtggctgcatttggtccattggctgtaatatgctgattcctaattctctgggtaactttagtgtttgattagctactagaagttaggttaaacttttgtattttacaggctaactttaataatcttaaagtaaaacttaacatagttcatggaaaggaaatagaaattttaccctagtactcttttttttttttttttttttttttgaggcagagtctccctctgtcacccaggctggagtgcagtggtgggatcttggctgattgcaacctcctcctcctgggttcaagcaattcttgtgcctcagcctcccgagcagctgggactacaggcacgcaccaccacacctgactgatttttgtatttttagtagagacagggtttcgccatgttggccaggctggtcttgaactcctggcatcaagtgatcctcccatctgagcctcccagtgtgctgggattacagacgtgagtcactgtgcctggtctctagtattttttttttttttgagacggtctcactgttgccaggctggagtgcagtggcgcgatcctggctcactgcaacctccgcttcccggattcaagcgattttcctgcctcagcctcctgagtagctgggactatgggtgcacaccaccacgcccagctaatttttgtatttttagtagagacggggtttcaccatgttggccaatatggtctcaatctcttgacctcgtgatctgcccgtctcggcctcccaaagtgctgggattacaggcgtgagccactgtgcccagctgtactttttaagataagaattgcagggtatatatttttaccaacttaataacttataattttaaaaagctaattacttggctagaatataatgcgttacatattctttacactcagttcagtccatatctgaaaggcaaatagaattattttctgctagtacattgtgtagtccctatgttcctagtgtataaggactgttacctagttcacatttatctgggttgttgacagattttcctggtccctttggacagtgcatggccatgttggcaaaagctgtcaaaattgaaacattgacaccatgagaattgtgtgttttccagtctgctaaaatcaaaagtgggagggttcagtaaggtgaataacagaagcagagttttcggggtatctgttactcctcattcggcttttctgctctctgggggtctcaatttaaatataatgtgaaaattagttttacgaacctaaaaatgttgagtgattcatttcctggttttgttgttaatttctagatatttaaattaattgttagaagaaccccgttaaagaatgctttgcaaaacaacctccttatgtgctatgtctctgtttaatagtagttgagtttgtgtacatgagatcaatattttgaactatagctttttatgagttaaaaattgacggaacagttactgtgcacttgctgtgcaccatggtagtctcccaagtagtggtttttctgcatttcaatagtacatgagataggctgtgggtggcaaggtttcttgagaaagtgagggatgcacagttgggttttagaatacatcttgttcctccatgcccttccccaccaaaaggctggtagtcttgcatttgtatatagttagggtatttgatgtgttgcttccttgacagagttttgcaagaatttgcagatttaacaggaacaaaaacttacttaaaacaaaatctcttagtaaaagcatagtctagcaagatttagaatgatactttggctaacagtactttctctatatggagtgctttgtttccatagcctcacaagtatgttttcagataatagttgagttgaaaatgttgtcaatctcttgattttaaaaaatttacatatttaaagttgtatacttttgttcctacgtattttcagttgttcttaaagtttaataagtgacatttgaaaatgagtatatgtgtataaaaacaaaagtaggctaggcacggtggctcatgcctataatcctagcactttgggaggctgaggcaggcggatcacaaggtcaggagtttgagaccagcctgggcaatatggtgaaacccccctctactaaaaatacaaaaattagctgggtgtggtggtgcatgcctgtagtcccagctactcaggaggctgaggcaggagaatcgcttgaacccggaggtggcggttgcagtgagccgagattgcaccactgcagtccagcctgggcggcagagcgagactccatctcaaaaaaaaaaaacaaaaaaagaaaaagttaaaaaaaaacaaaaaacccccacaaaatgagtatatgtggcaacaagtcctattctcaaaaaaattattgtgtgctagttaagagcttaatgagtagccagtcggtattaaatatctgtttcagctatattttatctttaaaaattatctacagattttggaatgtgaaaaactagtgttttgtttcataggtatatactgtaggcattttaaaaataagagccagtgccagtggtttacagtgtacacaaggataatgttctcatgttctcttgatgtcagtatgactttaaagcatattatcaagaaataactaagtctgaaaaactgtggtaaataactggtactctaaaacctaagtttcttattactaaaaataagaaatggtaaaagtcaccctgtgctgttaattatatgagccactgaggtcctgacactgaattcttggtggtggataataatctcttctttttaattattggcttccaattctctctgcattgctggaaacaaaaatcatatatttcactattggtggtggggatgctgtcactgaaaaagtagacacattcatattgattttagaaataagttaaaatcaaaatttgcttctgctaaattagtagaggaccaatactgtttttctccttcatagtatgttttggtacttctacattgacattataacttttttttttttaaacagaaatagaagtttacattcttagaaaatttatgaaaatatgagcttttacctggtttgtgtgtgtgcgtatatatatacacatatttttaaatttcttacattgattttcaaattgaaagagaaccatttgtgaaagtatcttaacagagctcatgctttacattttacatgctacaaagttattttagtgccttaaattatttatgttgcttattaatgaaaattttggatacataattttttcaagacaaaggtaaaaataataaaccctttccttctgaggattaatgataaatataaactttaaaacgattaaaaaaatttttttagagacagggtcttgctctgttgcccagactgaagtgcagtggtgcagtcatagctcaatgaagcctcaaactcctgggcccaggcaaccctcctgcctcagccttttgagtagctgggacttcaggctcatgccaacatgcctaatttatcttatttttagtagagatgaggtctcaaactcctggcatctcttgccctctcaaagtgctggtactacaggcattagtcaccacacctgacacttaaaatcttttatatacaggtgtaagtgggtatctaacttaaagtgccaacgaatgtagttgaaagtttgtagttggcttagctaactagttaactaaattgattccattaaaaataagataagactgctcttagaatataatgatttttgttattcgttaaatataaatatatcactggatagtatatgttaatgacttgagatacgcattttaacatataatcacgttacttaaatgcctgcctttgaactgaaacttaacattatgaatttaaattaaagtttgactttagaggtaaatttctgtactttactaaagcagttcttaatataattctgagatttctaaaaattagtgtgccctaaagaattgaggtgtgtttttcttaactactgtaggcagtagatgtacagatgacttctgcatgcaaaaattaagccctagccattggtttacttcaactaatacttagttgccaattctctgtgtgtgattgaatttaaaactgcaaatggtactggtgatacattaactttttaggtgctaggtccactttgttacatttggttcagtagaaacattgatgttaccaatctcagaaagctaaaatatgtatgccaatccccaaattaggtaatttattcttaattttaagataaaagaatagaattcccttaaaattaaatgtggagtaaaatataccagctttaaaaaatattcacctttctgttagaagaatgaacataatattacatcttttaatttgcactatatatagattaatatttctgtgtatttctctgtgcccctactttgatggtatgcttttctgaacaaactagcagcacagttaactaagcactttgccccgtttgatgactgcctaattttctagattggaaaatattaaaaacttttatctccatatggccaatatatgattgtacctgttgtcatagctctcttatgtttaagcaagaaaaaccctattaagagtatttaaattagaatggaaggcacacagccagtatgattgaacactgttctaaaaattatttttaagacttgtagtaaggccaggtttggtggctcatggctgtaatcccagcccttaggaggccaaggtgggcggatcacttgtgctcaggagtttgagaccagcccgggcaacatggcaaaaccctgtctctacgaaaaatacaaaaatcagtcaggtgtggtggtgcttgcctgtagtcccagctatttgagaggctgaggcagggggatcacctagcctgggaggtcgaggctgcagtcatgatcgtgccattgcactccatcctgggcaacccagtgagaccctgtctctaaaacaaaaaaataaaaaaagaacttgtagtaaggatacaaaatgctcctattttgtgtgtgtcctttaattcatgatgtttttatattatggtaagcagctctcatttaagattttaataatgtaattaaacatgtacagaagacccagtctcagcttcacttgtataccctggaaatagactgaaaggtgttaaaatttaagataaaactcaaggttccagtttcttgactcacctttgagattcttttatgtttttgttgttttttaacaaaggtttcacgtccatattttaccatttttcttctcattctcccctggaggagggtgtgggaatcgatagtatataaatcacttttttcctaagtcaaagaagtaatttaaagctaacttcagtttaggctttaattccaggactagcaaactaaaatggttgcattaattgacaaacagatgctaatacctgtgtttaggcttgtcataatctctcctaattcctaatttaaaaattttaaaatttaattccattagaaaacaaaactgacttttaagaacaaaccaggattctagcccatattttaaaactgcatcctcagttttattcaaacagtctgatgtctgtttaaaaaaaaaaaaatctcaagctcataatctcaaacttcttgcacatggctttcccagtaaattactcttaccaatgcaacagactttaaagaagttgtgttttacaatgcagagagtggaggatgctttttatacattggtgagggagatccgacaatacagattgaaaaaaatcagcaaagaagaaaagactcctggctgtgtgaaaattaaaaaatgcattataatgtaatctggtaagtttaagttcagcacattaattttggcagaaagcagatgtcttttaaaggtaacaaggtggcaaccactttagaactacttaggtgtagtattctaacttgaagtattaaaagataagaaacttgtttccataattagtacatttatttttaatctagtgggaattaattataattgagacaattttgatggctgtagtagactaatctatatttggcataaagtctaatgatttaatgagtcttaagtaaactaaatatttggaaactgatatttacctttatttttaagggaaaagttttgagataatcagcagcttttttttttttttttttttttttagtagggagaaaaagatatgagctatagtagacagcagtaatattgaatggcccagaaggtgggaaaaagccactcttaaatgtattttttcttttggatattttacaagcaaataataacttctgcctaagttcgccatctcagtggcatcagcagcacagcactttcttatcccagtgagaaacctgggaattttaggatgactcctaccgccctcttttccccctggtttggaagtatccacaaattcctgtgacgttacattctgtgtcttttatgtcatcattagttcaggcccctatcatttcttgttggactgttagaacctcctatttggtttaccagttgctgccatcattcattgtgaaaccggagagatacactttaaagaaatgtcatttttggccgggcgcggtggctcacgcctgtaatcccagcactttgggaggcctaggcgggtgatcacctgaggtcaggagttcaagaccagcctggctaacatggtgaaaccctatttctactaaaaatacaaaaaattagccgggcgtggtggcacgtgcctgtaatcccagctacttgggaggctgaggcaggagaattgcttgaacctgggaggcagaggttgcagtgagctgagaatgcaccattgcactccagcttgagcaacaagagcgaaactctgtctcaaaaaaaaaaaaaaaaagtcattttagctatagaataaaatctcatgttccacatgtgttgcagatagtccttactaccttcccaccactccagctcttttttggtcttatatctaaaaacgtcatcttgcctgaatttcttttgttcttctataaataaataccatgttatttcctaccttcccttgagtcttggctcttgtttggaatgccagtatttttatccctagtcttactaattagctaacactctcatgattccccagtctcctactctctaaaaacctttctttaaacccttagactaggcatggagcccttcctgtgtattcccagaatactattcttaactattatatgcttcccatgttatgttgaaataactaacctcttctgtttcattcctatattacttgacagcaaaatcttagccagaattacatatttttaatctttgcacacccattgcctagtaaggttcctgggacatagtaactacccagtaaatatttattgcgtggaattctcattttcgtttctaaacccgtattaaactctgtcttgctcagaaaatacttcactaggtatcataaagttcatggcagagcttaagctttggatgcatattgtttgtaatatatcatgttcttaagaataggcaataaaattacagttttcaaaaactactacatttattatatttattacaagttggtgttctttattacatgaattttaggtatttcccaaaagtataaaatatacatttgaatagtagactcaatcccaaaagatactacgtggtgtactaatctactaaactcagaaacaaagcatgactggcattaatttttgttgaaatttatgaactctgaatgtttttgaatatcattctgtaaagcaatattttgcaattaaagcaattttgcatgttaaattttaccacaacctctaaaatattgcaaatttaacaatacagtttgaaaagttacacattttaaataacagtaccatgaccagatttaggtggtggttttaattttttattttctcctcctattgtctcaccattagatgattttaaaaatagaattgtttagagtaaaataagtgttatgctctaatttatatttaaaatgaaggtttaagcacgtactattctaaaatttctaatttgtgcaaattatgttttatacagtgactgtaggtgaatgtcacaattgtttgatgtgacgaatccttgtttttcagtacacgtggaagtaattcatataaaagagaagtatacttggtaattaaaaatttaaaattaaatacaatttaaaaaaaaatttatttgacaagctggctgtggtgtgtgtgcctgtagtatcagctgcttgggagcctgaggcaggaggattgcctgaccccaggagtttgaggttgaagggagctatgatggtgccatggcactgtagcctaggcaacagaaagagactccatctcttaaaaaaagtaaaaataaaaaaattttggcacagggacagtggctcacacttataatgccagaactttaggagtccacagcgcgaggactgcttgaggccaggagtttaagaccagactgggcaacgtaatgagaccccacctttaggaaataaatacataaataaaaatttgacaatgataaacatatataaattagcttttcttagtcctgaaaaagataatgttatgtgtatgtgtgagaatgattagttctcatatgagaaaaaaagaattcattgctctgtgtaggttgtgacatttccttcacgattgaaattaattaatttttttttattacttatttatttttaaaatagagacaggttcttgctgtgttgcccaggctggtctcaaactcctggcctcaagcagttctcctgcctcagcctcccaaattgctgtgactgtaggtgtgagccactgcactgggccaaaattacttaattttaacaagatgatgtagagaggagagttcattgcaacataagcctagaatctttgtcagaatcttaggaagtaatgttttcaaattctgtgttttcaccataaaatgtgtcttctctgtgtccatcacatggtttttcattgttttctgctttaccattttagtaccattggcatttttcttcattgtaaaagtagtagaaatggagtagattacataaggatgtgatcagagggaatttattcattcagggtaagggagttagatcctcttttaagattctatcacattctaagggtttatgattctaaactgtcaagtaaattgtcaagtgctggcaagctacagaataatttttattgtatcattggaaattttcccctctatatgtgttaaagagtttagcctgaagggatacatacacatacatatatgtaatcaaaccttgatggtattgtattgctgataaattatttcttaccacttttcctttctcctgtgggagaaacaaaagcatatgtttgtgtagtatcagtaatgatattagagagtgggaaacatcagtgagtgcagtttggggactttattggagactttcactagtgctcaaataaataatgctggtttttatcctactgtttgcttaatgtggactagcctcttattcccattctatgtttacctctcttaaaatattggtcacgctttcttgaattatagatctattaggaaaattcatgaactgtagctaattttcattgttcatgctccagatttattttgaaatatcgttaatcttagtagtacagtaaaggagaaataccacttaacattttttgtttttttttctttgagacagagtcatgctctgtcacccagtctggagtgcagtggtgctatctcggctcactgcaatgcacttcgcctctccgggttcagcaattctcctgcctcagcctcctgagtagctgggattacaggcacctgctaccacacccagctaatttttgtatttttagtagagacagggtttcaccatgttggccaggctggtctgaaactcctcacctcaagtgatccacccgtcttggcctcccaaagtgctgggattacaggcttgagccaccgcaccccgcccacttaacattttaaattaatttcaagataatatcacttgaatatttttacacatataatttttttaatacatttatttacacagtttataatatcctacaaagtgattacaatgagtaaaaacccagttttcattgttcctaaagtggcttgatttatacaacttaatgtgttgggtatttgtttctaagactccctctgctgtctaggtttggaagtattgtgaggttaacagattttctttttatagttactactcagttgaacaggctttaaaatacagagagaatcatattttttcttcattttttgcttttatttatatttttcttttaattggagacatgacaagaattgacttgtgtatggatcttgcataatttaagtactgcaggtttaaaatctactaccagtttgagagtgccatttttcacactgtagattattaggttgaaaagtattatggcttaaaatcgcttttagccattaaatttaaataaccttgctttaatcataaatagatggtggtcacaatgactaactgttaaactctttgaagacaggatatttggctttatatggcaagcttttgaatacaacagaaattaaaactttatgggatagaaagaatctcctccaaattggtaaactataagacctttcaaatgatttagctaatttctccacaaatctgaggtattagtgttttttttaaagtggtattctcctgtgttggggtcactttaaacctttttcttaatgataaatatatgaattgaaactaatcccttaatatatatcatttgaaaactgaaataatatgtttagatactgtttacttgttgataaattattggaataggatgttcgaatactgtttacttcttggtaaatttttaaatccaatggattttacgtaagtatagaactggagctcaaatactgttactgtgtgtgaagatatatgaacatagtttacagttgcatggcttatatctaaagtccagaaacataaggacaattaagtgtacacacacacacatgcatttggattttgatgacttaggtttgccaatgtggaaaaaatagtagcaaattaagttctcctgtgaaaaagtcgttaccttatttaaaattctgtgccattggttatccttgtcttttgtgaaaattagtgttcctgtttataatattgacaaaacacctatgcggatgacatttaagaattctaaaagtcctaatatatgtaatatatattcagttgcctgaagagaaacataaagaatcctttcttaatattttttccattaatgaaatttgttacctgtacacatgaagccatcgtatatattcacattttaatactttttatgtatttcagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 4 - DNA, 190753 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgagtgctggcgccaccctgccgccctcccgactccgggctcggcggctggctggtgtttattttggaaagaggcggcggtgggggcttgatgccctcagccaccttctcgggccagctccgcgggctgggaggtgggcatcgcccccgtgtccctctccgtcatgcagcgccttcctacgtaaacacacacaatggcccggggggtttccctggcccccaccccagatgtggggattggggcagcggtggttgagcgggaggctatcaatagggggcgaaactcagggttggtccgagaaggtcacgattggctgaagtatccagctctgcatctctgtggggtgggggcggcggcggcctcgacgtggaggatataggttagttgctggggctgagacaacagcccgagttactgtcgcgtgtaattcttacatggtcgtggggatgatggggctcatcatttcctctctcctctcccggactgccccccttctcagtccgctgccctttttcacttttctatttggggatttctcttcacctgttttacccagcaaattattttgatttagtctttactttttcaatcctaaatcgcagtttccgatgccttttctggtctctggtcctctgttcctaatgtttgtcagcgctctgtcgctgattggtaacccccattctattcccatctaccgcccgctcattttccagttgtcggacctgcctgccttctaaccccagctcccacttaagagcatttttgcacttctcttaccctggtcctcttgaggctctgtacttgatctcaccactccctaacattgttgtctgttgttatcttcacaaatcctcctggacactttggagctacttgttttctgagcccagaagctgtcaagattccatcaggtttcacttggctcttttcgcgcttgcactactggcactttttggctagtcgtccattgtgcattcacacctctttattcctacccatttttataggtctgattgatttcttagtgttgtcctcctttttgtcctatttttttccttttcctttttcctctccagtccttgcttctctcagcctgtttttgcattagtcagcctcttagcactgtgtcaaattatttacgtttttttattacataaaatttattacaaatatttggtattttattacagaaaataatactttattatgctttacaaataagatatggtataataattgtggtttacagttattgattaggtaatgtgacttactctgttgactttgctcgaagttctctttgctacttactattaacatctaatttctcaattctcataacatctcattctctctgcaattttttttttgcatcatcatctttggaaattcatccaatatgcttgctttattcagcatcagcttgtttatgataatgtttgttttctactctttatatcatctttgttacatgcccaaaatgtgttctgtaccatcatttgatctgttctaaaatttctcatttttaagtttcttaaaatcattccacttttcagtatgcatttttgcttagatcagtttcctctcatatctgttcctttcccccagcttcttgatttctaaggagaaagctcttctctacttcaatttcctagtttattctgtttcccttgtttccagttaccattcattttgccttgtttcctggcttttggtacttaactttctgaagcttcctcttttcttctccacacctccacgttccttcttatttataaacatctttgtttcctttgacatggaaatttatttttaggatacattgtttttaatggataaatactaggggtcacatctgctgtctgttttctccaggaatcggatatgcctttgtcttaaccaggcacaggtgcctctggattttattttactctgtaatagatgtgtagttttgttgaattgtatcttgtttgaagactactacagagtggaacaatgagtgaagtaataagtaggggttatgaaattgtaattctctgattataaaattgtttatcttgggaactttgctgcagagttattagaaccgtttgcaattctgtaaagaaggcttttgtgaagtaaaatctctacccttctattttatttgaaagggccagattgtttggaactgtaccccctgaagagtctgatttagtaagtgagagcgagggccatggatttctgtatttggcacatgtcttgagcagttcccatgtaccaatccttgagaacctctaggctagctgaatttaagtataaattgccagtaattggaaagcatattcatatcttctgaaactataaggatactctcattttacttggttaaaaaacaagtgtttcctactgtcctctttacccaggttttaatgtttagtggtgaacagtagttttccctctacatttttttctgaactgataataaatgtatttggctgggagggtgacattgattaaaaaatgtatctcttgaatgtaaatatcagtattacagatgataaaataaattcctccaagaaataattttaaatttgaagttgatattcagtggaaactgaaatgtgctgtggtcttttatttgaagtcttccttacattcacttaaagggatcttttactgcaaattacatggaaagaatgaaaaggtttgcttgtgtgtaatgacacattttattctgaagatttattttacctaacagtaaaatgtaggttttttttttttaaataaaagtttcccagagggaaatttcatctaaaaaaaaagtctgatttcaaagggaaagcaagtcattatcaaaaattagaaaactataagtacaaaaagtaaaaaatcatcagtaattttgccactaagatattattactatagacattttggtgtattccatctgttcttttttaatgcttttataacactatgtagttttgtattttaaaaaacttaaagcaaaaatttctacgtattattagacatactgtgatttatttaactaatcatttttttggggtgttaggttgtttttaattttttactgccatcaaacatcttgaacataggatgtagattttagtctttaaaatatgttggggaatgaacaaatttcacatcctgtatttgtagtattaatactttgtaggtgctcaaaatagaatattctggtaaatgattagtgcttattaaatatttatcaaatgaatgtacttgtacttttggcattaaacattaacatctgaccatttatatttacctgattttttttctatggccatatggtatgaaatagtgtatggtataaattaaccatatggtataataaatacatttttttaagtgtgataccagagtgatatttattaactgttcttcctgtgctgtttctgtagaagggagcttctcacaattgcattagaattacaattttattatgttctgttttcaagatctctgatcgtcagtcttaaactgtttaattataataatgtattgactagggaatattctgggatataatctcctttataatgaggtccactgtattaaaatacatctttgcaagccacaccaggttggattgcatcataaccctgaaaagtggtattctcattaatgcaggtgcttgtgcagttttggctattgctgttaatacttatacagatatattcacaggtgcccttgtggcaaaaatcataaaatagttgtttgtctttggtatttctagtgttcactttctatattcttttctctctccttatttactgaactcccttctttaggcatccactcactcctttttctgtttagaatattatctgtcagtcattttatatgttggccattaaaggaataaactgtcagtaaacagctaagaaaggaatgttggactgggtgcttgaatccttgaatgtagtaaatgtgagtgcaaacttgatttaattgtacatgtatttggataataggccagaaaaattacattagggtaacaggctagaacagtctgacttttcttgtttttctatcccttgctttcttgattagaatgaataggaggtgggtctggatatagcagctggaaacctgtgttccatgagtgatggggaagagagggagggaataggttcctctgatttttggcattttctaagacctgatgcccaccttgtcagagaatgcgatgactacttttgtgttcttccttttccctttttctcccaattataaaattgttttctctttcagaactgcagaagtgcattttgtttctttgacactttgatgttgttaatttagctgaatacctagtgaacattttgtgtcataatccccttgttttatgaaatccagtatggtctagtcaccttacatttctgcctcatattgtccttaagcctttttttgtcagtagctcttactagattttgtcttcatcagaagttaaagtgttttaagtcctttactcattctgtttctctattttaacttacattggttattctgtaaagtcagatgtggcagtagggctggtcgtggtggctcacacctgtagtcccagctacttgggaagctgagatgggagcatcacttgagccctgaagttcgaggctgcagtgagccatgatcgcaccactgcactctagcctggcaacagagtgagaccctgcctcaaaacaaacaaacaaacaaacaaaaaaaaaacaaaaaaacttgcttgtagaacttctgaattcaaaataggtgggcctatttgggagcttttctgtttttaaggtgtcaagtactgctttttaaaatcataaggttatggataacttcatgttagtgtaagaagaaaaatatagcctcatttgttccatttctttcttaaattttttgttttcattgccatgtttttatttttcgatttcaatttttccagcctaaatcactaacatacttaattagcatggtaatcagaagatactctttaatacagtctccaccctaacattaagcaattatttttcccccctaccctctgagattatttttgtgtccatgttttctcttgggcttaaaaaaaaaaactattattctaattccttcctgtatcaagactatgcatatagagggaactcaatgcccagtaacttctttttctgggccctggtgatgtagaatataaaaattgctttgaactcaattaactttatatcttctggaagctctgtaacatcggataaagcgtcgttttcattcttgtaatgtagctgcagttcctgacagcacgtttgggacaaatgtactgtgggacggtggttttcaaagtacgccagagctctaggagaatttttcgaaaacattctatcattgtaaataataattttttttttttttttttttgagacggagtctcgctctgtctcgcagactggagtgcagtggcgcgatctcagctcactgcaagctctgcctcccgggttcacgcctttctgcctttctcctgcctcagcctccccagtagctgggactacaggcgcctgccaccacgcccagctaattttttgtatttttagtagagacggggtttcaccgtgttggccaggatggtctcaatctcctgacctcgtgatctgcccgcctcagcctcccaaagtgctgggattacaggcgtgagccaccacacctggccagaataaaaattccaaattgcactaatgcatatgtgaaactgtttttgtctgttttgtagttttaaaatcttcatctataatagtacctggcacataggtactaaaatatttggtgaaagaattagtgaataaaaccttactggatatgaggtgatctgattttctgtaacattctattctttttttctgccggtcacaaaatcaccttatgaaattgacagccaaattggctgcaacaggcagtttgaaaaacactgttttggggtttcaaggaccttcttcagaggttaccccagggctctgtttagtgcctctataccaggggcccccaacccctgggccacagcctcgtatctaccagtccgtgacctgttaggaacctgactacaccgcaggaggtgagtggcaggcaagtgagcattaccgccaaagctccacctcctgtcagatcagcggcaggagcgtgaaccctatcagaaactgtgcatgtgagggatctagattgtgtgcttcttgtgagaatctaatgcctgatgatctgaggtggaacaatttcatcctgaaaccccctctacccctgtccatggaaaaattgtcttccatgaaattggtcactggtcccaaaaaggttggggagtgctgccctataccataactattgaagttcttcatttatctgctttacatgttagtttccttgaaaaaagggttttatggctgtaaaaattttagagctactatgtatggaagagaggtttgtgctggcataaatcttctcaggtatcatctgtgtagaaaatttcaacaacttgtgtcttaaggcagaggtcagcaatcttaagggtcagatggcaaatattttaggcattgtgggacatacagttcctctcacaattcctcaacaatactgtagtagctcagaagtagctaatagacaatatttaaacaaatgagtttgactctgttccagtagtcattttcaggacactgaaatttgaatttcatatcattttcatgtgtcatgaaaatcttcttttgattttttttctaccacttaaacatgtaaaaagtattcttagcttgtgagctatacaaaagcagatagtgggccagttcatgggctgaactgttctgactcctggtctaagggagactatatatgttatatttgaggttctcaaagtaagatgtaggttagaggtctatagaaagttcataattgcttttgtaaaaactagattgatttatttgagagggagagagaggtggagtttcactgtattgcccaggctggacttgaactctgggctcaagcaatcttccagcctcagcctcccaagtagctaggactacaggcatgcaccaagcccagctcctagttcctctttgcactcagtttcatcttctaccctcagcccctggcaaacactggtctgatctctgtgccctttccagaaagtcatataaatggagtcatataaaagtcatatcagtggggccgggcatggtggctcacacctgtaatttcagcactttgggaggctgaggagggcagatcacctgaggtcaggagtttgagactggcctggccaacatggtgaaaccccctctctactaaaaatacaaaaattagccgagcatggtggtgggcacctgcaatcccagctacctgggaggctgaggcaggagaaacacttgaacccgggagagggaggttgtagtgagccgagatcgcgtcattgcactccagcaattgtgaagcagtggttaaggttcattcattattttacatatggatgtccagttgtttcacatttatcaaatttcttttgaatcaccttggcacttttattgaaatcaattgcttatgtttgtgagttttggattctactgttttattgatctgtgtgtgttttcttaggctaataccccactaccttagttatgatagctttatagttaaatttaaaatcaggtaggtccagttgagaagatttttagaactttgactttttaatttcttgttaccatacttttggaaggagaagactcataattttattcaggttttcaaaggtgtcttctacccccaaatacttaagacttcagtatatagttaatatatctctgtcttgaattatgtggtggtgaggttcatgaaaagtgacaggtaaaagttgcaaccaaaattaatttccttataagaaaggacatattttagtaggttgcacagaaaagtcaatcacgtcctttttaccgtgttgcattatgaggtgtaatcaaaatgtgtataggggaaaatgaatatgttaagatgtttattcaccaaaaaaagtcaccgaaagtgtcaattaggttgtgaaagatactagtttacaatgtgttattctccatcttcattagaagagttcttccatttgaaacctatgtcatgttctgtgatatttcagagttagtatctttatgaatcttaggggcatcattaaatcattatgtttcttctaaggaaaggcatatggtagtagttggtcatatttctacctttttcagtggggcctatagggcccctttactttgttttaaactgcactgatagctttttactggttagcccagggaattatccctacagtgtaaaatgatgtttctcatcatgattttcatttaataatttaaaaacaaaataacaggaaacacatgtttctagagaggaagtgtggaatacctgactagtagagggtcaggtgacaattgcgaaggtagaaactgactaagttttgaccatgttgcaaggagtatcgttccagttttgttgggtcaccttgcagagtttttaaaaaaaatccttgggttttttttgtttgtttgtttgtttgttttggtagagatgctgtgttgctcaggctagtctcgaaatcctgggctcaagcagtccttcccagagtgctgggattacaggcatgagccaccgcacccagcatcaccttgccgattttgaaactacacttccagggagagagtaatacaatatcttggcagactatttttaactattgttccaaaattaatctgtttttaaaaagtaaagaagtgtggatatctttaaaagatactttaattaggctggtcacggtggctcatgcctgtaatcccagcactttgggaggccaggatgggtggatcacctgaggtcaggagttcgagaccaacctgaccaacatggagacactccgtctctactaaaaatgcaaaaaattagccgggcatggtggcgcatgtctgtaatcccagctacttgggaggctgaggcaggagaattgcttgaacctgggaggcggaggttgcagtgagccgatattgcgccattgcactccagcctgggcaacaagagtgagggaaactccgtgtcaaaaaaaaaaaaaaagatactttaattatatttaagttggggaatacttttgttttatattttttcttgcactaaatatgtaacctttaagttaataacaccagaaaatttttactttatagttttagaactgcttaattgaaataaaatgttaggatactttgaattagtcacttattttggcatattttaaacagtttaatgtactgcttggggttgtttctcaattgacttgatatttttaaactgtattttttaccttgtaccatttattttgcaaaataaatttctattttggcttatagatgtatttttaatattaaaggggattattggtgtcagccataaaattgtaattaagtcttaagtaggtatggtttttttttttttttttttttggagacggagtctcgctctatcatccatgctggagtgcagtggtgtgatctcggctcattgcaacctccgcccccccccccccccgggttcaagtgattctcctgcctcagcctccagagtagctggcactacaggcgcatgccaccatgcccggctaatttttttgtatttttggtagagatgggatttcaccatgttagctaggatggtctcgatctcctgacctcatgatccgtccgcctcagcctcccaaagtgctgggattacaggcatgagccaccgtgcctggctgattatttttatagagctcttgttagcgtaatttctggtaatgttttatggaggtgacttaattcccatcataaaaatatcccatcttttctgtgactagcaagcagttattgcctttataacttttttttaccataaaaagataaagtactattgataattactcctaattaactcagaacttttttgttttacacacattaatatatacttccatgggaatagtgtcagagaacatcaaatagggaagagattatgattcagagtggtctttatattcctattctagagccacagaaaatgttcatctccctttagtttttgcaggattgcctctaacactgatgatattccactcatattcttcctgcacatgccttctcatactaacagtaagtcacacaatctcaaataagtttcattatacatgagaactcagttgaatgcttgcctttcactggcatctcttgatcctcctccccctccttatggtatgcactgaacttctagtaggccatatgtgttaagtagatcataggagtgctatgaaaataaagtgaaatgatgaatatgtaaaagccttcacaaaattatagtagtgttttctgttgatttttaagacaaaagataaatactatgtatgtaaaaatttcctttcagaaatctttgttcttttttttgttttgaatgttcaatatgcttagcacagggctctagttaacacttttggcagttcttaatgtgggactgactgatgattgtcctagaactgtttcagtgttaactacattctattaatgttactttaaaacattatttaattaaagaaacatgaagtggcaacatactgattcatgtgttcagtaagcaattcatgggaaagaggtaagctttcttaataacagtagaaagactgttccatttataggaaaactggtaattatgacttgtgttttggtatttaaaagctgtggttggccgggcgcagtggctcacgcctgtaatcccagcactttgaaaggccctggcgggtggatcatgaggtcaggagattgagaccatcctggccaacatggtgaaaccccgtctctactaaatatacaaaaaattagctgggcctggtggcatgtgcctgtaatcccagctactctggaggctgaggcaagagaatcgcttcaatcagggagtcggaggttgcagtgagccaagatcgcgccactgcactccagcctggctacagagcgagactctgtctcacaaaaaaaaaaaaaaaaaaaaaaaaaaaaaagctgtgattaacatttgctttgtcattcatccaaaactacattggtgacttttgtattgagtcatttcttagggcaacaggtattcatgtattcagtaaatatttgagtgcctactatatgccaggtagtgatctaggtgcttagtagtacacttgaaaacaaaacaaaggtctctacccttatgtagctgctgtccagtggaggggtgtgtgtgtattgggggatggggctgagaaaccttagacatacagaaaggaaattatgtagtatgttcaaaggtaataagtgctgtggagcaatgaaagttaaacagtttagggctgggatgggggtaggtagcaatttaaatagggaggtcagggtaggcctcacttgagaagggggtatttgaacaaaaatttgagaaaggaggaggaggcatttcagataaaccaattagttcaaagattctgtgtcgggaatgtgccttgcctatttaagaaacagcaggaggccaaagttgctggggcaaggtagagactaggggattagggaaggatctctttcagttatctaggccatattggtgatagcagaaatactgagaagtagtcagattttggatgttttgaaagtagattcatcggggcttggtggctcacgcctgtaatcccagcactttgggaggccgaggcgggcagatcacccgaggtgaggagttcgagaccagcctgaccaacatggtgaaaccctgtctctactaaaaatacaaaaattaggtgaaatattgaaggagatgttttgattgaagtgattttaagagagaagaggaggggaagtaaagatggtgaggaattatcctgtaaaggggaacagagaaatggggccagagctagtgaggaaagtggggtcaataaatttttaatgataagaaaaagaagagcgtatgatgataggaatgagccattagagagtaaaacgtttcaggagggagagagaagaattgctgaagcactgtcttagaagaggtaagagggaaagggatttagtgtataaataggaaggattggctttctataggagcatctatactttatgataataggccattaagcagagtatgtggttagaaatgctgctaggaaggtcgacgtgattggtggagtctgtacacgttctgttgcagttgctttggtttttttcagtgaagtaagacttgaggttatcagccgagaatgaggattggggatatgtgaaagtagctccgcagcagtatgggagcataaatgaactggagacaaatagtaaaattaatgggcaattttttttcctttaagagatgggggtctcactctgctacccaggctggggtgcagtggcacagtcatagctcactgcagcctccaactcctgggctcaagtgattttctcacctcagcttcccgagtagccaggactataggctaatggcaatattaagttattttatgagttgtctagacagcattatgagtctcctaactttttggtactgatcttcagatcagagttaaatgtaacttgcccaggcaatttaaacactcaatatgagtcattttcatttggactcaaacatggaatcattgggaaatagaacatgaatttattactccttaatgaagtacctgccactatcctgccatgaatgtaggctaaatttggagtggtctggtaactgcttttcttttaaaaaaattttcttcatcttttctgtatcaaatacttactggtttttctatgtagaataacataatctcatcattactttctttcaaacactctccaaatttgacttgtctttgctcatgtttttcctaccacctgaaatacagatttcctcccccatcccaccctccaaaccttccagacttacctcacctaccgtttattgtaggaagcttttcttaacctctttccaagtcctagtttgatgcctctgctttgtgcttttgtagaatcccagagtttaccttgttttactcactatatcgtattgtggggtttttttgttaataggtatttctttttcctctagacctgcactgttcagtcatactttctgtgatgatggaaatgttcagtgtcgtcttaatatggtagacagtagccgtatctatgtggctactgtgcacttcaaatttgcatagcaggactgaggaactaaattttatttcattttaattaatttaaaaataactagcctgatggctagtggctgctttaatcagtgcagttctagaccccttcaaagtcaagagtgtgtgggattcatacttattctctcgttgcttaacaataatactgtctaatacttaaatgactgaattctttttcggtactcttagtctgcatagttataactcacctagaatatggcagttttgttctctagatttctgtctttaaccatggcttttcagtttgttttcaagattatgttgatttacacagcactgagttcttcagtcctgcgaagttagcgttttggttggatgtggtggctggcatctgtaatcccagcaatatgggaggctgaggcgggacgactgcttgaagccaggactttgagatcagcctgggcaacatagcgagcccctgtctctacaaaaaattagctgagtgtggtggcatccacatgtagtcccagctacttgggaggctgagggcgggaggattgcttgaacccagttcacggctgcagtgagctatgatcacaccactgcactcctgcctgggtgacagagcaggagtcaccctctcaaaaaaaaagtgtattttttcccctttaggactgaaaaaattgggtgttacaagattacctcaaggactggtctgagaactggggatggtaaggaagaaactcaagtggccagcctctggtttgtgggggtaggtgggcaatttctgtttcaaccaaagcagttctacttcataaattaatatattggaattgtgcttgggatttcatttggaggggaaaaaagtcttctaaacaataacactgttaattgaagagacaaagcatgcatatggcagcacgtgattaaccaccaaagtggataacagatcaagaagacatgggaagttgttatgggctagtgaggtcttgatggaagttaaggtttaatttaggtaggtagaaggaagacaaaaggatgtaataggcagtgggaatagaatttgcaaagaattggagttggaaatacatatgttagttttggttaagaaacaatgagctgattatagttgagcaaattgtatgaaataaaatttgacaaaattagatgggactggattgtaggaaaaagtgataggctgacaaaaagtgcactttatattgcatgcaatgagatgaaatactcggaggtattttggttgttacaaagatggaggaagaggacactattagcatttaatgagaaaggatcagggaagtcagccatcccacaatgttcaggacagtttgcacatttaagaattgttccaaatcaccacatgacctagaatgacttgctggactttgatgatgtaggttaaaaaaaaaaagtgatcataatgatgtgagcatagacagtaactccattttgtgtatgaggcacttttttgttgttgttgttgttgttgttgttgttgttgttgttttgagacggagtttcgctcttgttgcccaggctgaaatgcagtggcaccatctaggctcactgtaactgaggcacctttaatatacactaagattttcaggaattcagttaccacaatattggaggaatgttaaagtttttgttcagaactttatcaggagtactccacccctttagaaaatcacgtaattgatggcagtgtcacttgtgctgtattggtgacgcaaatactcttcagtctgcagttgtaactgccacagtcacagtgcttgttgtatatatgtacaaagtaccatcagtgtgttatttattatagtaatagcctaaatattataaaattgcactaaggtagattatctctgaattacatttattttttatttttatttttgtgacggagttttgctcttattgcccagggtggagtacaatggcgtgctcaccaaaaccttcgcctcccgggttcaagcgattctcctgcctcagcctcccaagtagctgggattacaggcatgtgccaccatgcccggctaattttgtatttttattagagacgggggttttccatgttggtcaggcttgtcttgaactcccgacctcaggtgatctgcccgcctcggcctcccaaagtgctgggattacaggcatgaaccactgtgcccagcctctgaattacatttaagggtagtatagagaatgtacaaaatagttgttgtaaaaaggagaaattgatttctgttttcagtattggaagactgggttattcagatgaaaacaattaaaattcttggaaacagtattaaaaaaaaaaaaaaaaaaagaaaacgtagagcagttgcagagctgaaaagatagtggggagctgccaggccaaattctaggaataaacaagaatccaaataaataagtggatgaagtagcttttgccctaaaggcagttgccaatctgtacaagttgggctttggttttggtggaccattggggtgaagaggacagaaatcaagtcctagagttcatctaaagtgacagtcaaaaagaataacctcagttttagatgggaccccagtgactctatcgttaggttaagggtgaaacaactgaactgtctacacactcccatatccatgtgattgcagggaaggttgaatggagcaggaggaggaaaaggaaattaagaaaaataaacctttcaagttgtgaccacagttttagccttcacagtgatttgccttgagggtttgctctgtctgggtggtccagggaccctccatgataaatgaccctccagccatttatcatggttactggtttcagactagtattacttccatggcctggcagatggaaaggcaaatcacgtgtggagaaaggcctttcattctagggaattgcttccagtaatttttcaaggacaatgaacagtacattgtcacaagtaatcaaacatagtaaaaataaaaaaattagcaagcaaaattacctgagagatatataatatgtaatatatatgtagaattctttcagcaatgtaattaaaatttgtgtgggtttctttatgtatatatacctaaaattacgtttttacagaggaaaaataataaccaagcatgcatccaattggcactgtgaataagaagagagcagaaacagatctataaaatcttcaaatgttgcaaatatcagatggatttaaaacaatcactgttaaatttaaagaattggagacaaacttgaagaacaaaagactcttataaagtgacttagggccaggcacggtggctcacgcctgtaatcccagcactttgggaggccaaggcaggcggatcacctgaggtcaggagttcaagaccagcctggccaacatggtgaaactttgtctctactaaaaatacaaagattagccacgtgtggtgaggtgcacctgtaatcccagctgctcaggaggctgaggtgggagaatcacttgaacctgggaggtggagattgtagtgagccgagatggcaccactgcactccagcctggcctataagagtgaaactgtgtctcaaaaaaaaaaaaaagttactttggttcatgcctgtaatcccaatactttgggagactgaggtgggagggtcacttgaggccaggaatttgggacccgcttgagcccaggagtttgaggctgcagtgatgtatgattgtgccacactgcactccagcctaggcaacagaatgagaaactgtctcaaataaaggaaaaaaaatggctgggcacggtggcttatgcctgtaatcccagcactttgggaggctgaggccgacagatcacgaggtcaggagattgagaccatcctggctaacacagtgaaaccccacctctactaaaagtacaaaaaattagctgggcatggtggcaggcacctgtagtcccagctattcgggaggctgaggcaggagaatggcgtgaaactgggaggtggagcttgcagtgagctgagatcgcaccactgcactccagcctgggtggcagtgcaagactctgtctcaaaaaagaaaagggggggaaaaacccaacttaatagatttgcaaaaaaccaaatagaaattccagaagtgaacactttaccaaatatacctaagagattatgcctagctgaagaaagagttcattgcctgggagacaaggcagaagaaactgtttagagtgtagcacagaataaaaaagaaaatattgaagagaggtaaagagacatggaagacagaataagatctaatttctttaatcagagctctggaaagagaggagaaagaatggtacagaagtaatatttcaaaagatatttctggctgaaaattttatagatccaatgagaaaccagttgattgatttaagaaggttaatgaatttctagcaatataaatagaaatctacacccagacaaatcataggaaaactgcataaacccagatacaaggagaaaagtcttgaaagtagccagagagaaaaaaagatgtttttcaaagaagcaactatggactgatggttgacttttcaatagaaaattacatatattctcaaaataactgccaatctagaattctgtaattagcaaagaattatccctctacaatgagggtaaaatacttagttgaacaaactccatcagctctttctaaaggaaattatgaagtatacattaatacttaaggcagaaagattctagataaaagtctgaggtgcaaaatggaataaagagcaaagagagtggcaaatatgtggatgtattaaaagaaacgttgactgtataaagtactagtaagaccttaattaaaatatgtgacaagaagctgggcatggtactttgagaggctgaggcgggcagattgcctgagcccaggaatttgagaccagcctgggcaacatagtgaaatcccgtctctacaaataatataaaaattagctgggtgtggtggtgcatgcccatagccccagctactcaggtggctgaggtgagaggatcgcttgagcccaggaggttgaggctgcagtgatccttggtcgtgccactgcacaccagcctgggcaacagactgagaccctgtctcaaaaaaaatatatgacaggcgaaggccgggttctaagacctttgtattgtcagagagaaaggtagaaagtattaattgacttgaccttgataaattatatgttttaatttctttttttttttttttttttttgagacggagtctcgctctgttgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggcctatgttttaatttctaagttatcttctaaaaatgtagaaaccagacttttaacttctcaaccaacagaagataacaaatgattaataaaaattaatcctgaagaagtgaagaaaagaaagaaccagtaggacaagtagcacaaagatgggtagatttaaatctaaacatatcaccagctacattaaatacaaaatggattaaattattcagttaaaagccaaagattgttacactgaatttccaaaaaaattcagttatatggggtttataaggaacatatctgaaacctaagaataaagaagatcaaaagtaatcatcacaataagacataccatgcatattctagcagacagtatggtacagttaatatcaaaggtggacagtaaggcagaaagcattattggcagaagagtcacctcaaatgataaaatgaccaattcactgtgaagatttaatagccttagtaatatagtataacctgaaatatagctttagaatatttatagcaaaagttaaacaaaactacaagaaatagacagatttctcagtcttaatggggtatttttaaacagctctttaagtaactggtataagaagcagacaggttagttaggatataaaatatttgtataacacaatgaacaagtttaacccagtgggtgtatagaacccattctacccaacagtggcaggctacacattcttttcaagcatgtaggattttggggggaaaattgactgagtaataatgttgtaaaacaagtttcaacaaatttcaaaggattgaaaccaaaaaagcatttttttctgtccattttcattaaagatctctatcaatagggtaatttttaaagcttcatgttagaaattgagcaactattaatacttggaaataatctggtcaggcgcagtggctcacacctgtaatcccagcactttgggaggccgaggcaggcggatcacgagggctggagttcgagaccagcctggccaacatggtgaaaccccgtctctactaaaaatataaaaattagctgggcgtggtggtgcatgcctgtagtcccagctacttgggaggctgagacagaagaattgcttgaacccaggaggcagaggttgcagtgagccgagatcgtgccattgcactccagtctgagagagcgagactccctctaaaaaaaataataatccatgcatcaaagaagaaatcacaatggaagttagaaaataccttgaactagatgattaaaaatttttgattgatcaaattccacaacttgatatatcttagaccattgaaagtgagagaatcaaatgttatgtctttagatacgaagtttcttgacacccctccctgcctcccccaaaaaagaaatacctaccacctatgaaagattcttgattggaaaaaaaaaaaataactgagctggagtttcattaaacttctagatctactgggaaattcacagtcatttgatctttcaatgagtcataattgttttgctgtggatggtcttgcctcgatgttgatggctgctggttcatccctggtggttactgaaggataggatggctatgccagtttcttaagacaacagtgaggtttgcgacatcagttgacttcttttcacaaaagatttctctgtaccaggcgatgctttctggtagcatttgaaccacagtagaactgcttttaaaattagagtcagtcctctcagatctgccagtgcttcatcaactaattctatgtaatattctaaatcctttgctgtcattttaacagtgtttgctcacagcatcttcagcaagagtagagttcatgtcaagaaaccactttttttactcatccataagaagtaacttcccatttgttcaaatttcatcatgatattgtagcaattcagtcacatctttaggttccactcctaaatgtagttcttctgctgcttctactacacctacagttccttcctccattgaagttttgaactcaaagtcatccatgagggttagaatcaacttcttccaaactcctgttaatgttgccagttttacctcctcccatgaattacaaatgttctttatggcatcgagaaaggtgaattctttgcagaaggctttcaattaatgttgcccagatccatcagagtaataacgatatctatggcggctatagcccaatgaaaggtacttcttaaataaggaatgttgaaagtcacaattacttcttgatccatgggttgcaagatttatgtgttagcaggtgagaaaacgttcatcttcttgtagctctccatcagagctcttgggtgaccaggcacattgtcaatgagtagtaatgtgttagaaggaatctttttttctgagcggtgggtctcaacagtgggcttaaaatattcagcaaactatgttgtaaaagaatatgctgtcatccaggcttcattgttccatttacagagcacagacagtaaattttgcagaattccaaaggccctaggatttttggaatgataaatgagcattggcttcaacttaaagtcaccagctgcattagcctctaataagagtcatcgtgtcctaggccaggcgtggtggctcatgcctgtaatcccagcactttgggaggccgaggtgggcggatcacctgaggccgggagttcaagaccagcctggccaacatggagaaaccctgtctctactaaaaaaatacaaaattagccaggcatggtggcacatgcctgtaatcccaggtactcaggaggctgaggcaggagaatcgcttgaacccgggaggcagaggttgcggtgagccgagatcatgccattgtactccagcctaggcaaaaagagcgaaactccatctcaaaaaaaaaaaaaaaaaaaaagtcatcctgtcctttgaagctttgaagccaaccattgacttagggaaatgttgtggctggtttgatcttctatccagatcactaaaactttctttatatcagcaactaggttgttttgcttgcttgcttttcttaaactatttttcttttaattcttagagggtctcactgtgttggctaggctggtctcaaactcctggcctcaagcaatcctcccaccttggcctctgaaaatgctgggattataggcatgagccgtagtgcccggcctgttttgctttcttatcattcatgtattcactggagtagtgcttgtagtttccttcaaaaactctgcctttgcatttacaatctggctgtttggtacaagaggcctgcctttcaacatgccttcttcactaagcttaatcatttctagcttttgatttcaagagagagacatgtgactcttggtttcacttgaacacttagaagtcattgtagggtttttaattggcctaatttcaatattgaaatctcagggaataggaggcccaagaaggggttagggcagtagctggtcagtggagcagtgagaacacacacaacatttactgaagaagttcactgccttatatgggtgtggttcgtggcaccccaaaagaattacaatagtaacatcgaagatcatagaacactaaaatagacagcataataatgcagaagtttgaaatactatgagaattactgaaatgtgacagagacatgaagtgagcatatgctgttggaaaaatggtgccaacagatttgctcgatgctgggttgccaccaaccttcagtttgtaaaaaacaaacaaaaaccattatccgctaagagcagtaaagcaaagcacaatatgatgaagcatgcctgtacagtttatacttcacagtcattggttaaatttactcattcctttaagcataaaaaataaatatttagcacctttgtgctgagttgtatgctaggcactgggatatagaaatgaatgatagtttgtcttcaaggagcttacagttaagtggtagaagtagataaataaagaattaaggacagtgtactgtgatgaaagtgtgccccagatgcagaagaatcacagaaaaggagcacaattcaatatgcttttaggggatggaagacagatttcctaggaaaggagatacttaaactgaattttaaatacttcagatatacaaaatctttataaagaagagtaattaaaccttatttagagacagaccacctaaataaatggtacataccctgtactgagtgtggattggaagactgagtatgttaaaaatgttagtatttctcaaattgatagtggatttattgaaatccaaatgcaaattcctgtaggtttttctatggaacctaacaacctgattcaacaatttctgtggaagctttaaagaggcaagaatagccaaaatatactttaaaaacaataaggtaggaagacttctttaattcctttttccactcggcaaatttttaatgctctatggtaggcactgctcatcctggttattacctttgtgaatgtgagatctggctcccacccaaagctcacattcagatccacttgtagtggtatctagggttttgaatagaacacttcctgaaatacaggttaagtactgggactaccaacaaacaaacttacaacactaattttattatctcatatttgtatttacaatttttcttgatcatattttttcatacaaactttaattagtttctattaatttcttttaacataggaagcttagaaatagacacactttgcttataatagatctttaatattatgtcagttggactatggctttgttagaaaaggagaggagaatctactgctgagtgctgtccaaagagcagttacccaactgagcagagagggaggatgaccttgagggtctcacaggcatgggtttttaggcccagcctgtggagcgtctgagggacttataaaaggaaaagggacccaggtgcagtggctcatggctgtaatctcagcactttgggaggccaaggcggttggatcacttgaggccaggagttcgagaccagcctggccaacatggtgaaaccccgtctctactaaaaataccaaaattagctggctgtgatggtgcacgcctgtaatcccaggtactcgggaggttgaggcccaagaatcacttgaatccgggaggtgaaggttgtggtgagctgagattgtgccattgcattctagcctgggcaacagagcaagactcagtcttaaaaacaaacaaacaagcaaacagaaaacaacagaaagaggagatgaataacaaaatggcaggtaatactaaataatatggatgaattttatattcttacatatgggtaggtggagggacacctgctcttgtggcagattagttgggaaaggatacacttgctggaacaatgcctaccatattaaataaatgaagctagacttccccatctgttatgcataaaaatcaatttcttgtaggttaaagattcaatacaaaaggggaaaccctgaaagcttttagaaaaataaagggacattatttaaggcagagaagaatttcttaagactcaaaaaactagccataaaaaatggacaaatttgactacattgaaaatagaaacttttttcattaagccccattaagacactcaaaaggcaaggcaaggaatagaagatacttataacacacactattagttatctattgttgtgtaattaccgaagacttagcagtttgaaataacatttctctcacagttctgtgggtcaggaaactgagagtggcttaattggatgtttctggcttagggccttgaggctgtaatcagtcaatggctgcagtcatttgaaggcttgactgctttgtgctccctcatgtggatgtagcaggcctcagagctggctttctagaagggagtaagagaaaacacccaagatggaaaccacagtattttagtcttggaagtggcaccccatcacttctgccatattctttttgttagaagcaaggcaataactccagcttacattcaaggggagtttgtacaaggcaccaccaggagggtgaggatcattgggagccagtttagaggctacctactacaacatgtaaagaatgaactggtatgaaaaatatacataaaaaattcctatagatttctaagacaaagacagaacaccaaattggaaaaagggcaaaaaatcctgagcaggcatttgaattaaaaaaaaatttaaatgattaataaacatatgaaatgacccttaatctagttaacaattagggaaatagaaattaaaaccacaatgagagaccatttcatatagattggcagaaatgaaaaaatctgacaatattaaatgttggtgaggctatgaagcagtctgaactgtcttccactgatggatgggaatgtaaattggggggaaaaatcctttggaaaataataggatatcctataaccagcaataccactcactcttaggcgtttgccctagactaatggttcttgagtatgcatcaaagtcatctggagggcttgttaaacccagctgttggggctacccttagagtttctgattaagtaggaattgggtggagcctgatagtttgctttgcatttttaacaagttcctgccgggagtgggctgaggggtgggggggtgggatgttggtgctgcttgtccaggaatcatactttgagagtcagccattgctttagacatagagaaatgtgtgaatatatgaagtaggaagcatatatataagattgctcataacagtattattcatattaaccccaagatagaaaaaaagttcaagtatcctcagtagtgcagtagatatcattgtgtgtttacttatggaacaaaaaactagttcaaatagctaaggaaaaaaaccacactacaaacagctcctgcctgcatcaatatgtatgaatctcacaaatacattagtcataccttagaaccaagtcacaaaagattaagtgcaacatgattctattcgtaaacagtttaaaaacaaatgcagttaactgtattgtttaggaatacaaatataggtactagaattataagtagagatactagaattataagaaaaatcaagggaataatgatgataaaagtgagtgatagtgactacctcagggaggagagaaagtggatgtgattaaggacaaatgggagacttttaagataattggcaatgctatttcttaccttgggttgtggggtttgctttacagttatgctttaatggaacgatgttttgcaaacttctctacatagctttataataaagggaaaatttatctcatttaattttcatctctgattactagtgaggttgagtatgttttcatatgttagcctttgggttttttcttaaattgtttatagacatgccatttgtagattatgctatttgtttttagaacttatttttaggtattctgttatggatagtcttctttatccatcatatgtcaaatatttccttctagtccttttttatttgtatttttatttatttttatttcttttttaagaggcaggcctcccactctattgaggcctaggctatggatagtcttctttatccatcatgtcaaatatttccttctagtccttttttatttgtatttttatttatttatttttttaagaggcaagcctcccactcttttgagacccaggctggagtgcagtagtgtgatcatagctcactgcagcctcctgggctcaagcagtcttctagcctcagcctaagtaactgggacaaatgcacaccactgtgcccaactaatttgaaaaagaaaaaatttgtagagatggagtcttgctgtgttgcccaggctggtctcgactcctggcttcaagtgattctcccaccttggcctcccaaagtgctgggattacagacgtgagacactgttcctgccctagcctttccttataaagacatatgttgttgttactgtttatgtggtcagatttagcagtcttttcatttatttatttattttgattttgattttgatttttttaagaaatctttcctggctgggcgcggtggctcacacctgtcatcccagcactttgggaggccaaggtgagcagatcatgaggtcaagagattgagaccatcctggcatggatgaaacatggtgaaaccccatctctactaaaaatacaaaaattagctggacgtggtggtgtgtgcctgtagtcccagatactcgatagcctgaggcaggagaattgcttgaacccgggaggcagaggttgcatgagctgagattgtgccactgcactccagcctggcgacagagcaagactctgtctcaaaaaaaaaaaaaaaaaaaaagaaaaaagaaatctttcctatccagagttaaaagcatattctctatattgtcttctgataaattgaaaattttaaaaattttgtttaggtatttaagcagtctagtatttgttatatgaagatgtcttgggggatgttattttcccaacaccatttgtacatatgtgcacagtctttttctcacttatttgtaaagcaacctctcatttctaccagattcccatggaccgatttctgggctacttcttaacctgtttgtctataacctgtttgtccacattactactttatatctttggttttggtaatgccagtctcttctctttgtttatttgcggcaaacatatgctcattcactcttccacgtgaccattagaagaattagattgtcaatgtcctctaagtctaggtatttggttaaaaaaaaaaaaaaagaattaggttgtcaaattctgtaaaaagtactattgggattttcattgtgattgcattaagtttatatgttaatgtagggacatatatattagagatttattatgatactgagttttccaattcatgaacatggtttttatcttcatttgtttataggtctcttccccccccacccctttttttttttttttttttgagatggagtctcgctctgtcacccaggctggagcacagtggcacaatcttggcttattgcagtctctgcctcccatgttcaagcagttctcctctcagccttccgagtagctgggactataggtgcacaccaccatgcccggcttatttttgtattttcagtagagacggagtttcaccatattggtcaggctggtctcgaactcctgacctcaggtgatccacctgcctcggcctcccaaagtgctgggattacagacgtgagccaccgcgcctgggccatgtgtttctatttttagtagagacaggatttcaccatgttggccaggctggtctcaaactcttgacctcaggtgatccacccgcctcagcctcccaacgtgttgggattacaggtgtgagccactactcccagctctgtttttttttccttaaaaggtttatagttttctacataatgttttcatccttttggatgtggaggattggtattatttgcgagaatctttttgtgctgttgtgaatgagatgtttagaaaaaaatatatttttttactggatattcatatacagaagaatgaaactagacccccacctctcaccctatacaaaaatcaactcaaaatggatcaaagacctacctgtaagacccaaaactacaaggtgaaaccacagtgtggtatcatctggccccaggtaggatggctgctataaaactgtagaagacttgggaaatgcaaatcaaaaccacagtgaggtattgtctcatcccagttagggtggctgttataaaaaagacaaaaaattaaaaatgctggtgaagatacagaggaaagagaactcttggacgctcttggtgggaatgtaaactagtacagacactgtatcagtatggaggttcctgtgatctagcaatcccactactggccgtttacccaaaggaagggaagtcagtacgtcgaagagacatttgcatccccatgtttactgcagcacactattcacagtagccaagatatggaatcagttcaacaacagaagattgggtaaagacaatgtggttgtatagcatccgatggaatgctattcagccgtaaaaaggaaaaatcctgtcatttgcagcaacatgattggaactggaggacatcacgttaagtgaaataagccagcaaaagaaagttaaaccctgcatgttctcactcttatgtggaagctaaaaagagttgatttcatagaagtaaaaagtataacagaggatactcgggctgggaatagtaggggtagagtggaagctctggggagatttattcatggatacaaaattacagctagataggaggaataagttctagtgttctgtagtagtatagggtgactatagttaataatatatagtttcatatagctaggaggaggatattgaatattcccaacacaaagaaatgacgaatgtttctcctgtctttagtgagaatgcttctgttatgcttggtgacttacagactttttatagataccctatatgaagtttaagaatgtttcttccatttctagtttgctgacagttgggcatttttaaaaatcctaaatggttgttgaattttgttagatttttttcgtacatttattgaggtgatcatgtttttttcctacattgatctatttaggtaagtaattgtattggtagacttcctagttttgagtaagcaactcttgtgtcactggatacattttattgtatattcttttaattgattgttgaattaggtttgcctaattcaggtttttatttagggattttacgactgtgtttacaagtgagctcagtctatacctctctgagctgttccatggttttggtatcaagagctgattcgttttctagagttttctgtgattttggccagtttttatatcagaccaaagaaaatgtttctcgaaagtaagttcattaggctttaagtaaccactgggcccattctttgactttcaacttttctgagtgagttttagatgtggatgttatatacagcatttgttcagggctttggtttgtgagctgagtttctttttttatttttttaatagatgagcttatctcatttatattttgttgataaggcagttcttccacattattaattatatttttctttgggggctttgtctcttatgggtatgtgtgtgtttcccatctaatagtttttgatgattttttataaatcttattctcttactaccttcatagatttaatatctgtaagcctccatttcttgatttgtgaaaataatcaggactattttaactccttgctaataaaggtaagaaaatcagtacacttatacctccctttccccagctaccattaaaaaaatttttttccatctcttcatatgagatataaaccttgtcattgaaaaaaatgcatttattttttactttatccttaagtctcagagttacatgtttgaatgggattaatattcacctttaattctttaaaaccatgacttcttgatttctaattctgttttatttatcttttcatcagcttgattttcctatcaagtagttctgttttttctgccaagaatggcttccttttttcttcttcagccatttctttctgtttccaccgtggccttcatttctgaagtggtctttttattaccttccatttctttcttgaaccctgctaactcacttttcatttcctatttcttttttttgagacagggtctgggtctctcactcaggctggagtgcagtgttgtgatcttggctcactgcaacctctgcctcctgggctcaagccatcctcccatctcggcctctcaattagctgggactacagacatgtgctaccacacctggctaattttttgcatttttttgtagaggcagggtttcatcatgttgaccaggctggtctcaaattcgccttggcctcccaaagtgctgggattacaggagtgagccactgcctcctgcctcatctcccatttcatctgttcttcaattttgttttttcagtcatttttttcccctaaaatgcatttgatagtgatagatggggatatatcagagacttcttttgtttccttgactaacttctggtgtttatctgtctttgcctattatgtttcttctctcatcccccactccctgttttattggtggtgtggcgggtggggttgttaacgtttctcctcatagaccctttgctggattgtttctttttattactcatccttgttgtctttgagtagagtacttctatttgagcctgctctttgcttaagaatgttatggggggctgggcacagtggctcacacctgtaatcacagcactttgggaggccgaggtgggcagatcacgaggtcaagagatcgagaccatcctggctaacatggtgaaaccccgtctccactgaaaatacaaaaaattagccaggcatggtggcgggcgcctgtagccctagctactcaggaggctgaggcaggagaatggtgtgaaccctgggggcagagcttgcagtgagctgagatcgcgccactgcactccagcctgggtgacagagcaagactctgtctcaaaaaaaaaaaaaaagaatgttatggggaacagcagggagaataagcctaagcaggcataacttttttctcagacatcttgtctcagaaagctcgttcaccaaatctgttgtgttctctaccctgggaatacatcactcctccatttttaatgtcttttttccccttcctgccattttgggccaagccaatcattcagtcaggatgaagactccatccaggcaggagatgggtgaggctgatagtgaagttggacatcaaatagattcctgttcgatgatttgatttttaatttgttctgctcagctgcctttagcttttaaccttttatatcttatgttcaatagttattctcacaaaggaactggttcccaacataaatttaaaattaaaagtgatcagttctctcctctagcaaactgtaactgcttttattttatgtgacttgatctgatgtattgttttgttccctggatcatttaggggtggggaagtaaagacagggaggtctgtgtttcactttaaaaacaaaatctaaaccagataaaaactcgtaaaatgtcatatttcaggtcctctgtgatcttgccctttcctacctctctacccctgcccacgtctatcagccatccttgctgtgtactaaatgctctagcacagtatttcctaaagtttatattgtggatactgaatattcaaagtgttctcccaaagataggttccatagtttacaattttgggaaactttaaatgttcctccttgtctaacctgttgagtttctaagctcaaatgaggggggatactgtgttatctaaatcttatttgatttctgtattttacatattaagggattcatctaaaaagtatctgaattgatttggcaagcaggtgataccctgtactatattatatagctccccttttgggatttcataattggatattagcatagtaatggttgagaagaactgtagtaaagaagcttacctttgttgatttattttttttttttgacacagattctcgctctctcacccaggctggagtgcagtggcacaatctcggctcactgcaacctccacctcctgggctcaagcaattctgttacccttccaagtagttgggactacaggtgtgtgccaccacacctggctaattgtttttaaatatttttagtagagatggggtttcaccatgttggccaggctggtcttgaactcctgacctcaaatgatccacctgcctcggcctccgaaagtgctgggattacagcattagccactgcacctggccttacctttgtctaaattagaattgtataaacttccttgactatggaacattgcttgtgtgtagtatatgctaatattctttgaaacattttagggaaatgctgtttaataccaatcaaatccttgcattttttcagggttgtttcctgttgtttctggccctttgtcctagaatgctctcttccttcattttctccataccacccaattttttttttcaatggtttctgaggtgatacttaccctgctgggaggcatttggaaatattttgattgtcatgcctggaggatgctatttgggatttaatgccctcaaatcgaggaacacaatgcactaaaagtcctacacagcaaagaattgccccacccaaaatgccacttctacagtggccctccatatctgcaagttctgcatcctaggattcacccaactgcagatcaaaagtattaggaaaacagtaaaaagtaatacaataatacaaatacaagataatacagtataacaactatttatataacatttacattatattagtcattataagtaatctaaagatgatttaaagtatatggaaagtaagttatatgcaaataccctccctttttatataagagacttccgcatccaaggatcatggtgttgtagggtagagtcctggaaccaatcccttgtggatatggaggaatgactgtatactcctttggagaacactgaactcagcaacttttactcatcctttagaatttagttctggttatcttttccaaaagctttctctaagtacttttgttttcccctctaccccacaccctctcattttctctgcgtggtactctcagagtattttaggcacatcttgattatttgaagtaaccactttgtagtataggagcctataactgtatacctctactgtactgtgagttcaaatctttgagggcaaagacattttgtatccttggtatatgatatataattagtcttttaaaaagtgcatgttacatgaatacattaataaataagttgtacttttactttaaatcagaattgtggtatgactcttaaactgttgcttagactgcattaataatcttgtgcaattgtggaaaataatctcattttactctcataatagaaaattaaaatatgcttccttttgctcagatatgggtgacagctgttagagtagtgttttatttgggttttgaccttgcctgtacatcagaagcacctggagaacttcagaaggtacagatgcctggggccctgcaccagaggttctggtttttgagggtgaggccttggtatctggatttttaaaagcttccaaggtgatttttaatctgaagttaagattgaaaagtcatttagtgagacactgatatgtagcattttgggaagtcagtaaacttaagagtgagaagttactaaatcattgaagaacagtgaaaggagtagggagttttagcttagaaaaaaaaaatctagatggagtatgtgagttctctgagggctataggaatttgtccgtcttgctcaccactgtatcctcaactcctagcatgagcctgagcacatacagggatttggtagatatttattggctgacagaagaatatgtatcattaggtgtttaaaagatagtcattgtagaaccacagagattatccccaaaaaagttttatagtaataatgtgccacttaattttatgattaaaaataatgaactggatatttaattcatttatcatttcagtttattatttactttgtgccagtcactgaggattccatttgattctctggtcaaatctatgaagtttgtatcattcaaataatagggctgtgtcaagaaactgacacttgggggttaagtaatttgcctaaggttacaaagctaagtgacaagagctggaattcttaaagtctgtactcttaactgctatgccttaccataagtcttcaccacaaccctaagagttagatatggtttagaaaggttatgcagcttgcctaagggcacattgcttgagtggaagatttgggacttggacccagacattgtgacactagagtccatgctgttaactgccatgctattggaacccccaaccccctcgcttcctattgtataatgtacaaccatctgttttcatgagattattaaccacagtggacagcttgtgttgctctccttataccttttggcatcctcactgacttttgactgatgggttgaatttggattattgtcttgaatctccttagtaccctgggctgtggtagtcctagaaaataaaacatttctttactaggttctttttcttcatttctttttccttttagttaggattttaaattagaattttaataaacttgcttgcattaatacactgatatctgttagcttctgttattttaagtcggtagtctccagacttaaaaattttgttctctatcataaaaaaaatttgagcacattacccctagtagatatctgttttatttatgctatatgtgtactactgaagaaaatggtaatattttaaaaaatatgaacttgttagcatgaatttttttaaagctaagctaaaaatgaagtgagtttaaaattatgaagggtttttgctgatgtttcaagtttagctaatgtttcaagttacaacataccattaggccaaggttcgttattataatagtgtgtacaaattcatattttaagtagcctggataatttttttaaaatagctagtttcttgtcagaaattaagtaacctggataattttttaaaagccagtttcttgtcagagattattagattagggtttctcaacattggcgctgttgatgttttgaaatggatgtaattcttgcttgtagggttatgatctgcagttgagtgaatcctacgatgtagaacttgcagatatggagggccactgtaaaagtggcattttgggtggggcaataatactatgtattgtaggatgtttagcagcttccgtggcctctgctcacaagatgccagtagtacccccaagtagaaacatcaaaaatgccgggagacatttccaaatgtcttgaggggcaaaattgcttctggttaaggaccactagattagaattttttttttttttaagacggagttttgcttttgttgcccaggctatagtgcaatggcgcaatctcggcccactgcaacctccacctcccagattcaagcgattctcttgccacagcctcctgagtagctaggattacaggtgcccgccaccgcacccttctaattttgtacttttagtagagacagggtttctccacgttggtcaggctggtctcgaactcccaacctcaggtgatctgcccgcctcagcttccagagtgctggtattacaggtgtgagccactgcacccgcgctagatcattgtttttatcctgtattatggatgacaagcagcttgtagtagagtagggaaagtgttaactttgattttttcccctctagcagcaataatgttttcttcagtatgaagtttgagatctgtttgtaggaattaattttaagtcacttgtccattctataaggtttagttaaaacttggtaacataatccatacgtttacttaaatcaatatatgtgagtcatagtatgtcacaatgagataaatgcaagaggagagccactgtcaagtgttctgcagtatggaatgcccagccttcagcagacctcttgactatatgtgtcacatctctgatactaaccctaagttagggtgcctgtgtaaatattaaatgctgaggccaggcacagtggcttacgcctgtgatcccagcactttggaaggccgaggtagacggatcacaaggtcaggagatcaagaccatcctgactaaacacagtgaaaccccgtctctattaaaaacgcaaaaaattagccaggcatggtggcatgtgcctgtagtcccagctacttgggaggctgaggcaggagaatcacttgaacccaggaggttgcagtgagctgaaatcatgccactgcactctagcctggatgacaaagcgagactctgtctcaaaaaaaaaaaaaaaattaaacgagcatggtggcatgcatctgtagtcccagctacttgggtggccaaggtgggaggattgcttgaacccaaaagtttgagcctgcggtgagctgtgatcacactactgcactccagcctgggcacagagtgaaaccctacatctcaaaaaataaatattaaatgctgactttttctaagtttctagatgaacacattaactaaataatgtagcctacctatatccccaaaaaagagtcttcctatgggccctcaggtgtatgtgcatacctattttgtaaaccacttgtttgaccatcacattgtaacttattgttcaatttgtgtcactgcagccatattacccaaaagggaaaagtaattttagctctctgaactgacctccacctaaccaactcatcaggttaactgatgttctccatttcaaaaaatatttgcggtcagattaggtgtgaatcatatttaaaggaagattgttggtcctatatcaaagattagagaatgaatgttcattttacagttttaagttaaaatgtttaaggacagtgtttaccattgcacatgattccctgatttaactgaccttttcagttaactagccactagacataattacattgactataggggcttcttttgtgccacaccagtgttggaattgtgttgatttacttgtggagttggaactacagtttctctcagcagcctgcttatctggttgtttaaacttgtagctgaattattatacaaagttttcaaactttctgttttggaatggtcagttactcaaaactgtggcttcctccttgctaccttacagggtagttgtgagagtattcctcaacaatagttcagactctcattacttcttgtctataattgtagcatgcttttaatgggattccctgcctccagtacctctctggcttgttggttaacataaacattattgccagattaaacatcctaaagtataatgtgtaatcagatcaaaagccatttataacaataaaaactacattaaaaaattacttatgtcctgggcttagatgtggtctctgcaatcagatgttcattaaacaaattatttaaaaaatacatacatacacacacacgcaaattctgaattgtgatatgtgccaaaagaaaaaatggggggtgatgtgagagctcagtgaggatcaaatatagattgggaagaagggtgatgaggaaagacttctttgagaaagtgagatttaaactatctgaagaagctgtgcttagtttagaaaagtgctaggcaaagagtgttccaggaagaaggaatagcatgtgtgaaggctctgctctgctgctgaaattgtggttcagtttattgagtatggcatagaatgaatagtgcctgatgaggcaggaggtaggctgggatgagatcttttcagatcttgcactctgtactaggaagtttaggttgtattctgagttactggggagccattaaagggtttaaggaaggggagtgttgtgaatcagttatgtgttttgcagattctactctgactacagaatggatgagagcagaatgggagttagagcaaaagaaatgaggaaaccagttagaatgcctttgtaagtagtaggtcagagagaaaggatgataattttgactagagtcatgacagtaaacaaggtaacacatgaagtgattatgtcataaactattaccattaccctgtgaaggaagtatgagcagaataagatttagaataaacttattccaagcccatctaataaagggtggtgtgaggaactgaaacaatgggtgtttcaggacattcggtccttgtctctaaaataaaatccaaactctgttgaagatttttcagcctactcttattatctttgtcttctcctaaactcttactcacctatgctataatcataccaaactattacagtgagtatcatgaacatttcctgtaccttgatgccactgatgattgtttactatggctggaatactcttactcccacatacacttaacaaaaccctactcattgtttaggatccagatcaaatatgaatgaagccttccaaatttgcctccagattgatattgcccatttatcttcttttaaccatagtagttcatattactatcacattatagtatattattcaacagatgtctatgtaattgattatgtgccaggtactatgtgtagacattatggatatatccacatacaaaaatagacacagttgccgcatttgtggaaactacattgtaacacagaccttgaacaaaatctgggcacaaatatgtaactaaatgttaataagaggtatgaaggaatacctgcaggtttctgaaaccctaataaggcccttgaggtcaaaattattttcataatcctactaagatactatttgccttttcattctcgttcttgtgagtatgtagtagttataggatgtatgatattccaacaaattgaatacagaaggtgggagaatccagatatcttttgttaaggcagacattaaagaaattggcaaaaaaaaaatttttttttttaaagccactttggggccaggtgcggtggctcacacctgtaatcccagcactctgggagtctgaggtgggcagatcacttgaggtcaggagttcaagaccagcctggccaacatgatgaaacctcgtctttaccaaaaatatggtagctgggtgtggtagcacatgcctgtaatccgtccctgtaatcggaaggctgagaggcgggagaatcgcttgaacctgggaggccaaggttgaagtgagctgagatcgtgccattgcactccagcctgggcgacaagagcaagactgtgtctcaaaataaatgaataaataaacaaacaaataaataaataaataaaaatgaataagacactttgccctctcattgttttttgttgttttggaaaatgtggttatttttttcattaaaaaatagatcacttttgttaattagtttttttgtcattttaaaatgaattttaaacatttcttggttataatttttaatgtggtaaatactgatagcgataaactacatagataaaagctttctagggtcctccataatttttaagagtataaaggggccaagtgtggtggctaacacctgtaatcccaacactttgggaggccaatgcaggaagatccaacatagcaagaccctatctctacaaaaaaatttaaaaattagccagacgtgatggtgtgtgcctgtagtcctagttacttgggaggctgaggtgagaggatcacttgagcccagagtttgagattgtggggagctgtggtcatgcactacattcctgcctgggctacagagtgagaccctatctcaaaaaaaaaaattatatttttatacacacacacacacacacacacacacacacacacacacacacacacatatcttatatatatataagagatcctgaaaccaaaatcagagtccctgtaatggggagctgatttaaattgggggcttctggagaagtaaaagatatcttatgtctctgttaaacacttatgttcttctagaaattaggagtgtggttttctccgtatttccaaaaactgcaagtctttttatataacgagtgtgttggttgaatctaattttaaaaaaaatttttttaatttaaaaattttttaatttttaaaaatatggagtgcttcatgaatttgcatgccattcttaggcaagggccatgcctatcttctctgtattattccaattttagtatatgtgctgctgaagcaagcaccaaattttgttatcaaagtctactgtagatggccgttctttctttgatttgtggtttggttgaaacctttgttggaaataccatcttatttcttattactgtaggaaatagatctggattcaaattcctcttggtatgaagctgggaagattacttaacctgctaaaccttaccattatttatctgtaacatggaatacatattatcttactgtggtgtaatgatttaatgaactaatgtatgtgacctgattcataagtgaccagaacagagacaatccataaaatggtaattctttttattttatatacacagcacatacatactggggttagtttcagttagtgagcagagaggtctgtggataaatcaggatataacaatcatcattcaaggatatattcaccattcaaacagtttatcagaaggcagaaagccagaatgatctattgtgtatggttattcatctttagaaaaaggaacagaaagtgtgggcttagaatattttaaataagtagctctatgttacaatgtagatgatttagggatggggtagagttttatgtgtctggcactacagcagtgcctcacatttactaaatgtttgaataatattaattaactagagattcttgaaaacacatttttagaagccttgaaaaagttatatacgacagattaaaggccaagcctgagaaagcttacatggctaactggaaaaataaataaaggtaccatagaggaaaaacaaaattgccctgtggggagaacatgtggtgtcatatggtgtgactaaataggatccagtaagataagacaaggtagagcatcttgggagtgattccatgtttcaaggttaaaatgttaactacattaaaggtagtaaaccagtgaaagaatcctcaagatcccagtgcagaatgtttgccaagagataagaagatcaactgttttggtattcatagcagaaagccataggaaaattatctttttgatattcttttttgaagaagataggtcctttatttatttatttatttatttatttatttatttactttattttattttattttttgagatggagtttcgctcttgttccccagctggagtgcagtggtgcaatcttggctcactgcagcctccgcctcccaggttcaagcaattcttctgcctcagcctcctgagtagctgggattgcaggcatgcaccaccatgcccagttaatttttatttttagtagagacggggtttcttctccatgttggtcaggttggtctccaactcctgacctcaggtgatccacccgcctcggcctcccaaagtgctggcattacaagcgtgagtcaccgcgcccagccaaagataggtccttttttaagacagatacttagggctgggtgctgtggctcatgcctgtaatcccagcactttgggaggccgaggcaggtagatcacctaaggtctggtgttcaagaccagcctgaccaatatggtgaaacccaatctctactaaaaatacaaaaattagctgggtgtggtggcgtgcgcctgtagtcccagctacttgggtggcaattcaaggagaattgcttgaacccccgggaggcagaggttgcagtgagccgagatcgtgcctctgccctccaacctgggtgacagagcaagactccatctcacaaaaaaaaaaaaaaaaaaaagatactttgataaagaaataatagttatttctcattttatttctcatttgagatgaactcaaagttggctaaagtgacacacagttttggacctataacttgcttacattttaaatattaggttggtgcaaaagtaattgtggtttttgccacccaatagaaaggattgtagacatttttttatttgacaactgtaaagcattgcaggaattatatgtggaattataggcttactttgttttattgtgtttcattttattgtactacacagataatgtggttttttttacaaattgaaggttggtggcagccttacatcaagcaagtctgttagcgccatttttccaacagcacatgctcactttgtgtctctgtgtcacattttagtaattcttgcaatatttcaaactttgtctgttttggtgttctgtgatcttgcatgttactattgtaattgttttggggtgccacaaaccacacccataataaggcagtgaacttaatcagtaaatattgtgtgtgatctaactgctccactgactggctgttcccccaactcttctccagcctccgcatgccctgagacacaacaatattgaagttaggccaattaataactctacaatggcctctaagtattcaagtgaaaccaagagtcacatgtctcttgaaatcaaaagctagaaatgattaagcttagtgaagaaggcatgtcaaaaagctatgccttttgtgccagacagctaggttgtgaatgtaaaggcaaaattttggaaggaaactacaagtgctactccagtgaatacacgaatgataagaaagcaaaacaggctcatgcctgtaatcccagcacttttaagaggccaaggtgggaggatcacttgaggctaggagttcgagaccagcctggccatcatagtgagacgtcgtctccattcatttttaaaaagcaggcaagcaaaacagcctaattgctgagatggagaaagttttagtgatctggatagaatatcaaaccagccacaacatttccttaagtcaatgacaggctttaaagcttcaaaggacaggctgactcttactagaggctaatgcagctggtgactttaagttgaagtcagtgctcatttatcattccaaaaatcctagggccctttggaattctgcaaaatttactctgtctgtgctctgtaaatggaacaatgaagcctggatgacagcacattcttttacagcatggtttactgaatattttaagcccactgttgagaccccctgctcagaagaaaaaaagatcccttccaacatattactaatcgttgataatatacctgatcacccaagagctctgatgatgtacaaggaagattaatgttctcttctgttttctatgtgtgtgtgctttttttgttttgagacagggtctcattcttgcccaggctggtgtgatcatagctctctgcagccttgaacccctgagctcaagcaatcctcctgcctcagctgcctgagtaggtgggactacaggcatgcaccaccatgcccagctaattttttttttttttttaatagagacagggtctatgtttcccaggctagtctcacaaactcctggtctcaagtgatcctcttgcctcagtaatgtttttcttaaccactaacgcagcattcattctgcagcccatggatcaaggagtaattttgactttcaattcttcttatttaagaagtatcttttattaggctatagctgccatagatattcctatgatggatctgggcaacattaattgaaaaccttctgcaaattattcacctttctagatgccattaagaatgtttgtgattcatgagaggaggtcaaaatagcaccattaacaggagtttggaagaagttcattctaatgcttgtggatgactttgaggggtcaaaacttcaatggaggaaggaattgcgggtgtggtagaaatagcaaaataactagaattagaagtggatcctgaagatgtgaccgaattgctacaatgtcatggtaaaacttgaagaaatgagaagttgcttcttatagatgagcaaagaaagtgattttttgagatggggtttactcctgctgaagatgctgtgaacactgataaaatgacagcaaaagatttagaattttacataaaattagttgatgaagcactggcagatttgagaggactgactccaagtttgaaagtagttctactgtggttcagatgctatcagaccatattgcctggttcagagaaatcttttgtgaaaaaaagtcagtcggcacagcagacttcattgttgtcttgttttaagaaattgccggccgggcacggtagcttacacctgtaatctcagcactttgggagaccaaggcaggcagatcacctgaggtcaggagatcaagaccagcctggacaacatggtgaaaccccatctctactaaaaatacaaaacttagcaagtcgtggtggcacgcacctgtaatcccagctacttgggaggctgaggcaggagaattgcttgaacccaggaggcagaggttgcagtgagccaaaatcacgccactgtactcccaccctgggcgacagagcaagactccatcttaaaaaaaaaaaaaagaaagaaattgccacagtcaccctaacctttagcaaccactgacttgatccggcagcagccatcaacactgaggcaagacccaccaacacaaaaatgatgactccttgaaggcccaggtaattgttagtggtttttaacactattttaaaattaagatatgtgcttttttttttttttttttttagaataatgctattggacattactagactacattatagctacacttttttgttgttaatttaaaaattgtgggccgggctcagtggctcacgtctgtaatcccagcactttgggaggctgaggcaggcagatcacaaggtcaagagatcgaaaccatcctggccaacatggtgaaaccccatctctactaagaatacaaaaattagctgggcgtggtggtgcacgcctgtagtctcagctactcgggaggctgaggcaggagactcttttgaacccgggaggcagaggttgcagtgagcagagatcccgccattgtactccagcctggcaacagagcaaaaccctatctcaaaaataaaataaaataaattatggccaggcgtggtggctcatgcttgtaatcctggctctttgggaggccaaggcaggtggatcacttgaagtcaggagttcgagactagcctgaccaacatggtgaaaccacgtctctactaaaaatacaaaaaaaattagctgggcatggtggtgcatgcctgtaatcccaactgcttgggaggctaaggcaggagaatcgcttaaacctgggaggtggaggttgcagtgagctgagatttgcgccactgcactccagcctgggtgacagagcaagactccgtctcaaaggaaaaaaaaatggatacataatgattatatatatttctggggtacgtgtgatattttgatataggtatacagtgcgcaatgctgaagtcaaggtgattgggatacccatcaccttaaacatttatctttgtgctggaaacattacagttctcttctagctattttgaaatatatgataaattgttaactgtaatttccctactatactgtgaaatactagaacttactgtttctgtccagttgtatgtttgtatccattaaccaacttccctttatccctttctccctccttttcttcccagactctgataaccactactctactgtctacctccatgagatcctttatgtattctggatatagatcctaattaaattcatgacttgcagctattttcttgcattctgtaggtttttttcactttcttgagaatattcattgcacaaaaggttttaattttgttgaagaatgatttgtcagtttttttttgttgctcgtacttttggtgtcatatctaagaatccattgctaaatccaaggtcattaagatttacccctatgttttcttctgagagttttattattttagctcctatatcatttattcattttgagggtttttaaaatatggtgtgaggtaggggtggacatttattgctataaattgtcctttgagcattgcttttgctgtatgccatcagttttggtatgtgtgttttttttgttttcatttgtctaaaagtattttctaatttttcttgtgatttctttttttgaccttgtatctatattcaagagggatattggtgtataattttcttttttgtacagtcttttgtattagtgtaaaggtgatgctggaatcataaagtgagttggaaattacttactccttttctgtttcatggaaggtattttgtagaggtggtcttatgtcttctttaaatacctgatagaatttgctactagagatttatttttagaaaggtttttaactatgagtttaatttccttaatagttacaagagtgttcagattatctgttttatcttgtgaatataaaagatatcttcagatatcttttatcctgtgagagttttgtttttctttggtttttgaggaattggttcatttttttctaacattttgaatttatgtagagtttttcatagtattcttattaacctttaaatgtctgtgtgtaggggagtctgtagttcattgtttttttattactgatattgttaatttgtgtcttccctttttgtcagtcttgctagagatttgtaaattgtattggtcttttcaacaaacaagcttttgttttcaacatttttttctgttttcaatctcatggatttctgctttttattatttttgtgttgctttctttaggtttatttagttcttcaagtttcttaagatagatttgcaactgtttttctttgctgatataatagtatcagtttaagagcagggcttggtggcacatgcctgtaattccagctatttgggaggctgaggtggtagcatcacttgagcctaggagtttgaaactagattgcgcaacatgcaagacccacccccccccccatctcttaaaaaaactaaaaaaaaaataatggcacaagcttaatgatattaattcactgtagcactactgtatttgcatcccacagatttagtatgctgtattttcattcagttgaatattgaactagccttgtggtcctgggattaaccttattgtcatgtttattatgccttttacaattgctggattctgtatgctaatattttactaatgattatgaggaatattagtatgtaatttggggcttccatttgactcaaagattatttagaaatgtgctgtttaatttccaggtgtttggagattttcctattaactttctatttctagtttaattctattatggtcagagaacaaacgtttgtggtttttttttaagcttgtgaaagtttgtcttatgactcagaatatggtctgttttggtgagtgttccatgtgcatttgacaagaacatgtattcagctgttaagtagaatgttatataaatatcaatcagatcaggtggattgatgatgttcatttcttccatattcttactgattttctgtctactagttctattactgaaaggagtgctgaagtcatcaaatataattaagaatttgttttcctatttgtaatgttctgtaagtttttacttcatgttctttgaagctccattattaggtgcatatatattagttatgctttctattatgaaaattatatttgaagtgaattactcgtagactacatatagttgggtcattttaaaaattcattctaacaatcttgtcttttaatttgtatctatagactattcacatttaatgtaattttggcatgtttagatttaggtttaccagtttagtaatttgttttctgttagctgcttctgttttccattactctgtctttcctgcattcttttagattgtttgaacaacttttagccattctgttttaatttacctgttgtggcttaaaaattcttaactctccatatagttttagtgatcactccagagattacattataaaaacttaacattttcaccacctgctttaaaataaattcatttctttaaatggattatggtccacttaaagaaatgttaaaactacgtaggtctctttatcttctctccaccttttctcttattgttggctgtatgttacgtttctattaattgaaagcttcattgggcaatgctattgtttttactttcaaccattaaacatatttaaggaaactaagaggagagggttaatgtatttttgtctgcatgtttaccattttgcttactcctcaacctactcttccaggtttccttccggtattatttcccttctgtgtgaggaatttcttttagcaattcttttagaacaggtctgctggtagcaaattctcttagttcccatttatctgaaaatatctttgttttatgattgctctgaaagatactttaactggatatagaattctacctttgatagtttttttctctttcagcactttaaaatgtgtaacttccttctggccttcatggtttctgctgagaaacctgttgtcatttgaagtggtgttcccctatattttatggattgtttttctctgttttcaagataatttttaggctgggcgcagtggctcccacctgtaatcccagcactttggaaggccgaggcgggtggatcaccggaggtcgggagttcgagaccagcctggccaacatagtgaaaccctgtctgtactaaaagtacaaaaaacttagccaggcgtgttcgcgggcacctataatcccagcttctaggaaggctgaggcaggaaaatcgcttgagcccgggaggcggaggttgcattgagctgagatcacgccattgtactccagcctgggcgacagagcaagatgctatctcaaaaaataatagtaattttttgtgtttcattttcaacactttatgatgtgtgagagtgtggaattctacgtatttatccatttttggattcaccgacctttttgaatctgtagttttatgtcatttgtcagatttgggaaggcttttgacattatttcttcagatatttttaatcatcctattctttgcttttagaagttcactgacacagatgttagatctttttgttactgtcccatagatccctgatgtattactctattcttgaattgctataaagaaatgcctgagactaggtaatttataaagaacagagattcagttggctcatggttctgcggactgtacgggaaacatagcagattctgcttctggggaggcctcaggaaacttaaaatcatggtgaaaggtgtaaaggggaagcaggcatatctctcatggctggagcagaagcaagagatggggagggtgccacatacttttaaatgaccagatctcataaaaactcactatcacaatgacagcactaagggggatggtgttaagccatgggaaaccgcttccatgatccaattttctcccatcaggccccacctccaacattggggattacatttgaacatgagatttgggtggggacatagattcaaaccatatcacctggattctgttcattttattttaatccccacagctacatcaattggagacctggttctctccctgctctcagaatgttggctcttttgaagatcctattgttgccgctgccaccatggggttacgtgagaaaagtgaggggaaaaaactgagacactatctttgaatgttatttctttgcctttttttttttttttttttactttttttcttttaaaaagtaaactttttttaggccagtcgcggtggctcacgcctgtaatcccagcactttgggaggccaaggcgggcagatcacaaggtcaggagatcgaggccatcctggccaacgtgatgaaaccccgtctctactgaaaatacaaaaattagctggacatggtggcgcgtgcctgtaatcccagctacccgggaggctgaggcaggagaattgcttgaatcagggagttggaggttgcagtgagttgaggtcgcgccactgcactccagtctggcgacagtgagactccatttcaaaaaaaaaaaaaaaaaagtatgtaaaatgtaaactcactgatggtttcatgatgattcagatccttgtcctctatcccaatcgtctactgctatttacttccagtgttgtcgattagctgctctatgtgttctgtccaagttttatagttggactaaatctttgaagtgtgttcccaccacccccgtaatgtgtgactactaatatttctgctcaatttgttctttttcccctccttgtttttatttttattcttggcttcctaggggttgctcctgtctttccatagcttaatgtcaagctaaagatttgtcagaggttttgttcaaatatttcaaggccagtaaggtttctattctttctttgtgtgtctatctgtgtatcagggagtatattcaaacttcaggccaccatgttgcctgacctggcttttgctttcctccgggctcctctgtgtctcctgtgtgcatgaacatgcagaggctcagtcagtcaaggatgtgtggaggtgtgggccctgtccagaccctgtagcacgtgcttgcagtgtccgttcaactagtggagtgtggaaagtgtattaagcccccaacttgcagtggaggttatcacttaaattcacagcactccaaatcaattgtcaacaccctcacacacacacagtctctagctgaacacatgccaacagagggagagattggtcagtcatctgaagaagggatggcagcagcctcaagcaaaaatgccacagatggctgggcatggtggctcacgcctcttatttcagcactttgggaggccgaggcagatggatcacctgaggtctggagttcaagaccagcctggccaacatggtgaaaccctgtctgtactaaagatacaaaaatagctaggcatggtggtgcatgcctgtaattccagctacctggaaggctaggacaggagaatcgcttgaatctgggaggcagaggttgcagtgagccgagatcgtgccattgcactccagcctgggcgacgagcagaactctgtctcaaaaaaaaaaaaaaaaaaaaaaagccacagactgtgatgttcttactcaggttcagcctttagctgaagtccagagcactgaaatggttgttttgacggttttgtccagctttatagttgcttttgggggagaggatttatcaatgtactcatttcatcatgccagaagtagtagtattgatatgattttttatatatatcttagaattttgaagatactgttttcttattttctagcatacaacaatctgattcttgttttttattccttagaattttattggtactttcttatttccttttgagtttaacttagcaggatttgtttaattttgtatccctccaagcacccaggctggagtgcagtggtacaatcatggctcaccacagcctccacctcatgggctcaagtgattcttcctctttggcctcctgaatagctggaactacagacacatgccaccatacctggctaatttttatttttttttatttttggtagagatgaggtctcgctatgttgcccaggttggtctcaagctcctgagctcaagcagtcctcccacctcaacctcccaaagtgctgggcttacaggcataagccaccatgcccagctcttattctttctttgttaaaaaaggaactttggttctgaagactcttatttgccttttggctcaaggagaaattcttaaattttctttgttatttcttcttcttattttaattctcataaaatttctcttagatgtatgtaggataatttaatctgtattctgggatattaactttactctcataatttaagctttcattttacttcatattttttttttaactttttgttaaagtaccatggactttagctttgtccattttgctagttaactccttttttaggtttatttcaacagacattttaaatttcaagcttgttctgtgcaatttatttatttatttatttatttattttgagatggagtttcactcttgttgcccaggctggagtgcaatggtgcgatctcggctcactgcagcctccaccttccagattcaaatgattctcctgcttcagcctcccaagtagctgggactgcaggcatgtgacaccacaaccggctaattttgtacttttagtggaaacggggttcaccatgttggtcaggctagtctcgaactcctgacttcaagtgatccacctgccgcggcctcccaaagtgctaggattacaggcatgagccaccgtgtctggcctctgtgcaatttagtatttttgttttctttagtgagtgcagctcctccataatagcactatatacaatataattttctgtgctatgcagtatattagccagtagctgcagtggctgggaatgtggcctgtgagagtgagaaactaaattttatattttatttaatgttagtttatataaaattaaatagccatacatgtctaatgattactagattggacagcacagctctacaatttaagaatccttatactttttggcttcaaaataattctgatgccttagttcatctcctcattcaattgcatgtctgttttttatgctcttggttttttttaggtgtctaaaggttaataaaggttaaaaatattggaagttagagtaggtttcctcaacacttaagtgagtttcttctggagaaagtgaatattgaatataaaaatccattaatttctagttctactaagtgagctaggcagtccttgctttagagaatatgggtacttcttctcaatatgtgtatgtgtgtagcctctgtccctgtggtggccagaatttactataattctgtctctggccatagtgtccagacagaaatccctgtaagctgattacccttttccaccaggtttagatcagatacaaatatatgggggcaaagactgagttactctttttttttttttttttttttttgagacggagtcttgctctgtcccccaggctggagtagtggcgcgatctcggctcactgcaagctccgcctcccggattcacgccattctcccgcgtcaccctcccgagtacctggtactacaggcacgtgtcaccatgcccagccaatttttttttgtatttttttttgtatttttagtagagacagggcttcaccgtgttagccaggatggtctcgatctcctgaccttgtggtctgcccgcctcagcctcccaaagtgctgggattacgggcgtgagccaccgcgcccagccagactgagttactcttgaacatgtgatttgcatctaaaccaaatttgttaaaagtcttttttttttttttttttaagtaagaaaaggcagtgattttgatttgtaatgtcttttgttaggagaagtaaaagaaaaaaaattcttgaagaaaagtgagccagaattactgcctagggagtgatagtcatgtaggttcacaatgaagtttagaagtgctcttcgtggttccgtttcctcttttgctttagtgttcaggaaactaaaaactgacagtagactagttggaagatagactaggttttttttttttttttctagttaacctaagtcagaagggtgatatcactagagacttggaagtaaaaagagctacctgcaaagacttgaagtactaagattacctagaatctgaatgccctgtgttactctgcagatagggcattttactgttatgaagagataagatctattgtaaatagtctaaactaataataacgagtaagtatggaattggcgtttttcctttaaagttttagcatataattttgaaatgttttaagaatatttttgaaatgtttctattttttaatttctcttttagaagtcttatctaaaataagcatgtatatcttacatgtaaggaattattaacttcattttttcataaaaataatcagagtaagactttcaacagatgtcaggatagctgaagttctccatcactgtcatacgtttcacttatgacaggtttgtaatttctgttagggaatgtcatctaagtgctctgcatctggtattttctcctgcaccctcatgacaagaaaaaagcctttgattatttctcttttctctctatttacatgttcaccactgccttccatgttcagatgtgatctacatgcaatgatgattactctttccttcctttcccccttgccctcttttatcgtctttctttcttcctttttttggttgaccctgtttaaatgggatctatcaaatataccaagtttcagtgaaagttacttccttgtgttaaaagtcatgactttctcttttatttatacacttaataacttgattatatatacttgaattttgttttagtgcagaataattttataatctttctgaaggtattttaaagtggcagttaattccaacatttgtaataccaacaatgcagataactgagttgaatgacaacagcatgaatagccttgattaagttgtacatgtgcagatagctactgtgtatatcatgcctgccatctggacaacagtgatttttttttctttttttttttttgtttgagacagagtctcagtctgctgcccagactggagtgcagtggcgtgatttcggctcaccgcaacctctgtctcccaggttcaagtgattctcatgcctcagcctcctgagtagctggaattacgggtgtccgccaccatgcctggctattttttgtatttttagtagagacggggtttcgccatgttggccaggtaggtctcgaattcctgacctcaagtgatctgcctaccttggcctcccaaagtgctgggattgcagacatgagtcactgtgcctggtcagaaaacagtgatttttaagatgtcatcaattgtatggtagaatctgaattcaaagatgttaagaaggggatgggggaaaggtgtctcttagaacatgtaaaagaacatgttatttctgtcaacttccaaacttttatctccagccacatctcttacttgatttcagatttgtatacccagctcctgttttacatctccactctggttaatgtgcctcccgggatattttaaatataatttttaattctgcatcctatgccttgtcctcccaacctcagtgttccttcttcattctttttgatttttggctcttacatcctttgacttaggttataaactgtagagtcgaggatgactcccttttatcatacctcacatccaatttaccagcaaatcctattgactgtacctttataatatatttagcatctgacacatcttatcatctcttccactatcagtctgataaaagccaccatcatctttcacttggccatcatttcccatctggtttcttggcatttacactatagttcatttccagtatagcagccagagtaatactttaaaaatataattgatcatattgtttcctttttttcaaaagtaacttcattgagttcacctaccatacaaatcactcatttaaagtgtacagtgcggaggttttagtgtattcagtgtgcacaacaaccatcaccaaatttaattttagaatgttttcattagccctaaaagaaaccccacacccttagccattctcccccaatccttatattccccccatttctaggcaatctaatttctgtctctgtggatttttctattctggaccattttatatacgtggaatcatacactgtgtggttttttgtgactgcttcttttacttagtgtaatgtttccaaggtttggtcatgttatagaatgtatcagcattttgtttctttttattaccaagtaaaattccactgaatggatatgcgacattttatttattcatcagctgatgaatgtttggactgttttaactttttggctattgtgaataatgctgttatgaatattcacaagtttttgtgtctcctcctgctgtggactactgtttccctaacagaacgtattaattttcttttgtacaaaagccactccaaagcatagtgcttaaaacaaccactgcttgggtcaatgagcacactatattctgcactgcactaactttggctgtgctcattcatgtctgtgaccagtggtggttttaggtagatggctagtctaggatggtcttgagtggacaacttggttctagtaaactagtccaggcatgttctcatagaaaggcacaggtgagagagtgagcaagttcaattgtacaagaggacaagacttgcatatttgcattttaagcttctctttgggtcatgtttgctaatactacattacaaggtatcacaagagcaaggagaagcccagggtcagaatggaaaggggattgcagagttacgggtaaaagatgtgcctacagagaagccattaattgggggtcattaatgctatgtctgttgcacataccaattttgcttctaccttaatgctttttgcatttgtttcccttctgtttgaactagttttctcttacatacctgcatgctgaacttgtttcacttccttcctatctctgttcatgtatcatcttaccaatgagaccttcaagttttaccatactacaaaatgaatagccacccaccccgatcctgctcacagtctctgtttttctctataactttttatgaccatctgacattgtctgtttttttttttagcgtttatcttcccttaactagaatgtatgctctatgaggtcagggactttagtttaattacactgtcttcattgcctagaaccctggcactcattaaatatttgttgaatgaaaaattaatctacattgatccatttttacctaaattggaattatcttaaagttctaacctgccttcatgctatggcatgtataaagtgtcagtcttttgattagaatacctaatgatggaatatttcttctacttcttatagagacatagaccatgcaatatatgagtaattctaccatttacttagctgtctgacttgggccaagttttttttaacctccctgtttcctcatctgtaaaataggggtaataatagtcccagcctcatggagcctggcacaaggtaggcactgtataaggttggtgcaatgtgtattgcataatattttttatcacctacttgttattggagtatatggcacagggaaaatggaattgaaagctggagcaatcttatattatggagaagttgcaaaatctttgaaaactattttttcaaagggacaaagcacataaagcaaatgaaataggtgatttttttttcattgaatgcatcataacattatatccttaaaacactgtcttacaaagtgctctctgaaaaatactgttctttatttcagggtttacgaaaaagtacataaaactagtgagcatacatcttaactctctgagagtcttataagtaccatcctagatgcatggagagaagataattcattacttacactaggtcatgccttaggtcgttaagacttaagtatctcatggaaccccagttgagaaagaacaaaaatataacatcttgactaaggtggctagtgcttctctgatcttaactattatttctctagcctcaggtgagccttcttgaatgtgatttcatcagtctcctctaagcttacccagaatatagaatttttaatattttatatagccagcacttataaactacctgcttcatgctaggtagtatacttaggtgctaggagtaaaactgatcaagtggggtaagttccaaattcacagagcgcacagtcctaacaggctgtcatgcctcagttgtttatttgtgacagtgtttcccacagacttttaattttggaatgataggatgtggtgctctggaatggtgtcccagggttttggagaactgagagtaacctgggaaagcaggttacagtgagagaaagttatgaccaggagttgtaccaaaacgcaggaaacatgatatgtatattgttatggtgcataaaaatacttacaattaaaagtcctgaacctacactacatctattgtttcacatattttccttcaatttaaatcctgctccaaatacagctcctttccgaaaattcagttttatgtgacagaaatttgatctgtttcactgaaaacccatttgaatagtaattgtgagcaattgttgaatttgtaaatgttacgtaaatttagtggatgactttgtggacagtttgacgttatggacagttattcttgatactgtttcccctcctttcccctgccatccctgaaactttagggcttaatctgcttttaattagccagaaaaaaatgtttgatcctcttattcagttttagattattttagatgatttctaagttaaactctaagttagaatagtttctgcttattgatttcatgaatatcacttctctatttctacttctcaccttctgccaggccaaaggaattgtggaactatgatcatcaaactcctaaatcatcagccttttccttaaaagacttaaaagggttctatctatgtagacacttgtctggctcctgattacagttttaccattctcaagtgatactatttgtttcatcacagtccacataattcagggtcaagatactattatgaaatgactgtaaaaattactaatacctttggctctctaatttttctctcttttcatcatatgggcctgacaaaatgcccgctacctgcctgcacttaggcaattgaatgtagtgctttaaatgtataccaaaaccctcaacagggcatcaacaatgtctggcagtgcagctgcttcttcactttcccaattaaaaatgcgtttgctggctgggcgcagtggctcatgtgtgtaatcccagcactttgggaggccgaggcaggtggatcacttgaggccaggagttcaagaccaggctggccaacatggtgaaaccccgtttctactaaaaatacaaaaattagctgggtgtggtggcgtgtgcctgtaatctcagctacttgggaggctgaggcaggagaatcgtttgagcccaggacgcggagtctgcggtgaaccaacatcgcactactgctctctggcctgggcaacagaatgagactctgtctcagaaaaaaaaaaaaaaattcatttcctccactcattgcaaacctctcacagctcccactcctatctgtggaattcacttcaaactttactgagaaattaaatgcagctcttgtcatcttttcaccaccaattctacaaacttgtctgcattggtcctcttgttctgtcttcctttcatttgttatcgaagaccattcccgacagtctgatttctctacttgtgactcagatttcatctttcctgcatctctgtcttccatatcattcttctcccctcgattaaattccctttagccacaaacatgctctattatctcccagccccaatacatctgcaaatgtatgtaaataggaatgaccaaatatgtacagatatacaattcctacctttcctaatgtcttctagccactattatgtgttcctattcttatctaataaattttcttaactgaatattttttccttctacctcagtgtctgtttctttcttttcagccatttttggtttctcttgtaaatgtttatgatccccagggctgagccttcttggcctatactctcttgtaggtctgcctcccagtatgctagtgacacccaaatatgtcttcagcctcatctctcctccagggttctaaacttgcataaccagctgccttcttgatcatttagtaagcctctcctgttggtgacataatgcttgatttctctccctcatggcatcactgtccacccgttgcacacatcagaatttcagaatcagccttaattcttagttttttctcactcttcctccatgtctaatccagtagccctatctccactgttttcaccttggtccaagcaatcatctcttgcttaacacaaccatagtttcaactagtcttcctccctgcattcactcttggcccaagcaatcatctcttgcttaactatggttgcttaacacaatcatagtttcaactagtcttcctccctgcattcattcttggcgcaccgtaatccattcttcactagagtgataattagaggtactataatatatactgctctagagttatattgtctagaatttactagctgagtggccttgagcaatatacttaacacttatgccacaatgtgctcgtctataaaatgggatggtatatttatctgttctctgattgctataaaggaatacctgagactgggtagtttataaagaaaagaggtttaattggctcacggttctgcaggctctacagaaagcatgatactggcatctgctcaccttctggggaggcctcaggaaacttacagtcatggcagaaggcaaagcaggagcaggtgcgtcttacatggcaggagcaaggggtagggggaggtgctacacacttgtaaacaatcagatcttttgagaactcactcatcaaaaggacagcatcaagaggatggtgctaaaccattcatgaaagatccacccccatgatctagtcacctccaaccaggccccacctccaacattggggattacaatttgacatgagattgggtggggacacagattcaaaccatatcagaagtaaattgttagtaaaatataaatcagattatgtcacacatacctgtttttgaaactttagattctcattgctcttagaataaaattaaaactctacttaccgtggctttcaggatccttcataacctggcatattgcctaatttttctgatacccatcttgtttctactctccccttgcttaccatatagccacagtcactatctttaactttctagttggaaacatggctttttgtggggtgtatttccttttactttctattgttttgggaccaacttatttacgtcagttttgtgtttgttttgttatatgtaaaaatagtgccttaaaaaatcagtttttttttttaacctggtgaatcatttgttttatctttataaatgctgggacgagaggcctactccttttttccaattttgtgagagattggtaaaattcttagaagtggaattgctaggtcaaaagatagaaatgttttcagatggatgattcctattgaagtaaattttttgagaacttaacatgtccaaaaaggttttcatttgccctcatgcttgactggtagtttgcctggatgtaaaattccagattcacagtcatttttgcccagacagtgaaaacattattctactatcttttaaaaaataacagttttattgaggcataattcatataccatactatgtgcaatttagtggtttttaatatattcatagagttgtatagccatcaccgcagtacattgtagaatatttttatcactcaaaaatgaaacccctggatttattagcagtcattctctgttcccaccaatttgcccaccccccagccttaagtcaacaagtaatctactttccgtcttcattgtcttttaatgttgctgatgaaaagtctgatgccaaattaattctagttataggaagcttttagagttttccgttttgaatttctggaatttaattatgtaaactaggatttatgtagatatgagtcttttaattcatccgccttagcctcactgagcccttttactcagaccattggtgtttttaactccaaatatttttagcataatctttatttattctattttcttcatttagaactcttactaaatgtattttgaacttcccaaacatattctttatatctcttcaactttttacttgtatcacccatttctttctttcttgctctctcttttttaacctctctaccttctgggacatttctttgtgttacatatcactactatgatctttatcaatactacttctgttatttaccccttctcctcaagtgttttcttagtaataatatttcagttttctccttgcccatttattctgtttgttcattgtgatctttttctttcctcccattggatattcatagtgtcttgtgattcttgtttattgaataaggactaaattgacaaaaactttgcagttacataggtctaggattcttttctaaatgggaaatgactacaagtgtcatgtattcattgactggctttcctccagggtgcagggattttaagaggcaggcttaaggccaccccagttaccaaagtaaggacagctttactatgtttattcctggaaggagctagcttacttacttagtatcctcctccttcagtatcttccctctttccctctcgccctcccacttttcctttctacttcaagtatatagtaaagtgtagggaataatataataactatgtacctattactcagttttgttgaattgtcttatatatgcttcagatttttataaacaaaaaaattatagacacagatatagcaccttacagtcttactttgatacttttctctgccctttctgccttttcagaagtaaccattatcctgaatttactgtttatcatgcccatgcacatttcagatacttttgctgtgggtgtagatccacaaacaatctagaacgttgattgcatgtttttgtgaaccctgaaagaaccaaccctttaaggcagattctgagtggctaacagtccaaattcaaaatagacccacgcgatcctttgcagacatgtagagatcatatgtgtactccgcattcctggaaaacctatacacccagtaactttaggactttcatagctgtctgttcctatttatgccacctgaattaacagctaccagaaaataccatttggccttttgtacctaacaaacactctgtgacctgcctcagccaatcagaactgaacaagtttgcacccctcatttgtatagtggaccagagtgggaacctgactgtgaactttctctgtaaatgacaaccccttttctttgttctctcagaaggcgcctttattttctaccaaggtacatctccacggtttgcaaactgtttgctggaataaagcctgtttcttttttaagaaagaaaatctttttctgtagattgttgacattttaaaagttgtatatatcctgaaacttgctttttcatcaatattatgtttttgagatgtattcatgttgataaatatagcgctagctttttgttttgacttaatatgtggtattttgttatacagatgtaccagtttgccattttttctctcttagggaacatttgagttggttctagttttttgctattataaacattgctgcagtgaaaatgtcttgtacatatgtgaatataagtagctacatgataggatatgctcatccttagctttactctagatattgccagtttactttcaaatatttatattataccacatgtagaatatgatagtgtttgttgctacatgtacatgattaccaactcttgtattattaggcccaatttttttgcagtctcactgatttttcatatgtatgtatgtattttttgagacaaggtcttgctctgttgcccaggtgggagtgcaataatgcattcctgtcttattgtagcctcaaacttctgggctcaggcaatcctcccacctcagcctctcagcctctgttagctgggagtacaagtatgtggtaccgcatccaactaattttcttttttttcttttttttttttttttctagagatggggtcttgctatgttgcacaggtgctgggattacagggatgagccattgtaccaggcctggtttttcattgcatttctttgattactaatgaggtcatttattttatgtgtttattagatattcgaattccctatagtgaactacctattcataagtcttttgcccattttcttttggattagttgaccttttccaaaaggatggccacttaccctggaaatatttattatatagtccgttctgttccccactaaattataatgccaatgtgaaccatatgctgtgtaaataaatacctgtgcctctgtttctgagccctctattctgtctctttgggctttctgtctctatgctagtatcatttctctgtaagtcttgttttcctggtaacaccagtcttaccctccttattattcaaaatagccaaaggctactattatacagtgcttccataaaaattttagaatcaggctgggcgcagtggcttacgcctgtaatcccagcactttgggaggctgaagcggacggatcacgaggtcaagagatcaagaccatcctggccaatatggtgaaatcctgtctctactaaaaatacagaaaattagctgggcatggtggtgtgcacctgtagtcccagctacttgggaggctgaggcaggagaatcacttgaacccaggaggcggaggttgcagtgagccgagatcatgctactgcactcgagcctggtgacaaagtgagactccatctcaaaaaaaaaaaaaaaaaaaaatttacaatcagtttttcaagttccatgggggaaaaattatttttggaattttgtttattgcactacactgaatttatttgtggaaaattgacatcattataatattgaatcttcccataaaaggatatttatgtaggtctttgtcaagtgaaggggaacacatctgcatgcacacatacatgtcatttggtgtaatgtgaaataaggtttagtggagaaaaataaagaagtattaaggcacacacaattccctaaggtctgttatccccatatagatgtgtgcactggccagacacatctagaactatgtactgtggaaagggctcctgtgtaataattggagctttgaggttggtgaaatggtcttactagggaaagccatcagtactctgtggctgatcagccttttcttgcatgaaacaaactcagacctcctgagccaactttttgataagctaccaaaacaggggtggaaacctctagagtcatctgactctgtctctcttcttagtagccttttgtgcacctgcacattattttgttgtttccgtcacaaaagttttgggaatcttttgttagacttttcttctagattctttataatgttactatcatgagaagcatctttttttaattggctctggctaatgtgttagaacaccatgtaattttatgtccttgattttatatccaacaaaccttgctaaactcttctgttagtttttatagttaataggtattagccctgatggtttgtagattctcttgggtttttctgtagatagcatgtttcctacaaataagttttttgtctctataattctaattcttatgcctctttatataaatatatatttatataaacacttatataaacactacctatactaccaagattcaatgattgttaacattttactgtgtgtatatctcctaagaaaaaggaaattatttcatttaaccaaaatatcatatgtaggaaggttaaaattctatcatcttacatctgatatgtgttcagatttccatggttgtcccaagaatgtctcttgcagctatcttccatccctaaactaagatccactctgcatgcactgaattatttgttgacttttagtagtagttatcttttagtcttaaactgaccacctactgttttgttctcccatgaaatgatttattaaagaatccaggacagttgtcttagaatgtcccacattttggattagtccaattgccaaggctttgactttcaaagagaagaaaaatgtcttagagaatgttccacaatctatatttgattatttcagtgtagcagtcttagcgtatttctctattccctgtgtttcttctaaaccagaagttaagtatgaaggcttgattagattcagtttaaatgttttgcatgtatacttcatgggtgatgtctcatgttgtatcacatcaagaggcatatgttgggttgttccactattaatgattctaagccacttgaaggtatgttttccccttcatctttaatctgtggcgaaatactttggcactatgtaagtatcctgttctccatcagcttttctcctaatgatttttagcatcatttgatcctttcctgaaccaattattttatattagctggcattcttttgtaaagaagcatttccccatatcaagtgggaataactaatttttcctaaaaaggcgggatacatgcataatttttttcatttaaagttcagttttctaagggtgtaataatcccctacagtgtgagcaactgctctcccacccttttaatttttatggaattttgaaggttttttgtttatttgtttgtttttgcataccatctgttatagtcaattacagtacttattctttttgctgaaaatctccaaatttggtcagtaggagtcccttcaagctggcctctgatgttgttctgatatctggcacaagatgttcctagatctctcttcttgaataataatagataggtttgaaatgctgtaatgctggccctttcccccattaatgtagtcattgtgttgttgttactttgtgtaatctaataatgcttattttgttaagacctaatatgcttattaataaactcattagtaaaacctaaatgcttatttaggtttactttcatgtatattagtctctagattctccatctgtgttcattttctatggctgcaataataaataacaacaaacttggtcacttacaacaccacaaatgtggtgtactctcttacatttctggaggtcggaaatccagaatgagtttgttttactgggcaaaaatcaaggtatcagcaagactttgctctctctagagacaataaggaaaaatctgatttcttgccttttccagctcctagagctgcactgcttgtatttcttggctcatggccacctcctccatcttcaaggtcagcagtgtagcatcttcagatctttctctgctgagtttttatatcacctaatcatctatagtaaaattcccttttatttccctcttataagtatgcttgtcgattacatttagggcttacccagatatccagaataatctccctgtttcacaatacttaatcacatctgcaaagttccttttgccacataggataacatttacaggttcttctgggtcttggtatatgtttagttatcttggtggggggccactattcagccctctcatgccactctttctttgatattacttcttgattcagtttcctttttctaggttgtcagttattttcttcttgcaccttaacgtcagttcattatcttcttgcttcttttactgatgttgagaagtatgccatcagtctgacttgtctttactttctggttgactttaagaactgaacttttttattttcacctgttttggaaaatttatggccagcttctgttcaaatatctccccattccctgtttacctcccagattaactcattttgtgcttcgtgtctttcatattttccattcatttatctctgccttttggctaattttctcagaatcaccttctagtcctttaattctctcttcagcagtaatctgttttacgttacctgaggttttaatttgatgactgtatgattcaaaactcatgtctttatagtttcctcatgcttcttctttatatgtgttgtctttaggcttgctgttcttccagagttatctcagtagaaatttacttttgtgttttggtctgcaatttattagctttgatttctttgtcaaactgattatattcgttttctgtttttcaggaatgttctgaatttttccaactaagtggttgatggtcctggcctccttgctttcagtactgtgattctttcaaccattttcttgaaattttaagaccacttctgtctctcccttccctccctctgttcctcccccaaccccacctttcgtaacaattatttgtttcttggaatctcttaggctagtcctgtagttttcccctttgacctattaagtattatgttactggattcctaattaatgattattcttgcttttaatatatatatttatatatactattatatatgtttatatatcatatatattaatttaagagtttgatgcctttcttcacagtttggattattttatgttttgtttgtttgtttgtttagagatagtcttgctctgttgcccaagctggagtatagtggcgtgatctcagctcactgcaccctccatctcccaagttcaagagattctcctgcctcagcctcccaagtagctgggactacaggcacgcacccacacctagctaatttttgtacttttagtagagaaagggttttgccttgttggccaggctggtctcaaactcctggcctcaggttatccacctgcctcagcctcccaaagtgctgggattacaggaatgagccattgtacgtggcctatttttttttatgttatctttgttgagttttgacatctaggttttgctagcttcttaaagtaatttagaagatatataagtatatatatgaatatatatataaaatcacctgttactaattgatattttaattacattttctttaagggatgaggggagccaatggctgtagttttttttttgtgccatagctccccttcatgtccttccatagcagtttgtctgcatttatattttaacatgggcatcttctaggattttttaaaaaaaattctcagtcttttttttggagaaggagttttgccatttgtaaagtcactaaaaatgatcagttatttactaagcttgcttggctgtaatagctgtggtctactgaagagattaaaacaggagaggagatcttttagtaactttcttgaagtttttagttttgtttttaattttttcatctcccttttaggattgtggtaacaggtttttcttttttttccaagaagatgttttaactatgtacctaccgttagcttttcagtatcttattctactttttaatatatctctattttataagagtcatcaaagttctttctctgactcttctggagattctgaaagactttctctcccaaacacattctttaaaattccaacaagggtcattgtgacttttttttattcttcggatataaaattactcttctttggggagataaaaaacctgacttccagacacttatggtctggccccactatacctagccaatattatcattttcttttactttttttttgagacgaagtcttgctcttgtcacccaggctggagtgcagtggcgcagtctcggctcactgcaacctccgcctcctgggttccagtgattctcctgcctcagcctcctgggtagctgggattacaggcacacgccaccacgcccggctaatttttgtatttttagtagagacggggtttcaccgtgttggccagctggtctcgaactcctgacctcaagtgatccacccgcctcatcctcccaaagtgctgggattacaggtgtgagccaccactcccagcccaatcttaaccgttttttataccccagcaagaaaattttattctaatcagctcattctcttcatttctcatctctgattattttcccccactttctctctcccatcctttgagagaaatagttctttatgctaactgcatatgagtttttatggaacattgaaaaaaaaaagggagtgtgggtaatattgtcctgaatttactggacctaagaaagaatcactgaactaggatgacctgttttctttcctaccctattctgaaaccagcttaaatcttcagctttaattgcatacctattatgactaagacaccaaaataaatcatgaggggacaaaaaaagaaaatgtttctgatcttaaagtagctaatagacatggaaaacataatcaactttatgttatcaaattatgacttagaaaacagttttgccgagttgatcagtgaacacatcatagagggatgctctaaggaaagatccatcctggacaatgtggcaaaaccccatctctactaaaaatacaaaaattagccaggtgtgctgctgcgtgcctgtagtcccaactacttgggagtctggggtgggaggattgatgccagggaggctgaagctgcagtaaaccaagattgtgccactgcactccagcctgggagacggagaccctgttttaaaaataaataaagtattcatttttttttaaacaaatatatgtttgcttattgtatgctaggcactgttctaggctctggggataaatcagaaaaaataagacaaaaacctatgccctcctggacttcattctgttggggagtgggaagacacacaccaataacaaaatgaacacgtaaaatatatatagtaaggtggtcacatatgctacggaacaaataaaattaacaaagggcatagaagctgattctgtgcctttaaggaatgggagggatagagcctttctaaatgagaaggtaagagaatgcttcattgataaggtgacattgggataaagagatgaatgaacttggtaagacagcaaactttatagaaatcttaggggaagagcattccaggcagaaggaatagtaaattcacaagtcctgcactgggagtatacttggcacgttcagttaggccaaatgtgattggaatgggatgagtgacgggagaacaataagaaatgaagctagacaaatagcagagggccaggtcatggctttggtgatttttagtggggaagccagtggagggttttgagcaagagaatgacatgtgatctgcagttatagttttgtttttgagatggggttcactctgttgcctaggttggggtggcgcgatcacagctcattgcagcctcaacttcccaggctcaagcaatcctcccacctcagcctctgggaccacaggcacacaccaccacgtctggctattttttttttttcttttttttttgtagatacagggtcctactatgttgcgatcctcccgtctcagcctcccaaagtgctgagattacaggcgtgagccaccatgcatggcctgcagttacagtttttgaagaatagctactgtgccattctaggcatgaaataggagaggccatttcagaaactcttgttaataaactaacagagaaatgaaggtggcttagaccagggtggtaaagttagagaaggtgactaatatctacatatattttgatttggagatggattgaaatggattgtcagatgtgagaaaagagtcaaggatgatgctgaagtttttggtttactcaaagaagggagcttatttcctgaggtaaagagactaggaaaaacaagttttgtgggggtgatgggggagatcagaagttagtctttgaacatgttaagtttgaaatgcctattaggtatactagtagacatattcagcaggcagttaggaatctggtgttgagaggaaaaggtagagttggattttaaattttgggcttcatcagttcatagttggcatataaagatacaagattggatgagatcacctagggagtaagtgtagcttgacaaaagaagccatctgacactttttggagatatgaaaaattagcaaaggcatcatgaaagaaaacattgaaatacctgactggaaaaacaatttggaacctctatcactgcttctcagactttaatatgcatatgaatcattttgttaaagttcagtagatctgagatggagcttgagcttctgctgtgtgttttgtttgtttgtttggtttttggtttttttttttttttggcaggttgtggggtggcggtagaaactgagtctcactatgttgcccaggccggagtgtagtggtgcactcatggctcaccgcagcctcaacctcctgggctcaggtaatcctcccacatcagccttctgagtacctgggactacaggcatgcaccaccatgcccggctaatttttgtattttttatggagatggggttttgctatgttgcccaggctggttgtgaactcctgggctcaagcaatccacctgcctcggcctcccagagtgttgggattacagatgtgagccactatgctaggccagcttctgctgttctcacagtctttcagaagatgctgctgcttctggtctgtggacccacactttgagtagcaagactttatatgacaagagtcatgaaaaacaattgaaagaccttaacatttgtagaagaataaagattaatatccagaataaagactatctataaatgaatatggaaaaaacaaacaactcaatagaataaaagggagtgcagtatagggaattttccaaaatctcaaatggccagtaagcataaaaagtgctcaactttgtaagaaatacaaattaaaacatgattgccttttccctttcagactggcaaaagtttagaagtgtgattgtgctgggtgttggtgagattgtggggaagagtggacttataccctgcagatagggacttgacttgatacatttcttcttttggaagaccatttggcatatttattaaattttaaaataggtatatccttcaatcctggaatcctatacctagataccaaaaaataaaacttgcacttttactctgaaaagcagttcagaactatttgttggagtattgtttgatgttgaggaaatggaataatctaaatgtcctttagtaaggaaattattaaatcaagagatttggaactcttttttttcatttatttcccagtacatttaaaaattgatatctgacattttttcattataactttgaatagtttaaaaggccataatttctactgtgtgttatatttatgttaaatacattttttggacaagccttaaagctgcagatttagatcattcaacttagaaacagaatcttccgtataacctaatagccagttctcactatcaaaccagacaaattggactgtttttcttttttattaagaaaaaaaacctgattacttatttatcttaaaacagatatactaatatatgccttttaataaccactaaacttctggattctagtctggctggctggtgatgggtaaggcttggagccttgccacaaatttgtttcattgataaaatatggtcctgctcttaatttttcccccttttctctcaaggaatttccctatttaattgtctatttgttaggtactttggaatttatacgtctcagaatggtgcatcttagtagtatttgaggtggaaaagaactttgcctttcttttataaagtggaaaataattattttaaaagaggaagtagacaaggagaaccagttcttaagcagatcaatcagggagcatacagataaaacttgaggatctggaaattctcttaaaattgtctatgcccacctaacccctggataccactgaagtttagagactgttgaaataagcagtgcaatgctatgaaatgaatgctatataaccattagggagtaaaatatatgcatattgacgtggagagacttctgagacattactccatgaaaaaagcaagtgttaaaagtattttatatatagtatttaattatatgtgtacatttcatatatgtatgtcttaatgttttttccatgagtaaaagggagacatggaaggctgctcaccaaaccataaacagaacttaacctttttgaaagggggcctgggaatgtgaggaagatgaagagagatttcatggtttattctgaatattcatgtattctttgaatcttttatagttgagaatacgttgtattactagtgtagaaaaaaatttaaagatgtttcataataaaagagaaaaaccactgtactaaatgatctctaagctcaaaatttgaggaacactggcagttactgtgatgtagttgtctatgttaaaatatttctaattcatatactaagtgatttttatttaaattcttttgaaatattttttaggtgtggaatatcaaacaaatgattaagttgacacaggaacatatagaggccctattggacaaatttggtggggagcataatccaccatcaatatatctggaggtaagcttttgagtatcatatctagtaattttgaaaagaaaaaaaatgaacttataaaaacatttgtactgataatcttgattattttaggaggtgggattaggaatagatttgggaagagagaattagctttgcctgtattatacttcttaatcttgttttgtgtcacttgttaaaataaacatgtattttataatttagaaaacatcagaactaagggggaagtgatataaatttcaaggtattactctgaattaaatttttatttttatttttattttatttatttatttttttttgagacggagtctcgctctgtcgcccaggctggagtgcagtgacacaatctcagctcactacaacctccgcctcccagcttcagacgattgtcctgcctcagcctcctgagcagctgggactacaggcgcacattaccacgtccacctaattttttctatttttagtagagatggggtttcaccatattggccaggctggtctcgaactgactttgtgatccacccacttggcctcccaaagtgctaggattacaggtgtgagccactgtgcctagccaattttttttttttttttttaaagagaaagcacacagttttggagctcagcaaaccagttaccatattggaaaatggggtaattttttaaaatattttttccagaatgagtttccccaaaattggtttctgcttccttataggtaaaatttgtaatttttaaatagctcaacattgtttagctttactgtttaataatgccttgaggtttgtgtcaagccttgagcataattgtaaatttatattcattaatcctatgagttagtaggtgctgttataatcacagatgagctttcttagcattagttttcttaagtagtcagtcagactccacagccaacaaggtgttacagaatctaggtggtaaggctctacagcccacactgctcactactaccctacacaccctttctttataaaagctggctcagctgtcatcactagtgaggcaaggagatggagaactctaaattagaaatgatcaggttgccatttggctccaaataccagagatttttaagcacagattgaaagacttctgaactaaatttgtaacttagtaatttaattactttttgaagggccagttaagtcttaaggattttatgagaaagttaagtttatatttctgaagtaaacttggaagtttggaaataacagctaatatgctgttttctttctcctcttcttcatgtggctcatcccagatcacactcctagttaatatcaaagctaggactagaattcttatttctcattactagttagtgtacttcctaatagcctgtgctatcttctatacatatattattaggaggctgatcaaaggtgtcataaggaagcaagtactgctaatgagaatggcaattttttattaatcagaattagacaatataaccagcagtttttcttccaaccatttctatgtcttggagactttgaacccatgctgagatgtaattcatttgcatcaatagctatgtccccactatagcagtgattcactgccccctcctcacataactcttcaggctttttactatttgaagactgatcactaacaacaaaccttagagcttcttattctgtgcccagtactgtgttaaatgtattttattgcttaactcatttcttatagttactttgagcaagatactaggtactggatatttttgttattcccattttacaagtgaaaaaacagagaggttaagtaacttaactaaggtgacactgctagttaggaaagaaggcagatatctaagtcagaaatctgtgttcttgatctctatcttattctacctttatcatgacattgtctcgctctttccccaccctcaccccccagacagcttttcttatttgggctaaatatttctattcccttcagccattcttagtaacataatttatggaccttttattagatacagtctttttggttggttcttttagaattagacacacaaaattgaactacttattaaaattaaatctgagtaggacagcatacatcagaccataactcacaggttatattattagcttgtggtaaaacagattcttatcttggggacttgtggtatacaatgtgtgtgtattatgtgtatgtagtatgcaaaggtatgcatatacacatgtacatacacacacactgaaggcagcagtataatataataattacgatgagccatggagtcatacagactccatgtttgaatcccaactctggcttagtttctgtactcaagatatccagccttcttataccccacctcattagtaaagtggaaataatatcacatcttgggtaagttaaggattaattgagtttataattatgaaataacatagttcctgattcaaatattaagaattctataaatagttgatttatcttgtactgctctaggccttggggaatagcaattgtctgtctttttttttttcctcctgagatggaatcttgctgtctcccgggctggagtgcagtggcacgatcttggctcactgcaacctccacctcccaggttcaagcaattttcctacctcagcctcccaagtagctgggattacaggcatgcaccaccatacccagctaatttttgtgtttttagtagagacagggtttcagcatgttggccagtctggtctcaaactcctgacctcaggtgatctgccccccttggcctctcacagtgctgggattacaggcgtgagccaccgtgcctggccgggaatagcaattttttaaaaaatggccaacaatctccgcctttttaagcttatattttaatgaggggatggatatatagacaatgaacatataaatgagttttagaacattgcacttatcaccattttatttttttattttttgttgagatggagtctcgctctgttgcccagtctggcatgcagtagcacgatctcagctcactgcagcctcctctcctgggttcaagtgattctcctgcgtcagcctctcaagtagctgggattacaggtgtgcgttatgactcccgactaatttttgtatttttagtggagacaggtttcaccatgttggccaggcttgtctcaaactcctgacctgaagtcatccacctgcctcggcctccctaagtgctaggattataggcgtgagccaccgtgcctggccatcaccattttatatactatgaatggactctaaccagcccaagagaaaagacctgtacatactgatatttaaaggcccatcagtgaagagctagatctactgattactcactgagacccaccgttctgcaggtttctcatctgccatttaagattcctttacactttttttttttattggggtaaaatatgcctaacaaaatttaccattttaaccatttttaagtatatggttctgtggcattaagtccattcatgtttttgtgtatccttcaccaccatccatttctagaaattttatcatccaaaactgaaactctctagcctttaaacactaaatctctatttccttctttctctagttcgtagcaaccaccattctactttctatctctatgatctatgaattggactactctaggaacttcatgttaatggaatcatataatatttgtccttttgtgactggcttatttcatttagcttaatgtctttgaggtttatccatgttgcaacaaggataatgcatatatcaaaatttttttcctttttaaggctgaataataatccattcattgtatgtatttaccatattttgattatccattcatccatcagtggacacttgggttgcttctacgttttggctattatcaataatgctgtgaacatgggtgtacgagtatctgagttccttttacttctttggggtatataccaggagtagaattgcaggatcacatggtgattttatgattattttttctggagttgccacacctttttctgtagcacctgccctgttttatattcctactggcatcgcatggggttctaattttttacatccttaccaacacttattattttcggttgattaaaaaaaccatagcctaggctgagcgcggtggctcacacctgtaatgccagcactttgggaggctaaggtgggcggatcacgaggtcaggagtttgagaccagcctgactaacatgatgaaatcctgtctctactaaaaaatacaaaaatttcaggtgtgtgtagtggtgcgcacctgtaatcccagcaattcaggaggctgaggcaggagaatcgcttgaatccaggagaagaggttgcagtgagccgagatcatgccactgcactccaacctgggggacagaataagactccgtctcaaacaaaacaaaacaaggcaaaacaaaaaaaacatagcctcatgtgcgtgaagtgataccttattatggtgctgatttgcattttcctgatgactaacaatgttgagccttttattatgtgcttattggccatttgtatatcttttttgagaaatgtctgatttaagttcttggtccattttttaattggattgtttgttttgttcttgttattgagcgtagttctttatatagtctagatatcagttcttattggatatttaatttgcaaatatttttcccattgtgtgggttccctttttactctgcttatagttttttagtccaaattatctatttttctttttgttgctcttgcctttgatatcatatccaagaaatcatcaccaaatccaatgccatgacattcttctcccaaatgctcttgcatttgggtctttgatccattttgagttaatttttatatgatgtaaggtaagggtccaacttcgttcttttgcatatggatacccagttttctcagcatcatttgtggaaaagactgaatggccttggcacccttgtcgaaaaccatttgactatatatgcaagagtttatttctgggctttctattccattggtctatatgtctgtctttatgctgtcatcatactgttttgattacactagctttgtgtagtaagttttgaaatcaggaaatgtgagttctccaactttgttctttttcaaaattgttttggctattcaggatcccttgagattccttttttttttttgagatggagtctcactttgtcacccaggctggagtgcagtggtgtgatcttggctcactgcagcccacctcccaggttcaagtgattctcgtgcctcagcctcctgagtagctgggattacaggcacctgccaccatgcctaccttttgtatttttagtggagacggagtttcaccatgttgaccaggccaatctcgaactcctgacctcaagtgatccacctgccttggcttcccaaagtggtgggattacaggtgcaagccactgtgctcagcctccatgtgaattttataaaggatgtttctctttctacaagtaggtcatcaggattttgatagggattggattgaatctgtagattgctttggttaatactgatatcttaagaattcttaactctgaaatatgagtggacagttaagaataaccagatatttaaagaaagctgtcaacaggaaagacagcagcagaaatggtcaacagaaactcagaaacagtgcagaaaacagaataaaacttaaaaataactataattagcatcagagataagatctattagtcctgtcttgctttgtagcaatactaaagaagcaaaagtaacagagaacaagaaagacctcttggaaattaaaaatacaatagccagaaatttatttttatttttatttttttttaattttctttttaattctcagcaaggcaagttacgtctatagaagggtgcgcccttacagatggagcaatggtgagcgcacacttggacaagggaggggaaggggttcttatcccatatgcatgtggctcttgctgctgtgtcattcccctgttggctagggttagactgcacaggctaaactaattccgattaataactagaattttttaatttttattattatttttctttttttgagatggagtcttgctctgtcacccaggcgggagtgcagtggtgcgatctcggctcacttcaagctccacctcccgggttcacgccattctcctgcctcagcctcccgagtagctgggactacaggcgcccgccaccacgcccatctaattttgtttttgtatttttagtagagactgggtttcaccgtgttagccaagatggtctcgatctcctgacctcgtgatccgcccgcctcagcctcccaaagtgctgggattataggcatgagccactgtgcccagcccagaaatttttaaaaatatatgtatatacagacatataggtatgtacatgtgtatatatgtatgtgttgtatatatataaataaatgttgaaaaataaatattggaagataaatgttctagaaagcagagtaaaagcagagagatgggaaattttagaggaaacatgagaaaaattaaggaataaatcccaatccataaaaattataaatgcgtttaactgcaagaaacagaacccttcaagaataagtttctggaacaaatagggattttcatttcctttcacatagcaagaagcgtagaggtaaattattgtaagactagttctacacaggtcagttgtcagatacttggttgaatcgctatagtccctttagcttttctgtcaagaccacagcagatttttaagatctttccctcatacaactcctcaaagcagaaagcagagaacagagaagttctcttgtatactaagctcttgctaggaagaaaatctttctcaaaaatcctctataactcttccttttatatctcattgactagaatgaggtcacatatcacccctatactagtgactatgcttacatttcctgaagtaagagtctaataagtcaaaaatttccagtgttcagtttttcttcagattagtttgtatgctgtcaagtagaagttgtgctatgattattttattctataacatttcaacagaaaccccttttaagtttctataggaagataaatcttcaactacttattcttgcttcttctcttcattgtagcaaaacagagaaattggttacaccaccatcagaaatcctcttgattttctttttcccacgtactaatttatttaaaagccatcttttcagttctagatgttctcctactggcagataacagttaacattctattgattcccttgatgataactacagtgaaatatgagatacagtactctaaagaatttcaaatttgtcttctcagttgatctttggttcccttttttctaccatcatccaaaggaagaaaagataaatttactctgaaaagttgttccccagagtttcttcatttcctctcagtgtttccatattcatttatccaacaaattattggaatgccttctatgtgtaagatataatgctagatgcttttctagtagtagattcttctttctgtacttgatttattggagcccagagatgaagggatactgtctctgtaatcaccctacagctgagtcctaactacttcagaatttatggttgaatcctacagtcccctgtagactgatgaagacagtaacagctcctgacatttactgagtatttactatgtatcaggtactaatcatatattagttcatttaatcttcagcctaccctttgagttacacttaactattctcattttcattttataaatgaagcaattcaggcacagagggatttaataatatggccaaggttacacaacttgtaattggtagccaaggtttgaatcccagacattctgaatttacagcccatgcgtttaatcaccgtatcattcttacacttggcagcctttctgatttttagtctatatagaacctagaataatacagaggcattgtgtcaaacccttcaatgaaattaatactggaagctggatgcttcctgtggaatgcagaacagtccattatatatcatttatgggcagtttgtaagatttcattgtatcttgtgagagtaagaataattagactaaatttaattaactaaatgataaaaaataaaatttttatttcgctgctttttctcattacattgaaattaaatagatccttagatttttttcccttctgttttaattgagaaataattcacatactatacgattaactttttaaaactgtgtaattcagtggtttttactatattcaccactatcttattccaaaacatttttatcactccccaaaagaaaccctttatgcattagtagtcattccctgcagcccctgacaaccactgatctactttttgcctttatggatttgctggttgaaatatttcatataaatagaattatattatgtggccttttgtgactggctgcttttacttaatgtaatgtttccaaagtttgtttatgtagtagtatgtatcagtacttcatttcttgttatggctgaatatttcatcatacacatatatcatgttttgtttatccattcatcaactgatgaacatttgggctacttatatgtttttgccattatgaataatgccaccatgaacattcacgtgcaagtttttgtgtagatatattttcatttttcctggctgtatgcctaggaatagaattgctgggtcttgtggtaactctgttttacactttgaggaactaactgccagacagtttttcaaagttgctgcactattttgtattcctaacagcaatgtatgagggttacagtttctctccatcctcatcaacccttgttattatctatttgtttttttaattaaagccatcttagtaggtgtgaattggtatttcatttggttttgatttgcagtttcctaatgactaataatatagaacatcttttcatgttcttgttagccatttgtatatcttctttggagaaatgtctattcaaatcctttgcccacttaaaaaaactgggtttgtccttttattactgagttgtaagagttctttaggccaggcatggtggctcacacctgtaatcccagcactttgggaggctgaggtggatggatcacttgagtccaggagttcgagatcagcctaggcaacatggcgaaaccccgtctctacaaaaaatacaaaaattatccaggcgtggtggtgcatgcctgtagtcccacctgcccgggaggctgagatgggaggatcgcttgagcctgggaggcggaagttgcagtgagccaagatggcagagtcacccaggctggaatgcaggggcaccatctcgactgactgcaacctctacctcctaggctcaagccatcctcccacctcagcctcccgggcagctgggactacaggcatgcgtcactgtgcctggataatttttgtattttttgtagagatggggatttgccatgttgcccaggctggtctcgaacacctggactcaagcaatcctcccgccttggcctcccaaagtgctgggattacaggtatgagccaccgcacctggccaggagttctttatatattctagatagtagatccatgacttgcaaatattttctcccattctgtggtattttttcccactctttcaattttgtcttttgaagtacaaaagtttaaatgtggatggaattccaatttatctattttaaagtttaaatgtggatggaattccaatttatctattttctgtggttgctgtgcttttggtgtatcatacatgagagaccattgcctaatcaaaggtcaggatgatttactcctgtgtttttttcctaagagttttatagtattagttatatagccaaaacaggtttagttgcttgctgcctgcagagtccaattagtaagagcaaagtctagtataaagtgacttttttattccaaagttagcttaaaggaagaagacgtacaggcttcctgccttaagggtactgcttccctgttggagcagaaagtgggtgcttttaaagaaggtgcctacacgggggcagaaatgagcgggtggaagatctgcatattcccttcggtgccttctttctcaggcagtcaagttggtggcttcatgggcaaaaatacctcagaggtggctgaaaactctagcagtcttacttttggttgtagatcaactattacctcttgaggcaacttcctgacgggtgagagttccactcaggattgtctaagcacataattagatcaacttgccttgtagggaatgtctggtgaaaaggagataaaaggccataattgcatttcttttattcttttatctttttctttttgaaacagaatctcattctgtcactcaggttggagtgtagtggcatgatctcggctcattgcagcctctacctcctgggctcaggcgatcctcccgcttcagcctcctgggttgctgggactatatgtgcatgccaccatgcccagccaagtttcatattttttttagagatggggtttcaccatgtttcccaggctagcctcaaactcctgggctcaagtgatctgcctgccttggcctcccaaagtactgggattactggtgtgagccaccacccctggcatataattgcatttctaaagagctaagtaggaagtggggaggaggaggaaagaaaaaaataattaaactttttcttagaaaaatgagggtgctcaattatataatagatatgtgacccattttgttttgtttttaatttttgtaaagatggagtctatgttgcccagtcaggtcttgaactcctggcctcgagtgatcctccagcgttgacctcccaaagagctgggattgcaggcgtgagccaacatgcctggcctatttcgagttagtttttggatatgttgtgaggtagtagcccaacttcattcttctgtgtgtggatattcagttgtaccagcgccagttgttgaagagaccattctttctgcattgaattgtcttgctggctttgtaaaaaaaaaaaatcaattgactgtaaatgtaaggttttatttcttgttctattgagaaataattcacatactatattattcacttttttaaagtgtgtaattcagtggttcttagtgtatttacacaatggacaactgtcaccactatctgattctaaaatatttttatcattcccacaaagaaacccttcatgcattggaagtcattccctccagcccctgacaaccactgatctactttttgcctttatggatttactagttgtaccttgttctattctgttctatatgtgtgttcttattccaaaattattgcgctgtcttgattactatagttttatagttaagttttggaatcaggaagtataagtcctccaacttcttttttcttttataagattgttttgataatttcaagtccgttgtatttccatatgaattttaagatcagcttgtcagtttctacaaaaaaaaattgagattttgagaaggattgcattgaatctgtagatcaacttgggaagcgttgtcattttaatgatattaagtcttcttagattcttttttcttttttgagatggagtcttgctctcgttgccaggctggagtgcagtggcgtgatctcaactcactgcaacctccgcctcccaggttatcaattattactaatacaaatatttattggatttttgctatgtggtagtctctacaataatttgttttacttgtgttatctcatttaatcctcacaaaattattttgaagttgagaaaaatttgccatataaagaagagtatgataaaatattctaaaggagataaagagaaaggatacctcttctggattttgacaaagagagcttagaattagactttttgaaaaatatttagagttttgatgagaaggaatagggtttttgaacaactgtctcctgaagaagtggtatctggtatctgcagcatcttgaataaccaaaagaaatggtggtggtggttgtggttgaggtttgcacatatgttggagtggcttgctcatattagtagagtgagaaaacagaggaaaagggaaaagtagatgatatgagagaaaactgagggaatggtagactaaggccctaaacacccaatattagcactgaagtgagagtagaagaagaatggggaaagatgtggagaattttgtagataaagaagtagagtaaacttttcatttgatgactttgatcttcagagtggatttgaaggtaatgttattttcagagtgaattaaaagttgaaggatgaagttaagatccagagaagaattaggaagattggaataactgttttgactgtaatatgatgttaaaaacaagcatgattttcaggcagtagtgtgaaggaccaggcaaatctaagttccataaattgatcatagaactaattagaccagttttattattttctccaacagtgtgtggtaacctgacaggaaaaaatagaacaattgaatggtcagacttacccaaaatttgtatctggcaagctagttgataaaataacaagaattagcctgaaaatcttaagatatcaccaatggcattgttgtagttgtgagccgcaaagtatgtgaaggaagacaacaatggccaaatcttagcaactgtttgagagattaatgttgaccttgaacagattaatgtaatgagatagacatttaagaagttgggataagtatgtgtgccccaggggtgcgtgaggttctaaatcgagatcatttcatcatttttattgcttttgtaaagactgtgatgcttctccgggtcttaaaggtagaatggaaataaatgcttttagaggtgagaggtttagagaaatctatggcagtgttaaaagagtaattgaagttcatattgaatgcagcaagggcagtaggtaaaatgagccaggggccttggtagattgcagctttgaggatagggacagatggaataagtagatgttttgtacttaacaaaaggaaaagctattgatagatgtggtgaagtgatagtttataatctgtcatggagggatcagtatactttcaaaaattaaagctaacattttggtaaggaaatatgtttggaaaattgttcttttcattacttttcctaaggtttattttgtctagtaactgtacttgctcaggtgacagtaaataagaatattttatccatgcaggaggaacctttgtgttccagactgcatttatgtgatcttcaaagagtaggaattgtgtttgtcttatgtgtggttgtagtctgagccctaacagtgccaagcacatgtatcagtatatatttactgagtgattgaaggctcaataaataaattttattgagagaagtatgcttaagtaattaggccttggtactgaaagctaaaccaaattgtagctcagaaaatttggaatctatctgtttataatgccctcatctactatgaaacagggtctgggtagggcaactagcaggctgtaaacaaagaagaccttttgatgaagtttaaatctcttcctcagtagctcttgttactattccccgccccccttatttgttcgaaagtatgtggtaagatgcagcatagcagggtcctgtggcccttattgaaatgaaatttctctttcattctcctctacattttataattctcccttcctcccttacattcagacttgatagcatggcgtatatactctctacaatctattccctttcaccttttcagccttattgtttctttacctatttttcatttaaatttctttctttcctttttaagaaaacagggatgctgctaaagcttcatttaaatttcacaaacatatattgatcttttgctatgtcccaggtattgtatgacattgagctatacctatctatagtttttcatgtgtcttaaactttctcgcctttgttacagaaaaaacaaactctttgttacagaatttttcccatatagaggattccttcctccctactttcttcatccatctaaatgccaacctttcaaggctgtcctccttcctcattattactttgtgtcaagaatattgtttatttctcatgtacatcagttacacaaataaaaatacacccatcctactccactgcccaacagtaaactccttgagagacgggaaaggccattttaaaatatttgcttctctctttcataaatgtttgtagtattgcatctatttgagatggtaggtagaaatagtagagcatggtgtgagtgaggggattaccaatgccaaaaagaactatggacttgtatattccaattcgttcaatgtcattctgttcttttaaaatttgatatcttgtatggttgcttgactttatagccttcatcaaatgaaacttctaggaagatactctgatggatagtagccccataatttcttggctgtagggtataagccacattttcaaatacaattctgttttttttttttcttttttcacaaggcttacagctttgcatactagttccaatctctaagcagcattaggtggggaaagaagtcatcaaacagccatactgagaagagtaaagtattttaactgataactattccagaaagaatgtggcagttacacatcttctaaaccctattaaggaggttttggtactaacagtgagatctggtactgtgtcctaaattttggggtttttttggacaaatccttaatacctcatgtccttttcaacttctttttctgttgagagagagagagaacgaatggaattcatatgcctacaacttttccgggacaaaataattcttttaatgggaggaacacttttccttgatacagtgttttatcttacgttagacatgatttggaaggtaaatataagtggaccagaaaattggaaatactgtgcttttttattattcatggaccatctagatatcacatatgtataggtcaaatttatcaggtgtaatatagtgtagtgtctaataacaaagaatttatcaatttaaaggtgaaagtattcatttatagtatttctggcacttattttgtaatttgaaacttaaaaccctatcaactggtgaaaagattataatcaaaggctaaaattttatattctcttttgttaatgtcaggacaaagtccggattgaatataagtctgctttattttataggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtgagtcagttttaatatcaccattttgctgtttctttgtatttttcagacagatcagttgttgaaaattaatatattattacttcatactcagaggtcatgcaaatagaggcatccatgtcatacaggaatgaaaaatatgtaattatttggtggcaaagtctatattccttaactgaaataaatggtattgtctctagtgttggatttgacatttaaacctgtctgccaaacttgaaccacagtcatgtctaagcactagtttaaaataaatcaggattttaagacttgagcttgttcattatctaatgtgtgtcaggtaagactcattcttcacccttgagaaatctcctttctgagttatattatcagatgttgaacgttcagtgtgattgatatgtttcccctcatctacttcatttttggttgttgaatattagtattaaccattggaacagcgtaccctgtaagtaacatttgaagcatttaaagagaaatctgtggaattattgtgggtggtctataatccttaagtgcaacgaatattgttagtagacttaataagtaacccatctgtatacatcactactttttaaatgtctgtggttacttttgacaataaaaattccaaatacaactgaagtcaaaatttttcattttttttctctgacaacagaaatcaaaagtgcaattggtcattgtttaatgttccaaaaattcctttctgacttgaaaaaaaaatgttattatagaggcattttactttcagaagttaagaattcctgcatatgagtttagaaaactaatggagttacgagttaccagcctgtaagtttttatcttaggaaatatggctttctaaaggcatcatttattgtcagggaataaaaagtaataaaataaaaagtcatactttttctgccctttttccatgtacagacaaaagttggttgtaaaaaatagactctaatttttcattgtacagaattacaaatcaatttgtaaaacaaatccagttctaccctttctctttattctgttgggtagaataaaattaaaattatttttcccacattaagatggggaattactaagcatatctttccagggatgccccacttaaaaggggaatatcaacatatgaattatctgtaaatagataatcttcgaagcagcctaacaaaactggcaaacatcccatccccagtagatcttaaattaattctttattgtctgtattcatgagcagaaagtaggaaatgtgttcttcagtcccaggcgtttcccttctcttcatcacggtattgttccttccaggttccactcaacttagtaattttgtgagtttctgtatacaaatgtaaaattggggttgttggagcctttcaaatttctcaacctaatgattaagtttaactagcctccggccgggcacagtggctcgtgcctgtaatcccagcactttggaaggctgaggcgggcagatcacgaggtcaggaggtcaggagatcgagacagtcctggctaacacacggtgaaaccttggctctactaaaaatacaaaaagattagcggggcgtggtggtgggtgcctgtagtcccagctaatttggaggctgaggcaggatagtgctgtgaacccgggaggcggagcttgcagtgagccgagatcgcgccactgcactccagcctgggcaacagagcgagactccatctcaaaaaaaaaaaaaaaaggtttaactagcctcctcatatttatgagagaggccaagagaaatttacaggtatcactaaagcttataatttcccttttaaaaagaaataaaaacaactgagactcttcaaagatgtaaaatgataaatgaataactattaggcaattgactaggcttaccttaattatcctggctaggcttagattaaataagtaacttgttcttgtccgtcatactcccagtttaacaattcgtattcaatactcagaaaaactgtatatcgtgaacttaaaaggccttatgaatcatcagtaaatgggtttgtggtattaaactctttcaagaacgttattttgccattctctcttttatctgaatctgttaacccttttcttaactacctcttttcctctccttttcttctctcaattcttattaacattaacagttactatttgagcatttgccatgctgtgttccaagttctgtgctaaggactttacatacatcttctcagccaggataattcacattctaagtaaaattgtttgtggtagtgaatgacagttcctctatcaaaatacatgtttctgtttgtcctgaacactcaggaacaaagggcagtagaacctactagcagcaaatgaaatcaagtaccaagaatgagattatgttttttaaagtggacttagatttgtagtccaagatagcacgctaaacacttgcctttatatctttgttcctttccagaaatttcattgaaatgatagtaaagatatgtaaaaagaataataaaacaggataggattctgtcagagaactagaaatttttaagaattcctggaagatggaaaatatatgaatggttacgtaaataagccaaaatagaaaaccaaacaaaatactaggagctagattcttacatgaagggagccattcttagcaaaatcctgatgaagcagatagcatagtcagcagattacaaagaagtggatttgcctcttgggtagtcgtcagagtaattagtcgaaggtcttgctctggcaggaagacaaagtcatgtgtcttcataaatcgagctggtgatataaatatgaatattttgaaggtgtgtgttagttgtgagtcctgtggagaaccaaaacaaacctcaactaaaaacattgaaattctgaattcaaaaaaaattttttaaaggtcatggcctgatatttttagacatctgataatgggttgctgagtcctgagtccccagtatagccttacattgcacatgtccatagagatgtgaatgttgcctcctggagttgtacaacaaggggatcctgttagttcctcttctgtatatatggaacaaataatttgtagcatttattccatgaaaaagctctaaaagtcgttttctaaacaaaatttctgcctggagagttctatgtgactgctgtgtgtgtttaaatggtggaactgaacatcagatgtccctgaacctcagagaaatccagtggggtaaaaagtaaacgttaatctaccgaggagtaaaataatactctgcttccatctggaatataccagacttcttctcacgtctgtacccattagtaatctgtttggaaaacaaactgctttcccctactatactgtcaacacaacaaatttttgtgactagatgtgtgggggtgtttttccccaccaagcaatctccagttctctgtagataatcaactgggttctccacccaattgtctgcaattgttgttaagttgccttcaatttaactcaattctgacactatctacccaggtatagcatagaccccacaggttaagagctcagtcccacaagactgtccccctcttgagatgctagtcccaagttccaggttgtgacttacacttctgaccagctggctataaatcaaggattcccacatcttctaatctttgatcatttgctagaatagctcacagaacttcaagaaacacttaacatttactggtttgctataaaggatgttacaagaggtaaaagacgaacagccagatggaagaaatgcatagggaaaggtatgtgggaagggatgcagagcttctgtgctctctttaggagcatcaccttccactacctccaaatgttcagcaccctggaagctctaggaacattgcatttcagtgatttttatggaagcttcatcatataggtgtgatatttactaggtcaatctccacgacctccccacttctcagaggttggtggatggggctgaaagttcacattatggcttggtctttcttttgaccaccttccattcaggaacccaccaaaactcaccttgttggaacaaaagatgctcctaccacctaggaaattccaagggaatcagagctctgtgtcaggaaccaaggtcaaagacaaatatgagaacaaaagatgcacctggcaccctgatcactcaggaaattacaagagttttaggagctctgtgcctagaactggggttggaggccaaagggttgaagaccaaaatatatattattataaaccacaatatcagagtttgtcaacataataaaccacttgccatttgttggacctatacaactgtacaggagtaagtccacaccaaatcaacctaatttttatttataaatatgactagaacaccaacagcaacagagaaaactcaagaaaagaaagcaactagaagatgtaaaaatattatagctcaggatgtacttcattcttgaagccaaatcagtttgatataaaaattgagtcattcagaggaccacagagtttgtgcaaattgaaagtatgattgttgacttttgttaaacagaaaggccaaacattaggatagctagagttaataagtattttggaaagtaaagctaaagacataccttttaagatatgagataaagtgggcaaatatgagaaaaaagataagagacatggagagagcaaaagttccaacttctgtttaataaagaagcaaacaaaaatagagggaagatgtattcacagacataatacaggaaaattttcctgtgttaaacaaaaaccagccttcagatagataggggcaactgactgtcaacaggaaaacaaaaacagagattgacctggatacttgctgtataattcgaaagttgaaagagaaccttttcagaaagataaactggtaatctgcgggagaatgaaaactggatcgttttctcattggcaaactgagggctagaaggcagtggatcagggccattaaagatctgatggaagttaatagccaagctaatattaaaagtaagacactttgggtatctagggactgaaaattatttcagtggaatataataggtatctagaaatagatcccagaatttcagaaaatgtaatatgaaaaaaatggttcaatcagtaagaagaaggtgaattatttaatttttggtgctggcatatgtagaagaaaataaaattgggcccacatcttagactgcttacaaaagtaaattctatcagaatcaagagattaaataccaaaaaaaaaaaaaaatgtaaatctttcaagaaaaatctagtaggctgcatgtaaaatctaggagttgaggtcactatcttaatcaaaacttaattagctcaaaagctgtaggagacagatggattaaatacaattttgaaattatgtatgcccccccccacaaaaaaaaaaccttaacagagtcaatagtctgtataaatttggaaacagcattggtgcccagataccaagaagtaaatagtcttctgaatatacaaagaacacttcaaaattggcaagaaaaagacaaaacccagcagaaaaatagggaaaagatataaataggcagttcgtataagatgaaatccaaatgaacagtaaagcatgtgaacactgttcagatgcagtcagtcagttgtgagtgtacaaattaaaactatgagatcatttcatctgtctggcaaaatataaattattattggcaggggtgtagtgagaaaactgctcccttagttaaggatagaaatgtgaagtatcacagcttattggaaagcactttgataatatctgtcaaaatttacaagaactgtatcttccaagccagtactccatctattgggaatttagcacataaaagcaccaatatttatacaaggatatttgttaaatcatcgtaataagaaaaaaatatagaatgtcatcagtagagaaatggttgtataaatcatggtacagccacaccatgtagtacaatgcagcctgtgaaaagacttgtacttgaaggtttttctgagaggtattataattgagaaaaggaaagctgttggaaactctgatattacttacttattctttatgtatatcacattgcaatattctagtaggtctagaaatatctctctcaactagaaatgctgaataaaatatgtttatttaaatgtatagctgagctcaatagaaaaacctatagagggtgaaaacaaagacggagttgaaaaaaccagaaaagcataggagctgatatgttaaggtatcgctgaactataaccctcaacaaatctaacccaccatctatttttgtatggcctgcgaactaagaatggattttacatttttaaatgcttgaaaaaagttaaaggaataatagtatttcataatctatgaaaatcacacgaaatacaaattttggccaggtacggtagctcatgcctgtaatctcagcatgggaggctgaggggagtggatcacctgaggtcaggagttctagaccagcctggccaacatggtgaaacccacatctctactataaatacaaaattggccgggtgtggtgccgcatgcctgtaacccagctatttgggaggctgaggcaggagaatcgcttgaacctgggaggtggaggttgcggtgagccgagattgcgccgttgcactccagccggggcaacaagagcaaaactccatctcaaaaaaaaataaataaataaaatttcagtgtccatcagtaaaatgttattggaacacaacaatgctaatctgtttatatgttgtctgtggctggtttcatgctttagcagtggtgttgagcagttgtacatcacagtgtaatctacagaacctctggttctttatggaagaagtttgctgacctctgcggtcgtggcctcaggtgggccagtttgtcactctggaacaaaggatgtgaatatcaatgtgtgggaatacaacactgggccttcagcctgtgtcacaaaggtagttggaacccagattctcacataaagctgaccctttgaaggacattaccttcagtgaaaggctgaaatctattaccagaatacataaagataacaataaacttgactgtctttgacagtactctaaataaagtttcccctgtgattttttaactatggacctattctcatcattgtgtgggattcaaatttatactgcctgcattgtccaggaaccctcaagccatgaagtggacataacgactggtcttggagtggtaatagcactcaggcactgcacagaagcaatggaaaccctccctcagcccgtctaggtctcatagaattcacagaaataatgctttactgactatctcaagatctcaagttgtaagacatgcaaggaaacagtctacaatcagtgagttagcagacttaaaacagcagggttagactcctaagaaagaaccaagtagaacttttaggtagtcttgatttaaacctcatcatctatgaagtattcttgcttggattgggaccttctattttataagaaacacaggggatagaggaaaaaggtaagcaccatgaggaaacaattagacaattcagaatgttgacttcctgtaagacaatagccctagactccttgataagttaatgtcttggacaataaaaaggttggggattattccacactgtacaagaccaagtgcacataacaaccaaatgcagtgtgtaatcctgaggggggaagaatcattcttgggacttgggaaatttttaacattgattgaatactacatattgttatggaattaacgttcttaaatgtgaggatggtaacgtaattatataggagaatgtccttgttcttaggaattacatgtataagtatttaggagtaaagtgttaggatgactgcaacttcccccgcaaacccacgcttttttgagatggaacttatacacagtagagggtacaaatctgaagtatatcgctcagagtttttacatgtgtaactaccatgtagattaagatgtaggacatttcctgatgctttagagggttctcctgtgcccctcccaattcactaaaaataaccactatttcatttctatctctgtcagttacttggaaacttaacttcaaatggtgtgatggaaatcaacatgcagagaaagagaaaagcaaatacgaaaagataatcttagaatctaagaaaatttcgatgttaattgtaccatttgttcagattttctgtctgaaattttttgtaatagagttggaaaaattgaaaacatagaaaaaaagaaatgtagaaagttcaaaagaaacagttatctctaggaaaaaagagtgaacatactgatactcatttgaggatgtagcaagataacactttaagtaaaatttcagtatgtcaagttagttacagcaaattacataggaaatgtttaaatgtcacatagaaattcaaatatttatttaaaaaactcagtgaataaggtaaagctagattaaacccagccaaagaatgaattagtgaactctggaaagtgaagaaagagagttatagaaaatataaaagagaggttaagcagtatgaaaaatggaatgagcattatatgcctcaaagagttccaaaaggtaagaatagaaataaatgagtagagaaaaattcaaataaattagggctaacatttacaaatgacataaatctaacgatttgggaagcacaatctctgagttggacaaacaatttgaatccaggatagacataataaaaccaaagaatattgttttaaaagcactcagggaaaaaattatatagaaaataattaaaattagactaggaaaatttgttatggcagttgtagaaatcagaaaacaatagaataatatactcagagtactgagaggtacctgtaaatctctcagctaagttaagctataaatttgattaggttgacttgggcaaatgcaccataaagtcctttccagagaaggagttcacttttcataggttctacttgaaggaattgtccaagaaggtttactgtataaaggaaattgaacacagaaggaatgaaaagtaaaaattaaacctaaaatttgcagaagaaaacactacagaaaatttttgtgacctggggtttggcaaagatttcttaaatatgacaccagaagcacagtctataatgaacgtattagtttattgggttaatcaaaatgtaaaacttctgctcttcaaaagacagtattaagagaataaaaagagaaaccacagattgataatctttgtaagccatgtatctgatcaaggacatgtagaatatataaagaactctgaaagctcaatagtaagaaaacaaatggctgggtgcggaggctcatgcctgtaatcccagcactttgggaggctgaggcaggtggatcatgaggtcgggagttcaagaccagcctggccaagatggtgaaaccccatctttactaaaaatgcaaaaattagctgggcgtggtggcaggcgcctgtaaccccagctgctcgggaggctgaggcagaaaattgcttgagctcgggaggcagaggttgcagtgagccgagattgcgccactgcactccagcctgggcaacagagtgagactccatctcaaaaaaaaaaaaaaagaaaaaacaacccaattaaaaacggacaaaggatttgaacagtttcatcaaagatatatggatggtaataaacgcatgagaagacgctcaacattagtcattaggaaaatgcaaattaaataggcacgcgatacccttacatgcctcctagaatggctgaaattagagtgaccataccaagtattagtggggatgtgaaagaactagaatttccgtacactgctgataggagtgtttaattggtacaaccactttggaataaaatttggcagtttatttaatgaaaccttttcaaaatcccaacgggctttttttttttttcctcagaaatagaaagtccgtcctaaaattcataccgtatctcaaggaatcccgtatagccaaagcagtcaaaaaaagtacagagttgggggcatcatgcttcctgatctcaaaacttactacaaagttagagtaatcaaaactaagtggtactggcatgcagacagatgagagtcctgaactaaatcttcacatttatggtcaaatgatctttgacaagggtaccaggaccactccatgaagtaaagagaatctgttcagcaaatgatgctggcaactgaatatccacatgcaaaagaatgaagtggtacccttcccttataccatgtacaaagaataacccaaaatggatcaaagacttgaacataacagttaaagccataaaacttttactacaagaaaacataggagaaaagcatcataactttggatttgtcaatgatttcttggccgtgatgccaaaagcccagggaacacaagaaaaaatggaaaaattgtactattatcagaatttaaaacttctgatcatcaaaggatataatcaacagagtgaaaagccaacctggaatgggaaaaatatttgcaaatcttgtatctgataaggggttaatatccagaatataaagaactcctgtaatagcatacaaccttgttaaaaaatgggcagaggacttgaacagacatttctttaatgaagatatccagatggccacgagcatatgaaaagatgcccaacattgctaattattaggaacatgcaaatcaaaaccacaaggagataccacctcacacccattaggatgtcggctatcagacaaaaaagaagataacaagagttggcaagggtgtggagaaattggacccctgttgcactgttggtggaaatgtaaaatggagcaactgctatggaaaacagtatagaggttcctcaaaaaactaaaaatagaattaccatataatccatcaattctccttctgtgtatatacccaaaataattgaaagcaggatctcaaagagatatttgtacactcatgttcatagcagcattattcactatagtagccaaaggcagaagcaacccagatttctgttgatagaggagtggatgaacaaaatatggtatgtatatacattggaatattattcagccttaggacattctaacacatactacaacatgagtaaatctcaaggacattatgttaagtgaaatgagccagtcataaaacgacaaatactgtatgattccacttattgaagtacctagagtgttcagactcacagagacacagagtagaatggtgcttgccaggggctgggggaaggggtgctggggagttgtttaatggttatagagttttagttttgcaggatgaaaagagttcaggagattggttgcacaacattgtgaatgtacttaacactactgaattgtacacttaaaaatggttgaaatgttaagctttatgttagatatattttaccataattttgtgaaataatagtatggtagttatgtaaaacgttaggctggacacggtggctcacacctgtaatctcagtgctttgggaagtcaaggcgagagaatcacttgagaccaggagttccataccagccagggcaacatagtgagaccccatgtctacaaaaaaaatttttaattagccaggagtggtggtgtacatctgtagtcctagctacctgagaggctgaagcaggaggatcacttgtgcccaggagttaaaagctacattgtgctatgtgcaccattgcactccagcctgggtgaaagagcaagaccctgtccacccccacaaaaagtcagatgtacacttagcatatcacacagcccttctactcctcggtatttacccaagagaaaagggagcatatgttcgtagaaagatttgtatacgaatgttcttagttgctttgtcatagccccaaactggaaataacccaaatgtccataaacaggtgaatggttggattgtggtgtatctataaaagggaatactactcagtagaaaggaatgaactgctaatgcacacaacatagatgaatctcaaagtaattatgctgagtgaaagaagtcagacaaaaaatgattacacgttttaaaattcagtttacataaaattctagaaaatgcaaactcttctatagtgacaacagatttgtatttgcctgggcacaggactatggcaggatggaagatttacataggagcacaaggaacctttcagaaagtgatgggtatgttcattatcttgaatgtgatgatggttgcatgggtatatacgtaatctcaaaatgtatcaaattgtgtactctaaatcattgcagtttattgtatgtcagttgtacctagataaatctgtttgtttgttttttaatagcctttaagaaagctgatagtactgtggaaatattatatgaaatagactttgggagaaagaattattattaaaatttttttttcaacttttattttagattcaggaggtacatgcacagctttgttatctgggtatattgcatgatgctgaggtttgatgtacaaatgatcccatcatccaagtactgagcatagtagccaatagtttttcaacctttgccctccccgctctagtagcctccggtttctgttattgctgtctttatgtccatgagtacccaaagtttagctcctacttagaaatgagaacatttgatatttggttttctcttcctgtgttaatttgcttaggataatggcttccagctgcatccaggttgctgcaaaggacatgatttcattcatttttgtggctgcatagtattccatggtgcatatgcctttgctattgtgaatagtgctgtgttaaatatgtggttttttcgtagaatgattcattttcttttggatgtatatataccgagcaatgagactgctgggtcgaatggtagttctattttaagttctttgagaaatttccaaactgctttccacagtgcctaaccaatttacattcccaccaacagtgtacaagccttcccttttctctgcagctttgccagcatctgttgttttttgccagcactttgggaggttgaggcgggaggatcacttgaggtcaggagttcgagaccagcctggccaacatggtgaaacctcatctctactaaaaatacaaaaaaaaaaattagccagccatggtggtgcacacctgtaatcccagttacttggagctgaggcaggagaattgcttgaccctgggaggcacaggttgcaatgagctgagatcccaccactgcactccagcctgggtgacagagcgagactctgtctcaaaaaaaaaaaaaaaaaaaaaaatgtaattcagacgctgggtgtagtggcttgcgcctgtaatcccaacactttgggaggccaaggcagaaggattgcttgagcccaggagttcaagaccagcctgggcaatgtaaccagaccctgtctctacaaaaaattagccaggtgtgctagcatgagtttgtagccccagctacttgggagactgaggcgggataatccaggattttgaggcttcagtaaggtatgattgtgctgctgcactccagcctgagcaactgagtgagtccctgtcttaaaaaaagactttctagatcctggagtatgtgcaataactaaaaagaataagcatatatgttccttgagatttttatttttaattttattttttgagagtgagtcttgcccagaatggagtgcagtggtataatcacagctaactgcagccttgacttctcaggctcaggtgatcctcccatctcaccctccaagtagctgagaccacaggcgtgtgccatgcctaatttttaaatttttttgtagaggtagggccttgctatgttgcccaggctggtctcaaactcttgggctcaagcaatcctccctctgtggcctcccatagtgctgggattataggcatgaaccactgcacctggccctgagatatatttgaataattgtaaaaatctgttaattggggttattggagggatggggatattccctgggacaagaaaaagggactgaccttttacttttatacttgatacccttatttcacataatgccaaggtgtctgttttttatgattgttcagggtgattatatatttattcatgtattgtttagagtagttgtatctcatttagctatttctttcttgatggataattaggtaatgttgcagtatatatttaagtacatatatcctgaaacactgtgggaatacttttattggggaagataaataccttgaaataattattgggtcatagaatatacacactgaaaactttgcgataacttaatatcagaaaagattttgattctcaacaagtgtatgaaacttccttttcccacaagatcctggccaacatggaatgttaacagatttttaaaagtattaccaatctaataaaatttaaagtatatttgacttgaatcctgtgtgtattgtttgtctgtatgacagttgtttgtattatatgacaaaaaattaaccaaaaaatattagactcacctgattaccccaaaagatggcttgtgttctaagcctgtttaggcttataagtcaaaagaaaatttctttaaaaaaattttatttgatggtttattgagatataattcaaaagccatacaatttacccatttaaagtataaaatgatttttgtatattcacaaagttctgtggccgtgataattcatttaaaaatattttttcatcccaaaaataaacactgtacccgttagcagtcacttcccattcctcccatccctatgtaaccacccatttgctccctatctctatagatttgcctattctctggcatttcatataaatgaaatcatacaatatgtgtttgtgactgacttcttttacttagcataatgttttcaaagtccatccacattgtaccctgtgtcagtccttcatgccttcttatggctcagtaatattccattggctgtatataccacattttgtttacacatttatcagctgatggacatctgtgtcgtttctactttttagctgttatgagcaatgctgctgtgaacattcgtattcaaggttttgtgtggacatatgatttcatttgtgcctctagtaaccttttaagagactggcagaatgtttttcaaaatggctacaccattttactttcccttgagcatgagggttctgtttttttcacatcactgccaaacttgtttattatttgtcttttttattatagccatcctaatgggtatgaaatggtatctcattttgggttttatttgcatttcccttatgactagtaatgtgaagcatctttttgtgtgattgttggcttttatatatctttgtggagaaaggatttgcttttatatagatagttttacatgcaattcatagataaataccaatgacctgagacgagaagaattacaaaggtttttgaacaatgctctaataatagaaatcggacagaaaagagaaagtagccaagattatttcctctagagatgggaaaatgtacaagttctccaaggatctatgacacaacaagagaaagaagtggagataacaagaataaaactgaatgctctagagacacaaaagggaaatactgaagataggtaagctctcttcagtatcttctttgtttttaagtttattcctaggtaagctatcttcagtgtaaggtagactacgtaagataggtaagctgtcttcagtataaggtagactagataagctatcttcaaaataaggtagactaagtcaaaggattgtaatatcaagaaatgtgatagaccctggatgtggtcttttacatcactaaatgaaaaccatacttaataagttgtatatagtattgtaaagataattgcctgttttaggtatttcaaaattggtgactttggtttagcatttagaaaatattgttcataaggttttttaactttttatttagaaaatttaaaacacttggccggtgctggcgtgttggctcatgcctgtaatcctagcactttgggaggccaaggcgggcggatcacctgaggtcaggagttcgagaccagcctggccaacatgatgaaatcctgtttctattaaaagtataaaaattagccaggcattgtggcgcacacctgtaatcccagctactcgggaggctgaggcgggagaattgcttgaacctgggaggcagaggttgcagtgagccaatatcgtgccactgcactccaggctgggcaacagagcaagactccatctcaaaaaaaaaaaaaaaattaaaatacttgtatggaaaaatataaaggacaatatattaaacagctgtttagtcacctacaatgaacaaatactaattttttgttatatttgcttcatattatacatagaagtacaaatttaggttccactaccagtcccattcctctgctggacacccatcgtcccatcttgtccccaaaaagacagtcactatgataaatgtgaggtaccttcaatcatatttttgtattttattatatatatgtgtagccataagccaatgtttttgttttttttttaatttgtaaggtgatacgatactatatgtatgtaatggtcagcatcttgtcctttttattcaaaattatgatttcaaggtgtatgtatttatatgttaacacatacagatctatttcattcaatgctattatcaagtattctagcttcttaatataccacagtttatctttttctccattgatgagcatttaggttatatttggatttttttgttatgacaaacagtgctgtaataagaacattcttgtgcttgtctccttgtacacatataacaaggtgtcacaaacctgaaagtggaattcctgggacagggagtatcatttgaagagactgtcctttccccagtgtatgttcttggcacctttgtcaaaaatgagttcactgtagatgtatggaattatttcattgttctctattctgttccattggtctatatgtctgtttttatgccagtaccatgctgttttggttactatagctttgtagtataatttgaagtgaggtaatgtgattcttccagttttgttctttttgctttggctattctgggcctttggtagttccatataaattttaggattatttttctatttctgtgaagaatgtctttggtgttttcatagggattacattgaatctgtagattactttgggtagtatggacactttagcaatattgattcttccagtccatgaacatggccggtctttccattttttcatgtgttctctccagtttcttgcatcagtgttttatagttttcattgtagaaatctttcacttctttgtttttaagtttattcctaggtattttatttgtagctattgtaaatggagttactttcttgatttttgtttcggattgtttgctgttggcatgtagaaatgctgctgacttttgtatgttaattttgtatcctgcaactttactgaatttgtcagttataattgtttttttggtggagtctttaggtttttccaaatataagatcatattatccacaaacaaggataatttgacttattctttccaatttggatacccttttttttttctcttatccaattgctctagctaggacttccaatagtgtgtcgaataacagtggtgaaagtgggcatccttgtcttgttccaggtcttagaagaatactttcaggtttttttcattaattatgattctagctgtgggtctgttgtctgatgaaaagtctgaaacggtacctaaaactactaaattaattttaacatctttgagttgtttttagttctgtgagaagagcatatctattttggtttactcagtgagtagagtagtgtctaatactgagtaaatgtactgtaagtatttttgaaagaatgagtctttgggtttacataccctggggtttgtaaacaaatatctgttgattggcattaatcctgatggtatccaaggtacaggaatggcaaagggaaaagatagggcaatactgactgatgcttcaaaatcatgccctagttatgctataatcaagcaggaaatgtttatggaatggaaagattaaggaaaaggtatgttcttattttagcaataaaacgaataccagaagctttaacattcaccagtacaaataaatagtttcaatggaataggtcgaaagtaaagggacatcactagagtaaatgctagaccttccctctccttttatttttagcaacagcaaagcagaaactaagatctacaagtgatcaaagagggtgatccattcagtttctgtgtagacaggaataataataataccttttacatattggtacagtttgtaaaaacactttcacttactcatttaatcttcatagcaacttgatgaggtagaatactataggaagcagtattagctcaggttggtacgtaaattactgtgtttaaatttcaataaaacagctatggaatccaagacattcttggcgcctaataaactgtattctttgccaacagtgaaagtgcttctctgttgcttggtaagttttttccccttagaatactaataaagtaattgattaactttcatttttattttgatttgattgggacagcaatttagcagtaaaaaatgtcacctttataaatcctgtggtttctggttcttggccagttaaattcaacctgaccaggaggcacgcttaattctaaaattgcttttaccttctgaagtttttgtggtatagacatcctcctttttctactttaatgaaagcatgttataagcagatcataacaatttttttttctttaaaacaatattgtaattaggccagttgcagtggctcacacctgtaatcccagcactttgggaggctgaggcaggcgggtcacttgaggtaaggagttcgagaccagcctgaggaacatactaaaaccccgtctttactaacaatacaaaaaaattagccgggcttgctggcacatgcctgtaatcccagctgctctggaagctgaggcatgagaatcccttgaacctgtgaagtggagtttgcagtgattctaggtcgcaccattgcacaagcctgggtggcagagcaagaccctgtctcaaaaaaaaaaaaaaaaaaaaatggctcacacctgtaatcctagcactttgggaggctgaggcgggcgaatcatgaggtcaggagatcgagaccatcttggctaacaaggagaaaccccgtctctactaaaaatacaaaaaaaaacaaaattagccaggcgtggtggcagacacctgtagtcccagctactcaggaggctgaggcaggagaatggcgtgagcccgggaggtggagcttgcagtgagctgagatcacgccactgcactgcagcctgggcgacagagcgagactctgtctcaaaaaaaaaaaaaaaatgtaattgatgtaatagtcccaaaaaagaacttggcattaagttaaattataaaatcagaaagctatgtaatttaaatttgtattcaaaatctgtatattggcatgtatattctgtgccagtttatttaagatgttactgtatcatgaagcttacttaaggcatataatcgtctgcactgtaaaacaaactaccaaattaatgtactatctcaaagaattaaacatataacaattttgatgaccacctaaattttagaacaactgtttttttaaaaaactttttactatggaaacttttatatatataatatatataaattatatatgttatatataatatatatatgttatatataatatatatgttatatataatatatatgttatatattgtatataaattatgttatatataaattatatacattatatataaagtataaattatatgaattttatatatatatattttttgagatggagtctcactctgtcacccaggctggagtgcagtggcacagtcttggctcactacaacctccgcctcctaagttcaagtgattctcctgcctcagcctccctagtagccaggatgacaggagcctgccaccatgcctggctaatttttgtgtttttaatagagatggggtttcaccatgttggccaggctggtcttgaactccagacctcaggtgatctgcctgccttggcctcccaaaatgctgggattacaggcatgagccaccatgtctggccaactatggaaaattttaaacatacataaaagtagagtagtatatgaatcattgtgagctcattaacaaaagataatttcagttcactttaaaaattgacgtgtgaaagtttggactcatacattttgttttggtctaaagtttccttggcaaatattcacatggttccattgactctggctcctttttaagataggatgcttttttatttttttaagagaaaaattattgctctgacaagctaatcaaagatatttaattttggaatttggaggaaaggcataaacctagtttattacaaaaatacctctttttagttttcctagccttaaagtaggagacaacctccctccctttacacaggttatactttcatatggtatttgcataaagaaatcttgttttcaccctggcctaaatatacatccaattttctctgcaccttttaggtaactcttttttaaggagggtgtgtgtgtgtgtgtgtgtgtgtgtatgtatgcgtccgcatgcatgtgcatgtgtgtacacgcatgtatggattttcatggtttttaaaatatagacataagtacaaatacatttatttccccactcttcatacataagatacataatactgtatatatcattctgtattttgtttttaacgtttatgtaggccattctggaaattttgtttcatacataattttttatatatatacaactacagttccattgtatagatgtactatagtgtatttaaccagtgatgtgtgtatggtcattcaggttctttctagacttttgatatacagtcagtactgtagtgaataatgttgcacatacattattcttatgtattgcaggcatatctgtgagatagattcccagaagtatgcttgactaggtcaaagagaaatttgcatttgtgactttgatagatacaaatttcgcttcatggatcttgcttaattatgagatgtctgtttatagcttcataccagtagaatatgttatcaatttttttttggatttttgccaatctgataaatgaaaaataacttcactagtttgggtttgctttcctctgagtgagactgaacatgttttcatatgtgtgaaggccatttctacttctgtttctgggacctgtctctcatatatttttccctttttctcttgggttattggtcttcatctcaatttttcaggagctctttgtgtatcaggaagctaacacatctgctaaatgagttgcaaatatatttttcaccatttgtactgtctttttacttcacttttaatgtgtttgttttgccatccaaaagtttttttttagctttatgtaggtgaatataatatttttttcctttatgacttgtagattttgcatcagagttataaaagcctttttcatttgaagatttgaaggtgccatgctttcttctagttcttttatggtttgtttcactctccttccctttcttccttccagcatttaaatatttgatccatttgtagtttattctggtatacaatatgaagtatggatgaacctttttttctagattagttccagttgtcccagcatcagttatttaaaagttcatctttaccccatttcagatgttgcctttatcttattcgcactttctttatgtgttagggtctattttggtctttctgttttgttccattgatttcttcatctgttgtacaggtactatcatgaaagaacaaatagtataaaaattcagcctacaaaagtggaattaagaaggactgatagatggcttgccttggtaatagagcagaaaaccaaatttttgcctgttgatgaaatatatatgcaacttaggatcagcatatgtagagaagcatgagggagcatagccatatttgaagtgcattactggagtgcctaagggaaattaccaaagaggctgaaggatagatagctccaagtcattggtggccttttatgacacattatggaattggaattttatcttacaaataatgggaaaattttaagcaaactagtgaaatttaagattatatattagaatgggttaaatgctgagttcagtgaactcagcgtgggcttcagattccacaaaccctatgaaattttgtttcagagagcacatttgaggctttcattagattctcagaagtctttctctctctccccctcccaccctcacacacacacacacacacacacacacacacacaggtgtgcatgcacatgcatgcccaaaagttaagaataccttcttaaaagtaaaaaattaaaaacaaagataaaaaagaaaaaaagaatgccttctttagaaagatttctatggtaacataggataggatacattggatgtggtttaaaactaaagatcggtcaccagtcaggaaattttggcattagtataggcggaagacgaatggcacagggaaaagtgagctttcacttaagtttattatttctgaaaggttacaaccagttctcattgttcgtagtagttatggtctataaattcactacaaacaattagtgaacactgaaccttgctcctaggggaagtacaaggattattattatttaaatgagccaaaaaacccctctgtatattggcctctaggttgtttcttccattacaacaggttgagacctattggcttaaaagtgtgcttgcaccaaactaaattttttgtacatctaattgttttaaatataatcctgataagcagatttttagccactgagagcctgcctactttgcagtccccacaaaactgctttgtactcaacatctattaggtataaataagatcaaatcccaaggctataaagattccaagctgctgttgccctttggagctctttgatacatagactccctgctgggttgtctaacattgtcagctagacatataagctccctctcctactcccctctcccctgagagtcccctagccctcctcttctgggtgatagccactccacctcagcttctggccagtcctgtttgggtagtggccactctgccttagcctctggacagtatgctttaaggacatcctctgcctgcagatctgtcaatgtcacccaataaagctatgcatgctactgccacctcgtggtcatatctttttccttgatcagaccccagatccttcacatttaaaatacagttaggttcctaccagcctctagtcacattttcatcagtcagtcgatacgtaaccttgttttgtgtgtgtttctgtttaaagacatattgttgattcattaacattgaactcacggctgacattactataactcatgcctgaaggaagctggcctaacacatgtacttactctgccagggacattatagccttcttatgcttaccagccctaaacagcacgtgagcactatgcttagggattattttaaacagaagaatcatcaacaagaagcacaaaagtgagaaaaaagtggcaccaaataggctctgcccaggatacttgtttatagtatgaaaattgaaacaagaaggcaagagaaagcttcactgttcaacctcagttggagaggtgcatgcattaggcgactcaaatatctcattgctctgcgcatgtctatgaaagcactaagagtattgactttagagttacaaataaattttagcaagtagagagatttcaagtatggactccacaagtaatgaggatcaactgtaccatccagtctctttaatacttccagtgatgagcccagtctctcatgacaaacttttaccttgctgggtaggtaaaagttcagggtttttctttgtttgtttgtgacagtatctcgctctgtcacccaggctggaatgcagtggtacgattttggcttgccgcaacctctgccttctgggttcaagtgattatcgtgcctcagccacctgagtagctgggattataggcgcccgccgctctgcccggctactttttgtgttttttagtagagatggggtttcaccatgttggctaagctggtctcgaactcctgacctcaaatgatccacccaccttggcctcccaaagtgttgggattacaggtgttagccactgcacccaacctggatagttttaatggttatattgcactaccctctgcgtcctgttatttctggtcatagatcctaattaggctttctggactctttcccccttcaatatattatttgaattcagctgtcttatatttacctttaccccctttttttcccctctagcataaatattcccaatttctttggaaacttttggtttgctaagcacctcaatttaaatgaatggaacttattgttacaaactaacatttccccttcttcttacacctcccaataaaaactgtgtcctctgtattcacactgcctcactgtagtctacaccttcagttccaagtaactcagatttgcctgtgggggatgaggcaaggaaaaggctaaagatagataactttaaatatacagagagattactttttttttccctttccctttccctccttctctgtctctctcttcccatgtcatctttgctgcttcgtatgttagcttcattctataacagattttctcagtgtggtggagaatatggccattgagggctccagatttgtatcttctgcaaatctacagaactacagtagaaacaaggacttttctctgtcagtgtccatgtgtcaatttagggaagaactcaggtactgtttgggtcatatatatagccctggaccagttgctcttgccgggggtatgggaaactgattagccagcttctgtcatctgtagtgacataaaagtagtgacccttgatagcttttctagtaccatgtggatttctagagaagggaatattcccagaggaaacaggggcaccaaacaacaaatatcaagtatacatgttaagacaggtttttttcttcccgctatgtttagggccagtaagaggtctcttaaggacagtcagtgtgattgaagggttatacagttttcagctttgaacagtattggatcaaaattgattttgcttttaatattgacatctattattgctcagtgatggatatactgcgttggtgggtatattgtagcagatactgttacttcttctttttatatgtttaaagtatttcataattttaataaaatagaaaattaactttgctttgatttaagttggtgaataataacaaatatttgggttataatttccctttagtattaagttagctgtagaaatggtgttgtatctgacctagtaacccatttgactttttaaagatgaattactaaatttttttaatgatatgaaaaaatgtaatttgctccctttacctcttatcaatatatttatgataccataggtacctgcaaggtgtggagttacagtccgagacagtctaaagaaagcactgatgatgagaggtctaatcccagagtgctgtgctgtttacagaattcaggatgggtatggtttgtatgtgacgtgaaattttgtttaaaaagaaaatcacacattaaactttgaagttttcttaggatctttaccaaaacctagggaattgaaagtgtactttaggaaaaagtattaaaataatactaagttagcctgaagaaatactgtaggccatatgaggagttaaataattgtatatgactgtagggtttgttactttgatcaaatgattttatttggaatttgagattcttacaatttttgaaccattcagagtgtgatttatttggataatagactcttacccccctcccatttttaatacaaactcatagtttcacaaaaggtatatcaaaattaacattttatattgacctacttttctttcagaaagtgtctaacattgttccaagaccctcacattttgaatcctctttaaaaaaaaaaaaattattttgggggcatgttgtccctgtcccttgagtactctttttccttgaatggatagataagtccgtacctgtgattttttttttttttttttttttttttggaccccaggaacaatccattttctgctgttgtaggtcttttctggagctgacttgaagaaaagagtacatctctttaccctgctgtttgtccaagagtgatacatttatttggggtaaacttaaaattaatttattgccatttaaatttctaacgatggaatattagggagccaaacctccctcactgttactagcccctcgataaccaattttcatatcttcagcatgaggtatatgaatatttttaggtgtaataaccaagaaaggcttgtgtctacatttttcagagagaagaaaccaattggttgggacactgatatttcctggcttactggagaagaattgcatgtggaagtgttggagaatgttccacttacaacacacaactttgtatgtatctttacatttttttttgaaatgtcaaaaatgtttagattttaatgaatgaatttttatttagggaatgtgaaatatggatgagtaattttggaactgacattttacctgagttgaaatcagttgttttctttaaaaacttgtatttaaacaagagtttaattttaatctttatactttctttttaatttaaaaaagtaaaatgtatgcattgttaaaagataattttgaatagtgcagaagtatgtcaagtaaaaaaagtgaaaataccctcctagaggccaacatttgtttagattaacagattattgttctgtcttccaaacttttttctgtgtacacaaacgtgtgcttgtacctgtaagcctaaagttttttcctttctttttttctctctttccttcattactttcttttcttttccctttcttaaatcaaagtagagccatgctatgtgatattctttgccttatttttttttaattcaacaggatgtcacggacatcttttcatgtcagtatacctggctttattttagtatgactatgtaataattcgtaagaataggaattcattatatttaaccatttctctattgatagacatttaagtttttgtattataaaaaatctgttacatacagggctaaacaaggtctttctgcatatatcttgacacacttgtacacacccttgtgtttctgaaggataggttaatggaaatagaatggctggatattaaactctccatgaggcttttttctttgtctggtttttgcatttgctagaacctagcataagcctaagggtcaccagcataaggcccggaatgtgggacctttccaccttagagatgaggatctacatagaacttgagaacttatcccctaaaatggcaggcagaagccaaaagttgtctctgaatcaggaatacatttcttacttttctctatgtatggaattttggccagagttttttctaggtggataattactgctaccctttaggcgtcaagtgttttcccatgtgtcttggtgattaatccagctgggctcttaaagcagatgattgattagattatttcctttggggtttatgctttcaagcctccattagtagtgaataatgaaatcatgtttgtgtttctgtaaggcattttttgggagaagtgtaagatacacctaatgtcaatcaaattttactgtgcagcaaataattttttaacatgttgaattttaagtggataaatcttaacatttttatttaagttcttaagaatacaaatacagttgagcatacatttcatagtgattactactctgtttcttaataattcttcttagctagaaattgaatatgaggagataaaagaaagacctaagttgctaagtgtgagaaaatagtattaaaatgatgcctcaggaaggtccttgccacagaaggattgtgaaagcaaactgcagattgattccagagtatggaaattgtcaccactcagttggatacctgccagtaaatttttattgagaactttccatttgccaataccatatcttacctagggatatgacaaatgaataagacagtgtcactgccatagagtagagagaagatttaataaattttcataaacattcataattactatatatgtataaagttctatagatgcacagagattcctaacctggcttttgaggagttaggaaggtttctcagagacagaaatatctaagcttaggcctgatggatgaggaagtaagtatcaagtaaagagtaccctaaacagagggaagagcaagaaagcatgttacctttcaaagttttgaaagaagttcagaatggatgatacaaggttgaagagtaggttgttaacagtattgtgggtagaataagagttgtgggtagaataagcagagtccagctcatgaaaatccttgttagccatattaaagatttggatttttctgtaaaataattagaaatgtaatagttgtaagcttggggagaaatgtgattagatttgtgtgttagaatgattgctctgagtgccatatagaggcaagatcaaaaacctatgttctgcttagaaaggaaaagtgttcttggctgtcttgggttttgcttggctattcaaggagggctgcttatgcctcatggtttcatattataaaagcaatcccttcagtatttctctatatcccaagagtccttgggaactgggaagtgggaaaacaagatttgaaacttttatatccaaaccttctcctttttctgcagacactcagtgtcttccttcacacagccccacaccttacaaattaatgcatgcaaattaccttgactgtgcctctcactaatttgccatacatatttatgtatactcagatactagattaagtgtaagctgtgacccaaaagaaagatatatcttcctgtgctcatctttattgacaaaggtatacttacagatacaggcatatattgcttaaaatttatgatcaaatgcatatccacatgttttctttccttcagctgttttggtcacctacctacttggtttggtgaataatggccacataaaaaattttaaagattttaaaatttcttgtatatccagagacaaatggaaaaagaacacaattagaaatagacatttacctgttttatatcccctagaaagtgatacataggaaaaaaggtgaagaaaataagagtcacttttaaaactaaatgtcctcaaaaagccagaatgtattatatatcaggatgtaattttcttgaaatattttcaataactttctattcttaatggaacagaatgtgtaaataaatgtgtattgaaaatggacttttggctgggcacagtggctcatgcctctaatcccttgagaggccgaggcaggcagatcacttgagcccaagagttcaagaccagcctgggtaacatgacaaaacctcatctctccaaaaaaatacaaaaagtaggtgggcatggtggtgtgcaccaatagccttggctattcagcctgaggtgggaggataacttgagcctgggaggcagactttgcagtgagtcatgattgtgccaccatactccagcctgggcaacagagcaagaccctatcaaaaaaaaaagaaaagaaaaagaaaagtagacttttgatgttgaaatctatttaatgtatcataaaaaaatttacatgtagcagaatagattaggaagttctaattcatgttgtatatagtcagggtaagtagtgttgtatgaatacagttatatatggagtcataatgtaaaatatcattatttgtgattaaaactctgaaaaactgggcacagtggctcacgcctgtaatcccagcactttgggaggctaaggtgggcagctcacgaggtcaggagttcaagaccagcctggccagcctggtaaaatcctgtctcatttgaaaatcttgtgagttgtaactggttttatacaaaatattgaagagtggaaattgtataattacaatcatgtaattaaaagtattaaccaccccccccaaaaaaaaaacctgtctctactaaaaatacaaaaattagccaggtgtgatggtgtgcgcctatagtcctagctgctcgggaggctgaggcaggaaaatcgcttgaatccaggaggtggaggttgtagtgagccgagatcgtgccactgcactccggcctgggtgacagtgagactctgtctcaaaaaaaaaatctctgaaaaactgaaatgaattaagaatatagaggccgagtgtggtggctcatgtctgtaacactctgggaagacgaggcaggcggatcacttgaggtcaggagtttgagaccagcctggccaacatggtgaaactccatctccaccaaaaaatacaaacgttagctaggcatggtggtgcatgcctgtagtccccagctacttgggaggctgaggcaggagaatcacttgaaaccaggaggcagaggttgcagtgggccgagatcctgccactgtactccaacctgggcgacagagcgagactccatctcaagaatacagagcaaagaacaaataatgaaatagaagtcacccatgctctcgccactctgaagtagccactcacattttgatatttattcttatattttcttattattatatacactaaataaatatattttaagcaatttctggctttagtgggatagattcttcctagtgcagttctgttacgtgactcatgttctacatcatttgcctttgacatggaattcttaacatgttgccttctaagtttcacctagagaagtgttcacaaataagtttatgtggcccaaacattctaatcctctaagaaattgatcatttgttagaaaaaatagatcttattgtcttttaggtgatttttctgtttcttattttttttagtaagattaggaagagctgtttcaattttcatatgattacttactagttttataaataattgtttttacatttttatccaaagttaaccattatgtttttggaccatagatcaggggttctgattctgtcagctattttgtttttgtttttgttttagctattatgtagattgtatttatagtctctctccctcccacccccaattccactctggaggaattcactgttaatttttaatggtttctgttttaagctcttttggtgattattttcatcttactaaatacagttacacattgcctgatgactgggatatgttctgagaaatgcatcaatggtgattttgttatgtgcttacacaagcctacatggtatagcctaactatacaactaggctatatggtagagcctattgcttctaagctaataacctgcacggaatgttactgaactgaatgctgtaggtaaccttaacacagtggcaagtttgtgtatctaaacgtagaaaaggtacagtaaaaatacagtataaaagatcaaaaatggtctacctgtgtaggatacctaatatgaacagagcttttaggactggaaattgctctggtgagtgagtggtgagtgaatgtgaaggcgtaggacattactatttgttactgtagacttttatgataaacattgtacacttaggctacagtacagttttaaaatttttctttttaaaaagtttataaagtaaaaaatttacattaaactaaggtttaattttttttaactttttgactcttgaaataacagtttcaaaagtttaaaaacattatatagctatacaaaaatattttatgtgcttattctataaacttctattttaaaaatttttaatttttcttttttactttttaaacttttctgttaaaaatgaagacacaagctgcacacggtggctcatgtttgtaatcccagcacttgaggaggcctaggcagacgcatcacctgaggtcaggagttcaagaccagcctggtcaacatggtgaaacccttcctctaataaaaaataggaaaattagccaggcctagtggcatgtgcctgtggtcccagctgctcgggaggctgaagcaggagaatcacttgaaccaggaggtggaggttgcagtgagccgagatcgtgccacagcactccagcctgggcgacagagccagactctgtctcaaaaaaaaaaaaaaaaaaaaaaagacacaaacatacacattagcctaggtctacacaaggtcaggatcttcaaggtatcactaggcaataggaattattcaactcctttataatcttatgggaccactgtggtatgaagtccatgattaactgaagtgtcattatgtgacacatgactgcaattatcttttagccacaatttcttgctttattaactttagatatcatatactgattactgattgtataaggaattagctcatttatagttcttcctctccctcctctcccccaatatttttattagtagttttttggttcttctattgggtgcctttgtaactttaatatatgcctttcttgttccgtcaacaccagtcggcatttcttaacctcccttctttgtatgataattaaagtataggcttcttcctttcacctctgtttcctcctccttttcatgtctacttttacttttatgttgtcaagcttgaaatcagttgccagccttttactcatttctcattttttaacttctgggtttttaagttagatagtggggtgggaaataaatatgtgtagtccatctattatcttgaacccccggttttcattttataatagtactttagtatgtgagtttcatgatataaatatattacaactttgttattttaaacaagagagtagatacgtcagtttctagaaagttttcttgtgagtttttgaaatctctgtgattttttactttgcaggtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgagtaagtaatccaaaaatatctcttttctacctaccattttacacttaaattttcttaatgtgaagctacgatgtctaaaagtctgtgagggtttttcttccatacgattgttatagagaattttttttaagtgtagttagagaataatatgtggaatggacagtatttctctcccaaattgtaatgctggttcagctatacagttaatttatattttatattatcgtttaattaatcaagacccctaacccatagaaaccatttttggatagtttctaggaggagagggagagttgtttcaattaaattaagcattatgattttgtaccacagatcaggtagtctgattctgttagctattttgtaaattacgtttatattcttcctctctctccttcatccccattcagctcccttcccccaatttatttgagtaagatgtaaaatttttgtatccagtatatatctctttctaaaatttctctttgctgtatgccagtttttctaatagattagactgagtctattatctctttttgtgtcattggtgctgctgctgttaaagtcttacttttcttgatcacctgagcaaataaaacttaactctgtactttaagtaaattataatgtcacctaatttacagtagatactttttattctcattctttaaccataaagcatgattttcatcttgtagtatgtagaaatttgctgaactgaatgattagttttaagttataagcatgcctttgaaagtgcaatacaattttttttaaaataagcctttagacaaaaatacaactaattgaattttaacagttgtttctgagaatggaatttgatctcagtttttttggttaactatgtattttggtatatgaagcttctgggttttgcacaagttaggtttgttttgttttgcctcacagtttgctgtttgtctccaagttctttgaacaccacccaataccacaggaagaggcgtccttagcagagactgccctaacatctggatcatccccttccgcacccgcctcggactctattgggtatggtttgacttctgctcttgggcgacatgctacttgaaccgctttcttttggatctcctggttaattagaaacctttccaatgtttaaattgttaaattaaggactttttccccaaataacttatcataccactcagatatttacatgcatttgatacaaataaatgggattaaagctgacatagactatttcagaatcagtcctgaaaaaatcaatattgggtatgatttgacttctgctcttcagtgacatgcttcttgaactgctttcttttggatctcctggttaattagaaacatttccaacatttaaatgcttaaattaaggatcttttccccaaataacttattttatcacaccagtcagatatttacttgcatttggtacaaataagtgggattaaagctgacatagactatttcagaaccagtcctgaaaaaatcatgcaacagatcattttgagtctacaccttgagttcatcttttattaggtatagaagtatatgacttccacttatgaagaagcattgatatgtgagacaatggcaaacaatgtaaaaatagtatataattataatctacaatttatgatggagtatattgaagtatgtgatgaggacataaatgtattcatgtttacagaaggaagaatagtgaggaaaaagagagtgctcaggaaaacttaatgaagaaggtggtatttgaactagactttaaagaattactacaatctgaacgggcctagggaatagaagcatggtgaaaggggaatggagaaacaacagatataaagggaataaacagatataaagggaatgaagatgttaggtttagaagctagtgaagaaaggtttatctaacttaagaactaccatgtgtaaaaccagattatggagagtcttggaattgaggccagaatttagacttaaaggtctttaagcagattactaacttgatgaaaatggctttaaagaaaaaatcaattagcagtgaaatacagatggattgacagaaaatttagggtgaagaaggccaacctaggatgttgttggtagtgaaaactgagagaggcagtgaagacaagttcaagtgctagaagtatggaaaagggatagatattcataaagcgtaaaagaaaaaaatgaacagtattattaatcagttgaggataaagctgagaagtgactttaaaaataatgcaaaggcagccgggtgcggtggttcacgcatgtaatcccagcactttgggaggctgaggcgggcagatcacgaggtcaggagttcgagaccagcctggccaacatggtgaaaccctgtctctactaaagatacaaaaaaaaaaaaattagctgggcatgatggcacacacctgtaatcccagctactcaggaggctgaggcaggagaatcacttgaacctgggaggtggaggttgcagtgagctgagatcgcaccattgcactccagcctgggtgacaggtgagattctgtctcaaaaaaataaaaataatgcaaaggcgtcatttaagcttcatagtaggaaataaaaaggaagacacaataaagatgagttaagtgggtatcagtttactttggaacatttctcgaactcctggcctcaggtgatcttcctgcctcagcctcccaaactgctagaattacaggcgtgagacactgcacctaattagctttggaacatttctgacacaggtctgtgtactctttcacattgaatttggggcagcgttatttaggctgcgtctggaagcacatgctttaaaaaaaaaaaaaaaaaaaaaggccgggcgcggtggctcactcctgtaatcccagcactttgggaggccgaggcaggcggatcacgaggtcgggagatcatgaccatcctggctaacacagtgaaaccccgtctctactaaaaatacaaaaacaaaattagccgggcgtggtggcgggtgcctgtagtcccagctactcaggagtcaggaggatggcgtgaacccaggaggtggagcttgtagcgggccgagatcacgtcactgcattccagcctgggtgacagagcaagactccgtctcaaaaaaaagaaaaaaaaagtccccatacagcattctgtcctagaatattcctgagatgttaagattagaaatatttattgtcagttttaaccctacttcctcccactcttcacagtgtaatgccatttcttcagatttctacctagagaaatcttttcaaaatcagttgacattttcagtacaactttgtaatttttacaaagccaacctttctggtgatctcatgggttagaaaattttgcaaaagaattttgtcttaagcagtgagtatacttacctatttggaacactggaaaatgcagccgttgtgttctcttgtcaggttgtagcagttatctcttctagtggagtcattttattcccgttggctctatcactacttgtgtgcagctgtgttttttttagatggtcaaatcaaaatgttattatttggttggtttttaattaatcttttggttataatattttctcctgacagattctgtagaccatgatgcctttctaatggcaggtcccagttctgtgagcagttataaaacaccctaatgaaattagcttggcctctctgaaggtttataatggtaatgatccaggacatttgccttccagtgaaggaatgctatcttaaaattaagaaaccatttgcctgtgccatgaatattttattggtaactgaactgaaatatattctgatcttgagcaaatgataagatgttcaaacttgtctgtaagtcattttcctgatgtttttatgacataactccatatggttgtctctgtaaagatagaagccaatcttgttcagtatcaaaactcttttgcagtttgttactagtcctgataacaataataatggtcatgaaacaagtgtaataatggatgtaatcaagaaatattggagaagcaatacattgcccagttttgaatacaaagtgagagatacctccttttctactttttaaaaatgctgtagaatgtataaatttgtgagaggtcaacatttaaaatgtacgtatcaccagaccaaagttttcaaaacttttaaatattctagattaagaatgagatctaaactaactggtaatttgctgaagggtattataattaaaattccttttgtctttaatttggtactgctttaaagaggtttactgcaagataggtacaggtatcattgggagaaagccagtttgccgactgccaagatacttcatagggtataagttaccttggttagtggaaaaatttcccagtatggtaatgtaattttatcgattcccagaaatgatgagattgggagttatactggtttgtattaactgaaatcagaaaaaaaattgaatcaggataccaggtaatatttttcagtgaaaaatatacctgttatgtaaattagacttcttgtgttgtgtgcgccaccactcactcttcttttgtaagagatttatgggaaatcaaattataatcagaagactgttttcgttaagcatagaattaggacatggctgagatattcaatgacatcagattatgatcacttcaagtgttcccttgtacttgccctgaaagctagagaagttgacttggtggaccaagacacaactattagataccaactaccaaattgagtttctctgattttgtataatacgcagatatcatcattttctaatatatacattctttagatatgatagtgaagtgtcttagattaaatctggtttttgtttttcttctggaccaatgtcttttgcatacttaactctgatttgttatctactacatgtttctgtcatattcctaacttggtgagtttcagaagtgacttactgccatctctgcctatcccagatcaattattacagtagactatcttatgcaattctagttattcatactttttccaattttaagccttttttttttttttttaagatggagtctcactttgtcacccaggcgagtgcagagacgtgatcttggctcactgcagtctctgcctccctggttcaagtgattctcctgcctcagccccctgagtagctgggattacaggcccttaacaccacacccagctaatttttgtatttttagtagagacagggttttactatgttggccaggctggtcttgaactcctgacctcaggtgatccacctgctttggcctccaaaagtgctgggattacaggcatgagccactgcacctggcctgccttgagactttaaatcagcctgtaaatggttgtcagtcagtcagtgccctttctaaaactttattgactaatgtcatttttgcattctttttcctgctcctaaaattttctagctatagacatatatttggctacctaaagcaaaaataaagacagctctgtcagaaaccaaaagtttctcaataatcagaaaaaataaaaaggacctagatggaacatgctaattttcctaaaggcttgtttcttacctataattctcattgagccgataccaatttttttttagtacataatattttattatttgcatatcaattctaagtggattcatttcattaatataaacacatgaagtcaaaacttctttccttatctttaataatatgcttcaaagaagtaaaattgtgaactggtgtggttcagattctgacatgttttattcagagactgactttcactgttaggcttccttggctcttcaaacctttattcattcctttcctactatatttttttcccattcctcacgtctcacaaaagtgtctttttattccctcaacattgtctttctagctgtgtcttagtaaccactaataattagtttgcataaaatagggtggaatgataaccaatatgtgaagagagcttattggcacttagccattcattggtcctgatggagttaagtgagacagcttacctcatctatcaagtgacactcatttccccactcctaggataccctttctgaggggctacatccttccaagtgtttacaatctagtctcaaaactttagtgttctctgtgagtgccaggttcattttagggtgagatatcatagactatgttatttagctaccataccgaaataggtatgtaacatattttggtgattttccaaatagcatacaaatgtaacattttggtggttttccaaatagcagttttcaaaaatatttgctttagtggttaatatatgattctcttgtgtctctgttatcaataatgggcatgataaaaaatccagaatatgagagatattggcactctgaggatcatcttctgaatttgaaaaggatttttcaatattgttctggattttcattcaactcctgtaaaggaacaagtacatcattcaggtcctgaaatatgcatttgtattctcaaaatatttataatttcttaatatgtaaaattttcattttagtaaattcagatgtcaagacaatgttagaaaaaaatggcaaattatattcagtcattctcagagcatttttatataacttcaaaggttgaacttcttcagttgatggccacaggtaatttctagccataagtaaatttccctagtgttttccaggtaagaatcagtggtcttatcattgatagttcctggagggcctacttgagcaaagcagctttggcagtattggatttttaaattaatacttttaaaagtcattactgctaggtttttaatgctttaatgattttgagaatataaaaacaagaaaatccttttatcttcctttttaaatattattacctttatatcgttactctgaatcttatcttccaatgacttcatttttccaggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgtaagtatccagcattgctagaactaaaaaaaaaccaagtatgtatctttatttttctgctataattataacttagatcagaaataagtgccatttttcatttatcacagttattttaagtgataagcttcttgtgaatcacaaatcagaaaagcttctggtttctctctgatgacattaaatatttcactgactccaggttatacagtcactctgatttttttcccttatgataccatctctataaaagtcatcttcaaatgaaaatggtttaaatatcaaaggactgatagaagcccttgacagaattaagttctttaaaacttttataaaaatgattatgattgtgctataagaggtggatatgaaattaagaatttcaggccaggcatagtggctcatgcctgtaatcccaacactttgggaggccaagatgggtggaacgcttgacccagaagttagagaccagcctaggcaacatagtgagaccccatctctaccaaaagtaaaacaaattagccatgcatggtgaggcatgcctgtagtcccagctactctggagggtgaggtgggaggattgcttgagcccaggaggtcaaggctgcagtgagctatgtttgcaccactgcactccagcctgagcaacagagtgagaccctgtctcaaaagaaaagaatttcaatttgtgctatcataagcttggcattatgaccaacaaaaacttgattttttctgtgtttattttaaaattagcatataattgaaactataaattttattaaatattaatataaaagaaaaacttataaattttaattttgtaattttaggtaaatttgcaaatcagactttcttcccactttttactaagaaattttctctatttttattgggttcattttaagtgacttttttctagtactagttttccttaactagcaaggttcacctctatctagcaagacctaaaaacaaaggaagaaaggggaaaaggagaatgtgatataagaaatcaaaccatatgtccaggttagggttgttctcagtctgtccaaaattgcaaccttcatctttactttgaaaactatcatccttttagactattccctttttctctgattgttaccactgttccctggtatgttgggcttcatttagagtgtcatctttgtatcttgccttttcatcctctctcgtaaccagtcacagggtatcaccttttcatgtgcatctcaaactggtttttacttattaccgctgccatttgggcccatattttttcacttgaattatatactacattaatctcctaaatagattttctgccattgatttctctctatctatttccattccttctgcaactgtcagaatttttttacacttctagcatcatctgttctcttgttccaaaacttttagtgacttactgttgattacaaggtaaagtgcaaactctttagcatttttattcatgttcaagcacttatgattccattaaaaagatttactaataactgtgggctatgcattgtgttaggcaattgggaattatttatgaattagcacatggcctttgccctcaaggaagtcacagtctaataggtgaatcaaacatttaaatagataattacaaaatatataatggtagtttagagaagggggtaataaactccatctggttggatctaggaacattgagcagagaagggacaacctttaagccagattatggtgaataagtaggagtacactacataagggggttaggaaagtcatttgttaaggaaatgaaaggcatagaagtgcctttacatggaatgtaattaatagttgatattgaagtttagggtccaggagaaggcttggggaatggtggaaggtgaaactaggcaggtaatatctacagtgaaaggctttgtgtattactctgaaatctaaagcagtgctagggaatctgaagaattttgattgggagaggaagtcatcagctctatattttagaaaaatctttgatggtagagtggaggatagatgaaatgggaaacacatagaggcaggactgtcaataatgtggtttttacagtatttcagacaagaaatgatatttaaactcaagtaatagcattggtgctgagaaagagtgtgttttggggagggagactatgaattagtgaattagtggtaagagtcttaggaatcatgttgaaaatgactactatttatgaatacttattacatacaggtactatgctaagtgctttacgtagactttcttatttcatcctcataaaaactcataggttatgtactatgattatctttattttactgttaagaaaattagatttacagaggttaagaaacatacccagattacactgctgataaattactgaagtgggttcaaacctggcctttctctcttacacttaaccactatactgttttgtagtagaggagaggagtgaaaaatatgagaagtagaggataatgccaggtttctggcttatagatacttagcttatagaccgagtttctggtaaatagcacagtttgttaaataccaaaggaaaacaaggtttgcagaagcagcaattttagctttttgggggcatatattgactttaagatgcctgtgggactttcaggtttagaaatccagtagcagttggatataaggaccttgagtagagatacagatttaggagtaattagcatatttatgtcagttaaagccatggatgtaaattgctcaaagagcatatgtaaattgaaaaggggagaaaatgtaaccctgataaacattaacattggaggtgcaggcagagactctctgtctcctttatgggctggctcttcctaagtctagccccagataactaagaacaagtgttgcagaagccaaaggaagaaaaggggtttcaagaataccaaagtagttggtgtcagttgccattacgattcaaatgagataaagactgaaaggactatcaattttggcaattgaaatgtcatctttacttcattgagagctgttttagagggccagtaagaggagaaggcagcaggtgaggaagtaggattaagtgttaattttgagaagcttggctgtgaaggaatgacaaaagaggatagcttgattcagggttgagggacaattttttttggtttttcgggttttgtttttgttttgttttgttttttgagatggagtcttgctctgtcacccaggctggagtgcagtggtgtgatcgctgctcactgcagcctccacctcccgggttcaagcaattctcctgcctcagcctctcgagtagctgcgattacaggctcccgccatcatgcccagctaatttttgtatttttagtagggttttgccatgttggccaagctggtcctgaactcctgacctcaagtgatccgcccacctccgcctcccagagtgttgggattacaggcgtgagccactgcacccagccaagggacaattgtttttaatgtaagagacattagtatatttgtaactggagaggaagaaggcagtgaagagaaattgaagataacaagagaggaaatgattgatgtagtaaaattccttgagcttggaaattatgtctctcagagactgtgaagaattaaagatggacatagccagctctgtaactctttacagactgtgtgatgttgggcaaattatttaatcctggggctagtagtgtttgccttccatagtggtttgaattgcttattatgcctggtacataataagaattcagaaattatagctaatattaatatgcaaatagttatagatattagagcagaaaagttgtttgatggcttttgttttctctattatgatgaagggaagggatgtaagtaagagaaggaactacaaaagagtgggaaaaaagttgaaatatccagttttcaaatgctagaagaacctttgtaacctagaatgagtagaaaagattgtcaaggagctttaagaacacattcggaatttaaaaatctaagtttatgttgttactagcagtaacttgtaagagtggagaaagcaaaatttggttaatccatagttgtggagagtttcagagctgatgcaacagaaaagaaagggatatggcctttgtcttgtagttccttccacctgaaagactctttgcttttctacatgcctatctctgaaaccccaactcagagtaattccttgactgctttatcagtgaccaagtcctatagttatcatacacagcactaaaaatcttatcggctgggtgcagtggctcacacctgtaatcccagcactttgggaggccaaggcaggcggatcgcctgaggtcaggagttcaagaccagcctggccaacatgatgaaaccccatctctaccaaaaataaaaaattagccgggcatggtggcaggcgcctgtaatcctagctactcgggaggccgaggcaggagaatcgctggaacccaggaggcgaaggttgcagtgagctaagatcgtgccattgcactccagcctgggtgacaagagcaagacttcatctcaaaatcttatcacctgtatcacttagttggcaatcaattgagcagcaaactttggcatctcttttattatattcttatgcaattattcttaaattatttgattttcacttacttccaatgtgtgcatcttacttcccatgagattgtaagctctcaagaatggaaagttaatgacatcactaggatttttatatttgttagtagccatataactcctgtcaccttcttttcaggtacgtatttgatttttctgtagaaaatgttgaagactttatatgatacattaaacatgatagaaatacatctttaaagaatttactttgttttagcctgtaaacaaaaagttgtctatttgcagagactattcagagatatttggggccattcaatccctcatatttaagttaaactaaataaacagactaatgcaagttctacccatcaaggcccaaattgcattaccagtagcgactgtccccactaccatcgttgttataaagagctaaatatatatatagttttttttttgttttttttctgtgatggagtctcactctgtcacccaggctggagtgcagtggtgcaatctcagctcactgcaacctccgcctcacaggttcaagcaattctcctgcctcagcctcctgagtatctaggattacagtcgcgtgccaccatgcctgtctaatttttgtatttttagtagagatggtgtttcaccatgttgaccaggctggtctcaaactcctaacctcgtgatccaccagcctcagcctcccaaagtgctggtattacaggcttgagccaccgcacccggcgcataaagagctatattttaataataaagacaaattttagtggccggttgcggcggcttatgcatgtaatcccagcactttgggaggctgaggtggacggatcacctgaggtcaggagttcaagaccagcctggccaacatggtgaaaccccgtctctactaaaaatacaaaaattagccaggcatggtggtgcgtgcttgtagtcccaggtattcaggaggctgaggcaggaggatcacttgaacccaagaggcagaggttgcaataagccaagatcacgccactgcactccagcctgggcgacagagcaactgagtctcaaaaaaaaggacaaattttaacaaaacctttctatgagccactttgtttctttcctcttctagtgtgcccttatccatccatatttttatgattgtaaccagtgtacttttaattttatatttttaaattatactataaacatgtttcatgattcaagcttcataattattttggtagctgcataatacttcattaaattgatacaccataattttcttaaccaaaatatgtcaaaatgcctataatagagaaataattatttataactttttagtatgatggataatgttgcactaaacatctttgtgcatatcactttttcttctgaattatttccttaagaaaagttcccagaagtagaattacagaatcaaaggatatgaacatttttatctctcttaatgtgcaccagtataattttttttaaggattgatgaagccattttttaaaaatttatttatttccaaagttcaggggtacatgtgcaggatgtgcagggttgttacgtaggtcaacatgtgccacagcggtttgccacacagattatcccattacctcggtatgaagcacagcatccattagttattctttctgttgctcttcctccttttaccatccaccctccaacaggccccagtatgtgttgtttcccccatgtgtccacttgttcttatcattcagctcccacttataagtgagaaaacacagtatttggttttctgctcctgcattagattgttgaggataatggaagccattggttttgaatggcctgaaatggacatcaacatttgattaggactaataattgtttcattataggtttacattggcaagtgcttcaaaatttagattgtattatgttcactagataattccaaattgttttgtgtaatagttataagatgtattgttttaattaataaaataattcttttaacgttagtggaaaattcagtgttatcgctactctctgattatatgcttgcttggaataaatatacattactatttatttgtaggacttgattagagaccaaggatttcgtggtgatggaggtaagtagtgatttcaggtttttttaaaaactcaaggaaactgcaattgctttgctgcttatttcctttatacttgcctctttcaagtaacagacacagagaaaaatgtgtagagaaacccaaaatttttttgtttttctgtagtgtttgtcatttacctctaataaaatgttaactagtttataacatgagtagaaaagatgactggacataaaaggaagtcttttttttttttttttttttttttgagacggagtctcgctctgtcgcccaggttggagtgcagtggcgcgatctcggctcactgcaagctccgcctcccgggttcacgccattctcctgcctcagcctcccgagtagctgggactacaggcgcctgctaccacgcccggctaattttttgtatttttagtagagacggggtttcaccgtgttagccaggatggtctcgatctcctgacctcgtgatccgcccgcctcggcctcccaaagtgctgggattacaggcgtgagccaccgcgcccggccaaaaggaagtcttaaaatgtattatctacagttttaaaatttcttccaggatcagacaagatcggacacgttcaggatggtatggccgtagactacagttttaaaatatcttaccaaggaaagatccttaatttttatacccgctttattaatttctaaccatcttgaaagctattgttgataaatttcctttgtggggctccactgatacttaaagattgaccttagaatcagataaaacttaactttgctaaatcattctgaagagggggtttgtcagacattatcaaccacttccttcaactttctggaagtgttttaaatgtacattttatagaacagacccataatggcaaagcccatttgtcctcttcttaggtcagtaaatacacaaatgagaaactgaattgagatttccaactgaattttcatctagtattcactctagcacataagacaacattgcttaagaaaatactttttgtaagcattaccctatataatgttttataagaggtgatatttgagactgtcttgaagtgttcttccaggaggtcctttacacttaccttccctgttgtcttctgcctagtaaggaagacctgtaataactgcttatcatgcttagagttgacctcttcactgtgaccttctttatcttcaaaatatctaagcccagactcaacaatattttacattgagtaaacattgttataaaccttcttttgttatgtttctgtatacccatgaagcaaccaaaataataataagcctgcattctatactctggacttggtattgatgttagcacatagttacacaagctttttttttcctgtttgttatttcatgaacctgccaattaatgttgctgccagtttgactttcgtatgtcttaatagctgtggcttttgataattttgcctaatacatccagcatttaaatgttgccatcatgttagcatcacaaaattaacttagtcataaacacagcctgcttagtacctaaaagcaagtggcatttcttgtccttttcatgagtcactttttaaaaaatcattgggattttatgaaaataagcagatttttggtccagaattattttatgaaacaggcttcaattcatcttgtttattccccatgacttctttcatttcttctgtgtgtctgtcttcctgtgtttgcctgcccctctctttctcttctaacagcccctttgaaccagctgatgcgctgtcttcggaaataccaatcccggactcccagtcccctcctacattctgtccccagtgaaatagtgtttgattttgagcctggcccagtgttcagaggtagttgggctcttctttcttgttttcacccaaagcaaactaaatataaaactacagatgctgtttgtgcctcaccctcacagcgtgtgtttgtaagtgtgaaagttttcagtactaaatttctgtttggcctggctggaatgctttgaatgtacgtctcacacgtactcactgccacaagctttctgtatgctgtctgtcataaatttttaaaagcaagaaaatcctgacctgagatttccatcttgttttttcgttattttattacttcttggtcttgataatttcttaaacttagtgggtgggaataaataaggtgggtggggaagagcttactggattcctttgattttaatgcatttaagtgattattcttgatgacttaatatttgttaattttgtggtttttaagaaaattaaagtgtcaatggaaacttctattatgagattttattaggcttttggccttttttcagattctgtaatactagcagtgttttttgggtttttctttcccccaatatgggatgtgtatatttttgtcaaaggtagggagctgttaaaaaagacaaaaaaaagatttataacatattttagatatttcagtgtacttcagaaatttgagaatttatccttttaattatgtcctaatagaagaaagtttacagtataatttcattctcccatttcatcttgccatgttttatttagtagttaaactgatttgtaaaaacttaagtcgggccaggcgcggtggctcacgcctgtaatcccagcactttgggaggccgaggtggatggatcacctgaggtcaggagtttgagaccagcctggtcaatgtggtgaaaccccgtctctacaaaaatacaaaaaaaattagctaggcatgatggcggatgcctgtaatcccagctacttgggaggctgaggctggagaatcgcttgaacccaggaggcggaggttgcagtgagccgagatcacaccattgtactccagcctgggtgacagagcaagactccatctcaaaaaaaaaaaagtaaaaattgggacagatgtctttctctaaatatttttaaagatttatatttactgactcttgctagttagtatctgttatatattctgaatgtagtaatggtgctttagatttttgctctctcagccctgctgtttctcagaaaatccatagaatgggatggaagtcatacagtagtgagtaatacaactaaattaagtgatacaataaaactacttagtagatcataaccgtgaagcctggtcaagcagtcgaggctttataatgttgaaaattatcaatggaaggtagaaaatggattgtgctctacttaatagacattgtggataccattttattttagaaaattgcatatgagataatgaaaattctacatggtgatataatatgatgtaataatggtaaacattttctacagattaaacatttaaatgtggttattggtatccttgttatctgaaagatagtggctctttttttccttaagagtagcagtcattttttaaaaagaatctattttcttgaggtcattttgttgttctgtatatagaactattgcctggacatctgagttctactcagctgtattcaggccccagtaagattcactgccctgaactcttctgaaccaggtgctactgtaccttaactcaggatgtttgccatgagaaaggtatgcaaccctgccaacagagatcacttccaaagagtatactcctcaggctcacttgacctatagaatatttgtatttatagtaacttggctgagaggccatagcgcttacttaacaaagctctcacttacaaaggcagagatttttcagaaagtcttgagaaatatgcccggctttatttacattaactttgttttgtaggtaacaaataatctttgtttaataatgtaagcctccaggaaccaatgatactgaccaatatctcttaaatagtagagcatgtagtttaggattatatttgagtttagtgattaatatgaataagtcagatattttcaacattatggccattattagaaaatgtttccatctggggatttccttttttttaatattgattggctgttgaggtaatattaaataattaattaaaaatgtatttgttatataggcttttacatttattttgctttttgatttttttcatcaaagaaacagaaacttgggagtatttttagtatttctgtcttgttttagagagattgtttttctcctagattttgcaccagtaaataaagtatgtgtctatgtctatcatcagatatcttaaaggtcattaaattggccagaaaactaaaagaaattatagttgtaatcaccaaatgaggcccctttttggcccatcctttccaaaaggtctatatttaaacatgcactacattttaaaattaagtctaaatatcccccaaccttctacccctgataaattaacatacttgctcctccttaatgtatacatttttcttttcactaatttaggatcaaccacaggtttgtctgctaccccccctgcctcattacctggctcactaactaacgtgaaagccttacagaaatctccaggacctcagcgagaaaggaagtcatcttcatcctcagaagacaggaatcgaatggtaagagtatatgatatctttttttctctgaattctttcttcttagaagtcacagccaaatgtaatattatcctttagatatattatgtccatatgtgacacagaattcccataattaaataaatttaagaactgatagttttttgcttaaagcatatttctacggcactgctttttgctgtcatctataatataatttagtaaaaggcagttttggaagagtaacagtattctgttctaaagtaaggaaaaagagagaaagctaatattagaaggcacgaaaaggctggtccagaattcagatatttcagatatctactgaaggacattcttccctatttaaaaaatcaactttcttctgcaaaatgaatccaccatggcacatgtatacgtatgtaacaaacctgcacattctgcacatgtatcccagaacttaaagtaaaatttaaaaataaaaaacgaatactgtttagccgtagtattgctactaattgttgaataagaggatcttttaccctaccaaagtaattttatatgttgattttttttttttttttggaaagaccgaattagataagatacatgaagaaatttagcactgattgaaaaagactacctagatgaattgtcagtagttaccacaggttaacttaaaattttttgtgatttagagccaaaactattcacaaatatagcagcacttatcttgctccttaaagtcttccagatgataaaaacattttacttatttcagtaatatacattcctgctcataccccataaataatttatattttttaataaattgtttccatcctaaccatccttctgagcaaagtatcacaaggacagaaaaccaaacaccacatgttctcactcataggtgggaattgaacagtgagaacacttggacacagggcagggaacatcacacactgggacctgtcatggggtagggggaggggagagggacagcattaagagaaatacctaatgtaaatgacaagttaatgggtgcagcacaccaacatggcacatgtatacataagtaacaaacctgcaagttgtgcacatgtaccctagaacttaaagtataataaaataaaaaataaaaataaattttttccatcctaatattgacttcagtcttaaatttaagttttgtattttaagagtcatacttttaactactattcttccagagaatttttcttaaggggatctcttcctgtatccctctcaggcataaggtaatgtacttagggtgaaacataaggttttctttttctgtttggcttgacttgacttttttactgtttttatcaagaaaacacttggtagacgggactcgagtgatgattgggagattcctgatgggcagattacagtgggacaaagaattggatctggatcatttggaacagtctacaagggaaagtggcatggtaagtatgtaatgtggtgacattgtgacaagtcataataggatatgtttaacaacttttattttgtaaaaaatatcatcaaaggaaatattcactgttcgcatcaataaactattttgattagtttcaggactcctccaaaagtttctaacaaaaattatgggaaataaaaactgttcacagcagtcgggactcctaccattttattacagtaataatttttaaaggggaattcctccaggttaactagtcctcaaaaggattttattttcttttagagtctttcagctgataattttatttgtattataagtcacaagtaaacatattaaaaatgtacttaatggctgggcgcagtggcttatgcctgtaatcccagcactttgggaagctgaggctggctgatcacgaggtcaggagatcaagaccatactggccaacatggtgaaaccccatctctactaaaaatacaaaaattagctgggtgtggaagcacgtgcctgtagtcccagctacttgggaggctgaggcaggagaatcactggaacccaggaggcggaggttgcagtgagctgagattacgccactgcactccaccctggtgacagtgagactccgtctcaaaaaaaaaaaattaacaaagaaatataagtggccagtaaacatatacaaaatgttcagccttactagttatcaaagaattgcaaattcaaaaaatagacatcattatttgcctcttagttggacaaaatctttttaaattggattatattaagagtagtggatgtattttcatcaaaggtttaatatcaatgaaaagtgaaagtgaacatgtatccaactaatagagaattggataaatttataccatcatatgtgattatataggagttaaaatggcatggtagaggtacatttattgatgtagaaaggtgtctttggtatatgaaatttttcaaagcagtatgtgtaagataccatattatggagctcatagaaatatataacataattttttatatgacagtattttaggccaggcacagtggctcacgcctgtaatcccagcactttgggaggccgaggcaggtggatcacccaaggtcaggagttcgagaccagcttggccaacatagtgaaacctcatctctactaaaaatacaaaaaattagccaggcttggtggtgggcgcctgtaatctcagctactcaggaggctgaggcaggagaattgcttgaacctaggaggtggaggttgcagtgagccgagatcccgccattgcactccaacctggataacagcgagactgtcttaaaaaaaaaaaaaaaaaagactgtgttttagtttttatctccttaatctatcttttcacaggtgttcataaatattcacactaaattcatgtaaaagcctaataacatataatgtcacttttgagtgacataattaagggaatttttttataccttcaaaatgtctttaaacttttcttaagtgctgtacagtattttatgatacaaacagtagaataagcactgtattactttgataattgaggaaaatcaatgttgatttaacttattaaaatatacatacaggttgagtatctttatttatttatttttgtttgttttgttttgttttgagacaaggtctcgctctgtcgcccaggctggagtgcagtggcacaatctcaactcactacaacctctgcctcccagattcaagcagttctcctacctcagcctcctgagtagctaggattataggcgcgtaccaccacccctggctaatttttgtattttgagtagagacgagttttgccatgttggccaggctgttctcaaactcctgacctcaggtgagccacccaccttggccttccaaagtgctgggattacaggtgtgaggcagcacacctggccaggttgggtatctttaatccaaaatcccaaacccgaaatgctccaaaatccaaaactttctgagtgctgacatgatgctcaaaggaaatgcttattggaggatttcatatgtttggattagggatgttaaactggtaagtataatcaaaatattccaaaatcagaaaaaatttgaaatttgagacacttctggtcccaggcattttggataagggatactcagcctgtgtataaaagtgcacataaattagccaggcatggtggcatgtgcctgtactcccagctattgaggaggctgaggtgggagaatggcttgagcccaggagttcaaggctgcagtgagccatgatcacaccactgcactccatccagcctaggtgacagagcaagactctgtctctaaaaaaattaaataaacagaacattactagcactctagaaacaccctcccatgtcctcttctagccaatcacctctctcccaagggtaaccaccactgtgattacaacaggaagtgcatagtgtgtactcttttgtgtcggcccttttcactcaacattgtttataagattcatctatattgttgtgtgaagttggaggtcattcattctctttacagtatttcattgtgtgactataacatgatttcttctttcatctgttgcaattggatcgtttccagtttggggctttgattgatgctggtgctgtaaacatttttagtgtatgtcttttggtgaacatgtaaccattgatgggtatatatacctaggacagaattgtgagaccacagggtatgcatatgtccagttttagtaatgctgccaacaatgttacaaagtagttgtaccaatttaaacacctactggcagtgttgacgttacagctgtttcacataaagttttttttttttgatgattttaataaaatatcattttctttttttattattattatacttttaagttttagggtacatgtgcaaagtgtgcaggttagttacatatatatacatgtgccatgctggtgtgctgcacccattaactcacatgaagttttttttaaattttagtgacagttttagtcattttcctaattgaaagtatcataagtaatccataaatttgaaaaaaatgttaactactctgataaaaaagttttatagtttcctacttttaagcaaaattccatagggcctggtaattgtagtttcaacattacttgcagtttcagttagtaaataaatattaagcctagtaagtataatttaatattgtcaaataatttggaaaataccatgggtacttaattgattttaccaaatttccatggaacaaacaaggttggctattttttggattgatattttgaaatactagtacaggaatatcattgttagttgaatttttagccttagaaaacaaatggagtttagatagctaaagtataatttatttgtgatttaataatggtatggagttagggctatgataattagtgaaaacacccaagaatgttttatacttttaaattttaaaaattgaaatgacacttggagtaacaattgccttttaggtgatgtggcagtgaaaatgttgaatgtgacagcacctacacctcagcagttacaagccttcaaaaatgaagtaggagtactcaggtgagcttgtgtgaattactcttttccagagaaagaagttatttttattagctcctggttcccagtggtagcaactattagctttacagatttactcaaaatgaataaatttgtagaaacagagtatgtctgagtatatttttgtctttaaccacattcttttaagtagtatgcaatgttatatggtatggctgatagaatacttagtcctagactgaattaatggaagtatagtattctgataatataaagtaatagttctacttatgaaaagaatactctccagttttaagcttatcagaatacatttagaggtggtatttagtcctgggctctggaattttagaaacattgacaaactaggatatgcctagtgaggaccacctaaatagggaagattctagaggtgtaacgggggaaaataatcaacagaactgaggatatttagttcacagaaggctgttatgttcaagagagtgcacagttattccagagtgcggaaaaaaaagttattccagagagcagaccagggaagcaagccagaggtgaaagttgtaagaaaatgattttgtctcaacacttggaaactttataataccagaaccacttaaataaagatatgagagtcagctacaactgagtgatgaacttcccatagttgaaggtatttaagcaacctctagttgcctgtcagatatatttaaaaagatatctctgcataaagtaggaggttagacttggcaattgccagtctcttctaaatgtatccttttgttgccttttttaaaaaaaaaaaagctttttctgacaacattttaccgacagactactttggttctcttttgtaagaattgctaaagtttgtcgacatttaatgtttactgtcacatttctttgtacaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggagtaagttccattcgttaaatgtcttgtaaattatttttgaagaccattgaggatgttttaaaggttttggctgctattcttttggattgcattttaaattactgtccaggaacataaggatgctaactaatggctggtaaataatatgatactaaaaaataaatgtctctgtctagtgcagccttcagaacatatatcaagtatttgataataaatacatgactgcaaacttaggcttagcactcagtgattgagctaagcaagagaggttcagaagatagaaacagcaaaaacctgctaaaaagttgttagcagttgtgcaagtaaacagaatggttgttagttactttttcaaatcagtttctctgagtgcccgtatttttggttgcaaaatggtcagttaataaagttaaagtgaaaaatctgcattctgaccctttttgaggatttcagagtgagttcctatctgttgaattttgctatgcaatttaaggagttattttataaagtttaccataagctaatatggggaactgactttgaaggataaattttaaattttgcaactcttaagtgcaaatgaataggtaaattaaaaggtaaaattaaacaaattttgaaagcacttaggtgaaaattataaactcagtaaaatatgaatttgaaagctctgtgagaagtttaaaaatagatatgatctgaattttgttttttacaaattgctttcacttacatagattattatgtcatttaatctttataatgttatgaaggagatcttttttctttttacagataaggaaattgaggcttttaagttccttgtctaagggcacacatttaataagtggcaccaaaggtgtttaactcaggatttctgactcccaatccagtattctttccccataaccactatgctacttttacattacaaaattagaataaaagagtaaaagggtatatatgtactaacacctacaactctaactgagtattgctcctagcaagtaagtatagagccaagactctaaaccagatctggctcttagatcttccaactataccaccttctctttctcaaaactaggcaatatatctataatttagattgtttacaagcctatattcggccaaaatacttattacagcaaattattaccttattcagtaacacccccacttacccctagacttgaaacaatctcaacgtttcagataagttagaatctctgaatctgttcgaatctaaaggcttttaaagaattaaaatcttggccaggcacagtggctcacacttgtaatcccagaactatgggaggccgaggcaggcggatcacctgaggtcgggagttcaagactagcctgaccaacatggagaaaccccgtctctactaaaaatacaaaatgagccaggcacaagcctgtaatcccagctacttgggaggctaaggcagaagaatcacttgaacccgggaggcagaggttgcagtgggccaagatcacaccattgcactccagcctgggcaacaagagcaaaattccgtctcaaaaaaaaaaaaaaagaattaaaatcttgtgaagagtaaaccatggcaaagattgtgaacattcagtgagaaacaaaggcttattctcttcccacaccactggtctctttgcaatttcttcagcaggccaagcgtgttccttctttaggacctttatgtttgttatattctctttaggatacacatgccacaaatatccttgtgtctcggttcatatgtccccttttaatagaaatccttgctcaccttatataactaacatgtccccacgtcactctgtcactctctatacccataggcttgattttctttataacgcatagctccatctgacttgtttcttgtcttttatccccactagaatgcaggctgtatgagagcaggggctttttttcattattttatgcctaatgcctagaatgggacctggcatactcagtacataactgttaaatgaaaatgattacacacaactgcatattattgatagatttacctttcagaagaaagagatgccaaatccttctcacatcacagctgagaaatgtggctgggcatgttggctcacacctgtaatcccagcactttggaaggccaaggtgggaggattgcttgagcctaagagttcaagaccagccaggaaacatagggagaccctcgtctctgtaaaaaataaaaaatttagcaaggtgtggtggcacttgcctgtagtcctagctactcactaggctgagttgggaggatcacttgagcccaggagtgtgaggatgcaaggagtcgtgattgtgccactgcatccagcctgggtaacagagcgagaccctgtcccaaaacaaacaggctgggtgtagtggctcatgcctgtaaacccaacactttaggaggccgaggtgggtggattaagacaagaagtaacagtaaagaagaacattatcaaattggaatagtgctgcagtctgaagaacagtcagtgaagaggtgatatattttcaaaatatcactttacagtttgggattatcagtgtaaactttagtcatctactctgaaacactttggttgtgtttttaaatagatcttaccatttaacatgatgcaatgagtgtacgatagtatgaacatagatcattccattcagtttatcctagattttagtaactgaaaaagtattaattccaagttttaagccctccagcagagtatactttttagtaccagtttaaaattaaccaaggaggatatagtttcttttctctttttgttttgtcttgtaagatcataacatacaactggttaaatgtaccgacacatcttcagtttctgaaggatttggcaggttgaaactcctcttattaacagggctatgagtttcagattaaggtgacagatttttgctccttcctggaactccactaaaactgtaataaaggaattttttttaaagcatggacacataaggatggggataacagagaggaatgagacaatatcagcaacattttgaaagctggagagcaggtggaaaagtgatcatagacttagaccccaaaaggctaaatgatcagtcagcagtggggaaatgaaagccaacctggtttataccgtagaatcctcaattctcaggaattggcaatatcagctatctcaggggatgaaagggttaaaatgaaaggcctgtttgaaaagctgttatttctctaaatctgttctcttactcaccaggtaactgctccatccctatcctagcagtagactggaagtttcttctctagagaggggaaaataaatatctctggactgggagaccctaatctatgtctaggacatgtatatctttcccaaaacatggggatttgatgactgtgtgcttactaaatgatgaagggagatttccccagccctctctttttatttgattcctgacatgctagcagccaaaccctactcttccgaaatgcagaagattcgaagagtcttgggtgaattttaccagctcaagaggaaagacccaaagaaagtgacatcagggattccacctagattactgtatagttcaaaaatagcaagcccatctgtatgcttaaagcctccaggcagcttgtaagtccctcacttagtctaagtaagagtatcgctggataactagatattggtggggaagtcttatgcaaataagagagactgtaaaacacatagagaaaaataggcattgggagaaacacatacaatgcaagattatctctcccctgcctcaccccacctccaaaaaacctatcagttttctcagagggagaaaagataactttatgtccactaaacaggaacagaggttttctaaggaacattcatgtaacagcagcaacaaaatgctaaaatgttgatatttacagtacactggaaattatgtcctttgcaattatttaaatgtaacttttaaatgttaatttaaaaggagttaacgtagtttctcagaattcttttaaggggtgttcagcaaaaaaagttttaaaactattatgttaaacactatatggtttaatattaaattcctatattatgcaacataattcggaagggacacttagataaattttttacaaaccaagttacataaaatatgtataattaattggaacacataaccagattgcatcagtgagtcttgaagtggatattcctgttttcttctctagtgtcaatgactaaagcacactattttcactactttttttaaattttgagacagagtctcactcctctgtcacccaggctggacaggctggagcgcagcgacactctctcagctcgctgcaacctctgcctcaagtgatcctcccacctcagtctccctagtagctgggactacaagggcgcaccatcacacctggctcatgtttttgtattttttgtagagacagagtttcgccatgtcacccaggctggtctcaaactcctgggctcaagcgagccactgcctccgcctcccacagtgctgggactacagatgtaagccaccaggcccggcctattttcagtacatttgattgaactttgttgttgttgttgttgttgttgttgttgttgttgttgttgtagttaaacaatctgttttaatcaggctgacagatgttagaacaaacaggatggtcctaggggtcatgaactggttcttcaaatcctaactgttgctgctgattttgtaacagattttaacatatcttttgacttctggtcactcatttcagcctcatgtttggttctctgttttaatttccccctatctattcccatggatcaaatggcacagatggaattccttacatacctaatttttcctccctatttccaatactgttcatcttgaatgccaaccatagtttcatcttgagggctttgttttggaattatctccagaagctattcccagttagaagcctgcttggataattaatattgcttcttagctccctccagctctctttccttctgtcccctggctctaccctgagcacaaatgataattctctgtgaccatcacagccaccatcttactgtgcctcctttcaactcttcatgtctatatcctcttccctgaccagcatcatctcacaaaggactgaccaagccagatatagtctatagctagacaataaggctatagggtatttaaatagcctcttaatatgtacttttgcaaagcctttatttcagaatagcctgccctatgttgaaagtgatcttttctgactgctcaccaaaattcattctaaagtctctggatataagcagaataggaacaaacggattcattttataaagtgctaaggttgcagttaactcataagataaaacctttaataattagaaaagttagtctggacacggtggctcacacctgtaatcccagcactttgtgaggccaaggcaggagtattgcccgagccaaggagttcaagaccagcctgggcaacggcaaaaccccgtctctacaaaaaatacaaaaattagctggatgtgatggtgtgcacctgtgttctcacctacttgggaggctgaggtgggaggatcacttgagcccaggaagtcgaggctgcagtgagccatgatcgtgccactacactccgccctggatgacagagcaagaccctgtctcaaaaataaataagaataattagaaaagttgaatcattaggactttcaaatgtgtcacctttattggattacagaatataagcaaaaaatggataggtaacatttttcctgtgtggttatatcttccatttgtacctcagtgaaaaactatttctgattcctaggtttacttgaaaaggagcagagctgttctaatggtagataattataaactcactctgaggaatcagggttggtaaagtatgttttatcatcttcttttttggttttttttttttttttttgagatggaatctcactgtcacccaggctggagtacagtggcacgaccttggctcactgaaacctccgcctctcaggttcaagcgattctcctgcctcagcctcctgagtaactgggattacaggcacccaccaccacacctggctaattttgtatttttagtaaacgggttttcaccatgatggccaggctggtcttgaactcctgacctcaagtgatctgcccgcctcggcctcccaaagtgctgggattacaggcgtgagtcactgcacctggcctgttttatcatcttttcacctgccagtcattgattcatcccaaggacccagatatcttaagaatactgttactaaagaaattccaggaatggtcagtacattgtgcctttttttttttttttttttggcaggccttataatttcagtataatatttatggtatgattttgaatttaactttatcaaaaaattaaatcacagaggcacatagaaaaagttacagcctatcgatatatttacagaagcattatattctcaaaataagatgattaaaaataatttggagataaatccttacaatttactttgttttaaacaatgatgagcatgcctcttttactcataagtgaacccagttgaagatagaaggactaattaaagctgaaaaaatggtgaacatgtattagtgattgataataattctaagtggccgaagaatatttaattatagtgaacataattttctggtcggtaaaaataataatagggtgctgataataataataatcagaaaatgcaaggtaaaacaaaaaggtaccactttccacccactggaattggcaaaatgcctgagttctgataagatcaaatgttcataggattagaggaattgcttcctgggtcatttctgatgcaaccagccaccttaacagcattctggaagtagctgttaaaataggaaaatgcttattctaaccccaagaaacattagcttttgttccaagtcgtgtatacagaaagagatgtattataggaaacaatataatagtgaaaaattggtctggatgcagtggctcatgcctctaatcccagcactttggaagactagggtaataggatcacttgaggccgggagtttgagaccagcctggatgacactgtgcgaccctgactctatgtaaaacttaaaacattaaggacatttttttaaaaaaagaaaaacttattgaaaaattggaaacatgttcatcaggagaagacttgataaataacatattggcacttacatacagtagaatcgtatatagtagttaaaagtggattatatatgtatcaacataaagctttaaaatattaatgttaagtgagaaaagcaagctgcagcatgagaccacttaaaaatttttaagcagaacattttttacatttgggctttaaaaagtggtgtgtatgtatatatgtaaaagtactgaaataaggattagaaagcaaagatcaagtaacatagtgattatctccaggaatcaagtacaaactttgaaaaaagactggaggtggccaagcacggtggctcatgcctgtaattccagcaccttggaaggccaaggcaggtggatcacttgaggacaggagttcgagactagcctggccaacatggtgaaacaccatatctactaaaaatacaaaaaatcagctgggcatggtggccggtgcctgtaatcccaattacttgggaggctgaggcaacagaatcgcttgaacccaggagacggaggttgcagtgagccaagatggcaccactgcactccagcctaggtgacagagcgagattctgtctcaaaaaaaaaaaagactggaggtgttttagtccattttctttactataacagaatacctgaggctgggtaatttgttgttttctgcaaaaagaaacttatttctcatagttctagaggctgggaagtccaagggcattggtgctaacatctgctgggcttctggtgagggctttcctactgcatggtaacatggtggagaagcagaagagggagtgggcacacacaaaaggggcagaacacaagggacagcctcactctatagcaaccccctttcacagtaactgtagaagtcactcctggccaggtgtggtggctcatgcctgtactactagcatattggcaggctgaggagggaggattgcttgagcccaggagtttgagaccagcctggacaacatagtaagacctcatctctacaaaaaaattttttttaattagccaagcatggtagcacactcctgtagccccagatactctggaggctgaggcaggaggatcacttgagcccagaagtttgaggctacagtgaggcatgattgagtccactgcactccagcctgggtgacaaagtgagaccctgactcaaagaaaaaaagaagtcagtgactcctgctttcatgagggcattcctcatgacccagacccaaatgcctcttaaaggtcccaccaactctcaacaccattacactggggccaagcctccacatgagttttgtggggacaagccatattcaaactgtagcaggaggcaaatgtataaaagttttaatgggtctgattatggtaagaatatgagtgacattatcctttgctactgcagtttttttaaaatttcaaaatcatgttacgaggaaatatgtaatcattataggaaattcagaaaatgtaaacataaataaaaagcaccagtaatccaccatcaagagataactgtcattaatattttgatgtgtattgtgtatcctttcagaatgatgtgttttaatactatacacaatggtttcttgctgttgttttaaatcatactgggttttcctttttactcttaaatatctctactggtcaataaatatctgataccagctctgctacatactgttaactcactgtctcccctttttctttatgccaactatgttctgtggggtttcttaaaatatactgtaatgtatttagcaatgatatgcacttactatgtgtcagacattctgtgtactttatatacattacctcatttactcctcaacaaccctgtgaggttgcagcttttattatatccttgttttatggatgaagaaacctggttatggaaggggcaagtaacttgccccaggtacttcagctccagggtgtattcctttaaccattaaagcatgttgcttccccaacttattcagtcccttcgaaaacttcaagtagacccttaatgttatagggcagttacactttcagtttcctagttcattcactgtcctcttttagacactgtcataccgcctcaaaacctcatatccttaagcctctaaaacctcttcttcaagaaatcagcgatgacattgcttgccatctctttaagacagttggaagtaaccattatgacatctacccacctgcctgcattcataccaggttcttctgccttatcactagtaaaagccaatccctccatttgtacactagattccatccccttacccctacccagagacatcttttgagtaaatctcccatcttgtttatcatcagtttttcctttcctactgaattttctcctattttaaaacatcttttggctgggcacggtggttcatgcctgtaatcccaacacttcgggaagctgaggcgggtggattgcttgagctcaggagttcaagatcagactgagcaacatagtggaacccatctctaccaaaaaatacaaaaattagccaggcgtggtggtctgtgcctgtggtcccagctacttgggaagctgagacaagtggatctctagagcctgggaagatgaggctgcagagagtcaagatcgcgccactgcattccagcctgggtgacagagcaagactctgtctcaaaaataagtaaataaataaaacatctttcactgagtgcagtggttcacacctgtaatcccagccctttgggaagctaacgtgggaagatcacttgagctcaggagttggagaccagcttgggtaacagagtgagtccttgtctcagaaaactaaagtaaaatttaaaagtagggcaggtgtggtggctcacacttgtaatccaagcactttaggaggctgaggctggtggatcacttgagcccaggagtttgagaccaccctaggcaacatggcaaaacccgtctctacaaaaaatacaaaaattatccagatgtggtggtgtatgtctgtggtcccagctactcgggaggctgaggttgcagtgagtggagattgcaccactgcactccagccagggcgacagagtgaaaccctgtctcaaaaaaaaaattaagaagtaacagtaataatgaaacatctttcttatgcacatttggcagaatgttgacatttgtggaatctatgtggaaggtgtgtgggtattctttccatttttctgtatgtttattttcttttttgttgttgttttgttttttttattattatactttcagttttagggtacatgtgcacaatgtgcaggttagtaacatatgtatacatgtgccatgcttgtgtgctgcacccattaactcgtcatttagcattaggtatatcttctaatgctatccctcccccctgccccaccccacaatagtccccagagtgtgatgttccccttcctgtgtccatgtgttctcattgttcaattcccatctatgagtgagaacatgcggtgtttggttttttgtccttgcgatagtttactgagaatgatgatttccaatttcatccatgtccctacaaaggacatgaactcatcattttttatggctgcatggtgtatatgtgccacattttcttaatccagtctatcattgttggacatttgggttggttccaagtctttgctattgtgaatagtgccgcaataaacatacgtgtgcatgtgtctttatagcagcatgatttataatcctttgggtgtatacccagtaatgggatggctgggtcaaatggtatttctagttctagatccctgaggaatcgccacactgacttccacaaaggttgaactagtttacagtcccaccaacagtgtaaaagtgttcctatttctccatatcctctccagcacctgttgtttcctgactttttaatgattgccattctaattggtgtgagatggtatctcattatggttttgatttgcatttctctgatggccagtgatgatgagcattttttcatgtgtcttttggctgcataaatgtcttcttttgagaagtgtctgttcatgtcctttgcccactttttgatagggttgtttgtttttttcttgtaaatttgtttgagttctttgtagattctggatattagccctttgtcagatgagtaggttgcgaaaattttctcccattttgtaggttgcctgttcactctgatggtagtttcttttgctgtgcagaagctctttagtttagttagatcccatttgtccattttggcttttgttgccattgcttttggtgttttagacatgaagtccttgcccatgcctatgtcctgaatggtaatgcctaggttttcttctagggtttttatggttttaggtctaacgtttaagtctttaatccatcttgaattaatttttgtataaggtgtaaggaagggatccagtttcagctttctacatatggctagccagttttcccagcaccgtttattaagtagggaatcctttccccattgcttgtttttctcaggtttgtcaaagatcagatagttgtagatatgtggcgttatttctgagggctctgttctgttccattgatctatatctctgttttggtaccagtaccatgctgttttggttactgtagccttgtagtatagtttgcagtcaggtagtgtgatgcctccagctttgtgcttttggcttaggattgccttggtgatgcgggctcttttttggttccatatgaactttaaagtagttttttccagttctgtgaagaaagtcattggtagcttgatggggatggcattgaatctgtaaattaccttgggcaatatggccattttcatgataactgattcttctacccatgagcatggaatgttcttccatttgtttgtatcctcttttatttcattgagcagtggtttgtagttctccttgaagaggtccttcacatcccttgtaagttgggttcctaagtattttattctctttgaagcaattgtgaatggaagttcactcatgatttggctctctgtttgtctgttattggtgtataagaatgcttgtggtttttgtacattgattttgtatcccaagactttgctgaagttgcttatcagcttaaggagattttgggctgagacagtggggttttctagatatacaatcatgtcgtctgcaaacagggacaatttgacttcctcttttcctaattgaataccctttatttccttctcctgcctgattgccctggccagaacttccaacactatgttgaataggagtggtgagagagggcatccctgtcttgtgccagttttcaaagggaatgcttccagtttttgcccattcagtatgatattggctgtgggtttgtcatagatagctcttaatattttgagatacgtcccatcaatacctaatttattgagagtttttagcatgaagggttgttgaattttgtcaaaggccttttctgcatctattgagataatcatgtggtttttgtctttggttctgtttatatgctggattacatttattgatttgcgtatgttgaaccagccttgcatcccagggatgaagcccacttgatcatggtgaataagctttttgatgtgctgctggattcggtttgccagtactttattgaggatttttgcatcaatgttcatcaaggatatcggtctaaaattctcttttttggttgtgtctctgcctggctttggtatcaggatgattctggcctcataaaatgagttagggaggattccctctttttctattgattggaatagtttcagaaggaatggtaccagttcctccttgtatctctggtagaattcagctttgaatccgtctggtcctggactctttttggttggtaagctattgattactgccacaatttgagatcctgttattggtctattcagagattcaacttcttcctggtttagtcttgggagagtgtatgtgtcgaggaatttatccatttcttctagattttctagtttatttgcatagaggtgtttgtagtattctctgatggtagtttgtatttctgtgggattggtggtgatatcccctttatcatttttttattgcatctatttgattcttctctcttttcttctttattagtcttgctagtggtctatcaattttgttgatcctttcaaaaaaccagctcctgggccagccgccccgtccgggaaggaggtgggggggtcagccccccgcccagccagctgcctcgtccgggaggtgaggggcgcctctgcccggccgcccctactgggaagtgaggagcccctctgcccagccagctgccccgtccgggagggaggtgggggggtcagccccctgcccggccagccgccctgtccaggagggaggtggggggggtcagccccccgcccggccagccaccccgtccgggagggaggttggggggtcagccccccgcccggccagccgcctcgtccgggaggtgaggggcgcctctccccggccgcccctactgggaagtgaggagcccctctgcccggccaccaccccgtctgggaggtgtacccaacagctcattgagaacgggccatgatgacaatggcggttttgtggaatagaaggaggggaaaagcggggaaaagattgagaaatcggatggttgccgtgtctgtgtagaaagaggtagacatgggagacttttcattttgttctgtactaagaaaaattcttctgccttgtgatcctgttgatctgtgaccttacccccaaccctgtgccctctgaaacatgtgctgtgtccactcagggttaaatggattaagggcggtgcaagatgtgctttgttaaacagatgcttgaaggcagcatgctccttaagagtcatcaccactccctaatctcaagtacccaggaacacaaacactgcggaaggccgcagggtcctctgcctaggaaaaccagagacctttgttcacttgtttatctgctgaccttccctccactattgtcctatgaccctgccaaatacccctctgcgagaaacacccaagaatgatcaattaaaaaaaaaaaaaaaaaaaaaacagctcctggattcattaattttttgaagggtttttttgtctctatttccttcagttctgctctgatttcagttaattcttgccttctgctagcttttgaatgtgtttgctcttgcttttctagttcttttaattgtgatgttagggtggcaattttggatctttcctgctttctcttgtgggcatttagtgctataagtgtccctctacacactgctttgaatgtgtcccagagattctggtatgttgtgtctttgttctcattggtttcaaagaacatctttatttctgccttcatttcgttatgtacccagtagtcattcaggagcagcttgttcagtttccatgtagttgagtggttttgagtgagtttcttaatcctgagttctagttagattgcactgtggtctgagacacagtttgttataatttctgttcttttacatttgctgaggagagctttacttccaagtatgtggtcagttttggaataggtgtggtgtggtgctgaaaaaaatgtatattctgttgatttggggtggagagttctgtagatgtctattaggtccccttggtgcagagctgagttcaattcctgggtgtccttattaactttctgtcgcgttgatctgtctaatgttgacagtgggatgttaaagtctcccattattattgtgtgggagtctaagtctctttgtaggtcactaaggacttgctttatgaatctgggtgctcctgtattgggtgcatatatatttaggatagttagctcttcttgttgaattggtccctttaccattatgtaatggccttcttggtctcttttgatctttgttggtttaaagtctgttttatcagagactaggattgcaacccctgcctttttttgttttccacttgcttggtagatcttcctccatccttttattttgagcctatgtgtgtctctgcatgtgagatgggtttcctgtatgcagcacactgatgggtcttgactctttatccagtttgccagtctgtgtcttttaattggagcattcagtccatttacgtttaaagttaatattgttatgtgtgaatttgatcctgtcattatgatattagctggttattttgctcgttagttgatgcagtttcttcctagtctcgatggtctttacattttggcatgattttgcagtggctggtaccacttgttcctttccatgtttagtgcttccttcaggagttcttttagggcaggcctgggggtggcaaaatctctcagcatttgcttgtctgtaaagtattttatttctccttcacttatgaagcttagtttggctggatatgaaattctgggttgaaaattgttttctttaagaatgttgaatattggcccccactctcttctggcttgtagagtttctgctgagagatccgctgttagtctgatgggcttccctttgtgggtaacccgacctttctgtctggctgcccttaacattttttccttctttcaactttggtgaatctgacagttatgtgtcttggagttgctcttctcaaggagtatctttgtggcgttctctgtatttcgtgaatctgaacattggcctgccttgctagattggggaagttctcctggataatatcctgcagagtgttttccaacttggttccattctccccgtcactttcaggtacactaatcagacgtagatttggtcttttcacatagtcccatatttcttggaggctttgttcgtttctttttattcttttttctctaaacttcccttctcgcttcatttcattcatttcatcttccatcactgataccctttcttccagttgatcgcatcggctcctgaggcttctgcattcttcacatagttctcgagccttggctttcagccccatcagctcctttaagcacttctctacactggttattctagttatacattcgtctaaattttttattattttcattaaaaaaaacttttggggtccctcccttaatcctgcttctctcttcagttattacctcattcctcttcttccttcgacagtaaaacttctcaaaagacttgttggtattcactgtccagttgctctccttcctttttatcttggaccccactcaaaccaggcttttgcccctgctgctccagaaaacatcttttttttttttttttttctctctctcaaggaagagtctcactctgtccctcaggctggagtacaatggcatgatctcagctcactacaacctctgtctccagggttcaagtgattctcctgccttagtctcccaagtagctgggattacaggtgcacaccaccacacccagctaatttttgtatgtttagtagagacagggtttcactatgttggccaggctggtctggaactcctgacctcaagtggtctgcccatctcggcatcccaaagtgctggaattacaggcatgatccactgtgcctagccaggaaacagcgtcttaatatgatagttactggtggcctccatgttgctagatccagcagtaaaattctcagtccacagtttatttgacctgtcagaaacatctgatacagttgatcacacacttcttcatgaaacaggttcttaatcccttgtttaattgtgttcttagtttcttgctgctcagaatgtggtctgagtagcatctgtaccacctgggagccttttagaaatgctaagttttagataccaccccagacctgaatcaaagactgcattttaacaagatccccaaatgattcgtgtccatattaaagtgtgagaaacactgctttcaggcatattctcctgattacccttctactcactgtctactcctcagctggttcctcctctccttctttggtgtttcgtggttggacaagtcatttaaaatacctgtaatcagggccgggtgtggtggctcacgcctctaatcccagcagtttgggaggccaaggcagatggatcacttgaggtcgggagttcaagaccagcttgaccaaaatggtgaaaccccatctctactaaaaatacaaaattagctggacgtggtggtacttgcctgtaatcccagctacttgggaggctgaggcgggagaatcacttgaacccgggaggcagaggttgcagtgagccaaaatcgtgccattgcactccagcctgggtgacaagggcaaaactccatcttaaataaataaataacctataatcagaaccagcaatacaaagtattatcaaggataaagaacaactggaactctcatacattgctggtaaagattttaaatgatacagccattttagggatcaggttggcagtttcttttaaagttaaatttaccatttgtgcaattctaatcctacctaattacccaaaataaatgaaaccatcagtcaacaacaaagacttgtatatgaatacagcatcttcttcatatttgccacaaactaggaacaacacagtgtccaataagtaaatagataagttgtgatatacccataccccgtaacactgcatgcagccagtgcgtgaatctcacaattgttttgctgcgcaaaagaagccaggtgattctgtttatatgaaatttgagaaaaggcaaaattaatctgtagtgacagaagtcattatcaatggtttcccaagcctaaagggggtgttactaatcgcaaaaatgtatgaagaaactttctggggtgacggatattctttatattgattgtgactggtcattacacaagtgtatgtgtgtgttgaaacttttaaaactgtccatgtaaaatgggtgcatcttgtgtgtaaactgtacctcagtaaatttcagtttttaaaagtgaaaaatacctgatggcttacacctataattctagcacttcgggaggccgaggtgggaggatggcttgagcccaggagttcaagaccagcctgggcaacatagtgagacccatctatacaaagcagttttttaattatccaggcatggtggcgcccacctgtagtcccagctacacaagatgctgagttgggtggatctcttgagcccaggaggttgaggctgcagtgagctgtgatcatgccactgtactccagcctgggcaagagagcaacaccctgtctccaaaaaaaaaagtgaaggaaactaaaaaatcaaggcaaaaatagaatttaggccactatggagcataactttaaaatatgtgaactttacctattcgatattttaattatttttaaagtgataaatgattactgactacagagaagtaacagaatgccattctcgttaatcttactttccagggttgtaataagggagcagagggcatcaaagtataaggaaggctgtaatttggcactgtcaccactatcatacctaccagtccaggtggtcacccaaggagagactcatcttgctgcctaaagtctgatgaacctttgtaaaattgtgcagtattaggtgaaagagggctgacatgcaaatgtctaagtaggtcagttctgtgttttaccaagagtttaaaatacttgaaaatggcagcagacttaagtgagagatgctagtggcttttacaaataagctaagttgataaattagccaaatggcatagcaatattatcaataggcattttaatgataatctctcattgttttcttaaggataagatcaagtgatgtaacatgaatgacaggatttaactgggttcatactggatgaatgatcatacctgaagacagcaaactataatgagattcctatcctcagtcctctcttgaatataattacgtatttagaagtaacttatgtgaggaattattagcatgccgatctttaaaaatctccttttttcagctctcacgtaagactcttgaaacaagaaaataaaaataacctatgttttattaaagaaaaatgaagcaggcaagaaaaaaccgacttttttagtgaaagctaaagttttaatctgaaattatagaccatgtctacaaaatttaaaatcctaaggctgggcgtggtggctcacgcctgtaatcccagcactttggaaggctgaggcaggcagatcacgaggtcaagagactgagcccatcctagccaacatggtgaaaccccatctctactaaaaacacaaaaattagctgggcatggtggcgcgcgcctgtagtcccagctactcaggaggctgaggcaggagaatcacttgaacctaggaggtggaggttgcagtgagccgatatcgcaccaccgcactccaggctggtgacagagtgagactctgtctcaaaaaaaaaatcctagtcaagtccaaaaaaagagaaatatacaagtataagatggggaaaatgttttataggagcaattcaagaataattattctaggattgtaattgaatataatcctttttgtgcccttttttaaaaattctatttattgtcataaattttgacccagcagcaggaagcatgtaattaaatataattttaattggactcagtagcatgactttggctgccataacaatcacaataatgtctgatttaatgttgtatgcccaccatgtgggagatgaagatgaggatctcaccatagtttgtgttggttagatccgatctgaagtattgcttttaatttggggcaccacattatgctctgtaagaatgtggaaaagctgaatcttggataagagcttgaagtagtgtgagaagaaattttaccagatgattttgtagctctaagattaaatggttctataggtggacactttttcttcaaggaatgagactgttaagagaaacctatcaagagaatgtgttgttcctttggttgatagcatatttgttctttcctagtacttctgtaaaactgtaatgctctccttaacatcttaccaaggaaagagggaggtccaatctaattatcctaattaatggattgacttctgtgtcatatatggcaaagtaaagttgatatattcttgttcccttccctactcccaaacagttatgtgagttatttgttttctgttatttttgtttgggttttttttttggcattttccattgtaactgggtggtgcttaggaaattacgttttagtgtccccctatatagaattatttagggtagtgattactgtgaaacattatcttatgtagggattcggttgtgtctcatcccatttctctagctgggggattagacccccttttccttccagcctttcattctacagacctggacttctactctttgctgcttttgttacagaaggattacagaaagagaattattcaatgagataaaaattaacaaagcagtttgtaagtaataaaggattacacaggtataaagtgttgtttcaggttgtatttttatgcaactcggcctataacaaatttatgctatataacttctatgctgggtatatcactggttccccaattacatttatatcttgttaattatctagtgctttcctggtcagaaaatttctctccccactctccccagttacatttctgtctactctattcttgctatattcctctgcatcaaatttgttgaaaggattaacaccatcccatggagaacacctttatcttcccctcttttatcttcagcatgcctctgtattttgatatggttgaagcccatttgtttttgtttttaagtcttgcaggaaaaagaacctcctttcaaagaccaacaccttggctgtatactagatcccaggtctctctgagtgcaggacctagctccaccaaccaacaccatcatcatttatccccctttctttctaatggagacaagatctcaccatcttaaagctttgattggggcccatttatttccaccagtttttaactttgtatgtcactgcagaactttgtaagcagtgagacctataaccgaaataacattttctttcagtgaaagagtgacaatatgatgtagaacactgagcctaaagtcatgacaagataattttcaattgtagtaaatgctatgaagaacataaactgtgatatgaatagtaattggagaagggattactttagataaatagtaatttgaaatgaaaattttatctagaaaatactttaaaatttttgatattgggaagaatgaggacgtttaaatgaagatgagacagcatgaataatcttggttatttgcaaatcttccacttttaacagtgtttattgcatgattttgattataccatttggattttaaattctcattttaaggtaaaactgtctaatgatcattgtcagatttcagaagtgtcatatagctattctctccggtattcaattaagaaaaataaccttgactagcacattatatgcatcttattgtagttgattcagtagacatgtattgaatacttaccacacataccaagcactgttctcggcactgggtatacaaagaaataaacaagacacagcccttttgagacaggaggatctcttgagcccaggaggtggaggttgcagtgagccaaggtcgtgccactacactccagcctgggtgatagagtaagactttgtctccccaaaaacaacaacaacaatgctgccttttacttcaatgaatgtagctaaagcagtttttaggggtaacatagatgattttaaagttatcgttgactttaaaatagacctactatggccattttagagaaagttttctcctcactagttttaagaaaatattttaagaatatatgacaactacctttaaaaatagttttaattaccaaactgcatgttctagtagtttacattccaaatagaagtgaacataggcacgttgaaaaaacacatgaatcaagccattattatgataattactacataagttgagcagtgaaagagcactttcagttcggttagtcatgggaaagcttcactcaggagttagaatttgtattcagttttaaaggatggatatgaataggtggaaaatagaacgaaagctaacttggagggttgggtgtaggaggggagcattgaacacagtggtgggattaaatatcaaggtatatttaagggacatggataaataggcttgactggagtgaaaggtttgtattggtaactagtaaaagataaagcaaagtcttttaatagaaaacaagattttgactgtaccattatagtgatatgttcctataatcttaaattcatttacagtctattttaatattctgtgaagggtttattacaatgtactattttcagttgtatcatgattctaaataagtctttacacccccaagtatgttctgtagatttcgaggccagagtcctttagccctactcaggttaaaatgatgttttgtttttcagttacttacacgccaagtcaatcatccacagagacctcaagagtaatagtatccttcctgaaatttgtctgcgaagtttgaaaacatcctgactttttcttctgcattttgtcttcacattatgtaaaaacagttttcatgctaagttcgatatactgtaaagagaattaataaaggattgtgcatgcatgtataggagagcaggataccacagcctgcttttggtttctcgacaactgaacattacaagaaaatctatcagaagtctttacaatagtaggagtttttgattgcttgcttacattttatcagcactataaaactgatagttttgtagctatctattagtccctttcagacctctgaccttgctcagtggtagttgagatataactgaagactctaaattatataacaatgaggtgagaaaaacataatatttctcttccctaagtgcagactaagatactatctgcagcatcttcattccaatgaagagcctttactgctcgcccaggagtgccaagagaatatctgggcctacattgctaaaatctaatgggaaagttttaggttctcctataaacttaggaaagcatctcacctcatcctaacacatttcaagccccaaaaatcttaaaagcaggttatataggctaaatagaactaatcattgttttagacatacttattgactctaagaggaaagatgaagtactatgttttaaagaatattatattacagaattatagaaattagatctcttacctaaactcttcataatgcttgctctgataggaaaatgagatctactgttttcctttacttactacacctcagatatatttcttcatgaagacctcacagtaaaaataggtgattttggtctagctacagtgaaatctcgatggagtgggtcccatcagtttgaacagttgtctggatccattttgtggatggtaagaattgaggctatttttccactgattaaatttttggccctgagatgctgctgagttactagaaagtcattgaaggtctcaactatagtattttcatagttcccagtattcacaaaaatcagtgttcttattttttatgtaaatagattttttaacttttttctttacccttaaaacgaatattttgaaaccagtttcagtgtatttcaaacaaaaatatatgtcttataaacagtgtttcatattttattcttaaataaatatgaacccttaaaacgaatattttgaaaccagtttcagtgtatttcaaacaaaaatatatgtcttataaacagtgtttcatattttattctaaattgtttaaagtattttgtgttcaaaatgttctgtgtaccctgttgaaaaaaaaaacaggtatgcaatttaaggcaggtgtgatccacagccattattatggttttgctaagagaactactccttttaacagagaagctgtttcgcaatcttatttaagcctaaattggaaagttacttcctttagactagaaagtatctcataattatggggcagctggaagaggaaagacaaaaaaaaatgagaggtagattaacagccttgtgctgtcttgcatagctctttctttcttcttgttttttgctttgtggaaaagaagaaagagaagttctaaaagaagggaacaaaaacttgtgtgcattgcagcaagctgtggaaagctcagtcatatgaatcattccctaaaacagcattcttaaaagggtccctcacaccgttttagagggtccacaagatcttccctttgtgagacaagattttctttatatccttcaaccaaaacaacacattgcaacagactgagtgcaaaagcaaatatgagaatccagctggctgctgttaagccagacattgaggagaatcacaggccactcatggtggctggagtccatagttccagctacctgggaggctagggcaggaggatcacttgatcccaggagttgactggcctgctcaacatagtgagaccccatctctaaaccataaaaggaggataattgtagtactattcttcttactaaacttttttttgataatagttatttttcattaaaaatgaatgatctgtgttaacatctacttgttattattttagtagttaaatgaattactagtttaatttctccattaaattttaatggtaaacatccacagatataatctacctaaacaaaagttctttatcatcctcaataatttttaagagtgaaaaagagtcctgagaccaaaaagtttgaaaaacacagctctaagctgaatacagcctttccaaaagtcttagtgcaattctaagctttaaataacttaatctgcactaagactttcgggcaccctgctggaaacagaaaagttgtaagggctttcaaagccacaaactttatgtagcagtctccagaaagggaagtccaagataggactcccaagtttgtcaaaataaacctagagttaaattgagctgtgatttcttatgacagtaagtggaattagggcaggtggcaaggtggaagaggggacattggacttagaagatctgtattctggtaccctatttggccattaacctgcaagtttcttaacctcctctaagtctacatctgaaatgttgagttggactagtagatttccaatgccccttttttctaagattcggtgactggagttagctagattttttccattatttaacatatgtttaacttttaattaataattataagtgatagaaaaattaataactataatctgatgtcaggcacctttattaaggcatacacaccttgtgaaaaagatggcagtgtatcctaaaaagatgagggaaaacagattttaagtgctgaaattgcaaaacctaaaattataaacaattgtcacgtgcttttaaagtatgttaatttttgactatgtgggagagttaggctcaatcaagtctccagttttgtccttactttttcaaaaaccttagtttatacagtttgtagattattatacataaagttttatattttcttacaattaattttttgttgatacctgtgtaaatgggtttctcctttattctttcatgctgagttttaagacgagagaataatagccaacagtaccctttattgttaaaccaatcctgggttgatactgccctcagaaacaaatatggaacatccgtatcataggagagaagagtttggtcataactaaggctgtgtggtcctttgaattatatacaatgttcttctcaactttgttttatttgtatcttaggagagaaagaaacctttgtgagagtttttacaaattaaatcactaaattgaagattcgtcatgcatttcttaaaataggtgacatgcaacttttcatcaaactgtctaccaacaacagtgtgagataaaactaaattaaaaaaaaaaagttcatctgatttctactccatctctcacaaaaattggtcaggctttgaacattatacctttgccataccagcctctcagtattctggcttgcttattacatcacagttatatgtggtttattttgtactgtattactaaaagcaactatgaacaaaatgtatgacaaagcgaagtagatacaaccttctccttcacttccatcctctcacgctcttcaggacatcccacagatcttccatcagtggttctcaactttggttggacactagaatcatctgtggagctttaaagactaattaatgtttgggtcccacccccagactctgttacgttgtgtgattggactggagtgtagtctggacgttgaggggtttaaggctccgcgggtcattctcatttacagctgttgctcagtgccattattgcctctccttgtgagatgcctgccttacccagagcaataaccaggaatcttgtcccaggtctttcagtcatttttgcagagttagtaccacttttgctgtcaaattgacattgtcacaaacttttcattaatatactgccatttcgattccttccaaatgaaacaatacagaagacgcaaggtgaaagaataactcttttaagctaataattaaccaactgtttattgtatttcatgtaaataagaaacctaattgtgcaatacaatgactgaaatgtgtaaaaatgtagcaaatatgattgtttcattgcccaagaagcagcagccagaagattctttataccatcttttactaaatctacctgctgtcttgcttttgttaataaagcatccatggcatgtttataccatatgcttattccatagtttgaaaggggatttgagtttatcagtcctgaaattctaccattattttctaaggtgtcctcagatgagaaaagttgtttgtaccaatgggaaaacttaaattgtaagacagttactacagtagttgtgctgctcctaagcatcttataaccacaagtctagtatttctttgctgaatcaggaatgggaagtgggaactgattctaatagggtaagtcatgggaagaattcatctggcaatgatggtattttctgcagaaagattgcctaaataattactaaattataaaatcttagtaatacaatacaaaatctctgctaatactgtctctttctgagtatgtagaggtttttttcttcagcttaatcagttttatttctttgactattaagagaattgattacatattagacaggtgttttaatggtaaaagcattgctctaggaattatagtaggttgtttttcagtctttattcaattgaagtgaagaatatttttctttgtatgttctaacaggcaccagaagtcatcagaatgcaagataaaaatccatacagctttcagtcagatgtatatgcatttggaattgttctgtatgaattgatgactggacagttaccttattcaaacatcaacaacagggaccaggtaaatatttaccacgtcttggtgtttattttaccgtctatatacaaggctccagttgtagaaaataagtgttaactcctgggtaagcgtgaaggatagatttcttgattttttgttaccagttttagaaatcgtttgtatacttttggcagtaatagcaacacgttaagtcctttcctcagaatatcagtcatgaatgttacaatggaataaaattcctgattttctgactagaaactacagttacaagaatggatatttcttgaccatcgcacaattagaagaatgagctctgctactatagcatctgggcattatacttttcactgtatcacagattgtgctgtcagaacaggtactcagctatgagatctttatattaaattcttttaaaattactaaggtttcttgaaatctttaatttttaaaactaggtaaatggaagaaatcatgctttgatttttctataatgagaatgctagtaggagggtaattttttacttcttttccttcctaagcaactattccctgtcccttaccttccaaaaggtactaacatagcctcatggagctcttagccttttctatctctgcctcagtaggctcgcttagacttttaaaattggccaagaaaagtgaattatctttactaaaattaccttggataactacattttaaaagatttgtgattgtgtgtgtagagagattacaacagttacactgttaatatattagaaaacactagttgaccacataaactctgattaaagaatatttcgactttatgctgtgatttgcctcagaatcacctggaaattttgcttaaaaatggagaaactggagcagattgtaattaaatggggagggtccaatcacctgcatttttttctgtttcccaggtgattctaatacagaccaaagttgagaatcaccaatctaaagattttttttaaaaacagtgcaggctttttaggtatttcataacttcccaacctaatgaaatggtagtttttgtatataaatgttactgcttcatttaagtgatttgtctgttttgtcaagagctcagctctatctttttggtgctagctctgagtagctttctcacttcacctattgtgatatcggagaaataatctcttaagttaccatgtactttgtgtgtgtacttactgaatcaccatatgccccttacacaacagtggtcttgactgtgtttgatggtttttaaaaatggctacatttctacccagttcattataaaactaaattttagttggtgttggcctttctaactacttcatagctttagaatgttgagtcttagctttgagtgtgtaacaacatgactttggatctgatgttttaagcctgcaggaaacttagattcaggatggactgaaatttcagctaaagaacataattgaaacattgtacttactgtacttgtgatagatctagatcactgaaatgattgatttgacaatggcagttggggtagcaaaatggtctctaatttacaagcttcatgtcctctgtcttttacagtcttatttattcaatcatacagcataacagcctgtaccatgttcacttttcctgtatataatatttttctggaggaattatggatttttagtttagtttcagttgatttatcatatagactacaaattaataaaaatttatgaacctaagatctggatgaaattcatttgtcagtgaatacgtttatcttagtacaataataaaattataatatagaaacaagtattatctccaaaatacagataaaagcatcccagagttcttctctcatccacttcttggcattttaggtgctttgtcctccatgggagtataataaatgatgtggcaagggcttactctccatgagaggaatgtgtgaccaacagaagggtaaggcctttactagttaattctttctaatagtatagttagaaccttctggaatttgctagtctgaaaccaagttaagtatttaatggagaagaaggaggtaaaagaatgatgtcctcttacaactacccccattctgcttcattaccccctagtctacttacgtcaaatagtactttctatgaaactccacattttgaagggttaactctggccatcctcagtgaagctgccccaggtattgctccattcatcctttgatttttcttttgctatctataatacttggatctccatagctctcagattagacttctgtttaagaatccaggaatattctttactatgcaatgtgaataccattcccctagactctcatacccatagtctgaggtggcagattttgcctgtaaattcagagcacagctggtaaagcagtgtgatgtaatggcaggccttggactcaggtaggtagactaggtctacctacctctctgggtatcttttttttttcttttttctttttttttttttccaagacggagtcttgctgtgtcgcccaggctggaatgcagtggcctgatctcagctcactgcaacctctgcctcccatgttagcgattcttctgcctcagcctcccgagtagctgggattacaggcacgcagcactacacccagctaatttttttgtatttttggtagagacaggggtttcaccatgttggtcaggctgggctcaaactcctaacctgatgatctgcccgcctcagcctgccaaagtgctgggattacaggcgtgaaccactgcacctggccttttttcttttttcaattacctgcaaaataagggaattcggctaagaatttcttccagcttcaaaaatcagattcttttctaaaatagttctcttagctctttgcaaagtagtgtgcctttttacctttattcaccctagcactaaagtctgggaaatcactttgtcatccccccactctctttatcattctaacatttttctctctaatcaccctgttcttccctccttcagtgcattttctcttgtaaattgggattaataatgtgctcacttaagtatattgactatacctttatggctttcgttccttggagtagaagtgccgtgtcttctttgaagaatagatagcatatatctgttatttcaagtgtattattccagttataattgctgcataacaaaccacccagaatgtagtagcttaaaatatgaatctgtaggttgggcttttctctgtgaggaagattcttacctgtttcacaccgtattgactagagtagatttacggggtgttaaaaggatctactcagaagtggctcattcatgtacctggcaagttggtgctagctagaagctagaagctcagttattctccacatgagcttttccttgtggacctcttcgcaggtcttcaaggacctcttcattggtctttctcacagcgtggttgctaggttctaagcacaagatctgaagaaacaggaagtaagtactagtctcttatcagtaggcaaattattttaatagaaaatgttttcaataccaactatatgcaaagcactatggaatatagaaggattcaaaatgaataaaacaaaacccaggctctgctctcaatggcatgtgcagttctgttgaggccgtgctgggggtgatactgaaaatgagcagacatgagctccagtaataccatggagtgaattgacagggtcgagacaccaggagtactgagtgtgtattgaaagtatgaaaaatcagtcccagaatttgaagctggggtattggtttcgttgtgtatgtgccacatgaatcttctgtccatgaataacatccaggaaaaacttcccttttttcttctttctgtttcttcatcagtcagcctctgcaccccaccccagcagatactgaaagtttcctgctttctgctctgcactaactaaattctgtgggatgtataaaaacatgagagttgtctttgattttttttctcacattcctcctgaaagactcttgaaaaactatgtatctccttgtatattttttaagttggcatctaaaagtttttatcttaagtttaaataattgcaaagaatgtaatttccagtatattctattaacattttaaaataagactactatattagatacatatatatccctctttttgaaatatatacatcaaaatctaaaaaccataccgattttatgcctattaaaaaatactgaagctcttcattaatttctggaagttttacatggttccttttttctcttcaaactatttgtattgtttctgtcacagaacttcatcctaatgtaacatatttgtatgttttaaagccttttattgattacctaagttacatctctgcaataaaagtatgtatataaatttaaatttagaattttacaaaattgtgacttcaagctctgattattaattttttaattctgaattatgtttttgttgcaagtaatatataactgatgaaagtagcatataagttttgatatatggaaacatagaatgtaaaattatattggaaatatgtatcttaatgaatgacatggggcttttcttttcccttcaattaatgtatgtatctgtggataaatacaacttttttgttaggacactagatactaagacttagtattgattctttccatatttcatttttaaagaggtaaatactgagaaattttatctaaattataagaagatgacaaaggcaggagttttgttataatcacatgcagagtttagtagtccccaagaccagcttcaggtttgttaaaaggacttgctgaaaactcactgaaagctgctcaagactcactgacaactgttacattcacagttacagtttattacaatgaaagaatacagatcaaaattaacaagggaagagactcataggacagaattcagaagcgttcttgttggagcttcctgtcaccctctcccaatggagttgtggacagagcttccagtagcaatgtgtgacaacacacatggattattgccaaccagggaagctcacctgagccttggagtctagagtttttattggggttcagtcatatgattaaccacctgcggggctgaccttagtttccagctcctgcagaggtcaagctgatgccacatgacccagagccctctataaatcacattgttagcacagtctgtctggcatggcccatggcccccagataaacagacactctgatcaggcaggacatttcaaggacttagtgattacctcccaggagccaagagcaaaggctagctctctctttgggcaaggttaattctttaccacatactatatatcactcaatcatctgagctcattcctataatgtaccaaaatttacatagtaacttgtcattaaaaatgttttaaaagctcagctgacatttcaattaatatttttgaaagtaaagaattggaaaccaacagactcgttacccattcgttggagcccagtttgacaccagtatttagaaatgtctctttgttgccccagaggtttttacaccctgggacaatgtatactatagttaggttacatatgggtaaaaggtgtgcctttttttttttaattaaagttttagggtacatgtacacaatgtgcaggttagttacatatgtatacatgtgccatgctggtgtgctgcacccattaactcatcatttagcattaggtatatctcctaatgctatccctccccactccacccaccccacaacagtccccagagtgtgatgttccccttcctgtgtccacgtgttctcattgttcaattcccacctatgagtgagaatatgcggtgtttggttttttgttcttgagatagtttaccaagaatgatgatttccagtttcatccatgtccctacaaaggacatgaactcatcattttttatggctgcatagtattccatggtgtatatgtgccacattttcttaatccagtctatcattgatggacatttgggttggttccaagtctttgctattgtgaatagtgccgctataaacatacgtgtgcatgtgtctttatagcagcatgatttatagtcctttgggtatatacccagtaatgggatggctgggtcaaatggtatttctagttctagatccctgaggaatcgccacactgacttccacaaaggttgaactagtttacagtcccaccaacagtgtaaaagtgttcctatttctccacatcctctccagcacctgttgtttcctgactttttaatgattgtgattctaactggtgtgagatgatatgtcgttatggttttgatttgcatttctctgatggccagtgatggtgagcattttttcatgtgttttttggctgcataaatgtcttcttttgagaagtgtctgttcatgtcctttgcccactttttgatggggttgtttgtttttttcttgtaaatttgtttgagttcattgtagattctggatattagccctttgtcagatgagtaggttgcaaaaattttctcccattttgtaggttgcctgttcactctgatggtagtttcttttgctatgcagaagctctttagtttagttagatcccatttgtcaattttggcttttgttgccattgcttttggtgttttagacatgaagtcctaaaggtgtgccttttgtaaagtggtagaagggcagttataacagggaaaatgggaaagcaagataagtgttacacttccacttgagtggttctctggcaaatcagttttttcaaaggggataccagtaagttgatagttgtagaaattaattcccttaaaaccaccatgttggctgggcgtggtggttcacacctgtaaccccagcactttgggaggctgaggcgggtggatcacttgaggtctggagttcgagaccagcctggccaacatggtgaaaccccatctctactaaaaaaatggaaacattagccaggcatggtggtgtgcacctgtaatcccagctacttgggaggctgaggcagtagaatctcttgaacccaggaaggggaggttgcagtgagccgagatcacaccactacactctagcctgggcaacagagcaagactgtctcaaattaaaaaacaaaaacaaaaaaaaccccaccatgtctatacacctctggcaaagtcttcctgtaaccccagggatacttgactctaatttaaagactatagacctatgacatggctgatcaactagcaaaaagttatcactcaccttaatgaacagttaacttaaacattgaaaacctcttgtgtccacaaggttatgttaaatactggaggtagtggtgttatagatattttttaagaatttttttttttttgagacagtctcgctctgttggccagtctggagtatagtgtggcatgatcttggctcactgcaacctctgcctcctgggttcaatcaattccttgcctcagcctcccacgtagctgggactacaggcacataccgccacacccagctaatttttatatttttagtagagacggggtttcatcatcttggccaggctggtcttgaactcctagacctcatgatccaccctcctcagcctcccaaagtgctgggattacaggcgtgagtcaccacgcctggcttctttaagaatttttttaaaatatgtatgtatgagtcacaatctctctgcctgagcacccatagtctcattggagaacttagataagatacatatcacaaaaagattaataaccatacaaggcagtaaatgatcatcacagctagtggtgggaagaaggaccattattacttctaggtgtgtaaagaaaggtatgataagcatggttacctttcagttaggcctgatcatctgggtttcaggtagctagagaagggtgagggagggcattataagcagagtaggagcagcggcaataaaaaagttaaaagtagctttgtattgggatagtcttctctacaaatcctgttacttacttaactgtattatctcgggcaagttacttaatttatttgagcattggattccttatctgtaaaacagagtcaacaccaaccttgtagaattcttttttgagtattagatgatatctagaatccaagtgtcccaacgttttttggcctttcagggccaccttttccactgatagccaagtaagaatacctgaattgctgtccataatatatgactataggaactccagatcctttctctcaactttgaagacccctgtttgaagtgctttacattctcctggctgttgcttgtcacttgttgctccaacatattattccaggttgctcccaaaattctaagactgctgtcttccattaaaatgatgatgttatgatggtgtagcttcagtagggggcattgtgaaagaaaactgtaatcccctcaattcagtaactctttccaaggttcatcctttaacctctactacatagaggccacattgcaatcaaagctttactaatgcctggcagttaaacaagggtcattacagcaccaaacaggaaataacacaatgtatcaagcttggcggtttctaaaattcatcagctctgccctgtagcagggatcttttatgggctttctccactttagcctctactctccccctgttgcacctattccctctaagaccaaaagcccttctctcaggctgtcctccctgagtcccaagactcctaacatctatccctgcctgccctcaaaagaggcaaaatattaggaaattaagaaattagaaaaaattataaatctctgaatccagggaagacaagcctttttatttcgttaaacagattttagctcctaatggcttccccttctctcctctggcggaggctctggtttgtataaccaagtggcccattagcgtttccacaataaaagggcttaagatgcagatgccaaaacgactgctaaaatgttaacctttttggttttccctacacatatatagaggacttaacacaacgtccaacaacaagaagtgctgaataaattttttttttttttaaagtaaagaagaggcactgtcagatctgttttagatggttaccctgtgagtgggaaaatggactggataaagagagaccaaaagcaggggagcaattagagaacctttgccacagtccaaggatcatgaagatttgagcttgcactccttttgtgggtttcccaccatctatgatgtggcattggttttttaaaactttttattgaggtgtaatacatacatcgtaagctgtaaatgtacagcttattgtgttttcaatgagtgtgaagtatatttttcaacttatctacaaggttgaattatttcccttttttttctctctccagataatttttatggtgggacgaggatacctgtctccagatctcagtaaggtacggagtaactgtccaaaagccatgaagagattaatggcagagtgcctcaaaaagaaaagagatgagagaccactctttccccaagtaagtaaaagcttcatgctatccaaaagaacagactaacattcatagacagatttctgagcacttttttgggcacacagtgtgtatttcatgagtttggattctatgtgcagactccagacaagaaaacacattaagatggcttcatgagggttgagcagtggcacacctagaaattttgggtcctaatacaaaatattcagaaagactttgcatttgtccatcagttctcagacttctcagtctttgaactcttttacacatttaaaaattatcgaagacccccccccccacaaagagcttttgtttatataagttttaattccttattagaaattaaaactattttttaaaatattaatgaattaaacatagtaaaccaatatgttaaataatatttagaaaaaaacagttctattatctaagagaaaaaaattagtgaggagttgtattgttttactttttttgcaaatccctttgcaaaaagaagacagctggattctccgcttttgcactcacattgcaatatctcacatcatgtcacccctaaaaactctacttaacgcttgtaaaataatgacagtaaaaaaggcaaatgatatcttattgttattacaaaaatagttttgacctcgtggatcccctggtggtccacggaccacattttgtgaaccactgctttatttcttcttggtggtaaacagattcaagctttcctttaatactgggtctttttgaggggcatttctgcctgatgcagaaaaaggaaaaggcagtaaggcatttgtcagctcagcctgcctttacctaattcttgataactcactgcttttttttttttttttccatattggaaggataaagccttaagttaacaaatttcaaaaagaactgtaactaaggccaggtgtcgtggcttacaaatctcaacactttgggaggccaaggcaggcagatcagttgaggtcaggagttcgagaccagcctggccaacatggtgaaaaccgtctctactaaaaatacaaaaattagctgggcatagtggcaggagcctgtaatcccagccactcaggaggctgagacatgagaatcgcttgaacccaggaggtggagattgtaatgagctgagatgacaccactgcattccagccagggcaacagaatgagactctgtttaaaacaaaacaaaacaaaaatatctaaatacctcaactagcttacagagtttagctgtagtagatattataatataaatgcaatgttttctgatacttagggagctcctgatagcactggataaatatgccttgatgaatcagtacagtttcaagtgggaagtgctatttcccatagtaaccctgctaccataattactggagtgttcatgtatgaactcttaggcctttgaatgcccagtcctagctgagtaactcagacagaagtcaatgttacaggaattagattcactcatcctttatttttaaatctaaaaaatgttattcttctctaaagaatgaaagaagataaaattgatggttttaacaagtctgtcagggtgttgcctaagaaaagaaagggaaagctaagtggttggctgggtagatataagtttcatttatcaggtttcaaagttactgttcctttgaggaagagtttttattttttctttttatttttttatttgtttctctaagtcaggaaacctctgggttgagactgtcctggtgagagacaagtgctgctgagcagcttcagcatgaccctgtcccatgttctcttttcacgtcatggtgctcgggttcttacttagaatgtttattgacattaataatgcaaagtacatccttaggcccactttttaaatgatattaaaaggggcaaaatgcttagctccatacttctaaatataacatttcattaattacatggtttcaaaaactgaagtgcatatgtgaactttccagattattggaggctatccctttaaagtgttattttgaaatttttgttacaggatatcttttcacaccatactgttatatgccacataaattttagatggctgaagatctatatgttttataaaatatgaaatcatttttatagttttgaggtaggaaaggctttcttaatagacaaaattcagaaggaaaaaattagcagatgtgagtacattaaaaatttttaaacttctatatagaaaaataacattgaaagataaaaagaataattgtagcatatataacaaaaagtaaatgtttataatatacaaaggactcctccaaatcaataagtaacaaacaatagaaaatgggcaaagggaatatgaacaggtgattcacataataaatacaaatggtgaatgaacttaggaaaaggtgtaaatatttccatggcagtcagtaaaatgcaaagacacaacagtgaaatatattttaatttcgcctctcaggttggtaatatccagcactgggaacaatgttgggaagtgaggaggagcattctatgtaaaattttaaaggtttggtggaaggcagcattttggaagaccttggtagaatccataaattcaaaatacttctaaaaatctgtgctatgtaacctatttcataagtattcagatatatataagaatgtttactataataagaaaaagacattaagattaagttagttttgtattgatgacatggatattggtgagagaaaaagaaaacaagacagaaaacaaaatgtagtatgatacctcattttttttttttataggctgggcctgttctgttgcccaggctggagtgcagtaatatgatcatagcccactgcagcctcaaactcctgggctcaagcgatcctctcacctcagcctcctaagtagctgggattgcaggtgcctatcaccacacccagctaatttcttgtggtggtggtggtggtggttgtagagatagattctcactatgttgccctggctggtcttgaactcctggcctcaagcgatcctctcgcctcagcctcccaaagtgctgggattacaggcgtgagccccagtgcccagcctgataaccactttaaaaggttaaacagaggccaggcacggtggctcacgcctgtaatcccagcactttgggaggccgaggcaggcggatcacctgaggttgggagttcgaaaccagcctgaccaacatggagaaactctgactctactaagaatacaaaattagccaggcgtggtggcacatgcctgtaatcccagctactcgggaggctgaggcaggaaaatcatttgaacccaggaggcggaggttgcggtgagccgagatcgcaccattgcactccagcctgggcaacaagagtgaaattccatctccaaaaaaaaaaagtggtgttcaggtgggccttgttttcatgtatgtatttttatacataaaaaaaggtactgaagaggccaggcgcagtgactcacacctgtaatcccagcactttgggaggccaaggtgggtggatcagttgaggttaggagttcgagaccagcctgggcaaaatggtgatacccgtctctactaaaaatacaaaattatccgggcgtggtggcacacgcctgtgatcccagctactcgggaggctgaggcaggagaatcgcttgaacctgggaggcggaggttgcagtgagctgagatcgcgccactgcactccagcctggacaatagagtgagactccatctcaaaaaaaaaaaaaaaaggtacagaagaaagtatagactctaacagtggttatccctggagagcaggatttgagagccttatactctttatacatttctatagtattttaatttttatttgcatgttatacttggaatttacaattttttgcaactgcttacttctttgtcttatactaatcatcataaagattactttttaaaaaaaatttaacttttaaaaacaattttcagccaggcatggtggctcatgtctgtaatcccagccctttgggaggccgaggcaggcagatcacctgaggtcaggagtttgagaccagcctggccaacgtggtgaaaccctgtctctgctaaaaatacaaaaatttagctgggcatggtggtgcgctcctgtaatcccagctactcaggaggctgaggcaggagaatcgcttgaacccaggagggggaggttgcagtgagctgagattgtgccactgcactccagcctggttgacaacagcgagactccgtttcaaaaaaaaaaaaaaattggtatctcaggacaataacaaaagtaataataatagctgctaaggttttattgagtgcttattataggccaggcattatgccaagccctttaaacatgtttcatgattatgaacatgcattatcatgctgtatgccttcaaggattataacctgtttctttgtgccttaaaattgtgaatttctgcattttatatattggggtctatttgtcgagttctcctatctttgctcttgggttgtcccctgtcacttctcatgtgctactagcactctgggtctgtgaggttctgctttcaattaggtgtatgtaaaacatttcccatggctaggtttctttaaagggcaagtagctgtgataattctgtttagagatagtcataaagtgctttacttatttatactccatcttcttcccaaaagagacttgtggtctataacaaaaaggtataaaattggttttaaatttctattatttactgtttcaagactaacaaatgatctaaaatataaataaaagctgactaagaattactctccccatttaatttacagagagagtttcttcttaagaaaaaataccaattatttacaaatattttcccaagcatttatgacaatgctgaaaacaatgtaagatttcaggtgctttcttgtaaagtgtgatgggactcttaaagatttataccacccagattttcattcttctttctgttttttctttttctttctttctttttttttcttttttctttttttttcttttttttttctttttttttttgtagattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaa

(19) United States
(12) Patent Application Publication
(10) Pub. No.: US 2012/0134995 A1
(43) Pub. Date: May 31, 2012

(54) METHOD FOR PREDICTING THE THERAPEUTIC RESPONSEIVENESS OF A PATIENT TO A MEDICAL TREATMENT WITH AN EGFR INHIBITOR

(76) Inventors: Pierre Laurent-Puig, Paris (FR); Jean-Louis Merlin, Vandoeuvre Les Nancy Cedex (FR)

(21) Appl. No.: 13/320,299

(22) PCT Filed: May 15, 2009
(86) PCT No.: PCT/IB09/53522

§ 371 Date: Feb. 16, 2012

(51) Int. Cl. A61K 39/395; C40B 30/04; A61P 35/00

(52) U.S. Cl. 424/138.1; 506/9

(57) ABSTRACT

The invention provides a method of predicting the response of a patient affected with cancer to a treatment with an EGFR inhibitor, wherein the said method comprises detecting the level of expression of the phosphoprotein pP70S6k in sample from said subject. The invention also provides methods of treatment of cancer and or predicting the outcome of cancer.

### FIELD OF THE INVENTION

The invention relates to the general field of molecular diagnosis, and particularly to molecular markers. In particular, the invention relates to a method of predicting the response of a patient to a treatment with an EGFR inhibitor, wherein the said method comprises detecting the level of expression of the phosphoprotein pP70S6k in sample from said subject. The invention also provides methods of treatment of cancer and or predicting the outcome of cancer.

### BACKGROUND OF THE INVENTION

Recent progress has been made in the treatment of cancer, namely colorectal cancer (CRC) with the introduction of new treatments targeting the epidermal growth factor receptor (EGFR) and the vascular endothelial growth factor. Monoclonal antibodies represent one of the most effective treatments for the inhibition of EGFR and more generally for a variety of cancer diseases.

Cetuximab is a chimeric IgG1 monoclonal antibody, which binds to EGFR with a high specificity and prevents the phosphorylation of the intracellular tyrosine kinase domain of the receptor which in turn inhibits activation of downstream signaling pathways like Ras/raf/MAPK, PI3K/AKT and STAT. Inhibition of these pathways restores normal cell proliferation control and induces apoptosis.

Cetuximab has proved to be active in irinotecan-resistant metastatic CRC expressing EGFR by immunohistochemistry (IHC). A randomized phase III study showed a significant improvement of progression-free survival (PFS) and overall survival (OS) with cetuximab when compared to best supportive care in 572 patients previously treated by fluroropyrimidine, irinotecan and oxaliplatin. Similar results have been obtained with the fully human IgG2 anti-EGFR panitumumab which was associated with a higher response rate and a longer PFS when compared to best supportive care in chemo-refractory metastatic CRC patients. However, only a subset (8-23%) of patients achieves an objective response and benefit from cetuximab or panitumumab. Furthermore EGFR expression based on IHC has failed to demonstrate any correlation with response to these antibodies. It is therefore a need to identify other predictive markers of response to an EGFR inhibitor.

KRAS mutations have recently been reported to be associated with lack of response to cetuximab and/or poorer survival in chemo-refractory metastatic CRC patients in several independent studies (1-6). The predictive and prognostic value of KRAS mutations was also confirmed in the randomized phase III trial comparing monotherapy with panitumumab with best supportive care (7). The hypothesis is that KRAS mutation could be responsible for an acquired activation of the Ras/MAPK signaling pathway downstream of the EGFR which would be independent of the ligand binding to the receptor, and which could therefore, induce resistance to anti-EGFR antibodies, as it has been demonstrated in vitro (1). On the other hand, only 28% to 64% wild-type (WT) KRAS patients respond to cetuximab (1-6), which suggest the existence of other molecular markers of resistance to this treatment.

Hence, given that a significant portion of wild-type (WT) KRAS patients does not respond to cetuximab, the question remains for medical practitioners to whom and when to recommend an EGFR inhibitor. There is thus a need in the art for reliable markers allowing the discrimination between (i) disease-affected patients to whom a treatment with an EGFR inhibitor will bring a medical benefit, which patients may also be termed “responders” and (ii) disease-affected patients to whom a treatment with an EGFR inhibitor will bring no medical benefit, but who may undergo side effects, which patients may also be termed “non-responders”.

As the key mechanism involved in resistance to a treatment with an EGFR inhibitor appears to arise from the constitutive activation of EGFR downstream signaling pathways, the aim of the inventors was to evaluate the expression of EGFR downstream signaling phosphoproteins of the MAPK (pERK1/2, pMEK1) and PI3K/AKT (pAKT, pP70S6k, pGSK3β) pathways as potential new predictive markers for a treatment with an EGFR inhibitor in CRC patients, and cancer in general.

### SUMMARY OF THE INVENTION

The inventors have now identified that phosphoprotein pP70S6k (“phosphorylated P70 ribosomal S6 kinase”) is a biomarker for predicting the response of a patient affected with cancer to a medical treatment with an EGFR inhibitor.

One aspect of the invention consists of a method of predicting the response of a patient affected with cancer to a medical treatment with an EGFR inhibitor comprising:

a) assessing in a sample from said patient the level of expression of phosphoprotein pP70S6k;

b) comparing the level of expression of phosphoprotein pP70S6k in the sample of said patient to a reference level of expression of phosphoprotein pP70S6k,

wherein a low level of expression of phosphoprotein pP70S6k in the sample of said patient with respect to the reference level of expression of phosphoprotein pP70S6k correlates with a higher probability that said patient is responder to medical treatment with an EGFR inhibitor.

The second aspect of the invention relates to a method of treatment of patient affected with cancer whose condition is likely to be improved by treatment with an EGFR inhibitor, which method comprises the steps of:

a) assessing in a sample from said patient the level of expression of phosphoprotein pP70S6k;

b) comparing the level of expression of phosphoprotein pP70S6k in the sample of said patient to a reference level of expression of phosphoprotein pP70S6k,

wherein a low level of expression of phosphoprotein pP70S6k in the sample of said patient with respect to the standard level of expression of phosphoprotein pP70S6k correlates with a higher probability that said patient is responder to a treatment with an EGFR inhibitor, and

c) if the patient is determined as a responder to a treatment with an EGFR inhibitor, administering to said patient a therapeutically effective amount of an EGFR inhibitor.

The methods are preferably carried out for patient affected with a solid tumor.

The cancer is preferably a cancer associated with a solid tumor, such as a cancer selected from the group consisting of breast cancer, bladder cancer, uterine/cervical cancer, oesophageal cancer, pancreatic cancer, colon cancer, colorectal cancer, kidney cancer, ovarian cancer, prostate cancer, head and neck cancer, non-small cell lung cancer and stomach cancer. More preferably, the patient suffers from colorectal cancer.

In certain embodiments the anti-EGFR therapy consists of an antibody specifically directed to EGFR

Most preferably, the EGFR inhibitor consists of a treatment with an antibody specifically directed to EGFR, when the patient suffers from colorectal cancer

The sample from patient consists preferably of a tumor sample.

In one specific embodiment, the patient's tumor is wild-type for the KRAS gene and optionally wild-type for the BRAF gene. The presence of wild-type (WT) KRAS gene is indicative that the patient may or may not be a responder. Furthermore, mutations in the BRAF gene were also previously reported to be associated with a lack of response of patients treated with anti-EGFR antibodies (1).

It also has been discovered that two phosphoproteins are independently predictive of shorter progression-free survival (PFS), and shorter overall survival (OS) in cancer patients. Namely, phosphoproteins pMEK1 (“phosphorylated MAP/ERK kinase”) and pP70S6k have been discovered as markers for predicting the survival of a patient with a cancer disease.

Thus the third aspect of the invention relates to a method for determining a prognosis of cancer in a patient, the method comprising:

a) assessing in at least one sample from said patient the level of expression of phosphoprotein pMEK1 and/or phosphoprotein pP70S6k; and

b) comparing the level of expression of phosphoprotein pMEK1 and/or phosphoprotein pP70S6k in said at least one sample from said patient to a reference level of expression of phosphoprotein pMEK1 and/or phosphoprotein pP70S6k, respectively,

wherein a low level of expression of phosphoprotein of pMEK1 and/or phosphoprotein pP70S6k in said at least one sample from said patient correlates with a positive prognosis of cancer in said patient.

In a preferred embodiment the sample from patient consists of a tumor sample.

### BRIEF DESCRIPTION OF THE DRAWINGS

FIG. 1: Levels of pAKT, pGSK313, pP70S6k, pMEK1 and pERK1/2 according to KRAS status; “mutKRAS” denotes mutated KRAS tumors; “wtKRAS” denotes wild-type KRAS tumors (individual value and mean value).

FIG. 2: pP70S6k levels according to the response status to treatment with cetuximab (individual value and mean value).

FIG. 3: A: Progression free survival according to pMEK1 (cut off value of 79.3 arbitrary units). B: Progression free survival according to pERK1/2 (cut off value of 42 arbitrary units). C: Progression free survival according to pAKT (cut off value of 37.9 arbitrary units). D: Progression free survival according to pGSK3 (cut off value of 59.3 arbitrary units). E: Progression free survival according to pP70S6k (cut off value of 16.3 arbitrary units).

FIG. 4: A: Progression free survival according to MEK1 and KRAS status. B: Progression free survival according to pP70S6k and KRAS status.

FIG. 5: Overall survival according to pP70S6k and KRAS status.

### DETAILED DESCRIPTION OF THE INVENTION

The present inventors have assayed for a possible statistical linkage between (i) the specific expression of EGFR downstream signaling phosphoproteins of the MAPK and PI3K/AKT pathways from patients in need of a treatment with an EGFR inhibitor, and (ii) the ability of the said patients to react positively to the said treatment.

To that effect the inventors have analyzed the level of expression of signaling phosphoproteins in samples originating from (i) patients who are responders to a treatment with an EGFR antibody (cetuximab or panitumumab), and (ii) patients who are non-responders to a treatment with the said anti-EGFR antibody, and found that there was a significant statistical linkage between (1) the level of expression of phosphoprotein pP70S6k in patients and (2) the ability of the patients to positively respond to the treatment.

As disclosed in the examples herein, the inventors have screened samples originating from a well characterized cohort of 42 patients affected with colorectal cancer (CRC) which were treated with antibodies directed to EGFR (cetuximab or panitumumab) in order to assess the pharmacologic effect of the level of EGFR downstream signaling phosphoproteins of the MAPK and PI3K/AKT pathways. Significant associations between the response to the treatment by said anti-EGFR antibodies and the level of expression of the phosphoprotein pP70S6k have been found.

Five EGFR downstream signaling phosphoproteins of the MAPK and PI3K/AKT pathways were analyzed in a cohort of treated patients. For each phosphoprotein, the level of expression using Bioplex® phosphoprotein array was tested for associations between the level of phosphoprotein expression and the clinical outcome (responder or non-responder to the treatment and for survival in the patient population).

Namely, 42 patients (24 males; mean age: 61.8 years) with histologically proven metastatic colorectal adenocarcinoma treated by cetuximab (n=41) or panitumumab (n=1) were analyzed. These 42 patients were selected from a previous series of 114 patients (6) for whom protein material was available. As they were all included in a previous study on KRAS mutation as predictor of response to the anti-EGFR antibodies, all the patients were assessable for tumor response.

One patient received panitumumab in monotherapy and 41 patients received cetuximab in monotherapy (n=2) or in combination with irinotecan (alone, n=37; FOLFIRI regimen, n=2) The anti-EGFR treatment was given as second-line, third-line, fourth-line, fifth-line or more in 16, 16, 4 and 3 cases respectively. One patient received cetuximab as first line without any additional chemotherapy. The median follow-up was 10.1 months.

The inventors demonstrated that a low level of expression of phosphoprotein pP70S6k in a biological sample from the patient correlates with a response to a medical treatment with an anti-EGFR antibody.

By determining the level of phosphoprotein before treatment with an EGFR inhibitor, the inventors further showed that the level of expression of phosphoprotein pMEK1 and/or pP70S6k correlates with the prognosis, namely the survival rate of patients with a cancer.

As disclosed in the examples herein, the inventors found that colorectal cancer patients with high pMEK1 or pP70S6k expression had a shorter PFS than those with a low expression. Patients with high pP70S6k expression further had a shorter OS (6.5 versus 21.6 months, p=0.003). In multivariate analysis, PFS was significantly shorter for patients with high pMEK1 or pP70S6k expression, independently of the KRAS status, as OS for patients with high pP70S6k expression. Therefore, WT KRAS patients with high pP70S6k expression had a significantly shorter survival than those with low expression.

The inventors demonstrated that the level of expression of these phosphoprotein pMEK1 and/or pP70S6k significantly correlated with the survival of patients with cancer and that this information can be used to select a group of patient that will benefit from the treatment by an EGFR inhibitor.

### DEFINITIONS

P70S6K (p70 ribosomal S6 kinase also known as S6K; PS6K; S6K1; STK14A; p70-S6K; p70-alpha; p70(S6K)-alpha; RPS6 KB1) is a serine/threonine kinase. As suggested by the name, its target substrate is the S6 ribosomal protein. Phosphorylation of S6 induces protein synthesis at the ribosome level. P70S6 kinase is included in a signaling pathway that includes mTOR (the mammalian target of rapamycin). mTOR can be activated in distinct ways, thereby activating p70S6K.

Human P70S6K exists as two isoforms: isoform alpha I which is 525 amino acid long and which sequence is shown as SEQ ID NO:1 (accession number P23443 in Swissprot database), and isoform alpha II which lacks the 23 N-terminal amino acids of isoform alpha I.

In the context of the invention, “pP70S6K” denotes the P70S6K protein (both alpha I and alpha II isoforms) phosphorylated at least on Ser441 and/or Thr444 as shown in SEQ ID NO:1.

P70S6K is herein referred to as a “marker” or “biomarker” in that it enables to predict the response to a medical treatment of cancer with an EGFR inhibitor and further to determine the prognosis of a patient with a cancer disease.

MEK1 (MAP/ERK kinase also known as: MKK1; MAPKK1; PRKMK1; MAP2K1) is a dual threonine and tyrosine recognition kinase that phosphorylates and activates mitogen-activated protein kinase (MAPK). MEK1 is in turn activated by phosphorylation. The major site of MAPK phosphorylation in MEK1 is threonine 292. Mutation of threonine 292 to alanine eliminates 90% of MAPK catalyzed phosphorylation of MEK1 but does not influence MEK1 activity.

SEQ ID NO:2 shows the protein sequence of MEK1, in humans.

The peptidic sequence of SEQ ID N° 2 which defines the marker of interest is known per se and is namely referred to as n° Q02750 in the Swissprot database.

As used herein “pMEK1” denotes the MEK1 protein phosphorylated at least on Ser218 and/or Ser222 as shown in SEQ ID NO:2.

MEK1 is herein referred to as a “marker” or “biomarker” in that it enables to predict the survival of a patient with a cancer.

The term “PFS” means progression-free survival: i.e. the period of time during and after treatment during which a patient is alive and its condition does not worsen. Progression-free survival is relevant to a clinical study or a trial in order to assess the potency of a new treatment.

The term “OS” means overall survival rate: i.e. the percentage of population in a study or treatment group which is alive for a certain period of time after diagnosis of cancer or treatment of cancer. The overall survival rate is often stated as a five-year survival rate, which is the percentage of a population in a study or treatment group still alive five years after diagnosis or treatment. “OS” is also called survival rate.

A “tumor” refers to an abnormal growth of tissue resulting from an abnormal multiplication of cells. A tumor may be benign, premalignant, or malignant (i.e., cancerous). A tumor may be a primary tumor, or a metastatic lesion.

The terms “cancer” and “cancerous” refer to or describe the pathological condition in mammals that is typically characterized by unregulated cell growth. More precisely, in the method of the invention for predicting the response to an EGFR inhibitor, diseases, pathological tissues, namely tumors that express EGFR are most likely to respond to anti-EGFR. In particular, the cancer is associated with a solid tumor. Examples of cancers that are associated with solid tumor formation include breast cancer, bladder cancer, uterine/cervical cancer, oesophageal cancer, pancreatic cancer, colon cancer, colorectal cancer, kidney cancer, ovarian cancer, prostate cancer, head and neck cancer, non-small cell lung cancer and stomach cancer. In a particular embodiment, the tumor is a colorectal cancer.

The term “biological sample” means any biological sample derived from a patient, comprising cancerous tissues or cells expressing proteins of interest, namely pP70S6k or pMEK1. Most preferred samples are tumor tissues or tumor cells obtained through a biopsy. Whole cell protein can be easily extracted therefrom. The biological sample may be treated prior to its use, e.g. in order to render protein available. Techniques of cell lysis, concentration or dilution of proteins, are known by the skilled person.

The term “patient” refers to any human subject to be tested.

The patient may be asymptomatic or may show early or advanced symptoms of the disease, namely a cancer condition.

The term “prognosis” means the assessment of the outcome of the condition i.e. consist of predicting the evolution of the condition.

A “responder” or “responsive” patient to a treatment with an EGFR inhibitor refers to a patient, or group of patients, who is affected with cancer, and who show(s) or will show a clinically significant relief in cancer when treated with an EGFR inhibitor.

The clinical relief may be assessed according to the standards recognized in the art. For example in the following example, tumor response was evaluated by computerized tomodensitometry according to the RECIST (Response Evaluation Criteria in Solid Tumors) criteria. For the analysis, patients with complete and partial response (CR and PR) were classified as responders and those with stable and progressive disease (SD and PD) as non-responders (10).

As intended herein “a higher probability to be a responder, conversely a non responder to an EGFR inhibitor with respect to standard responsiveness” means that the probability that a patient, e.g. a patient affected with cancer, which has a low level of expression of phosphoprotein pP70S6k will respond to a treatment an EGFR inhibitor is statistically significant higher, conversely lower, than that observed for a general population of patients with the same pathology with non mutated KRAS gene and optionally non mutated BRAF gene.

As intended herein a “general population of patients” denotes a population of unselected patients, in particular as regards their level of expression of phosphoprotein pP70S6k. Preferably, the general population comprises enough patients so that the ratio of patients who respond to the treatment can be considered as statistically significant.

KRAS (V-Ki-ras2 Kirsten rat sarcoma viral oncogene homolog also known as KRAS, C-K-RAS; K-RAS2A; K-RAS2B; K-RAS4A; K-RAS4B; KI-RAS; KRAS1; KRAS2; NS3; RASK2) is a protein which in humans is encoded by the KRAS gene. Like other members of the Ras family, the KRAS protein is a GTPase and is an early player in many signal transduction pathways. KRAS is usually tethered to cell membranes due to the presence of an isoprenyl group on its C-terminus. While the protein product of the unmutated KRAS gene performs an essential function in normal tissue signaling, mutated KRAS genes are potent oncogenes that play a role in many cancers.

SEQ ID NO:3 shows the genomic nucleic sequence of KRAS gene, in humans, as available in Genbank database under accession number NG_{—}007524.1. BRAF, also known as BRAF1; RAFB1; B-RAF1; FLJ95109; MGC126806; and MGC138284; encodes a protein belonging to the raf/mil family of serine/threonine protein kinases. This protein plays a role in regulating the MAP kinase/ERKs signaling pathway, which affects cell division, differentiation, and secretion. Mutations in this gene have been associated with various cancers, including non-Hodgkin lymphoma, colorectal cancer, malignant melanoma, thyroid carcinoma, non-small cell lung carcinoma, and adenocarcinoma of lung.

The genomic sequence of human BRAF is available in GenBank database under accession number NG_{—}007873. This sequence is shown in SEQ ID NO:4.

Reference Level of Expression of p70S6K and pMEK1

The expression level of a given marker (pP70S6K or pMEK1) is determined in a relevant biological sample of a patient and compared to a reference level of said marker.

The reference level of a marker is a value which may be determined by analysis of the marker expression in a standard control.

The term “standard control” refers to a pool of tumor samples non mutated for KRAS (in particular wild-type sequence on codon 12, 13 and 61) and optionally non mutated for BRAF (in particular wild-type sequence on codon 600). For instance a pool of at least 100 tumor samples, in particular of at least 100 colon tumor samples, wild-type for KRAS and optionally wild-type for BRAF may be used as a standard control.

The reference level can be a single value, such as percentage pP70S6K or pMEK1 protein as compared with total P70S6K or MEK1 expressed, or concentration of pP70S6K or pMEK1 protein, as determined in the standard control. Said single reference value may be for instance the median or the mean level of expression (e.g. as a percentage or concentration) of pP70S6K or pMEK1 in the standard control.

A “low level” or “high level” of expression of pP70S6K or pMEK1, in the sample of a patient, compared to a single reference value, means that the expression level of pP70S6K or pMEK1, respectively, in the sample of the patient is decreased, or conversely increased, versus this single reference value. For instance, a low level of expression of a marker may denote a level of expression which is at least 10%, 20%, 30%, 40%, 50%, 60%, 70%, 80%, 90%, 100%, 150% or 200% lower than the reference single value.

Where the reference level of pP70S6K expression is a single value, it is preferred that the single value is the median level of expression of pP70S6K. The level of expression of phosphoprotein pP70S6k in the sample from a patient is then determined as low if said level is below the median level of expression of pP70S6K in the standard control.

The reference level may also be established based upon comparative groups, such as where the risk or likelihood of an event in one defined group is higher or lower that the risk or likelihood in another defined group. The predetermined value can be a range, for example, where the standard control pool is divided equally (or unequally) into groups, or into quantiles, the lowest quantile (i.e. the quantile gathering samples having the lowest level of expression of a marker, i.e. pP70S6K or pMEK1) being iamples of individuals with the highest probability (i) of being responder to treatment with an EGFR inhibitor (pP70S6K marker) or (ii) of having a positive prognosis of outcome of cancer (i.e. longer PFS and/or OS depending on the pP70S6K or pMEK1 marker), and the highest quantile (i.e. the quantile gathering samples having the highest level of expression of a marker, i.e. pP70S6K or pMEK1) being samples of individuals with the highest risk of (i) being non-responder to treatment with an EGFR inhibitor or (ii) of having a negative prognosis of outcome of cancer (i.e. shorter PFS and/or OS depending on the pP70S6K or pMEK1 marker).

Accordingly, in the methods of the invention, the reference level may comprise multiple ranges of level of expression of pP70S6K or pMEK1 measured in a pool of tumor samples non mutated for KRAS and optionally non mutated for BRAF (standard control) said pool of samples being divided into quantiles according to level of expression of pP70S6K or pMEK1, the lowest quantile(s) being tumor sample of patients having the highest probability (i) of being responder to treatment with an EGFR inhibitor (pP70S6K marker) or (ii) of having a positive prognosis of outcome of cancer (i.e. longer PFS and/or OS depending on the pP70S6K or pMEK1 marker).

For instance, the standard control could be divided into 2, 3, 4, or 5 quantiles. The samples to be assayed having level of pP70S6K or pMEK1 expression corresponding to the level of the first, two or three lowest quantiles (if the pool is divided into 4 quantiles), or corresponding to the level of the first, two, three or four lowest quantiles (if the pool is divided 5 quantiles) would be considered as samples having a low level of expression of pP70S6K or pMEK1, as appropriate.

Where the reference level of pMEK1 expression is multiple ranges of level of expression, it preferred that a low level of pMEK1 expression denotes a level of pMEK1 expression below the threshold value that defines the 80^{th} percentile of the standard control (i.e. the threshold value which delimits the fourth and fifth quintiles). A high level of pMEK1 expression would then be a level above the threshold value that define the 80^{th} percentile of the standard control. Preferably, the standard control is a pool of at least 100 colon tumor samples, wild-type for KRAS and optionally wild-type for BRAF.

### Methods for Detecting the Level of Expression of Phosphorylated Proteins

The expression level of pP70S6k and/or pMEK1 is determined by assaying a sample comprising phosphoprotein pP70S6k and/or pMEK1 collected from the patient.

Generally, any source of pP70S6k and/or pMEK1, in purified or non-purified form, can be utilized as the starting sample. Phosphoprotein pP70S6k and/or pMEK1 may be extracted from cells, tissues and the like.

The sample is preferably obtained from a tissue biopsy. Non-limiting examples of cell sources available include without limitation epithelial cells, fibroblasts, or any cells present in a tissue obtained by biopsy. pP70S6k and/or pMEK1 may be extracted using any methods known in the art, such as described in Sambrook et al. (Molecular cloning: a laboratory manual/J. Sambrook, E. F. Fritsch, T. Maniatis, 1989).

Determination of the expression level of pP70S6k and/or pMEK1 can be performed by a variety of techniques.

The expression level of the pP70S6k and/or pMEK1 marker(s) may be determined by assaying the marker protein. Various methods for detecting and/or quantifying the expression of the marker proteins are described in greater detail below.

Such methods comprise contacting a biological sample with a binding partner capable of selectively interacting with the marker protein present in the sample. The binding partner is generally an antibody that may be polyclonal or monoclonal, preferably monoclonal.

Procedures for raising polyclonal antibodies are well known and disclosed in E. Harlow, et. al., editors, Antibodies: A Laboratory Manual (1988) that further discloses procedures for preparing monoclonal antibodies.

Laboratory methods for preparing monoclonal antibodies are well known. Monoclonal antibodies (Mabs) may be prepared by injecting the purified marker protein into a mammal, e.g. a mouse, rat, rabbit, goat, human. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. Also contemplated is the use of Mabs produced by an expressing nucleic acid cloned from a hybridoma.

The presence of the marker protein can be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation.

For instance, use may be made of the Bio-Plex phosphoprotein array (Bio-Rad, Hercules, Calif.) which is based on multiplex sandwich bead immunoassays.

Fluorescent capturing beads are coupled to antibodies directed against the studied phosphoproteins and incubated with biotinylated antibodies fixing each target protein. Streptavidin-phycoerythrin revealed solution was used. The analysis consisted in a double laser fluorescence detection allowing simultaneous identification of the target protein through the red fluorescence emission signal of the bead and quantification of the target protein through the fluorescence intensity of phycoerythrin.

The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound marker protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

Typically, a solid support is first reacted with a solid phase component (e.g., one or more anti-pP70S6k and/or anti-pMEK1 antibody(ies)) under suitable binding conditions so that the component is sufficiently immobilized on the support according to methods well known to those skilled in the art. After reacting the solid support with the solid phase component, any non-immobilized solid-phase components are removed from the support by washing, and the support-bound component is then contacted with a biological sample suspected of containing ligand moieties (e.g., pP70S6k and/or anti-pMEK1 molecules toward the immobilized antibodies) under suitable binding conditions. After a washing step to remove any non-bound ligand, a secondary binder moiety is added under suitable binding conditions, wherein the secondary binder is capable of associating selectively with the bound ligand. The presence of the secondary binder can then be detected using techniques well known in the art.

### Prediction Method of the Invention:

### a) Prediction Method of the Invention for the Response to a Medical Treatment

One aspect of the invention consists of a method for predicting the response of a patient affected with cancer to a medical treatment with an EGFR inhibitor comprising the following steps:

a) assessing in a sample from said patient the level of expression of phosphoprotein pP70S6k;

b) comparing the level of expression of phosphoprotein pP70S6k in the sample from said patient to a reference level of expression of phosphoprotein pP70S6k,

wherein a low level of expression of phosphoprotein pP70S6k in the sample from said patient with respect to the reference level of expression of phosphoprotein pP70S6k correlates with a higher probability that said patient is responder to a treatment with an EGFR inhibitor; and a high level of expression of phosphoprotein pP70S6k in the sample from said patient with respect to the reference level of expression of phosphoprotein pP70S6k, correlates with a higher probability that said patient is non responder to a treatment with EGFR inhibitor, with respect to standard responsiveness.

The method above which is based on the assessment of the level of expression of phosphoprotein pP70S6k within a tumor of the patient allows distinguishing (i) patients consisting of responders to a treatment with EGFR inhibitor from (ii) patients consisting of non-responders to a treatment with EGFR inhibitor, the said EGFR inhibitor consisting preferably of a antibody directed to EGFR.

In particular, the probability that a patient affected with cancer, e.g. colorectal cancer, who has a low level of expression of phosphoprotein pP70S6k will respond to a treatment with an EGFR inhibitor is higher than that observed for a population of patients with the same pathology but who does not have the same level of expression of said biomarker, i.e. who has a high level of expression of the phosphoprotein pP70S6k.

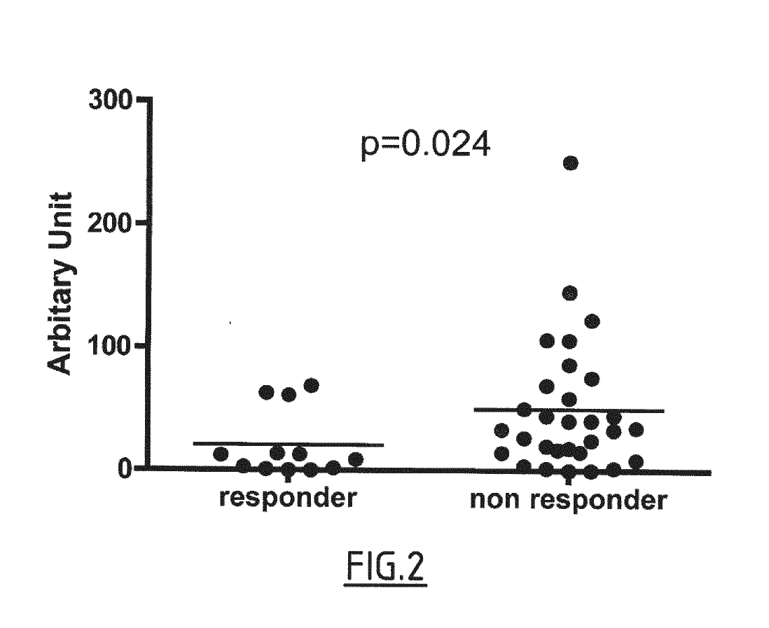

Namely, as disclosed in the examples herein, odds for a non-response to anti-EGFR therapy is decreased in patients that have a low level of expression of the phosphoprotein pP70S6k. Conversely, the risk that a patient consists of a non responder is increased in patients a high level of expression of the phosphoprotein pP70S6k (FIG. 2).

The prediction method according to the present invention is preferably performed on samples from patients affected with a cancer that may be prevented or treated with an EGFR inhibitor, preferably a colorectal cancer.

Typically, the prediction method of the invention is performed on peptide samples originating from patients that are affected with colorectal cancer that may be prevented or treated with anti-EGFR antibody.

In one specific embodiment, the KRAS status of the patient was determined, the presence of wild-type (WT) KRAS gene being indicative that the said patient may be responder to EGFR inhibitor. Conversely, the presence of a mutated KRAS allele in the tumor of the patient is associated with lack of responsiveness to treatment with EGFR inhibitors.

KRAS mutation status can be determined on tumor samples, preferably before the treatment with an EGFR inhibitor, by an allelic discrimination assay or checked by direct sequencing of exon 2 as previously described (6, 9). It may be checked in particular that the KRAS gene in the tumor cells is wild-type on codon 12, 13 and 61.

Optionally, the BRAF status of the patient was also determined, as mutations in BRAF gene have also been associated with lack of responsiveness to treatment with EGFR inhibitors. BRAF mutation status can be similarly determined on tumor samples, preferably before the treatment with an EGFR inhibitor, by an allelic discrimination assay as described in (9) or by direct sequencing, for instance. It may be checked in particular that the BRAF gene in the tumor cells is wild-type on codon 600.

Therefore, the method of prediction according to the invention is preferably carried out for patients whose tumor is (homozygous) wild-type for the KRAS gene and optionally (homozygous) wild-type for the BRAF gene.

b) Method of Prognosis of a Patient with a Cancer Disease

As already described previously herein, the inventors have further discovered that there was statistically significant relationship between the level of expression of phosphoproteins pMEK1 and/or pP70S6k, and the chance of survival of a patient with a cancer disease.

Another aspect of the invention therefore consists of a method for determining a prognosis of cancer in an patient, the said method comprising the following steps:

a) assessing in at least one sample from said patient the level of expression of phosphoprotein pP70S6k and/or phosphoprotein pMEK1; and

b) comparing the level of expression of phosphoprotein pP70S6k and/or phosphoprotein pMEK1 in said at least one sample from said patient to a reference level of expression of phosphoprotein pP70S6k and/or phosphoprotein pMEK1, respectively,

wherein a low level of expression of phosphoprotein of pMEK1 and/or phosphoprotein pP70S6k in said at least one sample from said patient correlates with a positive prognosis of cancer in said patient.

The probability that a patient affected with cancer, such as colorectal cancer, who has a high expression level of phosphoprotein pP70S6k or pMEK1 has a shorter survival, is higher than that observed for a population of patients with the same pathology but exhibiting an average low expression of phosphoprotein P70S6K or pMEK1.

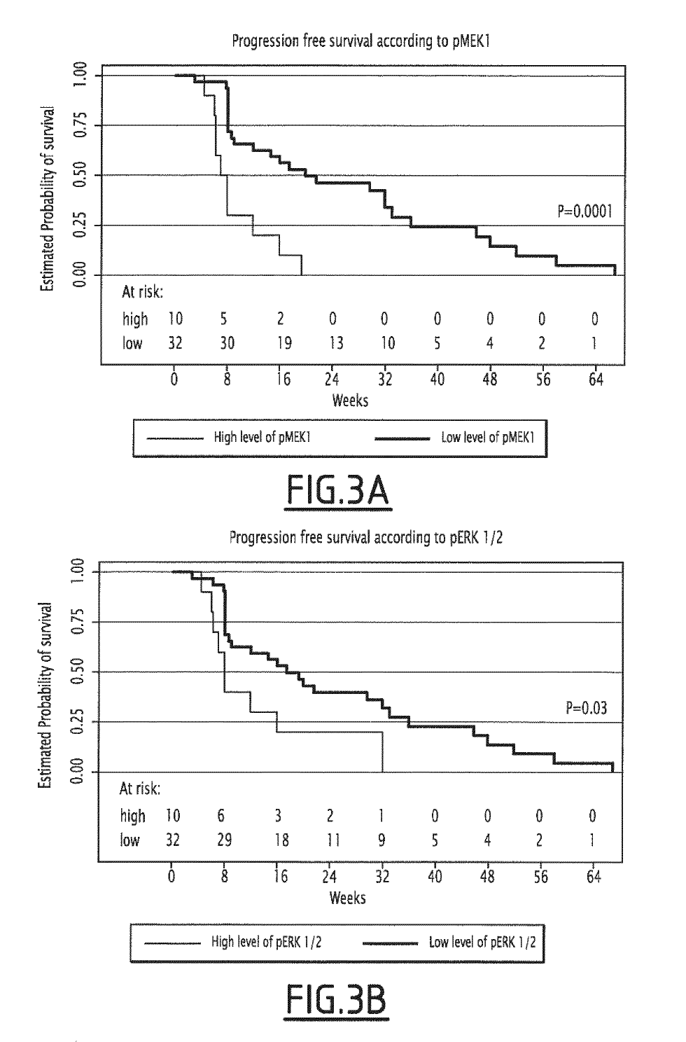

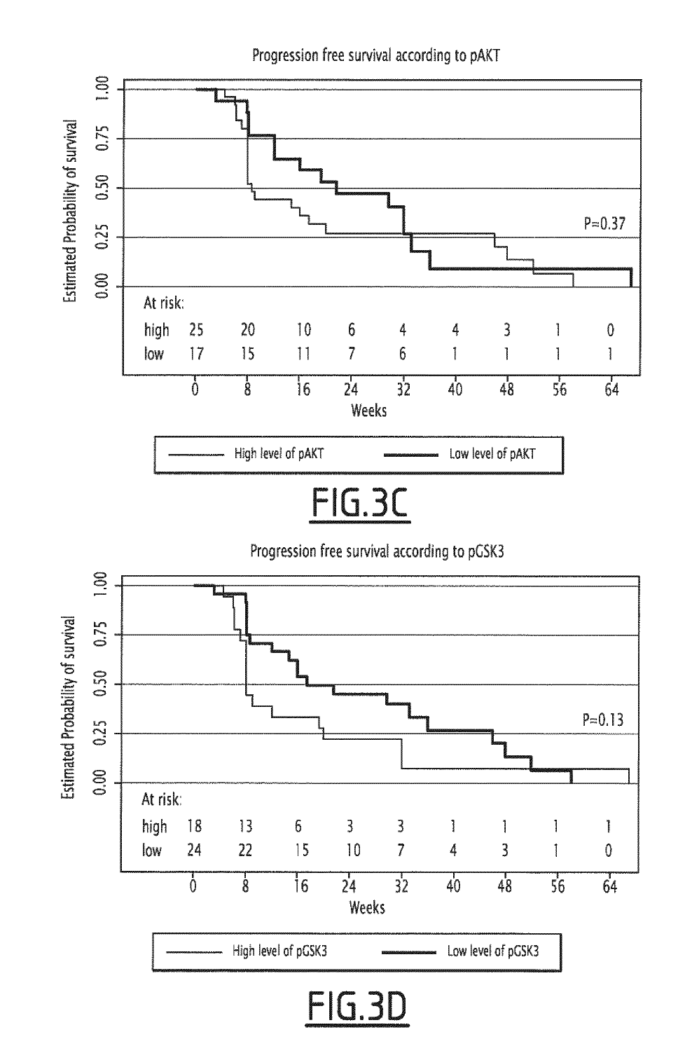

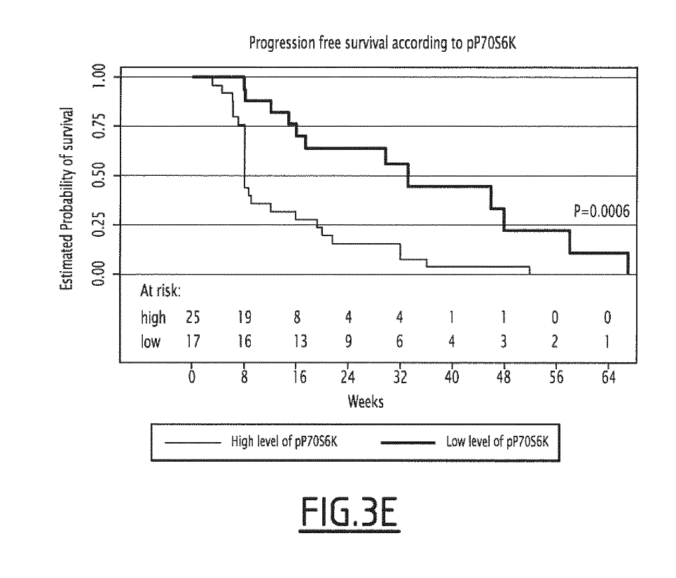

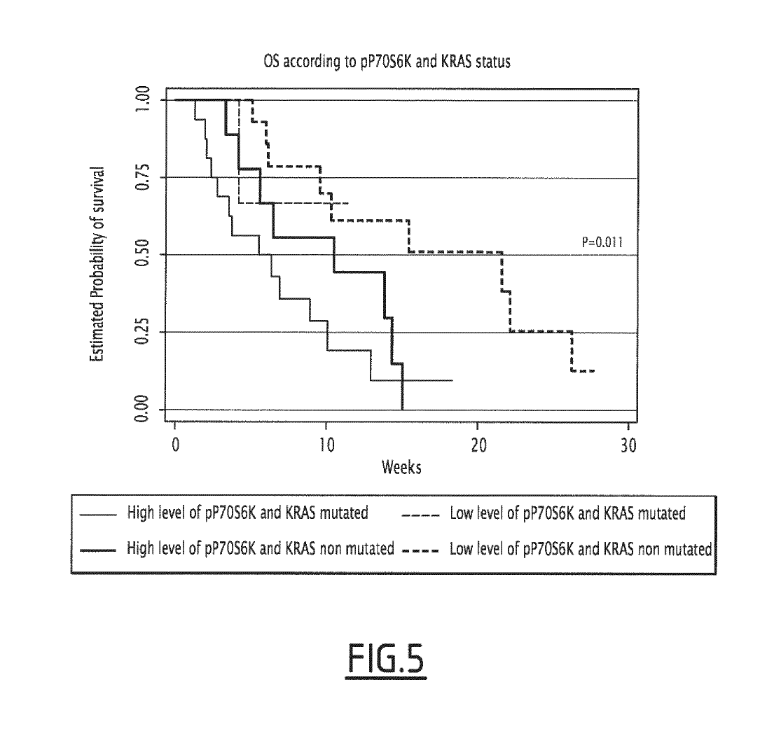

In particular, as disclosed in the examples herein, odds for a short PFS are increased in patients with a high pMEK1 or pP70S6k expression (FIGS. 3A and 3E) whereas odds for a short OS are increased in patients who have a high pP70S6k expression (FIG. 5).

In the above method for determining a prognosis of cancer, a low level of expression of phosphoprotein pP70S6k in said at least one sample from said patient correlates with a positive prognosis for PFS and OS of said patient (i.e. longer PFS and/or OS), and a low level of expression of phosphoprotein pMEK1 in said at least one sample from said patient correlates with a positive prognosis for PFS of said patient (i.e. longer PFS).

Preferably, the pMEK1 is used in combination with the pP70S6K for performing the method of prognosis according to the invention, i.e. the method of the invention consists of assaying at least one sample of the patient for the two markers, enabling an increased statistical relevance for predicting survival with respect PFS. Detection of the level of expression of pMEK1 and pP70S6K may be performed on the same sample or on two separate samples, preferably originating from a same tumor biopsy.

In one specific embodiment, the KRAS status of the patient was determined, the presence of muted KRAS gene being indicative that the said patient has a negative prognosis for survival.

KRAS mutation status can be determined on tumor samples, preferably before the treatment with an EGFR inhibitor, by an allelic discrimination assay or checked by direct sequencing of exon 2 as previously described (6, 9). It may be checked in particular that the KRAS gene in the tumor cells is wild-type on codon 12, 13 and 61.

Optionally, the BRAF status of the patient was also determined. BRAF status can be determined on tumor sample, preferably before the treatment with an EGFR inhibitor, by an allelic discrimination assay as described in (9) or by direct sequencing, for instance. It may be checked in particular that the BRAF gene in the tumor cells is wild-type on codon 600.

Therefore, the method of prognosis according to the invention is preferably carried out for patients whose tumor is (homozygous) wild-type for the KRAS gene and optionally (homozygous) wild-type for the BRAF gene.

### EGFR Inhibitors:

Preclinical and clinical studies have shown that targeting EGFR is a valid strategy for namely cancer therapy. Currently four treatment strategies for targeting EGFR and blocking its downstream signalling pathways have been developed, including 1) monoclonal antibodies directed against the extracellular domain of EGFR, 2) small molecules blocking tyrosine-kinase activation intracellularly (tyrosine-kinase inhibitors; TKIs), 3) antisense oligonucleotides inhibiting EGFR synthesis and 4) antibody-based immunoconjugates such as immunotoxins or immunoliposomes for specific and efficient delivery of anticancer agents to EGFR overexpressing tumors.

Most preferably, the EGFR inhibitor consists of an antibody specifically directed to EGFR.

Inhibitors of EGFR include, but are not limited to, tyrosine kinase inhibitors such as quinazolines, such as PID 153035, 4-(3-chloroanilino) quinazoline, CP-358,774, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d] pyrimidines, curcumin (diferuloyl methane), 4,5-bis (4-fluoroanilino) phthalimide; tyrphostins containing nitrothiophene moieties; the protein kinase inhibitor ZD-I 839, CP-358774; PD-OI 83805, EKB-569, HKI-272 and HKI-357; or tyrosine kinase inhibitors as described in International patent application WO05/018677; WO99/09016; WO98/43960; WO 98/14451; WO 98/02434; WO97/38983; WO99/06378; WO99/06396; WO96/30347; WO96/33978; WO96/33977; and WO96/33980, WO 95/19970; U.S. Pat. App. Nos. 2005/0101618, 2005/0101617, 20050090500; all herein incorporated by reference. Further useful EGFR inhibitors are described in U.S. Pat. App. No. 20040127470, particularly in tables 10, 11, and 12, and are herein incorporated by reference.

Other EGFR inhibitors include, but are not limited to, Gerainib available under the tradename IRESSA®; and Erlotinib available under the tradename TARCEVA®; the monoclonal antibodies cetuximab (ERBITUX®) and anti-EGFR 22Mab, or egf/r3 MAb, panitumumab/ABX-EGF, nimotuzumab (TheraCIM-hR3), EMD-700, EMD-7200, EMD-5590, E7.6.3, Mab 806, MDX-103, MDX-447/H-477, and the compounds ZD-1834, ZD-1838 and ZD-1839, PKI-166, PKI-166/CGP-75166, PTK 787 AEE788, CP 701, leflunomide, CM 033/PD-169414/PD-183805/Canertinib, CP-358774, PD-168393, PD-158780, PD-160678, CL-387,785 ((N-[4-[(3-bromophenyl)ammo]-6-quinazolinyl]-2-butynamide; BBR-1611, Naamidine A, RC-3940-II, BIBX-1382, OLX-103, VRCTC-310, EGF fusion toxin, DAB-389, ZM-252808, RG-50864, LFM-AI2, WHI-P97, GW-282974, GW2016, KT-8391 and EGFR Vaccine, EXEL 7647/EXEL 0999, XL647,′ AG1478 (4-(3-Chloroanillino)-6,7-dimethoxyquinazoline), AG879 (3,5-Di-t-butyl-4-hydroxy-benzylidene)thiocyanoacetamide), ICRI 5, ICRI 6, and ICR80, ICR62, CGP 59326A, BMS-599626. These and other EGFR-inhibiting agents can be used in the present invention.

In one embodiment, a treatment is administered that targets both EGFR and ErbB2, and/or IGF1 and IGF1 R. Some inhibitors of ErbB2 also inhibit EGFR and may be useful in the methods of the present invention. ErbB2 inhibitors include CI-1003, CP-724,714, CP-654577, GW-2016, GW-282974, and lapatinib/GW-572016, TAK-165, AEE788, EKB-569, HKI-272 and HKI-357, EXEL 7647/EXEL 0999 and the monoclonal antibodies Trastuzumab (tradename HERCEPTIN), 2C4, AR-209, pertuzumab (tradename OMNITARG), BMS-599626 and 2B-1. For example those indicated in U.S. Pat. Nos. 6,867,201, 6,541,481, 6,284,764, 5,587,458 and 5,877,305; WO 98/02434, WO 99/35146, WO 99/35132, WO 98/02437, WO 97/13760, WO 95/19970, which are all hereby incorporated herein in their entireties by reference. The ErbB2 receptor inhibitor compounds and substance described in the aforementioned PCT applications, U.S. patents, and U.S. patent applications, as well as other compounds and substances that inhibit the ErbB2 receptor, can be used in accordance with the present invention.

Increased levels of IGF1 have been associated with breast, prostate and colon cancer growth and metastases. Accordingly EGRF inhibitors may be administered simultaneously or separately in combination with IGF1 and/or IGF1 R inhibitors. Examples of IGF1 R inhibitors include BMS-7548077, OSI-906; Antagonists of IGF1 include octreotide acetate and peptides described in the international patent application WO 02/072780.

In another embodiment, compounds useful in the method of the present invention are antibodies which interfere with kinase signaling via EGFR, including monoclonal, chimeric, humanized, recombinant antibodies and fragment thereof which are characterized by their ability to inhibit the kinase activity of the EGFR and which have low toxicity.

Neutralizing antibodies are readily raised in animals such as rabbits or mice by immunization with an EGFR. Immunized mice are particularly useful for providing sources of B cells for the manufacture of hybridomas, which in turn are cultured to produce large quantities of anti-EGFR monoclonal antibodies. Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined. Humanized antibodies are immunoglobin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans. Examples of high affinity monoclonal antibodies and chimeric derivatives thereof, that are useful in the methods of the present invention, are described in the European Patent Application EP 186,833; PCT Patent Application WO 92/16553; and U.S. Pat. No. 6,090,923.

Thus, in connection with the administration of a EGFR inhibitor, a drug which is “effective against” a cancer indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

### Methods of Treatment

After being tested for responsiveness to a treatment with an EGFR inhibitor using the prediction method described herein, the patients that have been predicted to be good responders may be administered with an anti-EGFR inhibitor with a good expectation of success of the medical treatment.

Consequently another aspect of the invention consists of a method of treatment of a patient affected with cancer whose condition is likely to be improved by treatment with an EGFR inhibitor comprising the steps of:

a) assessing in a sample from said patient the level of expression of phosphoprotein pP70S6k;

b) comparing the level of expression of phosphoprotein pP70S6k in the sample of said patient to a reference level of expression of phosphoprotein pP70S6k,

wherein a low level of expression of phosphoprotein pP70S6k in the sample from said patient with respect to the standard level of expression of phosphoprotein pP70S6k correlates with a higher probability that said patient is responder to a treatment with an EGFR inhibitor, and

c) if the patient is determined as a responder to a treatment with an EGFR inhibitor, administering to said patient a therapeutically effective amount of an EGFR inhibitor.

The method is preferably carried out wherein the patient is affected with a cancer. The cancer is selected from breast cancer, bladder cancer, uterine/cervical cancer, oesophageal cancer, pancreatic cancer, colon cancer, colorectal cancer, kidney cancer, ovarian cancer, prostate cancer, head and neck cancer, non-small cell lung cancer and stomach cancer and in more preferable embodiment the patient suffers from colorectal cancer.

In certain embodiments the anti-EGFR therapy consists of an antibody specifically directed to EGFR

As KRAS and BRAF mutations in tumors have been associated with lack of response of the patient to a treatment with an EGFR inhibitor, if the status of the patient for KRAS and BRAF mutations is unknown, the patient's status for KRAS and optionally BRAF is preferably determined before administering the patient with an EGFR inhibitor.

KRAS and BRAF mutation status can be determined as explained above.

Therefore, the method of treatment according to the invention is preferably carried out for patients whose tumor is (homozygous) wild-type for the KRAS gene and optionally (homozygous) wild-type for the BRAF gene.

Details relating to the administration regimen of an EGFR inhibitor have been previously described in the present specification.

This invention is further illustrated in the example hereafter.

### EXAMPLE

### A. Materials and Methods

Patients

In the present study, 42 patients (24 males; mean age: 61.8 years) with histologically proven metastatic colorectal adenocarcinoma treated by cetuximab (n=41) or panitumumab (n=1) were analyzed. These 42 patients were those from a previous series of 114 patients (6) for whom protein material was available. As they were included in our previous study on KRAS mutation as predictor of response to cetuximab, all the patients were evaluable for tumor response. An analysis of EGFR expression was performed by IHC on at least one tumor fragment and was considered EGFR positive if at least 1% malignant cells stained (Zymed Laboratories Inc., San Francisco, Calif. or Dako Cytomation, Glostrup, Denmark). This retrospective study was performed according to the French ethics laws.

One patient received panitumumab in monotherapy and 41 patients received cetuximab monotherapy (n=2) or in combination with irinotecan (alone, n=37; FOLFIRI regimen, n=2) The anti-EGFR treatment was given as second-line, third-line, fourth-line, fifth-line or more in 16, 16, 4 and 3 cases respectively. One patient received cetuximab as first line without any chemotherapy. The median follow-up was 10.1 months.

Tumor response was evaluated by computerized tomodensitometry according to the RECIST (Response Evaluation Criteria in Solid Tumors) criteria (8). For the analysis, patients with complete and partial response (CR and PR) were classified as responders, and those with stable and progressive disease (SD and PD) as non-responders.

DNA Extraction and KRAS Mutation Analysis

DNA was extracted from tumor samples obtained from primary colorectal tumor or metastatic tissue as previously described (6). KRAS mutation status was determined on tumor samples before the cetuximab treatment by an allelic discrimination assay and checked by direct sequencing of exon 2 as previously described (6, 9).

Protein Extraction and EGFR Downstream Signaling Phosphoprotein Expression

Whole cell protein extraction was performed from tumor tissues using the Kit RIPA lysis Buffer 1× (Tebu-bio, Le Perray en Yvelines, France) prepared with inhibitors (PMSF, protease inhibitor cocktail and sodium orthonavate) (Dutscher, Issy-les-Moulineaux, France) according to the manufacturer's recommendations.

The expression of key phosphorylated proteins of the downstream EGFR signaling pathway (pMEK1, pERK1/2, pAKT, pP70S6k and pGSK313) was analyzed using phosphoprotein array (Bio-Plex®, Bio-Rad, Hercules Calif.) is based on multiplex sandwich bead immunoassays. Protein extracts were transferred into 96-well dishes and diluted with 25 μl buffered solution. Fluorescent capture beads coupled to antibodies directed against the phosphoproteins (phospho-AKT, phospho-GSK3, phospho-P70S6K, phospho-MEK1, phospho-RK1/2) were mixed, and added into each well and incubated overnight. Following incubation, the plates were washed and incubated with biotinylated antibodies fixing each target protein. Streptavidin-phycoerythrin solution was then added. The analysis consisted in a double laser fluorescence detection allowing simultaneous identification of the target protein through the red fluorescence emission signal of the bead and quantification of the target protein through the fluorescence intensity of phycoerythrin. Results were recorded as mean fluorescence intensities and compared to negative controls. Positive controls consisting of standard protein extracts from cell lines were added to each series. All results were normalized through the different batches of analyses by the same mutated tumor sample. The expression level of each phosphoprotein was given in an arbitrary unit

Statistical Analysis

Chi-square test was used to calculate the p value for association between KRAS mutation and response to cetuximab. Expression levels of each phosphoprotein were compared between group (i.e. KRAS mutated and non mutated tumors or between responders and non responder patients) by the Wilcoxon ranksum test. The PFS was calculated as the period from the first day of cetuximab treatment to the date of tumor progression, to death from any cause or to the date of the last follow-up at which data point was censored. The OS time was calculated as the period from the first day of cetuximab treatment until death of any cause or until the date of the last follow-up, at which data point was censored. We dichotomized each phosphoprotein expression, by choosing the threshold optimizing the logrank test for PFS and leaving at least 10 patients in both groups (low expression versus high expression). Taking into account the number of tests performed (23 thresholds tested per phosphoprotein), we retained a threshold of significance of 0.001 for the logrank test. Therefore with this threshold both PFS and OS were estimated by the Kaplan-Meier method and compared using the logrank test. A univariate survival analysis of these dichotomized variables was performed using a Cox model. A multivariate Cox model was used to estimate the effect of KRAS mutation and phosphoprotein expression. Analysis was carried out using the STATA software (College Station, Tex.). The level of significance was set at P=0.05.

### B. Results

Tumor Response and Survival According to KRAS Mutation Status

An objective response to cetuximab was obtained in 28.6% of the patients (CR: 1, PR: 11). A KRAS mutation was present in 45% of the tumors (n=19) and was significantly associated with the absence of response to cetuximab (p<0.001). In univariate analysis, PFS was longer when tumor was not mutated (median: 32 weeks, C195%[14.7-46] versus 8 weeks, C195%[6.1-9], log-rank P<10-4). The OS was also longer when the tumor was not mutated (median: 13.9 months, C195%[6.5-21.6] versus 6.4 months, C195%[2.8-10.1] logrank test p=0.02).

Phosphoprotein Expression and KRAS Mutation Status

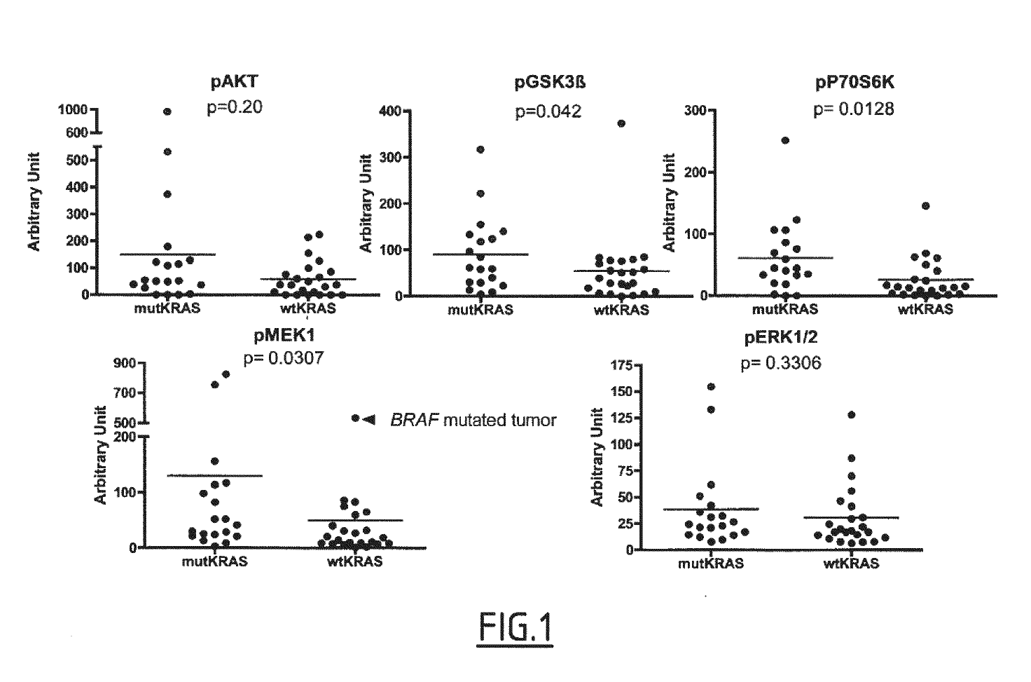

The median [range value] values of protein expression were 49.2 [0-963], 54.18 [0-373.7] 28.1 [1.3-826.3], 22 [6.7-154.8] and 25.7[0-251.9] for pAKT, pGSK3β, pMEK1, pERK1/2 and pP70S6k in arbitrary unit respectively. Phosphoprotein expression was evaluated according to KRAS mutation status. The expression of all the phosphorylated proteins was higher in KRAS mutated tumors compared to WT tumors, but the difference was statistically significant for pP70S6k, pGSK3β and pMEK1 only (FIG. 1).

Tumor Response and Survival According to Phosphoprotein Expression

A correlation between phosphoprotein expression and tumor response to cetuximab was found only for pP70S6k. The expression was significantly lower in responders compared to non-responder patients (20.5 versus 50 arbitrary units respectively; p=0.024) (FIG. 2).

In Cox univariate analysis, PFS was longer for patients with low expression of pAKT, pGSK3β, pMEK1, pERK1/2 and pP70S6k (Table 1). A cut-off value for dichotomizing each variable was determined in order to maximize the PFS difference between the 2 groups with a minimal size of ten patients in one group. The cut-off value was 37.9, 59.25, 79.3, 42, 16.3 for pAKT, pGSK3β, pMEK1, pERK1/2 and pP70S6k respectively. Considering the number of tests used for determining the optimal cut-off, we retained the threshold of significance at 0.001. Patients with a pMEK1 expression level higher than the cut-off value demonstrated a shorter PFS than those with a low expression level (median PFS: 7±0.1 weeks, C195% [4.4-12] versus 20±3.4 weeks, C195% [8.6-33]; p=0.0001). Patients with a pP70S6k expression level higher than the cut-off value demonstrated a shorter PFS than those with a low expression level (median PFS: 8±0.1 weeks, C195% [8-16] versus 33.1±0.04 weeks, C195% [14.8-58.1]; p=0.0006) (FIG. 3).

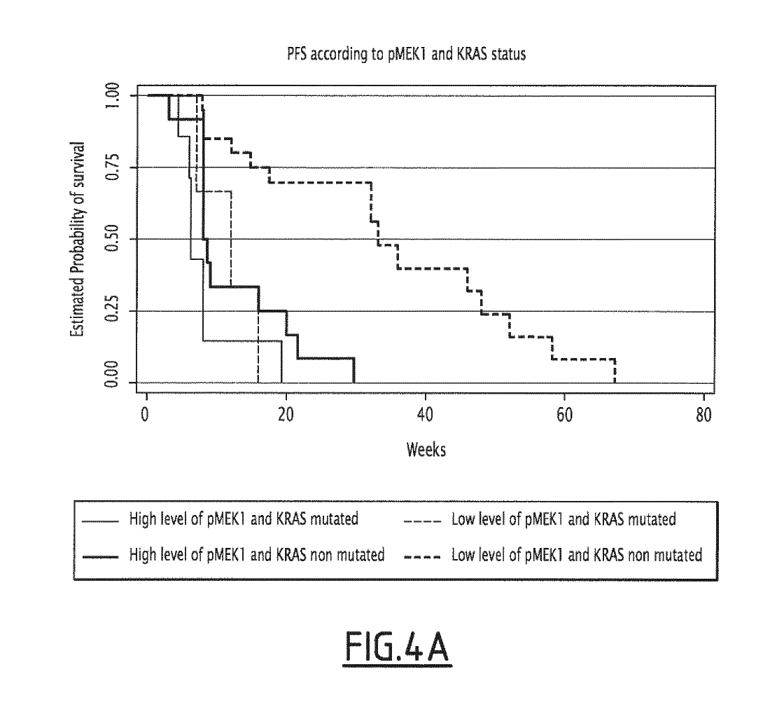

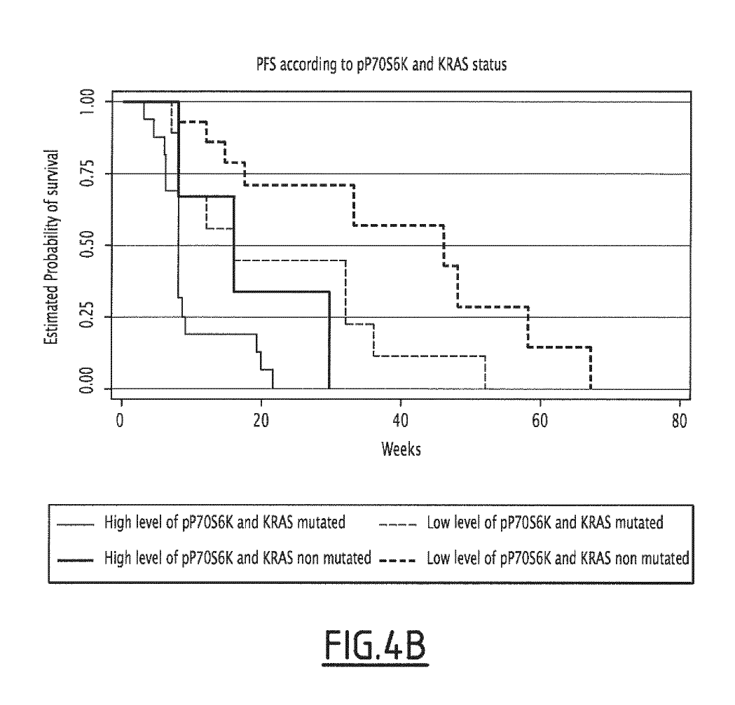

In Cox multivariate analysis the two models including KRAS and pMEK1 or KRAS and pP70S6k showed that the expression of these two phosphoproteins predicts PFS independently of KRAS status (Table 2). The inclusion of both pMEK1 and pP70S6k expression level did not significantly improve the model. Finally PFS was significantly associated with pMEK1 and with pP70S6k when stratified by KRAS status (p=0.002 and p=0.01 respectively) (FIG. 4). The expression level of pMEK1 and pP70S6k were investigated for association with the OS. In univariate analysis a significant association was only observed between pP70S6k and OS. Patients with low levels of pP70S6k were associated with better OS (median OS: 6.5±0.1 months, C195% [3.8-10.5] for the high expression level of pP70S6k versus 21.6 months, C195% [6.1-26.3] for the low expression level group of pP70S6k; p=0.003). The logrank stratified on KRAS status is still significant (p=0.01, FIG. 5).

TABLE 1
Univariate Cox analysis for PFS according
to phosphoproteins expression.

| Variable | HR ± Std. Err | P | 95% CI |
|---|---|---|---|
| pMEK1 | 1.003 ± 0.001 | 0.004 | 1.001-1.005 |
| pERK1-2 | 1.013 ± 0.005 | 0.01 | 1.003-1.005 |
| pAKT | 1.002 ± 0.001 | 0.03 | 1.000-1.004 |
| pGSK3β | 1.004 ± 0.002 | 0.03 | 1.000-1.008 |
| pP70S6k | 1.008 ± 0.003 | 0.01 | 1.002-1.013 |

TABLE 2
Multivariate analysis for PFS including KRAS
status and phosphoproteins expression.
First model including KRAS status and expression value of pP70S6k

| Variable | HR | P | 95% CI |
|---|---|---|---|
| KRAS | | | |
| Mutated | 1 | | |
| Non mutated | 0.27 | 0.003 | 0.11-0.63 |
| pP70S6k | | | |
| High | 1 | | |
| Low | 0.42 | 0.03 | 0.19-0.93 |

Log likelihood = −96.1, LR chi2(2 degree of freedom) = 20.80

Second model including KRAS status and expression value of pMEK1

| Variable | HR | P | 95% CI |
|---|---|---|---|
| KRAS | | | |
| Mutated | 1 | | |
| Non mutated | 0.24 | 0.001 | 0.1-0.56 |
| pPMEK1 | | | |
| High | 1 | | |
| Low | 0.34 | 0.01 | 0.15-0.78 |

Log likelihood = −95.6, LR chi2(2 degree of freedom) = 21.76

Third model including KRAS status
expression value of pMEK1 and pP70S6k

| Variable | HR | P | 95% CI |
|---|---|---|---|
| KRAS | | | |
| Mutated | 1 | | |
| Non mutated | 0.28 | 0.005 | 0.2-0.68 |
| pPMEK1 | | | |
| High | 1 | | |
| Low | 0.43 | 0.055 | 0.2-1.01 |
| pP70S6k | | | |
| High | 1 | | |
| Low | 0.51 | 0.11 | 0.21-1.18 |

Log likelihood = −94.34, LR chi2(2 degree of freedom) = 24.30

In conclusion, this invention demonstrates the importance of measuring EGFR downstream signaling phosphosproteins expression in addition to KRAS mutation analysis for the prediction of response to cetuximab and the survival of CRC patients. The expression of these phosphoproteins allows a global analysis of signaling pathways functionality, and is likely to offer great insight than single gene analysis for understanding of molecular mechanisms underlying resistance to anti-EGFR therapies. Such an approach offers valuable additional predictive information to the KRAS status of tumor cells.

### REFERENCES

1. Benvenuti, S., Sartore-Bianchi, A., Di Nicolantonio, F., Zanon, C., Moroni, M., Veronese, S., Siena, S., and Bardelli, A. Oncogenic activation of the RAS/RAF signaling pathway impairs the response of metastatic colorectal cancers to anti-epidermal growth factor receptor antibody therapies. Cancer Res, 67: 2643-2648, 2007.
2. De Roock, W., Piessevaux, H., De Schutter, J., Janssens, M., De Hertogh, G., Personeni, N., Biesmans, B., Van Laethem, J. L., Peeters, M., Humblet, Y., Van Cutsem, E., and Tejpar, S. KRAS wild-type state predicts survival and is associated to early radiological response in metastatic colorectal cancer treated with cetuximab. Ann Oncol, 2007.
3. Di Fiore, F., Blanchard, F., Charbonnier, F., Le Pessot, F., Lamy, A., Galais, M. P., Bastit, L., Killian, A., Sesboue, R., Tuech, J. J., Queuniet, A. M., Paillot, B., Sabourin, J. C., Michot, F., Michel, P., and Frebourg, T. Clinical relevance of KRAS mutation detection in metastatic colorectal cancer treated by Cetuximab plus chemotherapy. Br J Cancer, 96: 1166-1169, 2007.
4. Frattini, M., Saletti, P., Romagnani, E., Martin, V., Molinari, F., Ghisletta, M., Camponovo, A., Etienne, L. L., Cavalli, F., and Mazzucchelli, L. PTEN loss of expression predicts cetuximab efficacy in metastatic colorectal cancer patients. Br J Cancer, 97: 1139-1145, 2007.
5. Khambata-Ford, S., Garrett, C. R., Meropol, N. J., Basik, M., Harbison, C. T., Wu, S., Wong, T. W., Huang, X., Takimoto, C. H., Godwin, A. K., Tan, B. R., Krishnamurthi, S. S., Burris, H. A., 3rd, Poplin, E. A., Hidalgo, M., Baselga, J., Clark, E. A., and Mauro, D. J. Expression of epiregulin and amphiregulin and K-ras mutation status predict disease control in metastatic colorectal cancer patients treated with cetuximab. J Clin Oncol, 25: 3230-3237, 2007.
6. Lievre, A., Bachet, J. B., Boige, V., Cayre, A., Le Corre, D., Buc, E., Ychou, M., Bouche, O, Landi, B., Louvet, C., Andre, T., Bibeau, F., Diebold, M. D., Rougier, P., Ducreux, M., Tomasic, G., Emile, J. F., Penault-Llorca, F., and Laurent-Puig, P. KRAS mutations as an independent prognostic factor in patients with advanced colorectal cancer treated with cetuximab. J Clin Oncol, 26: 374-379, 2008.
7. Amado, R. G., Wolf, M., Peeters, M., Van Cutsem, E., Siena, S., Freeman, D. J., Juan, T., Sikorski, R., Suggs, S., Radinsky, R., Patterson, S. D., and Chang, D. D. Wild-Type KRAS Is Required for Panitumumab Efficacy in Patients With Metastatic Colorectal Cancer. J Clin Oncol, 2008
8. Therasse, P., Arbuck, S. G., Eisenhauer, E. A., Wanders, J., Kaplan, R. S., Rubinstein, L., Verweij, J., Van Glabbeke, M., van Oosterom, A. T., Christian, M. C., and Gwyther, S. G. New guidelines to evaluate the response to treatment in solid tumors. European Organization for Research and Treatment of Cancer, National Cancer Institute of the United States, National Cancer Institute of Canada. J Natl Cancer Inst, 92: 205-216, 2000.
9. Lievre, A., Bachet, J. B., Le Corre, D., Boige, V., Landi, B., Emile, J. F., Cote, J. F., Tomasic, G., Penna, C., Ducreux, M., Rougier, P., Penault-Llorca, F., and Laurent-Puig, P. KRAS mutation status is predictive of response to cetuximab therapy in colorectal cancer. Cancer Res, 66: 3992-3995, 2006.
10. Eisenhauer E A, Therasse P, Bogaerts J, Schwartz L H, Sargent D, Ford R, Dancey J, Arbuck S, Gwyther S, Mooney M, Rubinstein L, Shankar L, Dodd L, Kaplan R, Lacombe D, Verweij J. New response evaluation criteria in solid tumors: revised RECIST guideline (version 1.1). Eur J. Cancer. 2009 January; 45(2):228-47.

1. A method of predicting the response of a patient affected with cancer to a medical treatment with an EGFR inhibitor which comprises:
a) assessing in a sample from said patient the level of expression of phosphoprotein pP70S6k;
b) comparing the level of expression of phosphoprotein pP70S6k in the sample from said patient to a reference level of expression of phosphoprotein pP70S6k,
wherein a low level of expression of phosphoprotein pP70S6k in the sample from said patient with respect to reference level of expression of phosphoprotein pP70S6k correlates with a higher probability that said patient is responder to a medical treatment with an EGFR inhibitor.

2. The method according to claim 1 wherein the patient is affected with a cancer selected from the group consisting of breast cancer, bladder cancer, uterine/cervical cancer, oesophageal cancer, pancreatic cancer, colon cancer, colorectal cancer, kidney cancer, ovarian cancer, prostate cancer, head and neck cancer, non-small cell lung cancer, and stomach cancer.

3. The method according to claim 1 wherein the patient is affected with colorectal cancer.

4. The method according to claim 1 wherein the EGFR inhibitor consists of an antibody specifically directed to EGFR.

5. The method according to claim 1 wherein the sample from said patient consists of a tumor sample.

6. The method according to claim 5 wherein said patient's tumor is wild-type for the KRAS gene and optionally wild-type for the BRAF gene.

7. A method of treatment of patient affected with cancer whose condition is likely to be improved by treatment with an EGFR inhibitor, which method comprises the steps of:
a) assessing in a sample from said patient the level of expression of phosphoprotein pP70S6k;
b) comparing the level of expression of phosphoprotein pP70S6k in the sample of said patient to a reference level of expression of phosphoprotein pP70S6k,
wherein a low level of expression of phosphoprotein pP70S6k in the sample from said patient with respect to the standard level of expression of phosphoprotein pP70S6k correlates with a higher probability that said patient is responder to a treatment with an EGFR inhibitor, and
c) if the patient is determined as a responder to a treatment with an EGFR inhibitor, administering to said patient a therapeutically effective amount of an EGFR inhibitor.

8. The method according to claim 7 wherein the patient is affected with a cancer selected from the group consisting of breast cancer, bladder cancer, uterine/cervical cancer, oesophageal cancer, pancreatic cancer, colon cancer, colorectal cancer, kidney cancer, ovarian cancer, prostate cancer, head and neck cancer, non-small cell lung cancer and stomach cancer.

9. The method according to claim 7 wherein the patient is affected with colorectal cancer.

10. The method according to claim 7 wherein the EGFR inhibitor consists of an antibody specifically directed to EGFR.

11. The method according to claim 7 wherein said patient's tumor is wild-type for the KRAS gene and optionally wild-type for the BRAF gene.

12. A method for determining a prognosis of cancer in a patient, the method comprising:
a) assessing in at least one sample from said patient the level of expression of phosphoprotein pMEK1 and/or phosphoprotein pP70S6k; and
b) comparing the level of expression of phosphoprotein pMEK1 and/or phosphoprotein pP70S6k in said at least one sample from said patient to a reference level of expression of phosphoprotein pMEK1 and/or phosphoprotein pP70S6k, respectively,
wherein a low level of expression of phosphoprotein of pMEK1 and/or phosphoprotein pP70S6k in said at least one sample from said patient correlates with a positive prognosis of cancer in said patient.

13. The method according to claim 12 wherein the patient is affected with a cancer selected from the group consisting of breast cancer, bladder cancer, uterine/cervical cancer, oesophageal cancer, pancreatic cancer, colon cancer, colorectal cancer, kidney cancer, ovarian cancer, prostate cancer, head and neck cancer, non-small cell lung cancer, and stomach cancer.

14. The method according to claim 12 wherein the patient is affected with colorectal cancer.

15. The method according to claim 12 wherein the sample from patient consists of a tumor sample.

16. The method according to claim 15 wherein the patient's tumor is wild-type for the KRAS gene and optionally wild-type for the BRAF gene.
